SEQUENCE LISTING (416 sequences)

SEQ ID NO: 1 - DNA, 44 bp, synthetic construct
  misc_feature 1..44: note = primer
  source 1..44: mol_type = other DNA
aggtcgactctagaggatccccacaccgatggggccgtggtgga

SEQ ID NO: 2 - DNA, 47 bp, synthetic construct
  misc_feature 1..47: note = Primer
  source 1..47: mol_type = other DNA
aggtcgactctagaggatccccagacagatggctgagcatcactccg

SEQ ID NO: 3 - DNA, 44 bp, synthetic construct
  misc_feature 1..44: note = Primer
  source 1..44: mol_type = other DNA
aggtcgactctagaggatccccgagggggccgacttgggcagac

SEQ ID NO: 4 - DNA, 43 bp, synthetic construct
  misc_feature 1..43: note = Primer
  source 1..43: mol_type = other DNA
acgttgtaaaacgacggccagtgaagacgtgcagctggtggag

SEQ ID NO: 5 - DNA, 43 bp, synthetic construct
  misc_feature 1..43: note = Primer
  source 1..43: mol_type = other DNA
acgttgtaaaacgacggccagtgaagacgwgcagctgrtggag

SEQ ID NO: 6 - DNA, 43 bp, synthetic construct
  misc_feature 1..43: note = Primer
  source 1..43: mol_type = other DNA
acgttgtaaaacgacggccagtgaagacgtgctggtggtggag

SEQ ID NO: 7 - DNA, 43 bp, synthetic construct
  misc_feature 1..43: note = Primer
  source 1..43: mol_type = other DNA
acgttgtaaaacgacggccagtgaagacgtgcgattggtggag

SEQ ID NO: 8 - DNA, 43 bp, synthetic construct
  misc_feature 1..43: note = Primer
  source 1..43: mol_type = other DNA
acgttgtaaaacgacggccagtgaagacgtgcaactggtggak

SEQ ID NO: 9 - DNA, 43 bp, synthetic construct
  misc_feature 1..43: note = Primer
  source 1..43: mol_type = other DNA
acgttgtaaaacgacggccagtgaagacgtgcaattggtggag

SEQ ID NO: 10 - DNA, 43 bp, synthetic construct
  misc_feature 1..43: note = Primer
  source 1..43: mol_type = other DNA
acgttgtaaaacgacggccagtgaagatgtgcasstggtggaa

SEQ ID NO: 11 - DNA, 43 bp, synthetic construct
  misc_feature 1..43: note = Primer
  source 1..43: mol_type = other DNA
acgttgtaaaacgacggccagtgaacaggttytgctggtgcag

SEQ ID NO: 12 - DNA, 43 bp, synthetic construct
  misc_feature 1..43: note = Primer
  source 1..43: mol_type = other DNA
acgttgtaaaacgacggccagtgaagawgtgcagytsgtggag

SEQ ID NO: 13 - DNA, 43 bp, synthetic construct
  misc_feature 1..43: note = Primer
  source 1..43: mol_type = other DNA
acgttgtaaaacgacggccagtgaagatgtgcarstggtggag

SEQ ID NO: 14 - DNA, 46 bp, synthetic construct
  misc_feature 1..46: note = Primer
  source 1..46: mol_type = other DNA
acgttgtaaaacgacggccagtgaacagtctgtgctgactcagccg

SEQ ID NO: 15 - DNA, 47 bp, synthetic construct
  misc_feature 1..47: note = Primer
  source 1..47: mol_type = other DNA
acgttgtaaaacgacggccagtgaacagtctgtggtgactcagccgc

SEQ ID NO: 16 - DNA, 49 bp, synthetic construct
  misc_feature 1..49: note = Primer
  source 1..49: mol_type = other DNA
acgttgtaaaacgacggccagtgaacagcctgtgctgactcagccaccg

SEQ ID NO: 17 - DNA, 48 bp, synthetic construct
  misc_feature 1..48: note = Primer
  source 1..48: mol_type = other DNA
acgttgtaaaacgacggccagtgaacaggaagtagtgactcaggaaac

SEQ ID NO: 18 - DNA, 48 bp, synthetic construct
  misc_feature 1..48: note = Primer
  source 1..48: mol_type = other DNA
acgttgtaaaacgacggccagtgaacagtcggggccgaatcaaccatc

SEQ ID NO: 19 - DNA, 49 bp, synthetic construct
  misc_feature 1..49: note = Primer
  source 1..49: mol_type = other DNA
acgttgtaaaacgacggccagtgaacagtcggggctgactcaaccatcc

SEQ ID NO: 20 - DNA, 47 bp, synthetic construct
  misc_feature 1..47: note = Primer
  source 1..47: mol_type = other DNA
acgttgtaaaacgacggccagtgaatcctatgtattgactcagcccc

SEQ ID NO: 21 - DNA, 47 bp, synthetic construct
  misc_feature 1..47: note = Primer
  source 1..47: mol_type = other DNA
acgttgtaaaacgacggccagtgaatcctatgtactctctcagcccc

SEQ ID NO: 22 - DNA, 48 bp, synthetic construct
  misc_feature 1..48: note = Primer
  source 1..48: mol_type = other DNA
acgttgtaaaacgacggccagtgaacagcctgtggtgactcagccagc

SEQ ID NO: 23 - DNA, 48 bp, synthetic construct
  misc_feature 1..48: note = Primer
  source 1..48: mol_type = other DNA
acgttgtaaaacgacggccagtgaagatattgtgatgacgcagacccc

SEQ ID NO: 24 - DNA, 48 bp, synthetic construct
  misc_feature 1..48: note = Primer
  source 1..48: mol_type = other DNA
acgttgtaaaacgacggccagtgaagatcttgtcttgacacagacccc

SEQ ID NO: 25 - DNA, 46 bp, synthetic construct
  misc_feature 1..46: note = Primer
  source 1..46: mol_type = other DNA
acgttgtaaaacgacggccagtgaagaggccgtgatgacgcagacc

SEQ ID NO: 26 - DNA, 47 bp, synthetic construct
  misc_feature 1..47: note = Primer
  source 1..47: mol_type = other DNA
acgttgtaaaacgacggccagtgaagaggttgtgatgatamagaccc

SEQ ID NO: 27 - DNA, 48 bp, synthetic construct
  misc_feature 1..48: note = Primer
  source 1..48: mol_type = other DNA
acgttgtaaaacgacggccagtgaagcgatcacgatgacgcagtctcc

SEQ ID NO: 28 - DNA, 47 bp, synthetic construct
  misc_feature 1..47: note = Primer
  source 1..47: mol_type = other DNA
acgttgtaaaacgacggccagtgaagatgtcgtgatgacgcagaccc

SEQ ID NO: 29 - DNA, 57 bp, synthetic construct
  misc_feature 1..57: note = Primer
  source 1..57: mol_type = other DNA
acactctttccctacacgacgctcttccgatctggttttcccagtcacgacgttgta

SEQ ID NO: 30 - DNA, 58 bp, synthetic construct
  misc_feature 1..58: note = Primer
  source 1..58: mol_type = other DNA
gactggagttcagacgtgtgctcttccgatcttatgaccatgattacgccaagcttgc

SEQ ID NO: 31 - DNA, 46 bp, synthetic construct
  misc_feature 1..46: note = Primer
  source 1..46: mol_type = other DNA
acaaagacgatgacgacaagctcgagcaggaagtagtgactcagga

SEQ ID NO: 32 - DNA, 46 bp, synthetic construct
  misc_feature 1..46: note = Primer
  source 1..46: mol_type = other DNA
acaaagacgatgacgacaagctcgagcagtcggggccgaatcaacc

SEQ ID NO: 33 - DNA, 46 bp, synthetic construct
  misc_feature 1..46: note = Primer
  source 1..46: mol_type = other DNA
acaaagacgatgacgacaagctcgagtcctatgtattgactcagcc

SEQ ID NO: 34 - DNA, 46 bp, synthetic construct
  misc_feature 1..46: note = Primer
  source 1..46: mol_type = other DNA
acaaagacgatgacgacaagctcgagcagtcggggctgactcaacc

SEQ ID NO: 35 - DNA, 46 bp, synthetic construct
  misc_feature 1..46: note = Primer
  source 1..46: mol_type = other DNA
acaaagacgatgacgacaagctcgagcagtctgtggtgactcagcc

SEQ ID NO: 36 - DNA, 46 bp, synthetic construct
  misc_feature 1..46: note = Primer
  source 1..46: mol_type = other DNA
acaaagacgatgacgacaagctcgagtcctatgtactctctcagcc

SEQ ID NO: 37 - DNA, 46 bp, synthetic construct
  misc_feature 1..46: note = Primer
  source 1..46: mol_type = other DNA
acaaagacgatgacgacaagctcgagcagcctgtgctgactcagcc

SEQ ID NO: 38 - DNA, 46 bp, synthetic construct
  misc_feature 1..46: note = Primer
  source 1..46: mol_type = other DNA
acaaagacgatgacgacaagctcgagcagcctgtggtgactcagcc

SEQ ID NO: 39 - DNA, 46 bp, synthetic construct
  misc_feature 1..46: note = primer
  source 1..46: mol_type = other DNA
acaaagacgatgacgacaagctcgagcagtctgtgctgactcagcc

SEQ ID NO: 40 - DNA, 46 bp, synthetic construct
  misc_feature 1..46: note = primer
  source 1..46: mol_type = other DNA
acaaagacgatgacgacaagctcgagttctatgtattgactcagcc

SEQ ID NO: 41 - DNA, 41 bp, synthetic construct
  misc_feature 1..41: note = primer
  source 1..41: mol_type = other DNA
ctagaacggctagaaccgccgaggacggtcagatgggtccc

SEQ ID NO: 42 - DNA, 41 bp, synthetic construct
  misc_feature 1..41: note = primer
  source 1..41: mol_type = other DNA
ctagaacggctagaaccgccgaggacagtcagatgggtccc

SEQ ID NO: 43 - DNA, 41 bp, synthetic construct
  misc_feature 1..41: note = primer
  source 1..41: mol_type = other DNA
ctagaacggctagaaccgcctaggacgctcagatgggtacc

SEQ ID NO: 44 - DNA, 41 bp, synthetic construct
  misc_feature 1..41: note = primer
  source 1..41: mol_type = other DNA
ctagaacggctagaaccgccgaggacggtcagacgggtccc

SEQ ID NO: 45 - DNA, 41 bp, synthetic construct
  misc_feature 1..41: note = primer
  source 1..41: mol_type = other DNA
ctagaacggctagaaccgccgaggacggtcacatgggtccc

SEQ ID NO: 46 - DNA, 41 bp, synthetic construct
  misc_feature 1..41: note = primer
  source 1..41: mol_type = other DNA
ctagaacggctagaaccgccgaggacggtcaaatgggtccc

SEQ ID NO: 47 - DNA, 41 bp, synthetic construct
  misc_feature 1..41: note = primer
  source 1..41: mol_type = other DNA
ctagaacggctagaaccgccgaggacggtcagatgggtacc

SEQ ID NO: 48 - DNA, 41 bp, synthetic construct
  misc_feature 1..41: note = primer
  source 1..41: mol_type = other DNA
ctagaacggctagaaccgccgaggacgctcagatgggtacc

SEQ ID NO: 49 - DNA, 41 bp, synthetic construct
  misc_feature 1..41: note = primer
  source 1..41: mol_type = other DNA
ctagaacggctagaaccgcccaggacgctcagatgggtacc

SEQ ID NO: 50 - DNA, 41 bp, synthetic construct
  misc_feature 1..41: note = primer
  source 1..41: mol_type = other DNA
ctagaacggctagaaccgccgaggacggtcaggtgggtccc

SEQ ID NO: 51 - DNA, 44 bp, synthetic construct
  misc_feature 1..44: note = primer
  source 1..44: mol_type = other DNA
gcggttctggcggtggtggtcaggttttgctggtgcagtctggg

SEQ ID NO: 52 - DNA, 44 bp, synthetic construct
  misc_feature 1..44: note = primer
  source 1..44: mol_type = other DNA
gcggttctggcggtggtggtgacgtgcaactggtggagtctggg

SEQ ID NO: 53 - DNA, 44 bp, synthetic construct
  misc_feature 1..44: note = primer
  source 1..44: mol_type = other DNA
gcggttctggcggtggtggtgacgtgcagctggtggagtctggg

SEQ ID NO: 54 - DNA, 44 bp, synthetic construct
  misc_feature 1..44: note = primer
  source 1..44: mol_type = other DNA
gcggttctggcggtggtggtgacgtgctggtggtggagtctggg

SEQ ID NO: 55 - DNA, 44 bp, synthetic construct
  misc_feature 1..44: note = primer
  source 1..44: mol_type = other DNA
gcggttctggcggtggtggtgacgtgcgattggtggagtctggg

SEQ ID NO: 56 - DNA, 44 bp, synthetic construct
  misc_feature 1..44: note = primer
  source 1..44: mol_type = other DNA
gcggttctggcggtggtggtgacgtgcaattggtggagtctggg

SEQ ID NO: 57 - DNA, 44 bp, synthetic construct
  misc_feature 1..44: note = primer
  source 1..44: mol_type = other DNA
gcggttctggcggtggtggtcaggttctgctggtgcagtctggg

SEQ ID NO: 58 - DNA, 44 bp, synthetic construct
  misc_feature 1..44: note = primer
  source 1..44: mol_type = other DNA
gcggttctggcggtggtggtgatgtgcaggtggtggagtctggg

SEQ ID NO: 59 - DNA, 44 bp, synthetic construct
  misc_feature 1..44: note = primer
  source 1..44: mol_type = other DNA
gcggttctggcggtggtggtgatgtgcagctggtggagtctggg

SEQ ID NO: 60 - DNA, 44 bp, synthetic construct
  misc_feature 1..44: note = primer
  source 1..44: mol_type = other DNA
gcggttctggcggtggtggtgacgagcagctggtggagtctggg

SEQ ID NO: 61 - DNA, 44 bp, synthetic construct
  misc_feature 1..44: note = primer
  source 1..44: mol_type = other DNA
gcggttctggcggtggtggttacgtgcaactggtggagtctggg

SEQ ID NO: 62 - DNA, 44 bp, synthetic construct
  misc_feature 1..44: note = primer
  source 1..44: mol_type = other DNA
gcggttctggcggtggtggtgaagtgcagttggtggagtctggg

SEQ ID NO: 63 - DNA, 47 bp, synthetic construct
  misc_feature 1..47: note = primer
  source 1..47: mol_type = other DNA
tcacgaatatctttgctaccgaattctgaggacaccgtcaccagggc

SEQ ID NO: 64 - DNA, 47 bp, synthetic construct
  misc_feature 1..47: note = primer
  source 1..47: mol_type = other DNA
tcacgaatatctttgctaccgaattctgaggacactgtgactatggt

SEQ ID NO: 65 - DNA, 47 bp, synthetic construct
  misc_feature 1..47: note = primer
  source 1..47: mol_type = other DNA
tcacgaatatctttgctaccgaattctgaggacaccgtcaccaggac

SEQ ID NO: 66 - DNA, 47 bp, synthetic construct
  misc_feature 1..47: note = primer
  source 1..47: mol_type = other DNA
tcacgaatatctttgctaccgaattctgaagacacggtgaccagggt

SEQ ID NO: 67 - DNA, 47 bp, synthetic construct
  misc_feature 1..47: note = primer
  source 1..47: mol_type = other DNA
tcacgaatatctttgctaccgaattctgaggacaccgtcaccagggt

SEQ ID NO: 68 - DNA, 47 bp, synthetic construct
  misc_feature 1..47: note = primer
  source 1..47: mol_type = other DNA
tcacgaatatctttgctaccgaattcttgggagacggtgacctgggt

SEQ ID NO: 69 - DNA, 47 bp, synthetic construct
  misc_feature 1..47: note = primer
  source 1..47: mol_type = other DNA
tcacgaatatctttgctaccgaattctgaggagacggtgaccagggt

SEQ ID NO: 70 - DNA, 47 bp, synthetic construct
  misc_feature 1..47: note = primer
  source 1..47: mol_type = other DNA
tcacgaatatctttgctaccgaattccgaggacaccgtcaccagggc

SEQ ID NO: 71 - DNA, 47 bp, synthetic construct
  misc_feature 1..47: note = primer
  source 1..47: mol_type = other DNA
tcacgaatatctttgctaccgaattcggaggacaccgtcaccagggc

SEQ ID NO: 72 - DNA, 47 bp, synthetic construct
  misc_feature 1..47: note = primer
  source 1..47: mol_type = other DNA
tcacgaatatctttgctaccgaattctgaggacaccgtcaccggggc

SEQ ID NO: 73 - protein, 84 aa, Felis catus
  REGION 1..84: note = feline_VL_FW1-3_B5
QSGPNQPSSVSGTLGQTITISCTGVSVFVDWYQQIPGMAPKTVIYRNTNRLSEVPDRFSGSKSGITATLTITGLQAEDEADYYC

SEQ ID NO: 74 - protein, 89 aa, Felis catus
  REGION 1..89: note = feline_VL_FW1-3_A12
QPVVTQPPSVSGALGQTVTISCAGSANNNGIVGVNWYQQLPGKAPQLLIYANNRRPSSVPDRFSGSKSGNTGSLTITGLQAEDEADYYC

SEQ ID NO: 75 - protein, 89 aa, Felis catus
  REGION 1..89: note = feline_VL_FW1-3_D8
QSVLTQPPSVSGALGQTVTISCAGSGSNIGIVDVNWYQQLPGKAPSLLIYASDRRPSGVPERISGSRSGNTGSLTITGLQTEDEADYYC

SEQ ID NO: 76 - protein, 89 aa, Felis catus
  REGION 1..89: note = feline_VL_FW1-3_B8
QSVLTQPPSMSGALGQTVTISCAGSGSNIGVAGVNWYQQLPGKAPKALIYASTRRPSGVPERLSGSKSGNTGSLTITGLQPEDEADYYC

SEQ ID NO: 77 - protein, 90 aa, Felis catus
  REGION 1..90: note = feline_VL_FW1-3_C12
QSVVTQPPSVSGTPGQRITISCTGSSSNIGGGAAVSWYQQVPGMGPKSVIFWNNSKPSGVPDRFSGSKSGSSGTLTIIGLQAEDEADYYC

SEQ ID NO: 78 - protein, 84 aa, Felis catus
  REGION 1..84: note = feline_VL_FW1-3_B6
QSGPNQPSSVSGALGQRVTISCTGIDTYVGWFQQIPGMAPKTIIVGNTNRPSGVPDRFSGSKSGNTGTLTITGLQAEDEADYYC

SEQ ID NO: 79 - protein, 84 aa, Felis catus
  REGION 1..84: note = feline_VL_FW1-3_E3
QSGPNQPSSVSGALGQRVTISCTGLDSFVDWYQQMPGMAPKTIIYGDKYRPSGVPDRFSGSKSGSTGTLTITGLQADDEADYYC

SEQ ID NO: 80 - protein, 89 aa, Felis catus
  REGION 1..89: note = feline_VL_FW1-3_D1
QSGLTQPSSVSGALGQTVTISCAGSANNIGAVGVNWYQQLPGRAPKLLIYANNRRPSSVPDRFSGSKSGNTGSLTITGLQAEDEADYYC

SEQ ID NO: 81 - protein, 90 aa, Felis catus
  REGION 1..90: note = feline_VL_FW1-3_B9
QSVVTQPPSVSGALGQRVTMSCTGSSSNIGRGVYVSWYQQLSGTAPKLLIYGNTNRPSGVPDRFSGSKSGSTGSLTITGLQDEDEADYYC

SEQ ID NO: 82 - protein, 90 aa, Felis catus
  REGION 1..90: note = feline_VL_FW1-3_D3
QSVLTQPPSVSGALGQRVTISCTGISSNIGRGNYVSWYQQIPGIAPKTIIYVDNNRPSGVPDRFSGSKSGSTATLTITGLQAEDEADYYC

SEQ ID NO: 83 - protein, 87 aa, Felis catus
  REGION 1..87: note = feline_VL_FW1-3_G11
QPVLTQPPSVSVSLGQTARTTCGGNNIESKSVHWYQQRSGQTPVLIIYTDRNRPSGIPDRFSGTNSGNTATLTISGAQAEDEADYYC

SEQ ID NO: 84 - protein, 84 aa, Felis catus
  REGION 1..84: note = feline_VL_FW1-3_F9
QSGPNQPSSVSGALGQRVTISCTGVDTYVDWYQQIPGMAPKTIIYGNTNRPSGVPDRFSGSKSGSTGTLAITGLQAEDEGDYYC

SEQ ID NO: 85 - protein, 84 aa, Felis catus
  REGION 1..84: note = feline_VL_FW1-3_B11
QSGPNQPSSVSGALGQRVTISCSGAGSYVAWYQQIPGMAPKTIIYDNSNRPSGVPDRFSGSKSGNTGTLTITGLQAEDEADYYC

SEQ ID NO: 86 - protein, 84 aa, Felis catus
  REGION 1..84: note = feline_VL_FW1-3_E11
QSGPNQPSSVSAALGQRVTISCTGAGNYVGWYQQIPGMAPKTIIYGNSYRPSGVPDRFSGSKSGSTGTLTITGLQAEDEADYYC

SEQ ID NO: 87 - protein, 84 aa, Felis catus
  REGION 1..84: note = feline_VL_FW1-3_B1
QSGPNQPSSVSGALGQRVTISCTGAGSNVAWYQQIPGMAPKTIMYFNRNRPSGVPDRFSGSKSGDVGTLTITGLQAEDEADYFC

SEQ ID NO: 88 - protein, 96 aa, Felis catus
  REGION 1..96: note = feline_VL_FW1-3_A8
QPVLTQPSSLSASPGTTARLTCTLSSGFNVGGYYISWFQQKPGSPPRYLLYYYSDSDKHQGPGVPSRFSGSKDASANAGLLLVSGPQPEDEADYYC

SEQ ID NO: 89 - protein, 89 aa, Felis catus
  REGION 1..89: note = feline_VL_FW1-3_D2
QPVLTQPPSVSGALGQTVTISCAGSANNIGIVGVNWYQQLPGKAPKLLIYANNRRPSSVPDRFSGSKSGNTGSLTITGLQAEDEADFYC

SEQ ID NO: 90 - protein, 90 aa, Felis catus
  REGION 1..90: note = feline_VL_FW1-3_B7
QSVVTQPPSVSGTLGQRITISCTGGSSNIGGGEAVNWYQQVPGMGPKTVIYWSDNKPSGVPDRFSGSKSGSSGTLTITGLQAEDEADYYC

SEQ ID NO: 91 - protein, 90 aa, Felis catus
  REGION 1..90: note = feline_VL_FW1-3_C10
QSVVTQPPSVSGALGQRVTISCTGSSSNVGRGNYVSWYQQLSGTPPKLLIYGNSDRLSGVPDRFSGSKSGTTGSLTITGLQTEDEADYYC

SEQ ID NO: 92 - protein, 84 aa, Felis catus
  REGION 1..84: note = feline_VL_FW1-3_D7
QSGPNQPSSVSGALGQRVTISCTGAGSYVGWYQQVPGMAPKTIIYDDNKRPSGVPARFSGSKSGSTGTLTITGLQAEDEANYYC

SEQ ID NO: 93 - protein, 90 aa, Felis catus
  REGION 1..90: note = feline_VL_FW1-3_B10
QSVLTQPPSVSGSLGQRVTISCTGSTSNIGGGNYVTWYQQVPGTAPRLLIYEDDKRASGVPGRFSGSKSGSSGSLTITGLQADDEADYYC

SEQ ID NO: 94 - protein, 90 aa, Felis catus
  REGION 1..90: note = feline_VL_FW1-3_A7
QPVLTQPPSVSGSPGQRVTISCSGGSSNIGGGNYVSWYRQVLGSAPRLLIYENYRRPSGVPDRFSGSKSGNSGSLTITGLQADDEADYYC

SEQ ID NO: 95 - protein, 90 aa, Felis catus
  REGION 1..90: note = >SEQ95_>feline_VL_FW1-3_H6
QPVLTQPPSVSGALGQRVTISCTGSSPNIGRGNYVSWYQQLSGTAPKVLIYGNTNRPSGVPDRFSGSKSGSTGSLTITGLQAEDEADYYC

SEQ ID NO: 96 - protein, 89 aa, Felis catus
  REGION 1..89: note = feline_VL_FW1-3_H5
QSVLTQPPSVSGALGQRVTISCTGSANNIGIVGVSWYQQLPGKAPKLLIYANDRRPSSVLDRFSGSKSGNTGSLTITGLQAEDEADYYC

SEQ ID NO: 97 - protein, 84 aa, Felis catus
  REGION 1..84: note = feline_VL_FW1-3_F2
QSGPNQPSSVSGALGQRVTISCTGVGAYVDWYQQIPGMAPKTIIYYDSNRPSGVPDRFSGSKSGNTGTLTITGLQAEDEADYYC

SEQ ID NO: 98 - protein, 84 aa, Felis catus
  REGION 1..84: note = feline_VL_FW1-3_F3
QSGLTQPSSVSGALGQRVTVSCTGVASYVYWYQQLPGMAPKTIIYGDRNRPSGVPDRFSGSKSGSTGTLTITGLRAEDEADYYC

SEQ ID NO: 99 - protein, 84 aa, Felis catus
  REGION 1..84: note = feline_VL_FW1-3_F6
QSGPNQPSSVSGALGQRVTISCTGVGNYVDWYQQIPGMAPKTIIYYDTDLPSGVPDRFSGSKSGSTATLTITGLRAEDEADYYC

SEQ ID NO: 100 - protein, 84 aa, Felis catus
  REGION 1..84: note = feline_VL_FW1-3_F8
QSVLTQPPSVSGALGQRVTISCTGVGNYVGWHQQIPGMAPKTIIYDGTNRPSGVPDRFSGSKSGDTGTLTITGLQAEDEADYYC

SEQ ID NO: 101 - protein, 84 aa, Felis catus
  REGION 1..84: note = feline_VL_FW1-3_B2
QSGLTQPSSVSGALGQRVTISCTGVGSYVYWYQQIPGMVPKTIIYDNNNRPSGVPDRFSGSKSGFTGTLTIAGLQAEDEADYYC

SEQ ID NO: 102 - protein, 96 aa, Felis catus
  REGION 1..96: note = feline_VL_FW1-3_F4
QPVVTQPASLSASLGATARLTCTLSRDINVGSYSIYWYQQSPGSPPRYLLYYYSDSTTQLGPGVPTRFSGSKDASANAGLLLISGLQPEDEADYFC

SEQ ID NO: 103 - protein, 87 aa, Felis catus
  REGION 1..87: note = feline_VL_FW1-3_C1
QSVLTQPPSVSVSLGQTARTTCGGNNIESKSVHWYQQKSGQTPVLIIYSDSNRPSGIPDRFSGTNSGNTATLTISGAQAEDEADYYC

SEQ ID NO: 104 - protein, 84 aa, Felis catus
  REGION 1..84: note = feline_VL_FW1-3_C5
QSGPNQPSSVSGALGQRVTISCIGVGDYVYWYQQIPGMAPKTLIYGVRNRPSGVPDRFTGSKSGSTGTLTITGFQAEDEADYYC

SEQ ID NO: 105 - protein, 84 aa, Felis catus
  REGION 1..84: note = feline_VL_FW1-3_A2
QSGPNQPSSVSGALGQRVTVSCTGAGSYVGWYQQIPGMVPKTIIYDNGNRPSGVPDRFSGSKSGSIGTLTITGLQAEDEADYYC

SEQ ID NO: 106 - protein, 90 aa, Felis catus
  REGION 1..90: note = feline_VL_FW1-3_A1
QSVVTQPPSVSGTLGQRVTISCTGSSSNVGRGNYVSWYQQLSGSAPKLLIYGNTNRPLGVPDRFSGSKSGSTGSLTITGLQAEDEADYYC

SEQ ID NO: 107 - protein, 87 aa, Felis catus
  REGION 1..87: note = feline_VL_FW1-3_E12
QPVLTQPPSVSVSLGQTARTTCGGNNIESKSVHWYQQKSGQTPVLIIYSDSNRPSGIPDRFSGTNSGNTATLTISGAQAEDEADYYC

SEQ ID NO: 108 - protein, 84 aa, Felis catus
  REGION 1..84: note = feline_VL_FW1-3_C9
QSGLTQPSSVSGALGQRVTLSCTGIDSYVSWYQQIPGMAPKTIIYGNSNRPSGVPDRFSGSKSGSTGILTVTGLQAEDEADYYC

SEQ ID NO: 109 - protein, 84 aa, Felis catus
  REGION 1..84: note = feline_VL_FW1-3_C4
QSGPNQPSSVSGALGQRVTISCTGIGISVDWYQQVPGMAPKTVITRVTDRLSGVPDRFSGFKSGSTATLTITGLRAEDEADYYC

SEQ ID NO: 110 - protein, 90 aa, Felis catus
  REGION 1..90: note = feline_VL_FW1-3_D11
QPVLTQPPSVSGSLGQRVTISCTGTSSNIGSGNYVSWYRQVPGIAPSLLIYENFKRPSGVPDRFSGSKSGSSGSLTITGLQADDEVDYYC

SEQ ID NO: 111 - protein, 84 aa, Felis catus
  REGION 1..84: note = feline_VL_FW1-3_H3
QSGLTQPSSVSGALGQRVTISCAGVGTYVYWYQQLPGMAPKTIIFDNNHRPSGVTGRFFGSKSGDTGTLTITGLQAEDEADYYC

SEQ ID NO: 112 - protein, 84 aa, Felis catus
  REGION 1..84: note = feline_VL_FW1-3_A11
QSGPNQPSSVSGALGQRVTISCTGVGNYVDWYQQVPGMAPKTIIYGNRYRPSGVPDRFSGSKSGSTGTLTITGLQAEDEADYYC

SEQ ID NO: 113 - protein, 90 aa, Felis catus
  REGION 1..90: note = feline_VL_FW1-3_C3
QSVVTQPPSVSGALGQRVTISCTGSSSNIGRGSYVNWYQQLSGTAPKLLIYDDNSRPSGVPDRFSGSKSGSTGSLTITGLQAEDEADYYC

SEQ ID NO: 114 - protein, 84 aa, Felis catus
  REGION 1..84: note = feline_VL_FW1-3_D4
QSGLTQPSSVSGALGQRVTISCTGVGSFVYWYRQIPGMAPKTVIYHNINRPSGVPDRFSGSKSGSTSTLTITGLQAEDEADYYC

SEQ ID NO: 115 - protein, 84 aa, Felis catus
  REGION 1..84: note = feline_VL_FW1-3_F1
QSGPNQPSSVSGALGQRVTISCTGVGTWVGWYQQIPGMAPKTIIYGNNNRPSGVPDRFSGSKSGSTGTLTITGLQAEDETDYYC

SEQ ID NO: 116 - protein, 90 aa, Felis catus
  REGION 1..90: note = feline_VL_FW1-3_B3
QSVLTQPPSVSGALGQRVTISCTGSSSNIGRGNNVNWYQQLSGTAPKLLIYGNTNRPSGVPDRFSGSRSGSTGSLTITGLQADDEADYYC

SEQ ID NO: 117 - protein, 90 aa, Felis catus
  REGION 1..90: note = feline_VL_FW1-3_C2
QSVLTQPPSVSGALGQRVTISCTGSRSNIGRGNYVNWYQQLSGTGPKLLIYGNSNRPSGVPDRFSGSKSGSTGSLTITGLQAEDEADYYC

SEQ ID NO: 118 - protein, 96 aa, Felis catus
  REGION 1..96: note = feline_VL_FW1-3_E8
QSVVTQPASLSASLGATARLTCTLSRDINVGSYNIYWYQQSPGNPPRFLLYYFSDSSTQLGPGVPSHFSGSKDASANAGLLLISGLQPEDEADYYC

SEQ ID NO: 119 - protein, 90 aa, Felis catus
  REGION 1..90: note = feline_VL_FW1-3_E9
QPVVTQPPSVSGALGQRVTISCTGSSSNIGRGNYVSWYQQLSGTAPKLLIYGDKNRPSGVPDRFSGSKSGSTGSLTITGLQAEDEGDYYC

SEQ ID NO: 120 - protein, 89 aa, Felis catus
  REGION 1..89: note = feline_VL_FW1-3_G2
QPVLTQPPSVSGALGQTVTISCAGSRSNIGFAGVNWYQQLPGKAPKLLIYGNDRRPSGVPERISGSKSGNTGSLAITGLQAEDEADYYC

SEQ ID NO: 121 - protein, 90 aa, Felis catus
  REGION 1..90: note = feline_VL_FW1-3_D12
QSVLTQPPSVSGSPGQRVTISCTGSTSNIGGGNSVNWYQQVPGAAPRLLIYENNKRPSGVPDRFSGSKSGNSGSLTITGLQADDEADYYC

SEQ ID NO: 122 - protein, 90 aa, Felis catus
  REGION 1..90: note = feline_VL_FW1-3_D5
QSVLTQPPSLSGALGQRVTISCTGSTSNIGRGNYVNWYQQLSGAAPKLLIFGTANRPSGVPDRFSGSKSGNTGSLTITGLQVGDEADYYC

SEQ ID NO: 123 - protein, 84 aa, Felis catus
  REGION 1..84: note = feline_VL_FW1-3_A3
QSGPNQPSSVSGALGQRVTMSCTGVGMSVDWYQQIPGMAPKTVIYRNSNRLSEVPNRFSGSKSGSTATLTITGLQAEDEADYYC

SEQ ID NO: 124 - protein, 90 aa, Felis catus
  REGION 1..90: note = feline_VL_FW1-3_E2
QPVLTQPPSVSGALGQRVSISCTGSSSNIGRGNYVSWYQQLSGTAPKLLIYGNANRPSGVPDRFSGSKSGITGSLTITALQAEDEADYYC

SEQ ID NO: 125 - protein, 89 aa, Felis catus
  REGION 1..89: note = feline_VL_FW1-3_G12
QSVLTQPPSVSGGLGQKVTISCAGSGSNIGIAGVNWYQQHPRKAPKLLIYGSSNRPSGVPDRFSGSKSGNTGSLTITGLQAEDEADYYC

SEQ ID NO: 126 - protein, 89 aa, Felis catus
  REGION 1..89: note = feline_VL_FW1-3_H4
QSVLTQPPSVSGGLGQKVTISCAGSRSNIGTAGVNWYQQHPGKAPKLLIYGNDNRPSGVPDRFSGSKSGNTGSLTITGLQAEDEADFYC

SEQ ID NO: 127 - protein, 84 aa, Felis catus
  REGION 1..84: note = feline_VL_FW1-3_A4
QSGPNQPSSVSGALGQRVTISCTAPGSYVDWYQQIPGMAPKTIIYGNHNRPSGVPDRFSGSKSGNTGTLTITGLQAEDEADYYC

SEQ ID NO: 128 - protein, 90 aa, Felis catus
  REGION 1..90: note = feline_VL_FW1-3_D10
QPVLTQPPSVSGALGQRVTISCTGSSSNIGRGNYVSWYQQVSGTAPKLLIYGDTNRPSGVPDRFSGSKSGSTGSLTITGLQADDEADYYC

SEQ ID NO: 129 - protein, 84 aa, Felis catus
  REGION 1..84: note = feline_VL_FW1-3_E7
QSGPNQPSSVSGALGQRVTISCTGVDSYVGWYQQIPGRAPKTIIYYDSNRPSGVPDRFSGSKSGNTGTLTITGLQAEDEADYYC

SEQ ID NO: 130 - protein, 89 aa, Felis catus
  REGION 1..89: note = feline_VL_FW1-3_H1
QSVLTQPPSVSRGLGQTVTISCAGNRNNIGIAGVVWYQQHPGKAPKLLIYGSNNRPSGVPDRFSGSKSGNTGSLTITGLQAEDEADYYC

SEQ ID NO: 131 - protein, 90 aa, Felis catus
  REGION 1..90: note = feline_VL_FW1-3_H2
QPVLTQPPSVSGALGQRVTISCTGSSSNFGRGDYVSWYQQVSGTAPKLLIYGDNNRPSGVPDRFSGSKSGRIGSLTITGLQAEDEADYYC

SEQ ID NO: 132 - protein, 90 aa, Felis catus
  REGION 1..90: note = feline_VL_FW1-3_E6
QEVVTQETSLSTTPGGTVTLTCGSSTGAVTTSNYASWVRQKPYQRIQGLIGGTSYRNPGVPARFSGSLVGQKAVLTITGAQSEDEAEYYC

SEQ ID NO: 133 - protein, 89 aa, Felis catus
  REGION 1..89: note = feline_VL_FW1-3_A9
QSVVTQPPSVSGALGQAVTISCAGSANNIGITDVNWYQQLPGKAPKLLIYANNRRPSSVPDRFSGSKSGATGSLTISGLQAEDEADYYC

SEQ ID NO: 134 - protein, 90 aa, Felis catus
  REGION 1..90: note = feline_VL_FW1-3_E10
QEVVTQETSLSTTPGGTVTLTCGSSTGAVTTSNFASWVQQKPYQRFQGLVGGTSYRNPGVPARFSGSLVGQKAVLTITGAQSEDEAEYYC

SEQ ID NO: 135 - protein, 96 aa, Felis catus
  REGION 1..96: note = feline_VL_FW1-3_G8
QPVVTQPPSLSGSLGATARLTCTLSREVSVGVKGIYWYQQKPGSPPRYFLHYYSDSTNELGPGVPSRVSGSKDASANAGLLLISGLQPEDEADYHC

SEQ ID NO: 136 - protein, 84 aa, Felis catus
  REGION 1..84: note = feline_VL_FW1-3_A10
QSGPNQPSSVSGALGQRITISCTGVGSYVGWYQQIPGRAPKTIIFYDTNRRSGVPDRFSGSKSGSTGTLTITGLQAEDEADYYC

SEQ ID NO: 137 - protein, 89 aa, Felis catus
  REGION 1..89: note = feline_VL_FW1-3_B4
QPVVTQPPSMSGALGQTVTISCAGSGTNIGVAGVNWYQQLPGKAPKVLIYASSRRPSGVPERISGSKLGNTGTLTISGLQAEDEADYYC

SEQ ID NO: 138 - protein, 98 aa, Felis catus
  REGION 1..98: note = feline_VH_FW1-3_B5
EVQLVESGGDLVKPGGSLRLTCVASGFTFSNYDMSWVRQAPGKGLQWVAAIAYNGGNTGYADSVKGRFTISRDNAKNTLYLQMNSLKTEDTATYYCAR

SEQ ID NO: 139 - protein, 98 aa, Felis catus
  REGION 1..98: note = feline_VH_FW1-3_A12
QVLLVQSGGDLVKPGGSLRLTCVASGFTFSSYAMSWVRQAPGKGLQWVTDISRSGATTYYADSVKGRFTISRDNVKNTLYLQMNSLKTEDTATYYCAR

SEQ ID NO: 140 - protein, 98 aa, Felis catus
  REGION 1..98: note = feline_VH_FW1-3_D8
DEQLVESGGDLVKPGGSLRLTCVGSGFTFRSHWMNWVRQAPGKGLQWVSAIGGTGSRTLYADSVKGRFTISRDNARNTLYLQMNSLKTEDTATYYCAR

SEQ ID NO: 141 - protein, 98 aa, Felis catus
  REGION 1..98: note = feline_VH_FW1-3_B8
DVQLVESGGDLVKPGGSLRLACVASGFTFSNYGMDWVRQTPGKGLQWVAYIDNDGSSTYYADSVKGRFTISRDNAMNTLYLQMNSLKTEDAATYYCAR

SEQ ID NO: 142 - protein, 98 aa, Felis catus
  REGION 1..98: note = feline_VH_FW1-3_C12
DVRLVESGGNLVKPGGSLRLTCVASGFTFSSYGMSWVRQAPGKGLRWVAYIRYDGSTTNYADSVKGRFTISRDNAKNTLYLQMNSLKTEDTATYYCAT

SEQ ID NO: 143 - protein, 98 aa, Felis catus
  REGION 1..98: note = feline_VH_FW1-3_B6
DVQLVESGGDLVKPGGSLRLTCVASGFTFSDYDMSWVRQAPGKGLQWVAAISYNGGGTGYSDSVKGRFTISRDNAKNTLYLQMNSLKTEDTATYYCAT

SEQ ID NO: 144 - protein, 98 aa, Felis catus
  REGION 1..98: note = feline_VH_FW1-3_E3
YVQLVESGGDLVKPGGSLRLTCVASGFTFSRYAMNWVRQAPGKGLQWVSAISETGGDTYYADSVKGRFTISRDNAKNTLYLQMNGLKTEDTATYYCAR

SEQ ID NO: 145 - protein, 98 aa, Felis catus
  REGION 1..98: note = feline_VH_FW1-3_D1
DVQLVESGGDLVKPGGSLRLTCVASGFTFSSDYMHWVRQTPGKGLQWVAQITSSGGYTYYADSVKGRFTISRDNAKNTLYLQMNSLKTEDTATYYCAS

SEQ ID NO: 146 - protein, 98 aa, Felis catus
  REGION 1..98: note = feline_VH_FW1-3_B9
DVRLVESGGDLVKPGGSLRLTCVASGFTFSSFYMHWVRHAPGKGLQWVAQISGSGHSTKYADSVKGRFTISRDNAKNTLYLQQNSLKTEDTATYFCAM

SEQ ID NO: 147 - protein, 98 aa, Felis catus
  REGION 1..98: note = feline_VH_FW1-3_D3
DVRLVESGGDLVKPGGSLRLTCVASGFTFSSYSMTWVRQAPGKGLQWVAHIYTDGSSTNYADSVKGRFTISRDNAKNTLYLQMNSLKTEDTATYYCAR

SEQ ID NO: 148 - protein, 98 aa, Felis catus
  REGION 1..98: note = feline_VH_FW1-3_G11
EVQLVESGGDLVKPGGSLRLTCVASGFTFSTYYMTWVRQAPRKGLQWVAYIKPDGSSTNYADSVKGRFTISRDNAKNTLYLQMNSLKTEDTATYYCAR

SEQ ID NO: 149 - protein, 98 aa, Felis catus
  REGION 1..98: note = feline_VH_FW1-3_F9
DVQLVESGGDLVKPGGSLRLTCVASGFTFSNYGMSWVRQAPGKGLQWVAGISGSGITTYYADSVKGRFTISRDNAENTLLLQMNSLKTEDTATYYCAR

SEQ ID NO: 150 - protein, 98 aa, Felis catus
  REGION 1..98: note = feline_VH_FW1-3_B11
DVQLVESGGDLVKPGGSLRLTCVASGFTFSLYDMSWVRQAPGKGLQWVTTISGSGGSTYYADSVKGRFTISRDNPKNTLYLQMTSLKTEDTATYYCAR

SEQ ID NO: 151 - protein, 98 aa, Felis catus
  REGION 1..98: note = feline_VH_FW1-3_E11
DVQLVESGGDLVKPGGSLRLICVASGFTFTNYAMSWVRQAPGKGLQWVSAISFDGSGTGYADSVKGRFTISRDNAKNTLYLQMNSLKTEDMATYYCAR

SEQ ID NO: 152 - protein, 98 aa, Felis catus
  REGION 1..98: note = feline_VH_FW1-3_B1
EVQLVESGGDLVKPGGSLRLTCVASGFTFNNYDMSWVRQAPGKGLQWVAAISSNGGITGYADSVKGRFTVSRDNAKNTLYLQMNSLKTDDTATYYCTR

SEQ ID NO: 153 - protein, 98 aa, Felis catus
  REGION 1..98: note = feline_VH_FW1-3_A8
DVRLVESGGDLVKPGGSLRLTCVASGFTFSGYSMNWVRQAPGKGLQWVAYIRYDGNTIHYGDSVKGRFTVSRDNAKNTLYLQMNSLKTEDTATYYCSR

SEQ ID NO: 154 - protein, 98 aa, Felis catus
  REGION 1..98: note = feline_VH_FW1-3_D2
DVQVVESGGDLVQPGGSLRLTCVASGFTFSSYEMNWVRQAPGKGLQWVAYIGSSGGSTYYADSVKGRFTISRDDAKNTLYLQMNSLKTEDTATYYCAR

SEQ ID NO: 155 - protein, 98 aa, Felis catus
  REGION 1..98: note = feline_VH_FW1-3_B7
YVQLVESGGDLVKPGGSLRLICVASGFTFSDNAMTWVRQAPGKGLQWISVISFDGGSTAYADSVKGRFTISRDNAKNTLYLQMNSLKTEDTATYYCAR

SEQ ID NO: 156 - protein, 98 aa, Felis catus
  REGION 1..98: note = feline_VH_FW1-3_C10
EVQLVESGGDLVKPGGSLRLTCVASGFTFSRYGMSWVRQAPGKGLQWVAAISGSGDSTYYADSVKGRFTISRDNAKNTLYLQMNSLKTEDTATYYCAK

SEQ ID NO: 157 - protein, 97 aa, Felis catus
  REGION 1..97: note = feline_VH_FW1-3_D7
DVQLVESGGDLVQPGGSLRLTCVASGFTFSSYEMNWVRQAPGKGLQWVAYISSGGSTYYADSVKGRFTISRDNAKNTLYLQMNSLKTEDTATYYCAN

SEQ ID NO: 158 - protein, 98 aa, Felis catus
  REGION 1..98: note = feline_VH_FW1-3_B10
DEQLVESGGDLVKPGGSLRLTCVASGFTFSRYGMSWVRQAPGKGLQWVAAIGGSGGSIYYADSVKGRFTISRDNAKNTLYLQMNSLKTEDSATYHCVR

SEQ ID NO: 159 - protein, 98 aa, Felis catus
  REGION 1..98: note = feline_VH_FW1-3_A7
DEQLVESGGDLVKPGGSLRLTCVASGFTFSSYGMSWVRQAPGKGLQWVAAISGSGGSIYYADSVKGRFTISRDNAKNTLYLQMNSLKTEDTATYYCAR

SEQ ID NO: 160 - protein, 97 aa, Felis catus
  REGION 1..97: note = feline_VH_FW1-3_H6
DVQVVESGGDLVKPGGSLRLTCVASGFTFSNYGMSWVRQAPGKGLQWVAGITSGGNTYYADSVKGRFTISRDNAKNTLYLQMNSLKTEDTATYYCAR

SEQ ID NO: 161 - protein, 98 aa, Felis catus
  REGION 1..98: note = feline_VH_FW1-3_H5
DVQLVESGGDLVKPGGSLRLTCVASGFTFSNYAMNWVRQAPGKGLQWVSAISESGDSTYYADSVKGRFTISRDNAKNTLYLQMNSLKTEDTATYYCAR

SEQ ID NO: 162 - protein, 98 aa, Felis catus
  REGION 1..98: note = feline_VH_FW1-3_F2
DVQLVESGGDLVKPGGSLRLTCVASGFTFSSYYMHWVRQAPGKGLQWVAQISNSGGSTYYADSVKGRFTISRDNAKNTLYLQMNSLKTEDTATYYCTK

SEQ ID NO: 163 - protein, 98 aa, Felis catus
  REGION 1..98: note = feline_VH_FW1-3_F3
DVQLVESGGDLVKPGGSLRLTCVASGFTFNNFAMSWVRQAPGKGLQWVSALSESGHSTIYADSVKGRFTISRDNAKNTLYLQMSSLKTEDTATYYCAR

SEQ ID NO: 164 - protein, 98 aa, Felis catus
  REGION 1..98: note = feline_VH_FW1-3_F6
DVQLVESGGNLVKPGGSLRLTCVASGFTFSSSGMSWVRQAPGKGLQWVAHIYDDGSTERYADSVKGRFTISRDNAKNTLYLQMNSLKTEDTATYYCAR

SEQ ID NO: 165 - protein, 98 aa, Felis catus
  REGION 1..98: note = feline_VH_FW1-3_F8
DVQLVESGGDLVKPGGSLRLTCVASGFTFSTYYMNWVRQAPGKGLQWVAYINPDGSSTNYADSVKGRFTISRDNAKNTLYLQMNSLKTEDTATYYCTS

SEQ ID NO: 166 - protein, 98 aa, Felis catus
  REGION 1..98: note = feline_VH_FW1-3_B2
DVRLVESGGDLVQPGGSLRLTCVASGFTFSTYEMNWVRQAPGKGLRWVTGISSSGGATYYADSVKGRFTISRDNAKNTLYLSMNSLKTEDTATYYCTS

SEQ ID NO: 167 - protein, 98 aa, Felis catus
  REGION 1..98: note = feline_VH_FW1-3_F4
DVQLLESGGDLVKPGGSLRLTCVASGFTFSSYGMSWVRQAPGKGLQWVAAISGSGGSTYYADSVKGRFTISRDNAKNTLYLQMNSLKTEDTATYFCAR

SEQ ID NO: 168 - protein, 98 aa, Felis catus
  REGION 1..98: note = eline_VH_FW1-3_C1
DVRLVESGGDLVKPGGALRLTCVASGFTFSVYGMSWVRQAPGKGLQWVTAISGNGGDTYYADSVKGRFTISRDNAKNTLYLHMNSLKTEDAATYYCAR

SEQ ID NO: 169 - protein, 98 aa, Felis catus
  REGION 1..98: note = feline_VH_FW1-3_C5
DVQLVESGGDLVKPGGSLRLTCVASGFTFSNYGMTWVRQAPGKGLQWVAYIRYDGSSTNYADSVKGRFTISRDNAKNTLYLQMNSLKTEDTATYYCST

SEQ ID NO: 170 - protein, 98 aa, Felis catus
  REGION 1..98: note = feline_VH_FW1-3_A2
DVQLVESGGDLVKPGGSLRLTCVASGFTFSSHYMNWVRQAPGKGLQWVSWINTDGSTTSYADSVKGRFTISRDNSKNTLYLQMNNLKTEDTATYYCAR

SEQ ID NO: 171 - protein, 98 aa, Felis catus
  REGION 1..98: note = feline_VH_FW1-3_A1
DVQLVESGGDLVKPGGSLRLTCVASGFTFSNYYMSWVRQAPGKGLQWVSWINKDGSVTSYADSVKGRFTISRDNSKNTLLLQMNNLKTEDAATYYCAR

SEQ ID NO: 172 - protein, 98 aa, Felis catus
  REGION 1..98: note = feline_VH_FW1-3_E12
DVQLVESGGDLVKPGGSLRLTCVASGFTFSSYAMSWVRQAPGKGLQWVSAISEGDGSAFYTDSVKGRFTISRDNAKNTLYLQMNSLKTEDTATYFCVR

SEQ ID NO: 173 - protein, 98 aa, Felis catus
  REGION 1..98: note = feline_VH_FW1-3_C9
EVQLVESGGDLVKPGGSLRLTCVASGFTFSNYGMNWVRQAPGKGLQWVAAISGSGGSTYYADSVKGRFTISRGNAKNALYLQMNSLKTEDTATYYCAR

SEQ ID NO: 174 - protein, 98 aa, Felis catus
  REGION 1..98: note = feline_VH_FW1-3_C4
DVQLVESGGDLVKPGGSLRLTCVASGFTFSSYYMNWVRQPPGKGLQWVANINTDGSTTSYADSVKGRFTISRDNAKNTLYLQMNSLKTEDTATYYCAR

SEQ ID NO: 175 - protein, 98 aa, Felis catus
  REGION 1..98: note = feline_VH_FW1-3_D11
QVLLVQSGGDLVKPGGSLRLTCVASGFTFNSYAMSWVRQAPGKGLQWVADISGSGGATAYADSVKGRFTISRDNAKNTLYLQMNSLKTEDTATYYCAK

SEQ ID NO: 176 - protein, 98 aa, Felis catus
  REGION 1..98: note = feline_VH_FW1-3_H3
DVRLVESGGDLVKPGGSLRLTCVASGFTFSSFGMGWVRQAPGKGLQWVAYIRSTGSTTNYADSVKGRFTISRDNAKNTLYLQMNSLKTEDTATYYCAT

SEQ ID NO: 177 - protein, 98 aa, Felis catus
  REGION 1..98: note = feline_VH_FW1-3_A11
DVQLVESGGDLVKPGGSLRLTCVASGFTFSNYGMSWVRQAPGKGLQWVAANSGTGSSTYYADSVKGRFTISRDNAKNTLYLQMNSLKTEDTATYYCAR

SEQ ID NO: 178 - protein, 98 aa, Felis catus
  REGION 1..98: note = feline_VH_FW1-3_C3
DEQLVESGGNLVKPGGSLRLTCVASGFTFSNYDMSWVRQAPGKGLQWVAYIYNDGSGTSYADSVKGRFTISRDNAKNTLYLQMNSLKTEDTATYYCAK

SEQ ID NO: 179 - protein, 98 aa, Felis catus
  REGION 1..98: note = feline_VH_FW1-3_D4
DEQLVESGGDLVKPGGSLRLTCVASGFTFSSYSMNWVRQAPGKGLQWVAYIRYDGSRLYYADSVKGRFTVSRDNAKNTLYLQMNSLKTEDTATYYCAN

SEQ ID NO: 180 - protein, 98 aa, Felis catus
  REGION 1..98: note = feline_VH_FW1-3_F1
DVQLVESGGDLVKPGGSLTLTCVASGFTFNIYNMNWVRQAPGKGLQWVAYISRGGGSIHYADSVKGRFTISRDNAKNTLYLQMDSLKTDDTATYYCTG

SEQ ID NO: 181 - protein, 98 aa, Felis catus
  REGION 1..98: note = feline_VH_FW1-3_B3
DVQLVESGGDLVKPGGSLRLTCVASGFTFRSFWMNWVRQAPGKGLQWVSAISDSGTSTGYADSVKGRFTISRDNAKNTLYLQMNSLKTEDTATYYCTK

SEQ ID NO: 182 - protein, 98 aa, Felis catus
  REGION 1..98: note = feline_VH_FW1-3_C2
EVQLVESGGDLVKPGGSLRLTCVASGFTFRSYWMNWVRQAPGKGLQWVSAISDSGRSTGYADSVRGRFTISRDNAKNTLYLQMNSLKTEDTATYYCAG

SEQ ID NO: 183 - protein, 98 aa, Felis catus
  REGION 1..98: note = feline_VH_FW1-3_E8
EVQLVESGGDLVKPGGSLRLTCVASGFTFSNYYMSWVRQSPGKGLQWVAYISTSGGDFYYADSVKGRFTVSRDNAKNTLYLQMNSLKTEDTATYYCAR

SEQ ID NO: 184 - protein, 98 aa, Felis catus
  REGION 1..98: note = feline_VH_FW1-3_E9
DEQLVESGGDLVKPGGSLRLTCVASGFPFSRYGMSWVRQAPGKGLQWVAAIRGSGGVTYYADSVKGRFTISRDNAKNTLYLQMNSLKTEDTATYYCAR

SEQ ID NO: 185 - protein, 98 aa, Felis catus
  REGION 1..98: note = feline_VH_FW1-3_G2
DVRLVESGGDLVKPGGSLRLTCVASGFTFSSYSMNWVRQAPGKGLQWVAYIRYDGSSIYYADSVKGRFTVSRDNAKNTLYLQMNSLKTEDTATYYCAR

SEQ ID NO: 186 - protein, 98 aa, Felis catus
  REGION 1..98: note = feline_VH_FW1-3_D12
DVQLVESGGNLVKPGGSLRLTCVASGFTFSSYDMSWVRQAPGKGLQWVAYIYDDGSDTNYADSVKGRFTVSRDNAKNTLYLQMNSLKTEDTATYYCAM

SEQ ID NO: 187 - protein, 98 aa, Felis catus
  REGION 1..98: note = feline_VH_FW1-3_D5
DVQLVESGGDLVKPGGSLRLTCVASGFTFSSYGMNWVRQAPGKGLQWVAYIRYDGSSTNYADSVKGRFTISRDNAKNTLYLQMSSLKTEDTATYYCAM

SEQ ID NO: 188 - protein, 98 aa, Felis catus
  REGION 1..98: note = feline_VH_FW1-3_A3
DVQLVESGGDLVKPGGSLRLTCVASGFIFSRYGMSWVRQAPGKGLQWVAGISTSGGNTYYADSVKGRFTISRDNAKNTLYLQMNSLKTEDTATYYCAR

SEQ ID NO: 189 - protein, 98 aa, Felis catus
  REGION 1..98: note = feline_VH_FW1-3_E2
QVLLVQSGGDLVKPGGSLRLTCVASGFTFSDYAMSWVRQAPGKGLQWVSTVSESGSNTYYGDSVRGRFTISRDNAKNKLYLQMNSLKSEDTATYYCAR

SEQ ID NO: 190 - protein, 98 aa, Felis catus
  REGION 1..98: note = feline_VH_FW1-3_G12
DEQLVESGGDLVKPGGSLRLTCVASGFTLGSYDMTWVRQAPGKGLQWVAAIGHDGSTTAYADSVKGRFTISRDNAKNTLYLQMNSLKTEDTATYYCAR

SEQ ID NO: 191 - protein, 98 aa, Felis catus
  REGION 1..98: note = feline_VH_FW1-3_H4
DVQLVESGGDLVKPGGSLRLTCVASGFTFSNYYMEWVRQAPGKGLQWVAQISEGGGSTYYADSVKGRFTISRDNAKNTLYLQMNSLKTEDTATYFCAR

SEQ ID NO: 192 - protein, 105 aa, Felis catus
  REGION 1..105: note = feline_VH_FW1-3_A4
DVRLVESGGDLVKPGGSLRLTCVLPLDSPSVPTGSISIYWMNWVRQAPGKGLQWVAGISGSGDRTGYADSVKGRFTISIDNAKNTLYLQMDSLKTEDTATYYCET

SEQ ID NO: 193 - protein, 98 aa, Felis catus
  REGION 1..98: note = feline_VH_FW1-3_D10
YVQLVESGGDLVKPGGSLRLTCVASGFTFSSYYMHWVRQAPGKGLQWVAQISDSGGSTYYADSVKGRFTISRDNAKNTLYLQMNGLKTEDTATYYCAR

SEQ ID NO: 194 - protein, 97 aa, Felis catus
  REGION 1..97: note = feline_VH_FW1-3_E7
DVRLVESGGDRVKPGGSLRLTCMASGFNVRGYAMTWVRQAPGMGLQWVSSISSGGTTYYADSVKGRFTISKDDAENTLYLQMNSLKTEDTATYYCAG

SEQ ID NO: 195 - protein, 98 aa, Felis catus
  REGION 1..98: note = feline_VH_FW1-3_H1
DVQVVESGGDLVKPGGSLRLTCLASGFTFSSYGMSWVRQAPGKGLQWVAYIRYDGSSTKYADSVKGRFTISRDNAKNTLYLQMNSLKTEDTATYYCAT

SEQ ID NO: 196 - protein, 98 aa, Felis catus
  REGION 1..98: note = feline_VH_FW1-3_H2
DVQLVESGGDLVKPGGSLRLTCVASGFTFSTYYMHWVRQAPGKGLQWVAQINSGGSSTFYADSVKGRFTISRDNAKNTLHLQMNSLKTEDTGTYYCAS

SEQ ID NO: 197 - protein, 98 aa, Felis catus
  REGION 1..98: note = feline_VH_FW1-3_E6
DVQLVESGGDLVKPGGSLRLTCVASGFTFSTYGMSWVRQAPGKGLQWVAAISGSGGSTSYAESVKGRFTISRDNAKNTLYLQMNSLKTEDTATYYCAR

SEQ ID NO: 198 - protein, 97 aa, Felis catus
  REGION 1..97: note = feline_VH_FW1-3_A9
EVQLVESGGDLVKPGGSLRLTCVASGFTFTGDAMSWVRQAPGRGLQWVSTIDSGGNTHYIDSVKGRFTISRDNAKNTLYLQMNNLKTEDTATYYCAG

SEQ ID NO: 199 - protein, 98 aa, Felis catus
  REGION 1..98: note = feline_VH_FW1-3_E10
DVQLVESGGDLVKPGGSLRLTCVASGFPFSSYGMSWVRQAPGKGLQWVAYIRYDGSSTNYADSVKGRFTISRDNAKNTLYLQMDSLKTEDTATYYCTS

SEQ ID NO: 200 - protein, 98 aa, Felis catus
  REGION 1..98: note = feline_VH_FW1-3_G8
DVQLVESGGDLVKPGGSLRLTCVASGFTFNNFAMSWVRQAPGKGLQWVSALSESGHSTIYADSVKGRFTISRDNAKNTLSLQMDSLKTEDTATYYCAT

SEQ ID NO: 201 - protein, 98 aa, Felis catus
  REGION 1..98: note = feline_VH_FW1-3_A10
DVQLVESGGDLVQPGGSLRLTCVASGFTFNNYEMNWVRQAPGKGLQWVAYISFSGDSTNYADSVQGRFTISRDNAKNTLYLQMNSLKTEDTATYYCAR

SEQ ID NO: 202 - protein, 98 aa, Felis catus
  REGION 1..98: note = feline_VH_FW1-3_B4
DEQLVESGGDLVKPGGSVRLTCVASGFTFSTSAMSWVRQAPGKGLQWVSSVSENGGGTFYADSVKGRFTVSRDNARNTLYLQMNSLKTEDSATYYCAR

SEQ ID NO: 203 - protein, 106 aa, Felis catus
  REGION 1..106: note = feline_Fab_feline_CL_lambda_wild type
GQPKSAPSVTLFPPSNEELSANKATLVCLISDFYPSGLTVAWKADGTPVTQGVETTKPSKQSNNKYAASSYLSLSPNEWKSRSRFTCQVTHEGSTVEKSVVPAECS

SEQ ID NO: 204 - protein, 106 aa, synthetic construct
  REGION 1..106: note = feline_Fab_feline_CL_lambda_C105S
GQPKSAPSVTLFPPSNEELSANKATLVCLISDFYPSGLTVAWKADGTPVTQGVETTKPSKQSNNKYAASSYLSLSPNEWKSRSRFTCQVTHEGSTVEKSVVPAESS

SEQ ID NO: 205 - protein, 98 aa, Felis catus
  REGION 1..98: note = feline_Fab_feline_CH1_wild type
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTV

SEQ ID NO: 206 - protein, 98 aa, synthetic construct
  REGION 1..98: note = feline_Fab_feline_CH1_C15S
ASTTAPSVFPLAPSSGTTSGATVALACLVLGYFPEPVTVSWNSGALTSGVHTFPAVLQASGLYSLSSMVTVPSSRWLSDTFTCNVAHPPSNTKVDKTV

SEQ ID NO: 207 - protein, 18 aa, synthetic construct
  REGION 1..18: note = scFV_Linker
GGSSRSSSSGGGGSGGGG

SEQ ID NO: 208 - protein, 10 aa, Felis catus
  REGION 1..10: note = VL_FW4_a
FGGGTHLTVL

SEQ ID NO: 209 - protein, 10 aa, Felis catus
  REGION 1..10: note = VL_FW4_b
FGGGTHLSVL

SEQ ID NO: 210 - protein, 10 aa, Felis catus
  REGION 1..10: note = VL_FW4_c
FGRGTHLTVL

SEQ ID NO: 211 - protein, 10 aa, Felis catus
  REGION 1..10: note = VL_FW4_d
FGGGTHVTVL

SEQ ID NO: 212 - protein, 10 aa, Felis catus
  REGION 1..10: note = VL_FW4_e
IGGGTHVTVL

SEQ ID NO: 213 - protein, 11 aa, Felis catus
  REGION 1..11: note = VH_FW4_a
WGQGALVTVSS

SEQ ID NO: 214 - protein, 11 aa, Felis catus
  REGION 1..11: note = VH_FW4_b
WGQGAPVTVSS

SEQ ID NO: 215 - protein, 11 aa, Felis catus
  REGION 1..11: note = VH_FW4_c
WGQGTLVTVSS

SEQ ID NO: 216 - protein, 11 aa, Felis catus
  REGION 1..11: note = VH_FW4_d
WGQGVLVTVSS

SEQ ID NO: 217 - protein, 11 aa, Felis catus
  REGION 1..11: note = VH_FW4_e
WGRGALVTVSS

SEQ ID NO: 218 - protein, 84 aa, synthetic construct; identical to SEQ ID NO: 78
  REGION 1..84: note = VL_FW1-3_B6_mod

SEQ ID NO: 219 - protein, 84 aa, synthetic construct; identical to SEQ ID NO: 112
  REGION 1..84: note = VL_FW1-3_A11_mod

SEQ ID NO: 220 - protein, 84 aa, synthetic construct; identical to SEQ ID NO: 73
  REGION 1..84: note = VL_FW1-3_B5_mod

SEQ ID NO: 221 - protein, 90 aa, synthetic construct
  REGION 1..90: note = VL_FW1-3_C10_mod
QSVVTQPPSVSGALGQRVTISCTGSSSNVGRGNYVSWYQQLSGTPPKLLIYGNTDRLSGVPDRFSGSKSGTTGSLTITGLQTEDEADYYC

SEQ ID NO: 222 - protein, 84 aa, synthetic construct; identical to SEQ ID NO: 92
  REGION 1..84: note = VL_FW1-3_D7_mod

SEQ ID NO: 223 - protein, 90 aa, synthetic construct; identical to SEQ ID NO: 128
  REGION 1..90: note = VL_FW1-3_D10_mod

SEQ ID NO: 224 - protein, 90 aa, synthetic construct; identical to SEQ ID NO: 110
  REGION 1..90: note = VL_FW1-3_D11_mod

SEQ ID NO: 225 - protein, 98 aa, synthetic construct
  REGION 1..98: note = VH_FW1-3_B6_mod
DVQLVESGGDLVKPGGSLRLTCVASGFTFSDYDMSWVRQAPGKGLQWVAAISYSGGGTGYSDSVKGRFTISRDNAKNTLYLQMNSLKTEDTATYYCAR

SEQ ID NO: 226 - protein, 98 aa, synthetic construct
  REGION 1..98: note = VH_FW1-3_A11_mod
DVQLVESGGDLVKPGGSLRLTCVASGFTFSNYGMSWVRQAPGKGLQWVAAISGTGSSTYYADSVKGRFTISRDNAKNTLYLQMNSLKTEDTATYYCAR

SEQ ID NO: 227 - protein, 98 aa, synthetic construct
  REGION 1..98: note = VH_FW1-3_B5_mod
DVQLVESGGDLVKPGGSLRLTCVASGFTFSNYDMSWVRQAPGKGLQWVAAIAYSGGNTGYADSVKGRFTISRDNAKNTLYLQMNSLKTEDTATYYCAR

SEQ ID NO: 228 - protein, 98 aa, synthetic construct
  REGION 1..98: note = VH_FW1-3_C10_mod
DVQLVESGGDLVKPGGSLRLTCVASGFTFSRYGMSWVRQAPGKGLQWVAAISGSGDSTYYADSVKGRFTISRDNAKNTLYLQMNSLKTEDTATYYCAR

SEQ ID NO: 229 - protein, 97 aa, synthetic construct
  REGION 1..97: note = VH_FW1-3_D7_mod
DVQLVESGGDLVQPGGSLRLTCVASGFTFSSYEMNWVRQAPGKGLQWVAYISSGGSTYYADSVKGRFTISRDNAKNTLYLQMNSLKTEDTATYYCAR

SEQ ID NO: 230 - protein, 98 aa, synthetic construct
  REGION 1..98: note = VH_FW1-3_D10_mod
DVQLVESGGDLVKPGGSLRLTCVASGFTFSSYYMHWVRQAPGKGLQWVAQISDSGGSTYYADSVKGRFTISRDNAKNTLYLQMNGLKTEDTATYYCAR

SEQ ID NO: 231 - protein, 98 aa, synthetic construct
  REGION 1..98: note = VH_FW1-3_D11_mod
DVQLVQSGGDLVKPGGSLRLTCVASGFTFSSYAMSWVRQAPGKGLQWVADISGSGGATAYADSVKGRFTISRDNAKNTLYLQMNSLKTEDTATYYCAR

SEQ ID NO: 232 - protein, 123 aa, synthetic construct
  REGION 1..123: note = VH_Consensus_a
  VARIANT 1: note = x = asp, gln, glu or tyr
  VARIANT 2: note = x = val or glu
  VARIANT 3: note = x = gln, leu or arg
  VARIANT 6: note = x = glu or gln
  VARIANT 11: note = x = leu or arg
  VARIANT 13: note = x = lys or gln
  VARIANT 21: note = x = thr, ile or ala
  VARIANT 23: note = x = val or met
  VARIANT 24: note = x = ala or gly
  VARIANT 28: note = x = thr, pro, ile or asn
  VARIANT 29: note = x = phe, val or leu
  VARIANT 30..35: note = x = any amino acid
  VARIANT 40: note = x = ala or thr
  VARIANT 43: note = x = lys or met
  VARIANT 49..61: note = x = any amino acid
  VARIANT 70: note = x = ile or val
  VARIANT 72: note = x = arg or lys
  VARIANT 74: note = x = asn or asp
  VARIANT 75: note = x = ala, pro or val
  VARIANT 76: note = x = lys, arg, met or glu
  VARIANT 80: note = x = tyr or leu
  VARIANT 84: note = x = asn, thr or asp
  VARIANT 85: note = x = ser, gly or asn
  VARIANT 91: note = x = thr, ala or met
  VARIANT 97: note = x = ala, ser or thr
  VARIANT 98: note = x = arg, lys, asn, gly or thr
  VARIANT 99..112: note = x = any amino acid
  VARIANT 115: note = x = gln or arg
  VARIANT 117: note = x = ala, thr or val
  VARIANT 118: note = x = leu or pro
XXXLVXSGGDXVXPGGSLRLXCXXSGFXXXXXXXXWVRQXPGXGLQWVXXXXXXXXXXXXXDSVKGRFTXSXDXXXNTLXLQMXXLKTEDXATYYCXXXXXXXXXXXXXXXXWGXGXXVTVSS

SEQ ID NO: 233 - protein, 123 aa, synthetic construct; identical to SEQ ID NO: 232
  REGION 1..123: note = VH_Consensus_b
  VARIANT 1: note = x = asp, gln, glu or tyr
  VARIANT 2: note = x = val or glu
  VARIANT 3: note = x = gln, leu or arg
  VARIANT 6: note = x = glu or gln
  VARIANT 11: note = x = leu or arg
  VARIANT 13: note = x = lys or gln
  VARIANT 21: note = x = thr, ile or ala
  VARIANT 23: note = x = val or met
  VARIANT 24: note = x = ala or gly
  VARIANT 28: note = x = thr, pro, ile or asn
  VARIANT 29: note = x = phe, val or leu
  VARIANT 30: note = x = ser, arg, gly, thr or asn
  VARIANT 31: note = x = ser, arg, gly, asn, asp or leu
  VARIANT 32: note = x = tyr, his, phe or asp
  VARIANT 33: note = x = gly, trp, asp, glu, ala, ser or tyr
  VARIANT 35: note = x = ser, asn, thr, asp or his
  VARIANT 40: note = x = ala or thr
  VARIANT 43: note = x = lys or met
  VARIANT 49: note = x = ala, ser or thr
  VARIANT 50: note = x = ala, ser, tyr, gly, thr, gln or asp
  VARIANT 51: note = x = ile, asn or leu
  VARIANT 52: note = x = ser, gly, arg, asp, ala or no residue
  VARIANT 53: note = x = gly, phe, tyr, ser, asn, asp or arg
  VARIANT 54: note = x = ser, thr, asp, gly or asn
  VARIANT 56: note = x = gly, ser, ile, his, asp or ala
  VARIANT 57: note = x = ser, ala, thr, val or asn
  VARIANT 58..59: note = x = thr or ile
  VARIANT 59: note = x = tyr, ala, gly, asn or his
  VARIANT 61: note = x = ala, ser, gly or ile
  VARIANT 70: note = x = ile or val
  VARIANT 72: note = x = arg or lys
  VARIANT 74: note = x = asn or asp
  VARIANT 75: note = x = ala, pro or val
  VARIANT 76: note = x = lys, arg, met or glu
  VARIANT 80: note = x = tyr or leu
  VARIANT 84: note = x = asn, thr or asp
  VARIANT 85: note = x = ser, gly or asn
  VARIANT 91: note = x = thr, ala or met
  VARIANT 97: note = x = ala, ser or thr
  VARIANT 98: note = x = arg, lys, asn, gly or thr
  VARIANT 99..112: note = x = any amino acid
  VARIANT 115: note = x = gln or arg
  VARIANT 117: note = x = ala, thr or val
  VARIANT 118: note = x = leu or pro

SEQ ID NO: 234 - protein, 123 aa, synthetic construct
  REGION 1..123: note = VH_Consensus_c
  VARIANT 30..35: note = x = any amino acid
  VARIANT 49..61: note = x = any amino acid
  VARIANT 99..112: note = x = any amino acid
DVQLVESGGDLVKPGGSLRLTCVASGFTFXXXXXXWVRQAPGKGLQWVXXXXXXXXXXXXXDSVKGRFTISRDNAKNTLYLQMNSLKTEDTATYYCARXXXXXXXXXXXXXXWGQGALVTVSS

SEQ ID NO: 235 - protein, 123 aa, synthetic construct
  REGION 1..123: note = VH_Consensus_d
  VARIANT 99..112: note = x = any amino acid
DVQLVESGGDLVKPGGSLRLTCVASGFTFSSYGMSWVRQAPGKGLQWVAAISGSGGSTYYADSVKGRFTISRDNAKNTLYLQMNSLKTEDTATYYCARXXXXXXXXXXXXXXWGQGALVTVSS

SEQ ID NO: 236 - protein, 118 aa, synthetic construct
  REGION 1..118: note = VL_Consensus_a
  VARIANT 2: note = x = ser, pro or glu
  VARIANT 3: note = x = val or gly
  VARIANT 4: note = x = pro, leu or val
  VARIANT 5: note = x = thr or asn
  VARIANT 7: note = x = pro or glu
  VARIANT 8: note = x = pro, ser or thr
  VARIANT 10: note = x = val, met or leu
  VARIANT 12: note = x = gly, ala or thr
  VARIANT 13: note = x = ala, gly, ser or thr
  VARIANT 14: note = x = leu or pro
  VARIANT 16: note = x = gln, gly or thr
  VARIANT 17: note = x = arg, ala, thr or lys
  VARIANT 18: note = x = val or ile
  VARIANT 19: note = x = thr or arg
  VARIANT 20: note = x = ile, met or leu
  VARIANT 21: note = x = ser or thr
  VARIANT 23..36: note = x = any amino acid
  VARIANT 38: note = x = tyr or val
  VARIANT 39: note = x = gln or arg
  VARIANT 41: note = x = ile, val, leu, lys or his
  VARIANT 42: note = x = pro or ser
  VARIANT 43: note = x = gly, arg or tyr
  VARIANT 44: note = x = met, lys, arg, gln, thr or ser
  VARIANT 45: note = x = ala, pro or arg
  VARIANT 46: note = x = pro or phe
  VARIANT 47: note = x = lys, gln or ser
  VARIANT 48: note = x = thr, leu, ala, val or tyr
  VARIANT 49: note = x = leu, ile, val or phe
  VARIANT 50: note = x = ile, val or leu
  VARIANT 51: note = x = tyr, val, gly or his
  VARIANT 52: note = x = no amino acid or tyr
  VARIANT 53: note = x = no amino acid or tyr
  VARIANT 54: note = x = no amino acid or ser
  VARIANT 55: note = x = no amino acid or asp
  VARIANT 56..65: note = x = any amino acid
  VARIANT 66: note = x = asp, ala, asn or ser
  VARIANT 68: note = x = phe, ile, leu or val
  VARIANT 72: note = x = lys or leu
  VARIANT 73: note = x = asp or no amino acid
  VARIANT 74: note = x = ala or no amino acid
  VARIANT 75: note = x = ser or va
  VARIANT 76: note = x = gly or ala
  VARIANT 77: note = x = ser, asn, thr, gln, ile or ala
  VARIANT 78: note = x = thr, lys or ala
  VARIANT 79: note = x = gly or ala
  VARIANT 80: note = x = ser, thr, leu or val
  VARIANT 82: note = x = thr, ala or leu
  VARIANT 83: note = x = ile or val
  VARIANT 84: note = x = thr or ser
  VARIANT 86: note = x = leu, pro or ala
  VARIANT 88: note = x = ala, ser, thr or pro
  VARIANT 92: note = x = ala, gly or val
  VARIANT 93: note = x = asp or asn
  VARIANT 95: note = x = tyr or his
  VARIANT 97..108: note = x = any amino acid
  VARIANT 109: note = x = phe or ile
  VARIANT 111: note = x = gly or arg
  VARIANT 115: note = x = leu or val
  VARIANT 116: note = x = thr or ser
QXXXXQXXSXSXXXGXXXXXXCXXXXXXXXXXXXXXWXXQXXXXXXXXXXXXXXXXXXXXXXXXXXRXSGSXXXXXXXXXLXXXGXQXEDEXXYXCXXXXXXXXXXXXXGXGTHXXVL

SEQ ID NO: 237 - protein, 118 aa, synthetic construct; identical to SEQ ID NO: 236
  REGION 1..118: note = VL_Consensus_b
  REGION 1..118: note = VL_Consensus_b
  VARIANT 2: note = x = ser, pro or glu
  VARIANT 3: note = x = val or gly
  VARIANT 4: note = x = pro, leu or val
  VARIANT 5: note = x = thr or asn
  VARIANT 7: note = x = pro or glu
  VARIANT 8: note = x = pro, ser or thr
  VARIANT 10: note = x = val, met or leu
  VARIANT 12: note = x = gly, ala or thr
  VARIANT 13: note = x = ala, gly, ser or thr
  VARIANT 14: note = x = leu or pro
  VARIANT 16: note = x = gln, gly or thr
  VARIANT 17: note = x = arg, ala, thr or lys
  VARIANT 18: note = x = val or ile
  VARIANT 19: note = x = thr or arg
  VARIANT 20: note = x = ile, met or leu
  VARIANT 21: note = x = ser or thr
  VARIANT 23: note = x = thr, ala, ser or gly
  VARIANT 24: note = x = gly, ser or leu
  VARIANT 25: note = x = ser, thr or no amino acid
  VARIANT 26: note = x = ser, gly, ala, thr, arg or no amino acid
  VARIANT 27: note = x = ser, asn, gly, pro, glu or no amino acid
  VARIANT 28: note = x = asn, ala, phe, val or no amino acid
  VARIANT 29: note = x = ile, asn, val, ser or no amino acid
  VARIANT 30: note = x = gly, thr, val or no amino acid
  VARIANT 31: note = x = val, arg, ile, ser, thr, gly, ala or no amino acid
  VARIANT 32: note = x = gly, val, ala, asp, ser or thr
  VARIANT 33: note = x = asn, asp, gly, thr, ser, met, val, lys or tyr
  VARIANT 34: note = x = tyr, ser, gly or no amino acid
  VARIANT 35: note = x = val, ala or ile
  VARIANT 36: note = x = ser, asn, gly, asp or tyr
  VARIANT 38: note = x = tyr or val
  VARIANT 39: note = x = gln or arg
  VARIANT 41: note = x = ile, val, leu, lys or his
  VARIANT 42: note = x = pro or ser
  VARIANT 43: note = x = gly, arg or tyr
  VARIANT 44: note = x = met, lys, arg, gln, thr or ser
  VARIANT 45: note = x = ala, pro or arg
  VARIANT 46: note = x = pro or phe
  VARIANT 47: note = x = lys, gln or ser
  VARIANT 48: note = x = thr, leu, ala, val or tyr
  VARIANT 49: note = x = leu, ile, val or phe
  VARIANT 50: note = x = ile, val or leu
  VARIANT 51: note = x = tyr, val, gly or his
  VARIANT 52: note = x = no amino acid or tyr
  VARIANT 53: note = x = no amino acid or tyr
  VARIANT 54: note = x = no amino acid or ser
  VARIANT 55: note = x = no amino acid or asp
  VARIANT 56: note = x = gly, ala, ser, asp, glu, arg or tyr
  VARIANT 57: note = x = asn, ser, thr or asp
  VARIANT 58: note = x = ser, asn, thr, asp, arg, lys or phe
  VARIANT 59: note = x = asn, arg, lys, asp, glu, tyr or his
  VARIANT 60: note = x = arg, gln or leu
  VARIANT 61: note = x = pro, asn or gly
  VARIANT 62: note = x = ser or pro
  VARIANT 63: note = x = gly, ser or glu
  VARIANT 66: note = x = asp, ala, asn or ser
  VARIANT 68: note = x = phe, ile, leu or val
  VARIANT 72: note = x = lys or leu
  VARIANT 73: note = x = asp or no amino acid
  VARIANT 74: note = x = ala or no amino acid
  VARIANT 75: note = x = ser or va
  VARIANT 76: note = x = gly or ala
  VARIANT 77: note = x = ser, asn, thr, gln, ile or ala
  VARIANT 78: note = x = thr, lys or ala
  VARIANT 79: note = x = gly or ala
  VARIANT 80: note = x = ser, thr, leu or val
  VARIANT 82: note = x = thr, ala or leu
  VARIANT 83: note = x = ile or val
  VARIANT 84: note = x = thr or ser
  VARIANT 86: note = x = leu, pro or ala
  VARIANT 88: note = x = ala, ser, thr or pro
  VARIANT 92: note = x = ala, gly or val
  VARIANT 93: note = x = asp or asn
  VARIANT 95: note = x = tyr or his
  VARIANT 97..108: note = x = any amino acid
  VARIANT 109: note = x = phe or ile
  VARIANT 111: note = x = gly or arg
  VARIANT 115: note = x = leu or val
  VARIANT 116: note = x = thr or ser

SEQ ID NO: 238 - protein, 112 aa, synthetic construct
  REGION 1..112: note = VL_Consensus_c
  VARIANT 23..36: note = x = any amino acid
  VARIANT 52..61: note = x = any amino acid
  VARIANT 91..102: note = x = any amino acid
QSVPTQPPSVSGALGQRVTISCXXXXXXXXXXXXXXWYQQIPGMAPKTLIYXXXXXXXXXXDRFSGSKSGSTGSLTITGLQAEDEADYYCXXXXXXXXXXXXFGGGTHLTVL

SEQ ID NO: 239 - protein, 112 aa, synthetic construct
  REGION 1..112: note = VL_Consensus_d
  VARIANT 91..102: note = x = any amino acid
QSVPTQPPSVSGALGQRVTISCTGSSSNIGVGNYVSWYQQIPGMAPKTLIYGNSNRPSGVPDRFSGSKSGSTGSLTITGLQAEDEADYYCXXXXXXXXXXXXFGGGTHLTVL

SEQ ID NO: 240 - protein, 29 aa, Felis catus
  REGION 1..29: note = VH_FW1_Var1
DEQLVESGGDLVKPGGSLRLTCVASGFPF

SEQ ID NO: 241 - protein, 29 aa, Felis catus
  REGION 1..29: note = VH_FW1_Var2
DEQLVESGGDLVKPGGSLRLTCVASGFTL

SEQ ID NO: 242 - protein, 29 aa, Felis catus
  REGION 1..29: note = VH_FW1_Var3
DEQLVESGGDLVKPGGSLRLTCVGSGFTF

SEQ ID NO: 243 - protein, 29 aa, Felis catus
  REGION 1..29: note = VH_FW1_Var4
DVQLVESGGDLVKPGGSLRLACVASGFTF

SEQ ID NO: 244 - protein, 29 aa, Felis catus
  REGION 1..29: note = VH_FW1_Var5
DVQLVESGGDLVKPGGSLRLICVASGFTF

SEQ ID NO: 245 - protein, 29 aa, Felis catus
  REGION 1..29: note = VH_FW1_Var6
DVQLVESGGDLVKPGGSLRLTCVASGFIF

SEQ ID NO: 246 - protein, 29 aa, Felis catus
  REGION 1..29: note = VH_FW1_Var7
DVQLVESGGDLVKPGGSLRLTCVASGFPF

SEQ ID NO: 247 - protein, 29 aa, Felis catus
  REGION 1..29: note = VH_FW1_Var8
DVQLVESGGDLVKPGGSLRLTCVASGFTF

SEQ ID NO: 248 - protein, 29 aa, Felis catus
  REGION 1..29: note = VH_FW1_Var9
DVQVVESGGDLVKPGGSLRLTCVASGFTF

SEQ ID NO: 249 - protein, 29 aa, Felis catus
  REGION 1..29: note = VH_FW1_Var10
DVRLVESGGDLVKPGGSLRLTCVASGFTF

SEQ ID NO: 250 - protein, 29 aa, Felis catus
  REGION 1..29: note = VH_FW1_Var11
DVRLVESGGDRVKPGGSLRLTCMASGFNV

SEQ ID NO: 251 - protein, 29 aa, Felis catus
  REGION 1..29: note = VH_FW1_Var12
EVQLVESGGDLVKPGGSLRLTCVASGFTF

SEQ ID NO: 252 - protein, 29 aa, Felis catus
  REGION 1..29: note = VH_FW1_Var13
QVLLVQSGGDLVKPGGSLRLTCVASGFTF

SEQ ID NO: 253 - protein, 29 aa, Felis catus
  REGION 1..29: note = VH_FW1_Var14
YVQLVESGGDLVKPGGSLRLTCVASGFTF

SEQ ID NO: 254 - protein, 6 aa, Felis catus
  REGION 1..6: note = VH_CDR1_Var1
GSYDMT

SEQ ID NO: 255 - protein, 6 aa, Felis catus
  REGION 1..6: note = VH_CDR1_Var2
NNFAMS

SEQ ID NO: 256 - protein, 6 aa, Felis catus
  REGION 1..6: note = VH_CDR1_Var3
NSYAMS

SEQ ID NO: 257 - protein, 6 aa, Felis catus
  REGION 1..6: note = VH_CDR1_Var4
RGYAMT

SEQ ID NO: 258 - protein, 6 aa, Felis catus
  REGION 1..6: note = VH_CDR1_Var5
RSHWMN

SEQ ID NO: 259 - protein, 6 aa, Felis catus
  REGION 1..6: note = VH_CDR1_Var6
SDYDMS

SEQ ID NO: 260 - protein, 6 aa, synthetic construct
  REGION 1..6: note = Felis catus
SGYSMN

SEQ ID NO: 261 - protein, 6 aa, Felis catus
  REGION 1..6: note = Felis catus
SLYDMS

SEQ ID NO: 262 - protein, 6 aa, Felis catus
  REGION 1..6: note = VH_CDR1_Var9
SNYDMS

SEQ ID NO: 263 - protein, 6 aa, Felis catus
  REGION 1..6: note = VH_CDR1_Var10
SNYGMD

SEQ ID NO: 264 - protein, 6 aa, Felis catus
  REGION 1..6: note = VH_CDR1_Var11
SNYGMS

SEQ ID NO: 265 - protein, 6 aa, synthetic construct
  REGION 1..6: note = Felis catus
SRYGMS

SEQ ID NO: 266 - protein, 6 aa, synthetic construct
  REGION 1..6: note = Felis catus
SSYAMS

SEQ ID NO: 267 - protein, 6 aa, synthetic construct
  REGION 1..6: note = Felis catus
SSYEMN

SEQ ID NO: 268 - protein, 6 aa, Felis catus
  REGION 1..6: note = VH_CDR1_Var15
SSYGMS

SEQ ID NO: 269 - protein, 6 aa, Felis catus
  REGION 1..6: note = VH_CDR1_Var15
SSYYMH

SEQ ID NO: 270 - protein, 6 aa, Felis catus
  REGION 1..6: note = VH_CDR1_Var17
TGDAMS

SEQ ID NO: 271 - protein, 6 aa, Felis catus
  REGION 1..6: note = VH_CDR1_Var18
TNYAMS

SEQ ID NO: 272 - protein, 13 aa, Felis catus
  REGION 1..13: note = VH_FW2_Var1
WVRQAPGKGLQWV

SEQ ID NO: 273 - protein, 13 aa, Felis catus
  REGION 1..13: note = VH_FW2_Var2
WVRQAPGMGLQWV

SEQ ID NO: 274 - protein, 13 aa, Felis catus
  REGION 1..13: note = VH_FW2Var3
WVRQAPGRGLQWV

SEQ ID NO: 275 - protein, 13 aa, Felis catus
  REGION 1..13: note = VH_FW2_Var4
WVRQTPGKGLQWV

SEQ ID NO: 276 - protein, 13 aa, Felis catus
  REGION 1..13: note = VH_CDR2_Var1
AAIAYNGGNTGYA

SEQ ID NO: 277 - protein, 13 aa, Felis catus
  REGION 1..13: note = VH_CDR2_Var2
AAIGHDGSTTAYA

SEQ ID NO: 278 - protein, 13 aa, Felis catus
  REGION 1..13: note = VH_CDR2_Var3
AAIRGSGGVTYYA

SEQ ID NO: 279 - protein, 13 aa, Felis catus
  REGION 1..13: note = VH_CDR2_Var4
AAISGSGDSTYYA

SEQ ID NO: 280 - protein, 13 aa, Felis catus
  REGION 1..13: note = VH_CDR2_Var5
AAISYNGGGTGYS

SEQ ID NO: 281 - protein, 13 aa, Felis catus
  REGION 1..13: note = VH_CDR2_Var6
AANSGTGSSTYYA

SEQ ID NO: 282 - protein, 13 aa, Felis catus
  REGION 1..13: note = VH_CDR2_Var7
ADISGSGGATAYA

SEQ ID NO: 283 - protein, 13 aa, Felis catus
  REGION 1..13: note = VH_CDR2_Var8
AGISGSGITTYYA

SEQ ID NO: 284 - protein, 13 aa, Felis catus
  REGION 1..13: note = VH_CDR2_Var9
AGISTSGGNTYYA

SEQ ID NO: 285 - protein, 12 aa, Felis catus
  REGION 1..12: note = VH_CDR2_Var10
AGITSGGNTYYA

SEQ ID NO: 286 - protein, 13 aa, Felis catus
  REGION 1..13: note = VH_CDR2_Var11
AQISDSGGSTYYA

SEQ ID NO: 287 - protein, 13 aa, Felis catus
  REGION 1..13: note = VH_CDR2_Var12
AYIDNDGSSTYYA

SEQ ID NO: 288 - protein, 13 aa, Felis catus
  REGION 1..13: note = VH_CDR2_Var13
AYIRYDGNTIHYG

SEQ ID NO: 289 - protein, 13 aa, Felis catus
  REGION 1..13: note = VH_CDR2_Var14
AYIRYDGSSTNYA

SEQ ID NO: 290 - protein, 12 aa, Felis catus
  REGION 1..12: note = VH_CDR2_Var15
AYISSGGSTYYA

SEQ ID NO: 291 - protein, 13 aa, Felis catus
  REGION 1..13: note = VH_CDR2_Var16
CAIGGTGSRTLYA

SEQ ID NO: 292 - protein, 13 aa, Felis catus
  REGION 1..13: note = VH_CDR2_Var17
SAISFDGSGTGYA

SEQ ID NO: 293 - protein, 13 aa, Felis catus
  REGION 1..13: note = VH_CDR2_Var18
SALSESGHSTIYA

SEQ ID NO: 294 - protein, 12 aa, Felis catus
  REGION 1..12: note = VH_CDR2_Var19
SSISSGGTTYYA

SEQ ID NO: 295 - protein, 12 aa, Felis catus
  REGION 1..12: note = VH_CDR2_Var20
STIDSGGNTHYI

SEQ ID NO: 296 - protein, 13 aa, Felis catus
  REGION 1..13: note = VH_CDR2_Var21
TDISRSGATTYYA

SEQ ID NO: 297 - protein, 13 aa, Felis catus
  REGION 1..13: note = VH_CDR2_Var22
TTISGSGGSTYYA

SEQ ID NO: 298 - protein, 37 aa, Felis catus
  REGION 1..37: note = VH_FR3_Var1
DSVKGRFTISKDDAENTLYLQMNSLKTEDTATYYCAG

SEQ ID NO: 299 - protein, 37 aa, Felis catus
  REGION 1..37: note = VH_FR3_Var2
DSVKGRFTISRDNAENTLLLQMNSLKTEDTATYYCAR

SEQ ID NO: 300 - protein, 37 aa, Felis catus
  REGION 1..37: note = VH_FR3_Var3
DSVKGRFTISRDNAKNTLSLQMDSLKTEDTATYYCAT

SEQ ID NO: 301 - protein, 37 aa, Felis catus
  REGION 1..37: note = VH_FR3_Var4
DSVKGRFTISRDNAKNTLYLQMDSLKTEDTATYYCTS

SEQ ID NO: 302 - protein, 37 aa, Felis catus
  REGION 1..37: note = VH_FR3_Var5
DSVKGRFTISRDNAKNTLYLQMNGLKTEDTATYYCAR

SEQ ID NO: 303 - protein, 37 aa, Felis catus
  REGION 1..37: note = VH_FR3_Var6
DSVKGRFTISRDNAKNTLYLQMNNLKTEDTATYYCAG

SEQ ID NO: 304 - protein, 37 aa, Felis catus
  REGION 1..37: note = VH_FR3_Var7
DSVKGRFTISRDNAKNTLYLQMNSLKTEDMATYYCAR

SEQ ID NO: 305 - protein, 37 aa, Felis catus
  REGION 1..37: note = VH_FR3_Var8
DSVKGRFTISRDNAKNTLYLQMNSLKTEDTATYYCAK

SEQ ID NO: 306 - protein, 37 aa, Felis catus
  REGION 1..37: note = VH_FR3_Var9
DSVKGRFTISRDNAKNTLYLQMNSLKTEDTATYYCAR

SEQ ID NO: 307 - protein, 37 aa, Felis catus
  REGION 1..37: note = VH_FR3_Var10
DSVKGRFTISRDNAKNTLYLQMNSLKTEDTATYYCAT

SEQ ID NO: 308 - protein, 37 aa, Felis catus
  REGION 1..37: note = VH_FR3_Var11
DSVKGRFTISRDNAMNTLYLQMNSLKTEDAATYYCAR

SEQ ID NO: 309 - protein, 37 aa, Felis catus
  REGION 1..37: note = VH_FR3_Var12
DSVKGRFTISRDNARNTLYLQMNSLKTEDTATYYCAR

SEQ ID NO: 310 - protein, 37 aa, Felis catus
  REGION 1..37: note = VH_FR3_Var13
DSVKGRFTISRDNPKNTLYLQMTSLKTEDTATYYCAR

SEQ ID NO: 311 - protein, 37 aa, Felis catus
  REGION 1..37: note = VH_FR3_Var14
DSVKGRFTISRDNVKNTLYLQMNSLKTEDTATYYCAR

SEQ ID NO: 312 - protein, 37 aa, Felis catus
  REGION 1..37: note = VH_FR3_Var15
DSVKGRFTVSRDNAKNTLYLQMNSLKTEDTATYYCSR

SEQ ID NO: 313 - protein, 22 aa, Felis catus
  REGION 1..22: note = VL_FW1_Var1
QSVVTQPPSVSGALGQAVTISC

SEQ ID NO: 314 - protein, 22 aa, Felis catus
  REGION 1..22: note = VL_FW1_Var2
QSVLTQPPSVSGALGQTVTISC

SEQ ID NO: 315 - protein, 22 aa, Felis catus
  REGION 1..22: note = VL_FW1_Var3
QSVLTQPPSVSGGLGQKVTISC

SEQ ID NO: 316 - protein, 22 aa, synthetic construct
  REGION 1..22: note = VL_FW1_Var4
QPVVTQPPSVSGALGQTVTISC

SEQ ID NO: 317 - protein, 22 aa, Felis catus
  REGION 1..22: note = VL_FW1_Var5
QSVLTQPPSMSGALGQTVTISC

SEQ ID NO: 318 - protein, 22 aa, Felis catus
  REGION 1..22: note = VL_FW1_Var6
QPVLTQPPSVSGSLGQRVTISC

SEQ ID NO: 319 - protein, 22 aa, Felis catus
  REGION 1..22: note = VL_FW1_Var7
QSGPNQPSSVSGALGQRVTISC

SEQ ID NO: 320 - protein, 22 aa, Felis catus
  REGION 1..22: note = VL_FW1_Var8
QSGPNQPSSVSGALGQRVTMSC

SEQ ID NO: 321 - protein, 22 aa, Felis catus
  REGION 1..22: note = VL_FW1_Var9
QSGPNQPSSVSAALGQRVTISC

SEQ ID NO: 322 - protein, 22 aa, Felis catus
  REGION 1..22: note = VL_FW1_Var10
QSGPNQPSSVSGTLGQTITISC

SEQ ID NO: 323 - protein, 22 aa, Felis catus
  REGION 1..22: note = VL_FW1_Var11
QEVVTQETSLSTTPGGTVTLTC

SEQ ID NO: 324 - protein, 22 aa, Felis catus
  REGION 1..22: note = VL_FW1_Var12
QSVVTQPPSVSGALGQRVTISC

SEQ ID NO: 325 - protein, 22 aa, Felis catus
  REGION 1..22: note = VL_FW1_Var13
QPVLTQPPSVSGALGQRVTISC

SEQ ID NO: 326 - protein, 22 aa, Felis catus
  REGION 1..22: note = VL_FW1_Var14
QPVVTQPPSVSGALGQRVTISC

SEQ ID NO: 327 - protein, 22 aa, Felis catus
  REGION 1..22: note = VL_FW1_Var15
QPVLTQPSSLSASPGTTARLTC

SEQ ID NO: 328 - protein, 22 aa, Felis catus
  REGION 1..22: note = VL_FW1_Var16
QPVVTQPPSLSGSLGATARLTC

SEQ ID NO: 329 - protein, 13 aa, Felis catus
  REGION 1..13: note = VL_CDR1_Var1
AGSANNIGITDVN

SEQ ID NO: 330 - protein, 13 aa, Felis catus
  REGION 1..13: note = VL_CDR1_Var2
AGSGSNIGIVDVN

SEQ ID NO: 331 - protein, 13 aa, Felis catus
  REGION 1..13: note = VL_CDR1_Var3
AGSGSNIGIAGVN

SEQ ID NO: 332 - protein, 13 aa, Felis catus
  REGION 1..13: note = VL_CDR1_Var4
AGSANNNGIVGVN

SEQ ID NO: 333 - protein, 13 aa, Felis catus
  REGION 1..13: note = VL_CDR1_Var5
AGSGSNIGVAGVN

SEQ ID NO: 334 - protein, 14 aa, Felis catus
  REGION 1..14: note = VL_CDR1_Var6
TGTSSNIGSGNYVS

SEQ ID NO: 335 - protein, 8 aa, Felis catus
  REGION 1..8: note = VL_CDR1_Var7
TGIDTYVG

SEQ ID NO: 336 - protein, 8 aa, Felis catus
  REGION 1..8: note = VL_CDR1_Var8
TGVDTYVD

SEQ ID NO: 337 - protein, 8 aa, Felis catus
  REGION 1..8: note = VL_CDR1_Var9
TGVDSYVG

SEQ ID NO: 338 - protein, 8 aa, Felis catus
  REGION 1..8: note = VL_CDR1_Var10
SGAGSYVA

SEQ ID NO: 339 - protein, 8 aa, Felis catus
  REGION 1..8: note = VL_CDR1_Var11
TGAGSYVG

SEQ ID NO: 340 - protein, 8 aa, Felis catus
  REGION 1..8: note = VL_CDR1_Var12
TGVGNYVD

SEQ ID NO: 341 - protein, 8 aa, Felis catus
  REGION 1..8: note = VL_CDR1_Var13
TGVGMSVD

SEQ ID NO: 342 - protein, 7 aa, Felis catus
  REGION 1..7: note = VL_CDR1_Var14
TGAGYVG

SEQ ID NO: 343 - protein, 8 aa, Felis catus
  REGION 1..8: note = VL_CDR1_Var15
TGVSVFVD

SEQ ID NO: 344 - protein, 14 aa, Felis catus
  REGION 1..14: note = VL_CDR1_Var16
GSSTGAVTTSNFAS

SEQ ID NO: 345 - protein, 14 aa, Felis catus
  REGION 1..14: note = VL_CDR1_Var17
TGSSSNVGRGNYVS

SEQ ID NO: 346 - protein, 14 aa, Felis catus
  REGION 1..14: note = VL_CDR1_Var18
TGSSPNIGRGNYVS

SEQ ID NO: 347 - protein, 14 aa, Felis catus
  REGION 1..14: note = VL_CDR1_Var19
TGSSSNIGRGNYVS

SEQ ID NO: 348 - protein, 14 aa, Felis catus
  REGION 1..14: note = VL_CDR1_Var20
TLSSGFNVGGYYIS

SEQ ID NO: 349 - protein, 14 aa, Felis catus
  REGION 1..14: note = VL_CDR1_Var21
TLSREVSVGVKGIY

SEQ ID NO: 350 - protein, 15 aa, Felis catus
  REGION 1..15: note = VL_FW2_Var1
WYQQLPGKAPKLLIY

SEQ ID NO: 351 - protein, 15 aa, Felis catus
  REGION 1..15: note = VL_FW2_Var2
WYQQLPGKAPSLLIY

SEQ ID NO: 352 - protein, 15 aa, Felis catus
  REGION 1..15: note = VL_FW2_Var3
WYQQHPRKAPKLLIY

SEQ ID NO: 353 - protein, 15 aa, Felis catus
  REGION 1..15: note = VL_FW2_Var4
WYQQLPGKAPQLLIY

SEQ ID NO: 354 - protein, 15 aa, Felis catus
  REGION 1..15: note = VL_FW2_Var5
WYQQLPGKAPKALIY

SEQ ID NO: 355 - protein, 15 aa, Felis catus
  REGION 1..15: note = VL_FW2_Var6
WYRQVPGIAPSLLIY

SEQ ID NO: 356 - protein, 15 aa, Felis catus
  REGION 1..15: note = VL_FW2_Var7
WFQQIPGMAPKTIIV

SEQ ID NO: 357 - protein, 15 aa, Felis catus
  REGION 1..15: note = VL_FW2_Var8
WYQQIPGMAPKTIIY

SEQ ID NO: 358 - protein, 15 aa, Felis catus
  REGION 1..15: note = VL_FW2_Var9
WYQQIPGRAPKTIIY

SEQ ID NO: 359 - protein, 15 aa, Felis catus
  REGION 1..15: note = VL_FW2_Var10
WYQQVPGMAPKTIIY

SEQ ID NO: 360 - protein, 15 aa, Felis catus
  REGION 1..15: note = VL_FW2_Var11
WYQQIPGMAPKTVIY

SEQ ID NO: 361 - protein, 15 aa, Felis catus
  REGION 1..15: note = VL_FW2_Var12
WVQQKPYQRFQGLVG

SEQ ID NO: 362 - protein, 15 aa, Felis catus
  REGION 1..15: note = VL_FW2_Var13
WYQQLSGTPPKLLIY

SEQ ID NO: 363 - protein, 15 aa, Felis catus
  REGION 1..15: note = VL_FW2_Var14
WYQQLSGTAPKVLIY

SEQ ID NO: 364 - protein, 15 aa, Felis catus
  REGION 1..15: note = VL_FW2_Var15
WYQQLSGTAPKLLIY

SEQ ID NO: 365 - protein, 15 aa, Felis catus
  REGION 1..15: note = VL_FW2_Var16
WYQQVSGTAPKLLIY

SEQ ID NO: 366 - protein, 19 aa, Felis catus
  REGION 1..19: note = VL_FW2_Var17
WFQQKPGSPPRYLLYYYSD

SEQ ID NO: 367 - protein, 19 aa, Felis catus
  REGION 1..19: note = VL_FW2_Var18
WYQQKPGSPPRYFLHYYSD

SEQ ID NO: 368 - protein, 10 aa, Felis catus
  REGION 1..10: note = VL_CDR2_Var1
ANNRRPSSVP

SEQ ID NO: 369 - protein, 10 aa, Felis catus
  REGION 1..10: note = VL_CDR2_Var2
ASDRRPSGVP

SEQ ID NO: 370 - protein, 10 aa, Felis catus
  REGION 1..10: note = VL_CDR2_Var3
GSSNRPSGVP

SEQ ID NO: 371 - protein, 10 aa, Felis catus
  REGION 1..10: note = VL_CDR2_Var4
ASTRRPSGVP

SEQ ID NO: 372 - protein, 10 aa, Felis catus
  REGION 1..10: note = L_CDR2_Var5
ENFKRPSGVP

SEQ ID NO: 373 - protein, 10 aa, synthetic construct
  REGION 1..10: note = VL_CDR2_Var6
GNTNRPSGVP

SEQ ID NO: 374 - protein, 10 aa, synthetic construct
  REGION 1..10: note = VL_CDR2_Var6
YDSNRPSGVP

SEQ ID NO: 375 - protein, 10 aa, Felis catus
  REGION 1..10: note = VL_CDR2_Var8
DNSNRPSGVP

SEQ ID NO: 376 - protein, 10 aa, Felis catus
  REGION 1..10: note = VL_CDR2_Var9
DDNKRPSGVP

SEQ ID NO: 377 - protein, 10 aa, Felis catus
  REGION 1..10: note = VL_CDR2_Var10
GNRYRPSGVP

SEQ ID NO: 378 - protein, 10 aa, Felis catus
  REGION 1..10: note = VL_CDR2_Var11
RNSNRLSEVP

SEQ ID NO: 379 - protein, 10 aa, Felis catus
  REGION 1..10: note = VL_CDR2_Var12
GNSYRPSGVP

SEQ ID NO: 380 - protein, 10 aa, Felis catus
  REGION 1..10: note = VL_CDR2_Var13
RNTNRLSEVP

SEQ ID NO: 381 - protein, 10 aa, Felis catus
  REGION 1..10: note = VL_CDR2_Var14
GTSYRNPGVP

SEQ ID NO: 382 - protein, 10 aa, Felis catus
  REGION 1..10: note = VL_CDR2_Var15
GNSDRLSGVP

SEQ ID NO: 383 - protein, 10 aa, Felis catus
  REGION 1..10: note = VL_CDR2_Var16
GDKNRPSGVP

SEQ ID NO: 384 - protein, 10 aa, Felis catus
  REGION 1..10: note = VL_CDR2_Var17
GDTNRPSGVP

SEQ ID NO: 385 - protein, 10 aa, Felis catus
  REGION 1..10: note = VL_CDR2_Var18
SDKHQGPGVP

SEQ ID NO: 386 - protein, 10 aa, Felis catus
  REGION 1..10: note = VL_CDR2_Var19
STNELGPGVP

SEQ ID NO: 387 - protein, 29 aa, Felis catus
  REGION 1..29: note = VL_FW3_Var1
DRFSGSKSGATGSLTISGLQAEDEADYYC

SEQ ID NO: 388 - protein, 29 aa, Felis catus
  REGION 1..29: note = VL_FW3_Var2a
ERISGSRSGNTGSLTITGLQTEDEADYYC

SEQ ID NO: 389 - protein, 29 aa, Felis catus
  REGION 1..29: note = VL_FW3_Var3
DRFSGSKSGNTGSLTITGLQAEDEADYYC

SEQ ID NO: 390 - protein, 29 aa, Felis catus
  REGION 1..29: note = VL_FW3_Var4
DRFSGSKSGNTGSLTITGLQAEDEADYYC

SEQ ID NO: 391 - protein, 29 aa, Felis catus
  REGION 1..29: note = VL_FW3_Var5
ERLSGSKSGNTGSLTITGLQPEDEADYYC

SEQ ID NO: 392 - protein, 29 aa, Felis catus
  REGION 1..29: note = VL_FW3_Var6
DRFSGSKSGSSGSLTITGLQADDEVDYYC

SEQ ID NO: 393 - protein, 29 aa, Felis catus
  REGION 1..29: note = VL_FW3_Var7
DRFSGSKSGNTGTLTITGLQAEDEADYYC

SEQ ID NO: 394 - protein, 29 aa, Felis catus
  REGION 1..29: note = VL_FW3_Var8
DRFSGSKSGSTGTLAITGLQAEDEGDYYC

SEQ ID NO: 395 - protein, 29 aa, Felis catus
  REGION 1..29: note = VL_FW3_Var9
DRFSGSKSGNTGTLTITGLQAEDEADYYC

SEQ ID NO: 396 - protein, 29 aa, Felis catus
  REGION 1..29: note = VL_FW3_Var10
DRFSGSKSGNTGTLTITGLQAEDEADYYC

SEQ ID NO: 397 - protein, 29 aa, Felis catus
  REGION 1..29: note = VL_FW3_Var11
ARFSGSKSGSTGTLTITGLQAEDEANYYC

SEQ ID NO: 398 - protein, 29 aa, Felis catus
  REGION 1..29: note = VL_FW3_Var12
DRFSGSKSGSTGTLTITGLQAEDEADYYC

SEQ ID NO: 399 - protein, 29 aa, Felis catus
  REGION 1..29: note = VL_FW3_Var13
NRFSGSKSGSTATLTITGLQAEDEADYYC

SEQ ID NO: 400 - protein, 29 aa, Felis catus
  REGION 1..29: note = VL_FW3_Var13
DRFSGSKSGSTGTLTITGLQAEDEADYYC

SEQ ID NO: 401 - protein, 29 aa, Felis catus
  REGION 1..29: note = VL_FW3_Var15
DRFSGSKSGITATLTITGLQAEDEADYYC

SEQ ID NO: 402 - protein, 29 aa, Felis catus
  REGION 1..29: note = VL_FW3_Var16
ARFSGSLVGQKAVLTITGAQSEDEAEYYC

SEQ ID NO: 403 - protein, 29 aa, Felis catus
  REGION 1..29: note = VL_FW3_Var17
DRFSGSKSGTTGSLTITGLQTEDEADYYC

SEQ ID NO: 404 - protein, 29 aa, Felis catus
  REGION 1..29: note = VL_FW3_Var18
DRFSGSKSGSTGSLTITGLQAEDEADYYC

SEQ ID NO: 405 - protein, 29 aa, synthetic construct
  REGION 1..29: note = VH_FW1_Var15
DVQLVESGGDLVQPGGSLRLTCVASGFTF

SEQ ID NO: 406 - protein, 29 aa, Felis catus
  REGION 1..29: note = VH_FW1_Var16
DVQLVQSGGDLVKPGGSLRLTCVASGFTF

SEQ ID NO: 407 - protein, 13 aa, synthetic construct
  REGION 1..13: note = VH_CDR2_Var23
AAISYSGGGTGYS

SEQ ID NO: 408 - protein, 13 aa, synthetic construct
  REGION 1..13: note = VH_CDR2_Var24
AAISGTGSSTYYA

SEQ ID NO: 409 - protein, 13 aa, synthetic construct
  REGION 1..13: note = VH_CDR2_Var25
AAIAYSGGNTGYA

SEQ ID NO: 410 - protein, 10 aa, synthetic construct
  REGION 1..10: note = VL_CDR2_Var20
GNTDRLSGVP

SEQ ID NO: 411 - protein, 29 aa, synthetic construct
  REGION 1..29: note = VL_FW3_Var19
DRFSGSKSGSSGSLTITGLQAEDEVDYYC

SEQ ID NO: 412 - protein, 13 aa, synthetic construct
  REGION 1..13: note = VH_FW2_maturation_module
WVRQAPGKGLQWV

SEQ ID NO: 413 - protein, 112 aa, synthetic construct
  REGION 1..112: note = VL_Consensus _e
  VARIANT 91..102: note = x = any amino acid
QSGPNQPSSVSGALGQRVTISCTGSSSNIGRGNYVSWYQQVPGMAPKTLIYGNSNRPSGVPDRFSGSKSGSTGSLTITGLQAEDEADYYCXXXXXXXXXXXXFGGGTHLTVL

SEQ ID NO: 414 - protein, 37 aa, synthetic construct
DSVKGRFTISRDNAKNTLYLQMNSLKTEDTATYYCAN

SEQ ID NO: 415 - protein, 29 aa, synthetic construct
DRFSGSKSGSTGSLTITGLQADDEADYYC

SEQ ID NO: 416 - protein, 112 aa, synthetic construct; identical to SEQ ID NO: 413
  VARIANT 91: note = X can be any naturaly occuring amino acid
  VARIANT 92: note = X can be any naturally occurring amino acid
  VARIANT 93: note = X can be any naturally occurring amino acid
  VARIANT 93: note = X can be any naturally occurring amino acid
  VARIANT 94: note = X can be any naturally occurring amino acid
  VARIANT 94: note = X can be any naturally occurring amino acid
  VARIANT 95: note = X can be any naturally occurring amino acid
  VARIANT 96: note = X can be any naturally occurring amino acid
  VARIANT 97: note = X can be any naturally occurring amino acid
  VARIANT 98: note = X can be any naturally occurring amino acid
  VARIANT 99: note = X can be any naturally occurring amino acid
  VARIANT 100: note = X can be any naturally occurring amino acid
  VARIANT 101: note = X can be any naturally occurring amino acid
  VARIANT 102: note = X can be any naturally occurring amino acid

(19) United States
(12) Patent Application Publication
(10) Pub. No.: US 2025/0154688 A1
(43) Pub. Date: May 15, 2025

(54) FELINE ANTIBODY LIBRARY

(71) Applicant: adivo GmbH, Puchheim (DE)

(72) Inventors: Philipp BAER, Puchheim (DE); Andrea STERNER, Puchheim (DE); Markus WALDHUBER, Puchheim (DE)

(21) Appl. No.: 18/839,056

(22) PCT Filed: Feb. 17, 2023
(86) PCT No.: PCT/EP2023/054095

§ 371 Date: Aug. 16, 2024

(30) Foreign Application Priority Data
Feb. 18, 2022 (EP) 22157611.9

(51) Int. Cl. C40B 40/10; C07K 16/00; C12N 15/70

(52) U.S. Cl. CPC C40B 40/10; C07K 16/005; C12N 15/70; C07K 2317/20; C07K 2317/55; C07K 2317/565; C07K 2317/567; C07K 2317/622

(57) ABSTRACT

The present invention relates to and provides polypeptide libraries, such as synthetic antibody libraries which are suitable for selection of fully feline antibodies. The invention also relates to synthetic nucleic acid sequences which encode individual or collections of feline antibodies, i.e. nucleic acid sequences encoding feline antibody libraries. Methods for generating and using such libraries are provided. In particular, the invention relates to the preparation of a library of fully feline antibodies by the use of pre-evaluated synthetic sequences to generate a library of fully feline antibodies having favorable biophysical properties.

### FIELD OF THE INVENTION

The present invention relates to and provides polypeptide libraries, such as synthetic antibody libraries which are suitable for selection of fully feline antibodies. The invention also relates to synthetic nucleic acid sequences which encode individual or collections of feline antibodies, i.e. nucleic acid sequences encoding feline antibody libraries. Methods for generating and using such libraries are provided. In particular, the invention relates to the preparation of a library of fully feline antibodies by the use of pre-evaluated synthetic sequences to generate a library of fully feline antibodies having favorable biophysical properties.

### BACKGROUND OF THE INVENTION

In the following discussion certain articles and methods are described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

It has been more than three decades since the first monoclonal antibody (mAb), Orthoclone OKT3, was approved by the United States Food and Drug Administration (US FDA) in 1986 to prevent kidney transplant rejection (Ecker et al. 2015). Since then, technologies to generate and optimize human therapeutic mAbs have significantly improved, making these a predominant class of new drugs developed in recent years. The market for mAbs has experienced substantial growth and drugs for treating various human diseases, including many cancers, autoimmune, metabolic and infectious diseases are available. As of December 2021, over 100 therapeutic mAbs have been approved by regulatory authorities, and more than 80 are in late stage clinical development (Kaplon and Reichert 2021).

Companion animals such as dogs and cats develop comparable diseases than humans underlying similar or even the same pathologic mechanisms. Surprisingly though, the use of therapeutic mAbs in animal health only recently started to evolve and there are only a few approved antibodies available to date (e.g. https://www.zoetisus.com/products/dogs/cytopoint/#). The limited progress reflects the fact that the generation of species-specific mAbs is technically challenging and experience in the development of antibodies for companion animals is only gradually developing.

There are only a few technological approaches available to generate therapeutic antibodies for companion animals such as dogs or cats, namely the modification of existing compounds and the use of transgenic animals. Methods to “caninize” or “felinize” antibodies have been disclosed. For example, the generation of a caninised anti-NGF mAb from an existing rat-antibody is described that uses an algorithm for comparison of the starting antibody with canine immunoglobulin sequences to identify modifications necessary to make the sequence pet-like (Gearing et al. 2013). Similar methods were applied also for the generation of feline antibodies (Gearing et al. 2016). However, even subtle changes of the protein sequence of an antibody can result in significant loss of efficacy and altered biophysical properties, rendering such methods time consuming and prone to failure. More advanced technologies relate to transgenic rodents that express canine immunoglobulins (Wabl May 23, 2017). Disadvantages are the need to sacrifice animals for the initial antibody discovery process and that the immunization process is hardly controllable.

In this respect, in vitro selection methods such as phage display offer huge advantages as these allow a tailored antibody selection process. Only recently, a synthetic phage display library comprising fully canine antibody fragments has been disclosed (Tiller et al. Jun. 21, 2018). This library is built on pre-selected and tested heavy and light chain pairings based on germline genes, thus expected to yield antibodies that are not immunogenic when administered to a dog patient. Diversity has been introduced in the CDR3 of both the heavy and light chain, the CDRs were designed in accordance to the natural amino acid and length distribution of analyzed canine antibody sequences. The members of the library were also selected for advantageous properties, such as a high monomeric content and a high thermal stability.

Over the last years, there is growing interest in the cat not only as a companion animal but also as a model for specific diseases. To the inventors' knowledge, neither a fully synthetic feline antibody library with a predefined heavy and light chain composition nor any other reliable collection of feline antibodies has been disclosed yet.

Within the present invention, rational analysis of experimentally selected naturally occurring feline antibody sequences and sophisticated design of the libraries, led to the first fully synthetic feline antibody library that can be used for biomedical research.

The feline immune system achieves diversity within the antibody repertoire by V (D) J recombination, a process in which a series of variable genes (V), diversity genes (D), and junction genes (J) are recombined to create the variable domain of an antibody. Additional processes such as somatic hypermutations, insertions and deletions further increase the diversity of the antibody variable region. Also, felines show the canonical mammalian organization of the IGH genome and the IG locus has been described (Cho et al. 1998), consisting of 24 IGHV genes, 13 IGKV and 47 IGLV genes (Olivieri et al. 2014). Domestic cats have been studied in detail immunologically (Baldwin and Denham 1994; Kanai et al. 2000; Klotz et al. 1985; Schultz et al. 1974); Two allelic sequences referred to as feline IgG1a and 1b have been described (Kanai et al. 2000) which function similar to human IgG1 and are expected to induce strong effector function in vivo (Strietzel et al. 2014). The same authors report the presence of a rare IgG sequence, now referred to as feline IgG2. This additional IgG does not bind to recombinant fFcYRI or fFcYRIII and has negligible binding to C1q indicative of lack of effector function. Constant regions of feline kappa (AF198257.1) and lambda regions (XM 003994910.1, E07339.1) have also been disclosed.

However, the actual, expressed feline antibody repertoire is less well described. Next-generation sequencing (NGS) data of feline antibodies revealed some insights in the architecture and abundance of feline antibodies as well as to CDR3 diversity (Steiniger et al. 2017). Even more striking than in dog (Steiniger et al. 2014), the feline antibody repertoire is dominated by one heavy chain subgroup, homologous to human VH3. Around 99% of all analyzed sequences in this study clustered in this subgroup. A characteristic feature described for many domesticated species including cats, dogs, sheep, cattle, and horse is the preferential expression of lambda light chains (Sitnikova and Su 1998; Steiniger et al. 2017). These results were also confirmed by NGS analyses performed in-house, highlighting the striking bias towards the usage of VH3 type heavy and lambda light chains.

The HCDR3 length distribution of feline antibodies shows a nearly normal distribution (Steiniger et al. 2017) and is similar in length to canine repertoire (Steiniger et al. 2014).

Only few feline therapeutic antibodies have been described (Doki et al. 2016; Gruen et al. 2016; Enomoto et al. 2019; Walters et al. 2021). Of note, described candidates are derived from rodent antibodies that underwent modifications to make them compatible with the feline immune system. Due to their origin, the light chains are of the kappa subtype which is uncommon in cats.

In conclusion, the natural pairing of feline antibody heavy and light chains is hardly characterized.

However, the identification of stable VH/VL combinations is of utmost importance as these represent the basis for a functional antibody library. Naturally occurring immunoglobulin sequences encoded by felines are expected to be not immunogenic in cats.

### SUMMARY OF INVENTION

Heavy and light sequences were PCR-amplified from cat bone marrow cDNA. Obtained sequences were cloned into a phage display vector to produce phages comprising random pairings of VH and VL sequences in single chain format (scFv) and containing a FLAG-tag. Following phage preparation, an anti-FLAG pulldown was performed to enrich only phage particles displaying intact FLAG-scFv fragments. The phage output was cloned into a bacterial expression vector and expression levels of individual feline scFvs were determined by ELISA. Clones with high expression levels were subsequently sequenced but also converted into the Fab-format. Expression of soluble Fab-fragments was confirmed by ELISA. Following these selection steps, feline antibody sequences were identified that fulfill the following criteria: (i) these are compatible with phage display, show good expression levels (ii) as scFv and (iii) Fab fragments and thus represent ideal scaffolds to build a synthetic feline antibody library with pre-selected and tested framework regions. Selection of VH and VL sequences and respective pairings based on the described approach involving soluble scFv expression in a first step and Fab fragments in a subsequent step resulted in a library with a surprisingly superior conversion rate from clones initially identified as scFv format to expression as Fabs and also full length IgG antibodies. To our knowledge, this is the first experimental approach to identify a collection of stable feline heavy and light chain combinations to develop an in-vitro fully feline antibody selection technology.

Accordingly, based on the observed properties and determined sequences, the present invention in one aspect provides a polypeptide library comprising VL polypeptide members comprising VL FW1-CDR1-FW2-CDR2-FW3 segments comprising at least one sequence selected from SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, and/or SEQ ID NO: 137. Preferably the at least one sequence comprised in the VL FW1-CDR1-FW2-CDR2-FW3 segments comprised in the VL polypeptide is from selected from SEQ ID NO: 73, SEQ ID NO: 74, SEQ ID NO: 75, SEQ ID NO: 76, SEQ ID NO: 78, SEQ ID NO: 84, SEQ ID NO: 85, SEQ ID NO: 86, SEQ ID NO: 88, SEQ ID NO: 91, SEQ ID NO: 92, SEQ ID NO: 95, SEQ ID NO: 110, SEQ ID NO: 112, SEQ ID NO: 119, SEQ ID NO: 123, SEQ ID NO: 125, SEQ ID NO: 128, SEQ ID NO: 129, SEQ ID NO: 133, SEQ ID NO: 134, and/or SEQ ID NO: 135.

In a further aspect the invention relates to a polypeptide library comprising VH polypeptide members comprising FW1-CDR1-FW2-CDR2-FW3 segments comprising at least one sequence selected from SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, and SEQ ID NO: 202. Preferably the at least one sequence comprised in the VH FW1-CDR1-FW2-CDR2-FW3 segments comprised in the VH polypeptide is from selected from SEQ ID NO: 138, SEQ ID NO: 139, SEQ ID NO: 140, SEQ ID NO: 141, SEQ ID NO: 143, SEQ ID NO: 149, SEQ ID NO: 150SEQ ID NO: 151, SEQ ID NO: 153, SEQ ID NO: 156, SEQ ID NO: 157, SEQ ID NO: 160, SEQ ID NO: 175, SEQ ID NO: 177, SEQ ID NO: 184, SEQ ID NO: 188, SEQ ID NO: 190, SEQ ID NO: 193, SEQ ID NO: 194, SEQ ID NO: 198, SEQ ID NO: 199, and SEQ ID NO: 200.

Preferred VL and VH FW1-CDR1-FW2-CDR2-FW3 segments exhibited high expression levels as scFv and as Fab fragments.

More specifically the present invention provides a polypeptide library comprising at least one VL/VH polypeptide member combination comprising a sequence combination selected from SEQ ID NO: 73 and SEQ ID NO: 138, SEQ ID NO: 74 and SEQ ID NO: 139, SEQ ID NO: 75 and SEQ ID NO: 140, SEQ ID NO: 76 and SEQ ID NO: 141, SEQ ID NO: 77 and SEQ ID NO: 142, SEQ ID NO: 78 and SEQ ID NO: 143, SEQ ID NO: 79 and SEQ ID NO: 144, SEQ ID NO: 80 and SEQ ID NO: 145, SEQ ID NO: 81 and SEQ ID NO: 146, SEQ ID NO: 82 and SEQ ID NO: 147, SEQ ID NO: 83 and SEQ ID NO: 148, SEQ ID NO: 84 and SEQ ID NO: 149, SEQ ID NO: 85 and SEQ ID NO: 150, SEQ ID NO: 86 and SEQ ID NO: 151, SEQ ID NO: 87 and SEQ ID NO: 152, SEQ ID NO: 88 and SEQ ID NO: 153, SEQ ID NO: 89 and SEQ ID NO: 154, SEQ ID NO: 90 and SEQ ID NO: 155, SEQ ID NO: 91 and SEQ ID NO: 156, SEQ ID NO: 92 and SEQ ID NO: 157, SEQ ID NO: 93 and SEQ ID NO: 158, SEQ ID NO: 94 and SEQ ID NO: 159, SEQ ID NO: 95 and SEQ ID NO: 160, SEQ ID NO: 96 and SEQ ID NO: 161, SEQ ID NO: 97 and SEQ ID NO: 162, SEQ ID NO: 98 and SEQ ID NO: 163, SEQ ID NO: 99 and SEQ ID NO: 164, SEQ ID NO: 100 and SEQ ID NO: 165, SEQ ID NO: 101 and SEQ ID NO: 166, SEQ ID NO: 102 and SEQ ID NO: 167, SEQ ID NO: 103 and SEQ ID NO: 168, SEQ ID NO: 104 and SEQ ID NO: 169, SEQ ID NO: 105 and SEQ ID NO: 170, SEQ ID NO: 106 and SEQ ID NO: 171, SEQ ID NO: 107 and SEQ ID NO: 172, SEQ ID NO: 108 and SEQ ID NO: 173, SEQ ID NO: 109 and SEQ ID NO: 174, SEQ ID NO: 110 and SEQ ID NO: 175, SEQ ID NO: 111 and SEQ ID NO: 176, SEQ ID NO: 112 and SEQ ID NO: 177, SEQ ID NO: 113 and SEQ ID NO: 178, SEQ ID NO: 114 and SEQ ID NO: 179, SEQ ID NO: 115 and SEQ ID NO: 180, SEQ ID NO: 116 and SEQ ID NO: 181, SEQ ID NO: 117 and SEQ ID NO: 182, SEQ ID NO: 118 and SEQ ID NO: 183, SEQ ID NO: 119 and SEQ ID NO: 184, SEQ ID NO: 120 and SEQ ID NO: 185, SEQ ID NO: 121 and SEQ ID NO: 186, SEQ ID NO: 122 and SEQ ID NO: 187, SEQ ID NO: 123 and SEQ ID NO: 188, SEQ ID NO: 124 and SEQ ID NO: 189, SEQ ID NO: 125 and SEQ ID NO: 190, SEQ ID NO: 126 and SEQ ID NO: 191, SEQ ID NO: 127 and SEQ ID NO: 192, SEQ ID NO: 128 and SEQ ID NO: 193, SEQ ID NO: 129 and SEQ ID NO: 194, SEQ ID NO: 130 and SEQ ID NO: 195, SEQ ID NO: 131 and SEQ ID NO: 196, SEQ ID NO: 132 and SEQ ID NO: 197, SEQ ID NO: 133 and SEQ ID NO: 198, SEQ ID NO: 134 and SEQ ID NO: 199, SEQ ID NO: 135 and SEQ ID NO: 200, SEQ ID NO: 136 and SEQ ID NO: 201, and/or SEQ ID NO: 137 and SEQ ID NO: 202. Preferably, the polypeptide library comprises at least one VL/VH polypeptide member combination comprising a sequence combination selected from SEQ ID NO: 73 and SEQ ID NO: 138, SEQ ID NO: 74 and SEQ ID NO: 139, SEQ ID NO: 75 and SEQ ID NO: 140, SEQ ID NO: 76 and SEQ ID NO: 141, SEQ ID NO: 78 and SEQ ID NO: 143, SEQ ID NO: 84 and SEQ ID NO: 149, SEQ ID NO: 85 and SEQ ID NO: 150, SEQ ID NO: 86 and SEQ ID NO: 151, SEQ ID NO: 88 and SEQ ID NO: 153, SEQ ID NO: 91 and SEQ ID NO: 156, SEQ ID NO: 92 and SEQ ID NO: 157, SEQ ID NO: 95 and SEQ ID NO: 160, SEQ ID NO: 110 and SEQ ID NO: 175, SEQ ID NO: 112 and SEQ ID NO: 177, SEQ ID NO: 119 and SEQ ID NO: 184, SEQ ID NO: 123 and SEQ ID NO: 188, SEQ ID NO: 125 and SEQ ID NO: 190, SEQ ID NO: 128 and SEQ ID NO: 193, SEQ ID NO: 129 and SEQ ID NO: 194, SEQ ID NO: 133 and SEQ ID NO: 198, SEQ ID NO: 134 and SEQ ID NO: 199, and/or SEQ ID NO: 135 and SEQ ID NO: 200.

Based on an alignment (FIG. 1) of the preferred members as identified above, the inventors were able to derive a consensus sequence of preferred polypeptide VH FW1-HCDR1-FW2-HCDR2-FW3-HCDR3-FW4 as a further aspect of the invention, wherein:
FW1 is X1, X2, X3, X4, X5, X6, X7, X8, X9, X10, X11, X12, X13, X14, X15, X16, X17, X18, X19, X20, X21, X22, X23, X24, X25, X26, X27, X28, X29
HCDR1 is X30, X31, X32, X33, X34, X35
FW2 is X36, X37, X38, X39, X40, X41, X42, X43, X44, X45, X46, X47, X48
HCDR2 is X49, X50, X51, X52, X52a, X53, X54, X55, X56, X57, X58, X59, X60
FW3 is X61, X62, X63, X64, X65, X66, X67, X68, X69, X70, X71, X72, X73, X74, X75, X76, X77, X78, X79, X80, X81, X82, X83, X84, X85, X86, X87, X88, X89, X90, X91, X92, X93, X94, X95, X96, X97
HCDR3 is X98, X99, X100, X101, X101a, X101b, X101c, X101d, X101e, X101f, X101g, X102, X103, X104
FW4 is X105, X106, X107, X108, X109, X111, X112, X113, X114, X115;
   wherein X1 denotes the first amino acid position, X2 denotes the second amino acid position, and so on, and the nature of the amino acid at the respective position is defined as follows:

| | | | | |
|---|---|---|---|---|
| X1 is D, Q, E or Y, | X2 is V or E, | X3 is Q, L or R, | X4 is L, | X5 is V, |
| X6 is E or Q, | X7 is S, | X8 is G, | X9 is G, | X10 is D, |
| X11 is L or R, | X12 is V, | X13 is K or Q, | X14 is P, | X15 is G, |
| X16 is G, | X17 is S, | X18 is L, | X19 is R, | X20 is L, |
| X21 is T, I or A, | X22 is C, | X23 is V or M, | X24 is A or G, | X25 is S, |
| X26 is G, | X27 is F, | X28 is T, P, I or N | X29 is F, V or L, | |
| X30 is S, R, G, T or N, | X31 is S, R, G, N, D | X32 is Y, H, F or D, | X33 is G, W, D, E, A, S or Y, | X34 is M, |
| X35 is S, N, T, D or H, | or L, | | | |
| X36 is W, | X37 is V, | X38 is R, | X39 is Q, | X40 is A or T |
| X41 is P, | X42 is G, | X43 is K or M, | X44 is G, | X45 is L, |
| X46 is Q, | X47 is W, | X48 is V, | | |
| X49 is A, S or T, | X50 is A, S, Y, G, T, Q | X51 is I, N or L, | X52 is S, G, R, D, A or -, | X52a is G, F, |
| | or D, | | | Y, S, N, D or R, |
| X53 is S, T, D, G or N, | X54 is G, | X55 is G, S, I, H, D or A, | X56 is S, A, T, V or N, | X57 is T or I, |
| X58 is Y, A, G, N or H, | X59 is Y, | X60 is A, S, G or I, | | |
| X61 is D, | X62 is S, | X63 is V, | X64 is K, | X65 is G, |
| X66 is R, | X67 is F, | X68 is T, | X69 is I or V, | X70 is S, |
| X71 is R or K, | X72 is D, | X73 is N or D, | X74 is A, P or V | X75 is K, R, M or E, |
| X76 is N, | X77 is T, | X78 is L, | X79 is Y or L, | X80 is L, |
| X81 is Q, | X82 is M, | X83 is N, T or D, | X84 is S, G or N, | X85 is L, |
| X86 is K, | X87 is T, | X88 is E, | X89 is D, | X90 is T, A or M, |
| X91 is A, | X92 is T, | X93 is Y, | X94 is Y, | X95 is C, |
| X96 is A, S or T | X97 is R, K, N, G or T, | | | |
| X105 is W, | X106 is G, | X107 is Q or R, | X108 is G, | X109 is A, T or V, |
| X110 is L or P, | X111 is V, | X112 is T, | X113 is V, | X114 is S, |
| X115 is S, | | | | |

(Bold and underlined amino acids are preferred amino acid, ″-″ defines an empty position)

Accordingly, the VH FW1-HCDR1-FW2-HCDR2-FW3-HCDR3-FW4 segment may have the sequence:

(SEQ ID NO 232)
(D/Q/Y/E)(V/E)(Q/L/R)LV(E/Q)SGGD(L/R)V(K/Q)PGGSLR
L(T/I/A)C(V/M)(A/G)SGF(T/P/I/N)(F/V/L)XaaXaaXaaXa
aXaaXaaWVRQ(A/T)PG(K/M)GLQWVXaaXaaXaaXaaXaaXaaXaa
XaaXaaXaaXaaXaaXaaDSVKGRFT(I/V)S(R/K)D(N/D)(A/P/
V)(K/R/M/E)NTL(Y/L)LQM(N/T/D)(S/G/N)LKTED(T/A/M)A
TYYC(A/S/T)(R/K/N/T/G)XaaXaaXaaXaaXaaXaaXaaXaaXaa
XaaXaaXaaXaaXaaWG(Q/R)G(A/T/V)(L/P)VTVSS,,

wherein the amino acids of the HCDRs are designated as variable amino acids (Xaa). Xaa may be any one of A (Ala), R (Arg), N (Asn), D (Asp), C (Cys), Q (Gln), E (Glu), G (Gly), H (His), I (Ile), L (Leu), K (Lys), M (Met), F (Phe), P (Pro), S (Ser), T (Thr), W (Trp), Y (Tyr), or V (Val).

Preferably, the VH FW1-HCDR1-FW2-HCDR2-FW3-HCDR3-FW4 segment may have the sequence:

(SEQ ID NO 233)
(D/Q/Y/E)(V/E)(Q/L/R)LV(E/Q)SGGD(L/R)V(K/Q)PGGSLR
L(T/I/A)C(V/M)(A/G)SGF(T/P/I/N)(F/V/L)(S/R/G/T/N)
(S/R/G/N/D/L)(Y/H/F/D)(G/W/D/E/A/S/Y)M(S/N/T/D/H)
WVRQ(A/T)PG(K/M)GLQWV(A/S/T)(A/S/Y/G/T/Q/D)(I/N/
L)(S/G/R/D/A)(G/F/Y/S/N/D/R)(S/T/D/G/N)G(G/S/I/H/
D/A)(S/A/T/V/N)(T/I)(Y/A/G/N/H)Y(A/S/G/I)DSVKGRFT
(I/V)S(R/K)D(N/D)(A/P/V)(K/R/M/E)NTL(Y/L)LQM(N/T/
D)(S/G/N)LKTED(T/A/M)ATYYC(A/S/T)(R/K/N/T/G)XaaXa
aXaaXaaXaaXaaXaaXaaXaaXaaXaaXaaXaaXaaWG(Q/R)G(A/
T/V)(L/P)VTVSS,

wherein the amino acids of the HCDRs 1 and 2 are defined in the sequence.

More preferably, the VH FW1-HCDR1-FW2-HCDR2-FW3-HCDR3-FW4 segment may have the sequence:

(SEQ ID NO 234)
DVQLVESGGDLVKPGGSLRLTCVASGFTFXaaXaaXaaXaaXaaXaaWV
RQAPGKGLQWVXaaXaaXaaXaaXaaXaaXaaXaaXaaXaaXaaXaaXa
aDSVKGRFTISRDNAKNTLYLQMNSLKTEDTATYYCARXaaXaaXaaXa
aXaaXaaXaaXaaXaaXaaXaaXaaXaaXaaWGQGALVTVSS,
or
(SEQ ID NO 235)
DVQLVESGGDLVKPGGSLRLTCVASGFTFSSYGMSWVRQAPGKGLQWVA
AISGSGGSTYYADSVKGRFTISRDNAKNTLYLQMNSLKTEDTATYYCAR
XaaXaaXaaXaaXaaXaaXaaXaaXaaXaaXaaXaaXaaXaaWGQGALV
TVSS.

Also for the VL polypeptide, the inventors were able to derive a consensus sequence of preferred polypeptide VL FW1-LCDR1-FW2-LCDR2-FW3-LCDR3-FW4 based on an alignment (FIG. 2) of the preferred VL members as identified above, as a further aspect of the invention, wherein:
FW1 is Y1, Y2, Y3, Y4, Y5, Y6, Y7, Y8, Y9, Y10, Y11, Y12, Y13, Y14, Y15, Y16, Y17, Y18, Y19, Y20, Y21, Y22
LCDR1 is Y23, Y24, Y24a, Y24b, Y24c, Y24d, Y24e, Y24f, Y25, Y26, Y27, Y28, Y29
FW2 is Y30, Y31, Y32, Y33, Y34, Y35Y36, Y37, Y38, Y39, Y40, Y41, Y42, Y43, Y44, Y44a, Y44b, Y44c, Y44d
LCDR2 is Y45, Y46, Y47, Y48 Y49, Y50, Y51, Y52, Y53, Y54
FW3 is Y55, Y56, Y57, Y58, Y59, Y60, Y61, Y61a, Y61b, Y62, Y63, Y64, Y65, Y66, Y67, Y68, Y69, Y70, Y71, Y72, Y73, Y74, Y75, Y76, Y77, Y78, Y79, Y80, Y81, Y82, Y83
HCDR3 is Y84, Y85, Y86, Y87, Y88, Y89, Y90, Y90a, Y90b, Y90c, Y91, Y92
FW4 is Y93, Y94, Y95, Y96, Y97, Y98, Y99, Y100, Y101, Y102;
   wherein Y1 denotes the first amino acid position, Y2 denotes the second amino acid position, and so on, and the nature of the amino acid at the respective position is defined as follows:

| | | | | |
|---|---|---|---|---|
| Y1 is Q, | Y2 is S, P or E, | Y3 is V or G | Y4 is P, L or V, | Y5 is T or N, |
| Y6 is Q, | Y7 is P or E, | Y8 is P, S or T, | Y9 is S, | Y10 is V, M or L, |
| Y11 is S, | Y12 is G, A or T, | Y13 is A, G, S or T, | Y14 is L or P, | Y15 is G, |
| Y16 is Q, G or T, | Y17 is R, A, T or K, | Y18 is V or I, | Y19 is T or R, | Y20 is I, M or L, |
| Y21 is S or T, | Y22 is C, | | | |
| Y23 is T, A, S or G, | Y24 is G, S or L, | Y24a is S, T or -, | Y24b is S, G, A, | Y24c is S, N, G, P, |
| | | | T, R or -, | E or -, |
| Y24d is N, A, F, V | Y24e is I, N, V, S | Y24f is G, T, V | Y25 is V, R, I, S, | Y26 is G, V, A, D, |
| or -, | or -, | or -, | T, G, A or -, | S or T, |
| Y27 is N, D, G, T, | Y27a is Y, S, G or -, | Y28 is V, A or I, | Y29 is S, N, G, D | |
| S, M, V, K or Y, | | | or Y, | |
| Y30 is W, | Y31 is Y or V, | Y32 is Q or R, | Y33 is Q, | Y34 is I, V, L, K or H, |
| Y35 is P or S, | Y36 is G, R or Y, | Y37 is M, K, R, | Y38 is A, P or R, | Y39 is P or F, |
| | | Q, T or S, | | |
| Y40 is K, Q or S, | Y41 is T, L, A, V or Y, | Y42 is L, I, V or F, | Y43 is I, V or L, | Y44 is Y, V, G or H, |
| Y44a is - or Y, | Y44b is - or Y, | Y44c is - or S, | Y44d is - or D, | |
| Y45 is G, A, S, | Y46 is N, S, T or D, | Y47 is S, N, T, D, R, | Y48 is N, R, K, D, | Y49 is R, Q or L, |
| D, E, R or Y, | | K or F, | E, Y or H, | |
| Y50 is P, N or G, | Y51 is S or P, | Y52 is G, S or E, | Y53 is V, | Y54 is P, |
| Y55 is D, A, N or S, | Y56 is R, | Y57 is F, I, L or V, | Y58 is S, | Y59 is G, |
| Y60 is S, | Y61 is K or L, | Y61a is D or -, | Y61b is A or -, | Y62 is S or V, |
| Y63 is G or A, | Y64 is S, N, T, Q, I | Y65 is T, K or A, | Y66 is G or A, | Y67 is S, T, L or V, |
| | or A, | | | |
| Y68 is L, | Y69 is T, A or L, | Y70 is I or V, | Y71 is T or S, | Y72 is G, |
| Y73 is L, P or A, | Y74 is Q, | Y75 is A, S, T or P, | Y76 is E, | Y77 is D, |
| Y78 is E, | Y79 is A, G or V, | Y80 is D or N, | Y81 is Y, | Y82 is Y or H, |
| Y83 is C, | | | | |
| Y93 is F or I, | Y94 is G, | Y95 is G or R, | Y96 is G, | Y97 is T, |
| Y98 is H, | Y99 is L or V, | Y100 is T or S, | Y101 is V, | Y102 is L, |

(Bold and underlined amino acids are preferred amino acid, ″-″ defines an empty position)

Accordingly, the VL FW1-LCDR1-FW2-LCDR2-FW3-LCDR3-FW4 segment may have the sequence:

(SEQ ID NO 236)
Q(S/P/E)(V/G)(P/L/V)(T/N)Q(P/E)(P/S/T)S(V/L/M)S
(G/A/T)(A/G/S/T)(L/P)G(Q/G/T)(R/A/T/K)(V/I)(T/R)
(I/M/L)(S/T)CXaaXaaXaaXaaXaaXaaXaaXaaXaaXaaXaaXaa
XaaXaaW(Y/V)(Q/R)Q(I/V/L/K/H)(P/S)(G/R/Y)(M/K/R/
Q/T/S)(A/P/R)(P/F)(K/Q/S)(T/L/A/V/Y)(L/I/V/F)
(I/V/L)(Y/V/G/H)(-Y)(-/Y)(-/S)(-/D)XaaXaaXaaXaa
XaaXaaXaaXaaXaaXaa(D/A/N/S)R(F/L/V)SGS(K/L)(-/D)
(-/A)(S/V)(G/A)(S/N/T/Q/I/A)(T/K/A)(G/A)(S/T/L/V)
L(T/A/L)(I/V)(T/S)G(L/P/A)Q(A/S/T/P)EDE(A/G/V)
(D/N)Y(Y/H)CXaaXaaXaaXaaXaaXaaXaaXaaXaaXaaXaaXaa
(F/I)G(G/R)GTH(L/V)(T/S)VL,

wherein the amino acids of the LCDRs are designated as variable amino acids (Xaa). Xaa may be any one of A (Ala), R (Arg), N (Asn), D (Asp), C (Cys), Q (Gln), E (Glu), G (Gly), H (His), I (Ile), L (Leu), K (Lys), M (Met), F (Phe), P (Pro), S (Ser), T (Thr), W (Trp), Y (Tyr), or V (Val).

Preferably, the VL FW1-LCDR1-FW2-LCDR2-FW3-LCDR3-FW4 segment may have the sequence:

(SEQ ID NO 237)
Q(S/P/E)(V/G)(P/L/V)(T/N)Q(P/E)(P/S/T)S(V/L/M)S(G/
A/T)(A/G/S/T)(L/P)G(Q/G/T)(R/A/T/K)(V/I)(T/R)(I/M/
L)(S/T)C(T/A/S/G)(G/S/L)(S/T/-)(S/A/G/T/R/-)(S/N/
G/P/E/-)(N/A/F/V/-)(I/N/V/S/-)(G/T/V/-)(V/R/I/S/
T/G/A/-)(G/V/A/D/S/T)(N/D/G/T/S/M/V/K/Y)(Y/S/G/-)
(V/A/I)(S/N/G/D/Y)W(Y/V)(Q/R)Q(I/V/L/K/H)(P/S)(G/
R/Y)(M/K/R/Q/T/S)(A/P/R)(P/F)(K/Q/S)(T/L/A/V/Y)(L/
I/V/F)(I/V/L)(Y/V/G/H)(-/Y)(-/Y)(-/S)(-/D)(G/A/S/
D/E/R/Y)(N/S/D/T)(S/N/T/D/R/K/F)(N/R/K/D/E/Y/H)
(R/Q/L)(P/N/G)(S/P)(G/S/E)VP(D/A/N/S)R(F/I/L/V)
SGS(K/L)(-/D)(-/A)(S/V)(G/A)(S/N/T/Q/I/A)(T/K/A)
(G/A)(S/T/L/V)L(T/A/L)(I/V)(T/S)G(L/P/A)Q(A/S/T/
P)EDE(A/G/V)(D/N)Y(Y/H)CXaaXaaXaaXaaXaaXaaXaaXaa
XaaXaaXaaXaa(F/I)G(G/R)GTH(L/V)(T/S)VL,

wherein the amino acids of the LCDRs 1 and 2 are defined in the sequence.

More preferably, the VL FW1-LCDR1-FW2-LCDR2-FW3-LCDR3-FW4 segment may have the sequence:

(SEQ ID NO 238)
QSVPTQPPSVSGALGQRVTISCXaaXaaXaaXaaXaaXaaXaaXaaXaa
XaaXaaXaaXaaXaaWYQQIPGMAPKTLIYXaaXaaXaaXaaXaaXaa
XaaXaaXaaXaaDRFSGSKSGSTGSLTITGLQAEDEADYYCXaaXaa
XaaXaaXaaXaaXaaXaaXaaXaaXaaXaaFGGGTHLTVL
or
(SEQ ID NO 239)
QSVPTQPPSVSGALGQRVTISCTGSSSNIGVGNYVSWYQQIPGMAPKTL
IYGNSNRPSGVPDRFSGSKSGSTGSLTITGLQAEDEADYYCXaaXaa
XaaXaaXaaXaaXaaXaaXaaXaaXaaXaaFGGGTHLTVL.

To unify, introduce restriction sites, remove PTM sites, etc., the originally identified the VH and VL FW1-CDR1-FW2-CDR2-FW3 segments were modified as shown in FIGS. 9 and 10.

Accordingly, the invention in a further aspect relates to a polypeptide library comprising VL polypeptide members comprising VL FW1-CDR1-FW2-CDR2-FW3 segments comprising at least one sequence selected from SEQ ID NO: 218, SEQ ID NO: 219, SEQ ID NO: 220, SEQ ID NO: 221, SEQ ID NO: 222, SEQ ID NO: 224, and/or SEQ ID NO: 224 and/or a polypeptide library comprising VH polypeptide members comprising VH FW1-CDR1-FW2-CDR2-FW3 segments comprising at least one sequence selected from SEQ ID NO: 225, SEQ ID NO: 226, SEQ ID NO: 227, SEQ ID NO: 228, SEQ ID NO: 229, SEQ ID NO: 230, and/or SEQ ID NO: 231.

In a general aspect, the polypeptide library disclosed herein is an antibody library, preferably a feline antibody library. Preferably, the antibody library is a synthetic antibody library, more preferably a fully synthetic antibody library.

The feline antibody comprised in the feline antibody library disclosed herein may preferably be a single chain Fv (scFv), a Fab fragment, a F(ab)2 fragment comprising a VL polypeptide member, and/or a VH polypeptide member or a VL/VH polypeptide member combination as described herein.

Especially the preferred VL/VH polypeptide member combinations exhibited high expression levels as scFv and as Fab fragments.

Accordingly, the invention relates to a polypeptide library comprising at least one scFv comprising any of the VH FW1-CDR1-FW2-CDR2-FW3 segments, VL FW1-CDR1-FW2-CDR2-FW3 segments, or VL/VH polypeptide member combination described herein. In these embodiments the VL and VH FW1-CDR1-FW2-CDR2-FW3 segments or VL/VH polypeptide member combinations are comprised in polypeptide members which furthermore comprise the VL and VH CDR3-FR4 segments C-terminally of the respective VL and VH FW1-CDR1-FW2-CDR2-FW3 segments.

Accordingly, in a further embodiment the invention relates to a polypeptide library comprising VL/VH polypeptide member combinations disclosed herein in the form of Fab antibody fragments. More specifically, the VL polypeptide members comprise the VL FW1 to FW3 polypeptide sequence in a VL CDR1-FW2-CDR2-FW3-CDR3-FR4 polypeptide sequence with a C-terminally linked constant light chain sequence C-terminally and a VL FW1 to FW3 polypeptide sequence in a VH CDR1-FW2-CDR2-FW3-CDR3-FR4 polypeptide sequence with a C-terminally linked C1 constant heavy chain sequence. The respective library is a Fab antibody library.

HCDR3, the CDR3 in the VH polypeptide, and LCDR3, the CDR3 in the VL polypeptide, are the major paratopes forming CDRs in an antibody, significantly contributing to antigen binding and recognition.

In our analysis, the most frequent HCDR3-lengths are 11 and 12 present at around 16% each (FIG. 5). For clarification, the present disclosure refers to the numbering scheme as depicted in in Table 9 and FIG. 9 and starts counting after the preferred CAR motif at position 98. For clarification, the present disclosure refers to the numbering scheme as depicted in Table 9 and FIG. 9 and starts counting after the preferred CAR motif at position 98. These results are very much in line with recently published data where the mean amino acid length for HCDR3 was found to be 13.84±3.55 and 13.37±3.38 for two individual cat-samples (counting started after the C within the CAR motif). In contrast to the high variability of HCDR3-length distribution, light chain CDRs are much more restrained. The most frequent LCDR3 length observed was 11 amino acids for lambda light chains comprising >50% of all analyzed sequences (FIG. 5).

To provide libraries suitable to identify binders having diversified binding properties to different antigens, the libraries of the invention are diversified within CDR3 regions. For the 7 most favorable VH/VL combinations the LCDR3 and the HCDR3 regions were replaced by highly diversified LCDR3 and HCDR3 library modules, respectively. Restriction sites were implemented in the VH and VL sequences to enable library module insertion. For affinity maturation and framework diversification, the libraries of the invention further allow the combined diversification of HCDR1 and HCDR2.

The design of the HCDR3 is based on the in-silico analysis performed on naturally occurring antibody sequences. We chose the CAR motif preceding the HCDR3 as this represents the dominant pattern. Also, the length distribution was chosen to correspond to the natural distribution. The HCDR3 diversity module comprises HCDR3 lengths from 7-14 amino acids and represents the majority of all lengths identified in our NGS dataset. In certain embodiments of the libraries, very short or long sequences that frequently show issues with specificity or stability are excluded. Interestingly, for clinical stage human therapeutic antibodies, CDR3 lengths according to IMGT definition had a median length of 12 compared with 15 for the naïve human antibody sequence space (Raybould et al. 2019). If the same holds true also for feline antibodies to be developed as therapeutics, this is covered in the present library design. The amino acid frequency at each position is also based on our NGS dataset. Since each length has slightly adapted amino acid compositions, this was also taken into account in the HCDR3 design. A similar approach was used for the design and construction of the lambda LCDR3 diversity module, comprising lengths from 9-12 amino acids and representing the majority of all lengths in our NGS dataset. As for the framework regions, critical PTMs were omitted within the CDR3 regions. In summary, the term “LCDR3 diversity module” as used herein, refers to a variety of different LCDR3 polypeptide segments, or different nucleotide segments encoding such polypeptide segments, having a partially randomized amino acid sequences in from position 91 to position 99 (optionally including positions 97a, 97b, 97c). In the partially randomized amino acid sequence, certain types of amino acids may be excluded in a specific position and certain amino acids may be found in a certain position with a higher probability than another type of amino acid.

Accordingly, in an aspect of the invention the HCDR3s introduced by a HCDR3 diversity module and comprised in the polypeptide library of the present invention having a length of 7-9 amino acids may have an amino acid distribution as shown in FIG. 11, and HCDR3s comprised in the library of the present having a length of 10-12 amino acids may have an amino acid distribution as shown in FIG. 12, and HCDR3s comprised in the library of the present having a length of 13-14 amino acids may have an amino acid distribution as shown in FIG. 13. In summary, the term “HCDRR3 diversity module” as used herein, refers to a variety of different HCDR3 polypeptide segments, or different nucleotide segments encoding such polypeptide segments, having a partially randomized amino acid sequences in from position 96 to position 104 (optionally including positions 101a, 101b, 101c, 101d, 101e, 101f, 101g). In the partially randomized amino acid sequence, certain types of amino acids may be excluded in a specific position and certain amino acids may be found in a certain position with a higher probability than another type of amino acid.

Furthermore, in an aspect of the invention the LCDR3s introduced by a LCDR3 diversity module and comprised in the polypeptide library of the invention may have a design as shown in Table 10. Preferably, LCDRs comprised in the polypeptide library of the present invention having a length of 9-12 amino acids may have an amino acid distribution as shown in FIG. 14.

According to an aspect of the invention diversification of HCDR1 and HCDR2 for improved affinity maturation is achieved by introducing a VH maturation module comprising randomly or partially randomly HCDR1s and HCDR2s in the VH HCDR1-FW2-HCDR2 segment. A VH maturation module preferably has a design as shown in FIG. 15 The “VH maturation module” may also be referred to as “HCDR1/2 maturation module” herein. In summary, the term “HCDR1/2 maturation module” as used herein, refers to a variety of different VH HCDR1-FW2-HCDR2 polypeptide segments, or different nucleotide segments encoding such polypeptide segments, having a partially randomized amino acid sequences in from position 30 to position 35 and positions 49 to 60. In the partially randomized amino acid sequence, certain types of amino acids may be excluded in a specific position and certain amino acids may be found in a certain position with a higher probability than another type of amino acid.

When a HCDR3, or LCDR3 diversity module or a VH maturation module is introduced into the library, they replace the corresponding segments in the VL and VH FW1-CDR1-FW2-CDR2-FW3 segments as disclosed above.

Prior to library generation, any unfavorable posttranslational modification (PTM) sites were removed from antibody framework sequences to further optimize expression and biophysical properties of the respective VH or VL genes and corresponding proteins. It is important to be aware of post-translational modifications that occur in antibody formulations for therapeutic applications. These are likely not problematic in antibody samples produced for initial in vitro characterization, however posttranslational modifications might take place in antibody samples of high concentration and under long storage conditions, but also occur in vivo. Thus, PTMs can interfere with antibody stability and/or homogeneity and might lead to loss of antibody functionality. Examples of PTMs include but are not limited to oxidation (Met, Trp, His), deamidation (Asn, Gln), isomerization (Asp) or N-linked glycosylation (Asn).

The library design as described leads to various favorable properties, especially for the VL/VH polypeptide member combinations comprised in the library.

In one aspects of the present disclosure, the provided polypeptide library comprises VL/VH polypeptide member combinations, wherein each VL/VH combination is efficiently displayed on a phage. The degree of display may be determined by ELISA and Western blot analysis.

In another aspect the VL/VH polypeptide member combinations comprised in the library of the invention are substantially all expressed in E. coli in Fab format having a monomeric content of at least about 60%, at least about 70%, at least about 80%, at least about 85%, at least about 90%, at least about 95%, at least about 98% or at least about 99%.

The VL and/or VH polypeptide members, and/or VL/VH polypeptide member combinations comprised in the polypeptide library according to the invention may be well expressed in bacterial culture at levels of more than about 1 mg/L, more about than 5 mg/L, or more than about 10 mg/L in a bacterial culture or may be well expressed in IgG format in a mammalian system at levels of more than about 10 mg/L, more than about 15 mg/L, or more than 20 about mg/L.

Furthermore, in certain aspects the present invention, all or substantially all VL/VH polypeptide member combinations comprised in the library may be thermally stable. Said stable polypeptide member combinations may be characterized by a Tm of ≥60° C., or a Tm of ≥62° C., preferably a Tm of ≥64° C., more preferably a Tm of ≥66° C., even more preferably a Tm of ≥68° C., and most preferably a Tm of ≥70° C.

In a further aspect the present invention relates to a collection of nucleic acid molecules encoding a polypeptide library as describes above.

In certain aspects the invention relation relates to a collection of vectors comprising the nucleic acid molecules described above. Furthermore, the invention relates to a recombinant host cell comprising the nucleic acid molecules and/or vectors disclosed herein.

The libraries according to the invention may be used in a method to isolate a binder specific for an antigen, said method comprising the steps of:
(a) contacting a library disclosed herein with an antigen;
(b) removing those members of the library which do not bind to the antigen; and
(c) recovering those members of the library bound to the antigen.

Accordingly, in a more general aspect, the invention relates to a feline antibody or antibody fragment comprising a VL polypeptide member, and/or a VH polypeptide member or a VL/VH polypeptide member combination according as disclosed herein. Preferably feline antibody is a full antibody, a single chain Fv (scFv), a Fab fragment, or a F(ab)2 fragment.

In a further aspect, the invention relates to a method of generating a polypeptide library comprising the steps of:
(i) providing a feline cDNA generated from an antibody expressing tissue, preferably from splenocytes, lymphocytes, bone marrow.
(ii) amplifying VL and VH sequences, preferably by using at least one forward primer selected from SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28; and at least one reverse primer selected from SEQ ID NO: 1, SEQ ID NO: 2, and SEQ ID NO: 3; and
(iii) generating VL/VH combined sequences by randomly linking the amplicons obtained in step ii).

The method may comprise various additional steps including and/or required for the expression of the VL/VH combined sequences as scFv fragments or soluble Fab fragments, determination of the expression levels and determination of the sequences of the best expressing sequences.

In summary, a feline antibody library based on stable and naturally occurring VH and VL combinations comprising CDR3 regions mimicking the natural length and amino acid distribution found in cats is provided by the present invention.

Methods to select candidates from the synthetic feline antibody library include display of antibody fragments in either scFv or Fab format on phages, bacteria or yeast. In the preferred system, phage display, antibody fragments are presented as fusion proteins on the bacteriophage surface. Antibody-presenting phages are then brought in contact with a target molecule of interest to conduct an affinity selection. The antigens can be presented to phages in different forms, e.g. immobilized on the surface of a microtiter plate, in solution or on the surface of cells. Phages that carry specific antibody fragments will recognize and bind the respective antigen; in contrast, weak or unspecific antibody fragments cannot bind and phages are washed away during the selection process, commonly referred to as biopanning. After elution and infection of bacteria with bacteriophage, the genetic information of antibody fragments can be transferred to expression vectors and used for screening and sequencing purposes.

### FIGURE LEGEND

FIG. 1 shows an alignment of feline heavy chain FW1-3 sequences of clones with good expression in both scFv and Fab format.

FIG. 2 shows an alignment of feline light chain FW1-3 sequences of clones with good expression in both scFv and Fab format

FIG. 3 shows a consensus sequence based on an alignment of feline heavy chain FW1-CDR1-FW2-CDR2-FW3-CDR3-FW4 sequences of clones with good expression in both scFv and Fab format

FIG. 4 shows a consensus sequence based on an alignment of feline light chain FW1-CDR1-FW2-CDR2-FW3-CDR3-FW4 sequences of clones with good expression in both scFv and Fab format

FIG. 5 shows the length distribution analysis of feline LCDR3 and HCDR3

FIG. 6 shows the result of an expression check of scFv fragments with randomly paired natural VL and VH chains.

FIG. 7 shows the result of an expression check of Fab fragments with randomly paired natural VL and VH chains

FIG. 8 shows the correlation of expression levels of feline antibody fragments in the scFv and Fab format

FIG. 9 Schematic overview of the VH library building block for seven exemplary antibody scaffolds (including the FW1-3 regions from SEQ No: 225, SEQ No: 226, SEQ No: 227, SEQ No: 228, SEQ No: 229, SEQ No: 230, SEQ No: 231) Depicted are the HCDR1, HCDR2 and HCRDR3 regions (Xaa defines random amino acids; Xaa may be any one of A, R, N, D, C, Q, E, G, H, I, L, K, M, F, P, S, T, W, Y, or V), restriction sites MfeI and XhoI essentially flanking the VH region, NheI and PstI for the introduction of HCDR1/2 maturation modules and BssHII in combination with XhoI for the introduction of HCDR3 diversity.

FIG. 10 Schematic overview of the VL library building block of seven exemplary antibody scaffolds (including the FW1-3 regions from SEQ No: 218, SEQ No: 219, SEQ No: 220, SEQ No: 221, SEQ No: 222, SEQ No: 224, SEQ No: 224). Depicted is the LRDR3 region (Xaa defines random amino acids; Xaa may be any one of A, R, N, D, C, Q, E, G, H, I, L, K, M, F, P, S, T, W, Y, or V) and restriction sites BbsI and KpnI for the introduction of the LCDR3 diversity module.

FIG. 11 shows the amino acid composition of HCDR3 diversification modules having a length of 7-9 amino acids.

FIG. 12 shows the amino acid composition of HCDR3 diversification modules having a length of 10-12 amino acids.

FIG. 13 shows the amino acid composition of HCDR3 diversification modules having a length of 13 and 14 amino acids.

FIG. 14 shows the amino acid composition of LCDR3 diversification modules having a length of 9-12 amino acids.

FIG. 15 shows the schematic concept and amino acid distribution of the HCDR1/2 maturation module. Depicted are flanking regions 5′ to the HCDR1 region, the diversified HCDR1, FW2, the diversified HCDR2 and parts of the subsequent FW3. No length variations are realized in this module. Xaa defines random amino acids (Xaa may be any one of A, R, N, D, C, Q, E, G, H, I, L, K, M, F, P, S, T, W, Y, or V). FW2 is conserved for all library members.

FIG. 16 shows the screening results after a test-panning on eGFP in a dot-blot visualization.

### DEFINITIONS

Unless defined otherwise, all technical and scientific terms used herein have the same meaning as commonly understood by those of ordinary skill in the area to which this invention pertains.

As used in this specification and the appended claims, the singular forms “a,” “an” and “the” include plural references unless the context clearly dictates otherwise.

“About” in the context of amount values refers to an average deviation of maximum +/−20%, preferably +/−10%, most preferably +/−5% based on the indicated value. For example, an amount of about 20 mg/ml refers to 20 mg/ml+/−6 mg/ml, preferably 20 mg/ml+/−4 mg/ml, most preferably 20 mg/ml+/−2 mg/ml. This includes also the value itself without any deviation.

All ranges set forth herein in the summary and description of the invention include all numbers or values thereabout or there between of the numbers of the range. The ranges of the invention expressly denominate and set forth all integers, decimals and fractional values in the range. The term “about” can be used to describe a range.

The terms “antibody” or “polypeptide binder” as used herein include whole antibodies and any antigen binding fragment (i.e., “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region of an IgG, IgA or IgD antibody is comprised of three domains, CH1, CH2 and CH3, whereas the heavy chain of an IgM and IgE antibody is comprised of four domains CH1, CH2, CH3, CH4. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FW). Each VH and VL is composed of three CDRs and four FWs arranged from amino-terminus to carboxy-terminus in the following order: FW1, CDR1, FW2FW2, CDR2, FW3, CDR3, FW4. A polypeptide comprising FW1, CDR1, FW2, CDR2, FW3, CDR3, FW4 of a variable regions of the heavy and light chain may be referred to as a “VH or VL polypeptide”. The variable regions of the heavy and light chains contain a binding domain that interacts with an antigen. The constant regions of the antibodies may mediate the binding of the immunoglobulin to host tissues or factors, including various cells of the immune system (e.g., effector cells) via Fc-receptors and the first component (Clq) of the classical complement system. Amino acids are indicated in one-letter-code.

The extent of the framework region and CDRs have been precisely defined for human antibodies (see Kabat, 1991, J. Immunol., 147, 915-920.; Chothia & Lesk, 1987, J. Mol. Biol. 196: 901-917; Chothia et al., 1989, Nature 342: 877-883; Al-Lazikani et al., 1997, J. Mol. Biol. 273:927-948). The framework regions of an antibody, that is, the combined framework regions of the constituent light and heavy chains, serves to position and align the CDRs, which are primarily responsible for binding to an antigen. However, although feline antibodies can in part be aligned to human antibodies, the above mentioned numbering schemes are not ideally suited to describe amino acid positions within an antibody heavy or light chain sequence. In this invention, the following numbering scheme is used:

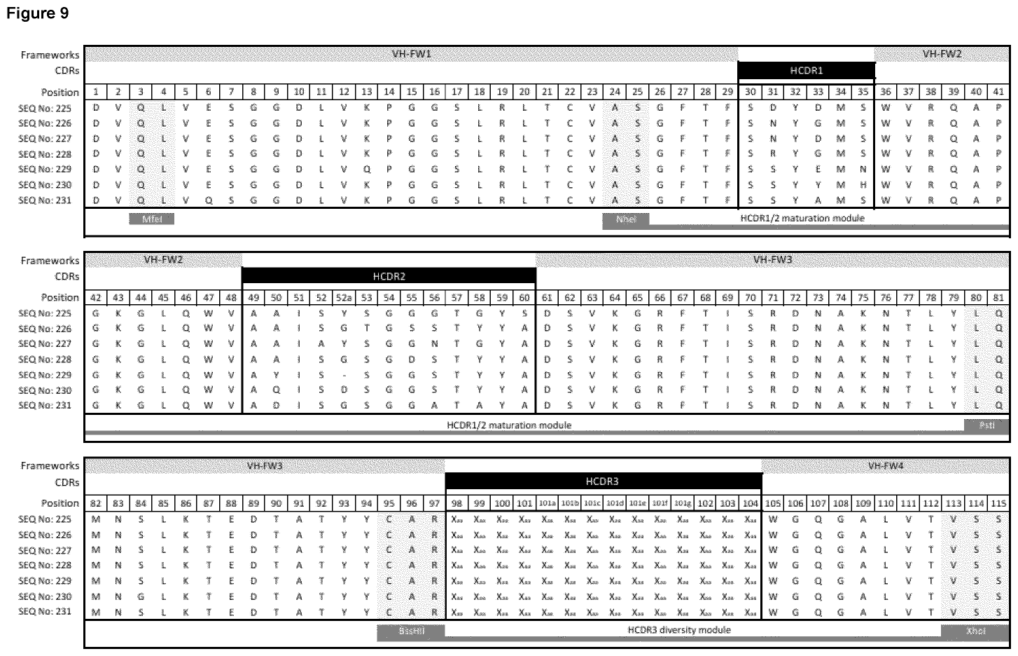

The antibody heavy chain is defined as VH FW1-HCDR1-FW2-HCDR2-FW3-HCDR3-FW4. Framework 1 (FW1) is composed of 29 amino acids (X1, X2, X3, X4, X5, X6, X7, X8, X9, X10, X11, X12, X13, X14, X15, X16, X17, X18, X19, X20, X21, X22, X23, X24, X25, X26, X27, X28, X29). The HCDR1 is 6 amino acids in length and defined from position X30 to X35 (X30, X31, X32, X33, X34, X35). Framework 2 (FW2) is 13 amino acids in length and defined from position X36 to X48 (X36, X37, X38, X39, X40, X41, X42, X43, X44, X45, X46, X47, X48). The HCDR2 is defined from position X49 to X60, with one length variation at position 52, for clarification, position X52 can be empty or having an amino acid (X49, X50, X51, X52, X52a, X53, X54, X55, X56, X57, X58, X59, X60). Framework 3 (FW3) is defined from position X61 to X97 and is 37 amino acids in length. The HCDR3 is defined from position X98 to X104 (X98, X99, X100, X101, X101a, X101b, X101c, X101d, X101e, X101f, X101g, X102, X103, X104). This CDR is diverse in length, indicated by positions X101a to X101g that can harbor an amino acid or not. For clarification, if one of these positions is empty, the subsequent positions until X102 are empty as well. This concept is also depicted in Table 9. Framework 4 (FW4) is defined from position X105 to X115 (X105, X106, X107, X108, X109, X111, X112, X113, X114, X115). The general concept of the numbering scheme is also depicted in FIG. 9.

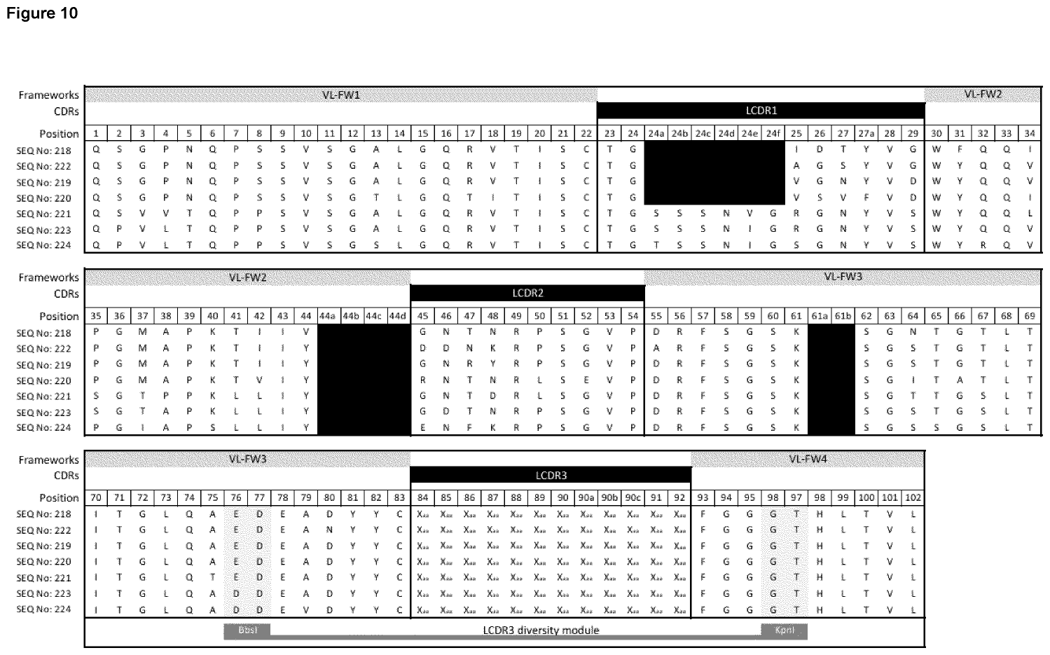

The antibody light chain is defined as VL FW1-LCDR1-FW2-LCDR2-FW3-LCDR3-FW4. Framework 1 (FW1) is composed of 22 amino acids (Y1, Y2, Y3, Y4, Y5, Y6, Y7, Y8, Y9, Y10, Y11, Y12, Y13, Y14, Y15, Y16, Y17, Y18, Y19, Y20, Y21, Y2). The LCDR1 is 6 amino acids in length and defined from position 30 to 35 (X30, X31, X32, X33, X34, X35). Framework 2 (FW2) defined from position 23 to 29 (Y23, Y24, Y24a, Y24b, Y24c, Y24d, Y24e, Y24f, Y25, Y26, Y27, Y28, Y29). This CDR is diverse in length, indicated by positions Y24a to Y24f that can harbor an amino acid or not. For clarification, if one of these positions is empty, the subsequent positions until Y25 are empty as well. The LCDR2 is defined from position 30 to 44d, with length variations at position Y44a to Y44d, for clarification, position Y44a to Y44d can be empty or having an four amino acid stretch (Y30, Y31, Y32, Y33, Y34, Y35, Y36, Y37, Y38, Y39, Y40, Y41, Y42, Y43, Y44, Y44a, Y44b, Y44c, Y44d). Framework 3 (FW3) is defined from position Y55 to Y83 (Y55, Y56, Y57, Y58, Y59, Y60, Y61, Y61a, Y61b, Y62, Y63, Y64, Y65, Y66, Y67, Y68, Y69, Y70, Y71, Y72, Y73, Y74, Y75, Y76, Y77, Y78, Y79, Y80, Y81, Y82, Y83). The LCDR3 is defined from position Y84 to Y92 (Y84, Y85, Y86, Y87, Y88, Y89, Y90, Y90a, Y90b, Y90c, Y91, Y92). This CDR has length variation, indicated by positions Y90a to Y90c that can harbor an amino acid or not. For clarification, if one of these positions is empty, the subsequent positions until Y91 are empty as well. This concept is also depicted in Table 10. Framework 4 (FW4) is defined from position Y93 to Y102 (Y93, Y94, Y95, Y96, Y97, Y98, Y99, Y100, Y101, Y102). The general concept of the numbering scheme is also depicted in FIG. 10.

The terms “antigen binding portion” or “fragment” of an antibody are used equivalently in the present application. These terms refer to one or more fragments of an intact antibody that retain the ability to specifically bind to a given antigen. Antigen binding functions of an antibody can be performed by fragments of an intact antibody. Examples of binding fragments encompassed within the term “antigen binding portion” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., 1989 Nature 341:544-546), which consists of a VH domain; and an isolated complementarity determining region (CDR). Preferred antigen binding portions or fragments of antibodies are Fab fragments. Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more “antigen binding portions” of an antibody. These antibody fragments are obtained using conventional techniques known to those of skill in the art, and the fragments are screened for utility in the same manner as are intact antibodies. Antigen binding portions can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based and polypeptides such as Fibronectin type III (Fn3) (see U.S. Pat. No. 6,703,199, which describes fibronectin polypeptide monobodies). Antigen binding portions can be incorporated into single chain molecules comprising a pair of tandem Fv segments (VH-CH1-VH-CH1) which, together with complementary light chain polypeptides, form a pair of antigen binding regions (Zapata et al., 1995 Protein Eng. 8 (10) 1 057-1062; and U.S. Pat. No. 5,641,870).

The term “feline antibody”, as used herein, refers to antibodies having variable regions in which both the framework and CDR regions are derived from sequences of feline origin. For example both, the framework and CDR regions may be derived from sequences of feline origin. Furthermore, if the antibody contains a constant region, the constant region also is derived from such feline sequences, e.g., feline germline sequences, or mutated versions of feline germline sequences. The feline antibodies of the invention may include amino acid residues not encoded by feline sequences (e.g., mutations introduced by random or site-specific mutagenesis in vitro or by somatic mutation in vivo).

The terms “monoclonal antibody” or “monoclonal antibody composition” as used herein refer to a preparation of antibody molecules of single molecular composition. A monoclonal antibody composition displays a single binding specificity and affinity for a particular epitope.

The term “library” refers to a non-naturally occurring collection of distinct molecules comprising typically more than about 10³, more than about 10⁴, more than about 10⁵, more than about 10⁶, more than about 10⁷, more than about 10⁸, more than about 10⁹ or even more than about 10¹⁰ members. While distinct molecules of a “non-naturally occurring” may naturally occur the entire collection of molecules establishing a library according to the invention does not naturally occur in a native, non-engineered organism. A library in the context of the present invention is a mixture of heterogeneous polypeptides or nucleic acids. The library is composed of members, each of which have a single polypeptide or nucleic acid sequence. Sequence differences between library members are responsible for the diversity present in the library. The library may take the form of a simple mixture of polypeptides or nucleic acids, or may be in the form of organisms or cells, for example bacteria, viruses, phages, animal or plant cells, transformed with a library of nucleic acids. Preferably, each individual organism or cell contains only one or a limited number of library members. Advantageously, the nucleic acids are incorporated into expression vectors, in order to allow expression of the polypeptides encoded by the nucleic acids. In a certain aspect, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants. The term “collection” is essentially used interchangeably with the term “library”.

Antibody libraries can be derived from immunoglobulins, or fragments thereof, that are biased towards certain specificities present in immunized animals or naturally immunized, or infected, humans. Alternatively, antibody libraries can be derived from naive immunoglobulins, or fragments thereof, i.e. immunoglobulins that are not biased towards specificities found in the immune system. Such libraries are referred to as “unbiased” libraries. In preferred embodiments, the present disclosure provides unbiased antibody libraries, i.e. the libraries are not pre-exposed to the antigen of interest. Due to the absence of any bias, such libraries comprise antibodies binding to any potential target antigen of interest.

Typically, immune antibody libraries are constructed with VH and VL gene pools that are cloned from source B cells by PCR-based (or related) cloning techniques. In the same way it is also possible to generate unbiased, naive antibody libraries. Unbiased, naive antibody libraries can however also be generated in a synthetic way in which the entire library is constructed entirely in vitro. Recombinant DNA technology is employed and may be used to mimic the natural biases and redundancies of the natural antibody repertoire. Such antibody libraries are referred to as “synthetic” antibody libraries. The term “fully synthetic” library refers to antibody libraries which are completely, i.e. fully, de novo constructed by DNA synthesis, e.g. by total gene synthesis, PCR-based methods, or related DNA technologies. In such libraries the entire DNA is constructed de novo, i.e. the part encoding the CDRs, as well as the parts (e.g. the framework regions) encoding the parts surrounding the CDRs of the antibodies of the library. The terms “synthetic” and “fully synthetic” therefore refer to the de-novo origin of the DNA. In contrast, in a “semi-synthetic” antibody library only parts of the antibodies of the library are constructed de novo, whereas other parts, e.g. certain CDR regions, are derived from natural sources (numerous reviews on this matter exist, see e.g. Sidhu et al.; Nat Chem Biol (2006), 2, 682-8).

In certain aspects, the present disclosure provides a synthetic antibody library, preferably a semi-synthetic or fully-synthetic library. In the most preferred aspect, the present disclosure provides a fully synthetic feline antibody library.

The term “germline” refers to fully germline sequences and in addition to germline sequences that have been modified or engineered with minor mutations in the amino acid sequence, such as, for the purpose of removing of undesired post-translational modification (PTM) sites, of removing undesired cysteine, optimizing the antibody (e.g. affinity, half-life) or introduction of desired restriction site, or modifications that result from errors in synthesis, amplification or cloning.

The term “VL/VH polypeptide member combination” means the combination (pairing) of one VL polypeptide sequence and one VH polypeptide sequence. The pairing may be between different domains of one polypeptide chain comprising the VL polypeptide sequence and the VH polypeptide sequence, as for example in an scFv, or between two polypeptide chains comprising the VL polypeptide sequence and the VH polypeptide sequence, as for example in a Fab fragment.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids are identified herein according to the commonly known one-letter or three-letter amino acid code.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length. A “substantially identically sequence” according to the invention may thus include any of the disclosed sequences having one or two or three or four or five amino acid exchange(s), preferably one to three, more preferably one or two amino acid exchange(s). Preferably, the exchanges may be conservative amino acid exchange(s).

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the programmed parameters.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “vector” refers to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “expression system” means a host cell and compatible expression vector under suitable conditions, e.g. for the expression of a protein coded for by foreign DNA carried by the vector and introduced to the host cell. Common expression systems include E. coli host cells (e.g. BL21, TGF1−) and plasmid vectors, insect host cells and mammalian host cells (e.g. HEK239 cells or CHO cells) and vectors.

As used herein, the term “substantially all” means that the component to which it refers is more or less pure. Only small amounts or other, different components do exits which do not limit or affect the advantageous property of the component. Depending on the nature of the component essentially all may refer to at least 60%, at least 65%, at least 70%, at least 75%, at least 80%, at least 85%, at least 90%, at least 95% or at least 99% of that component.

As used herein the term “randomized amino acid sequence” refers to a sequence wherein an amino acid at any position within a given sequence may be any amino acid, preferably any natural amino acid. “Partially randomized amino acid sequences” as used herein refers to a sequence wherein any position within a given sequence may be any amino acid except for one or more specifically excluded amino acids. Furthermore, in a “partially randomized amino acid sequence” some positions in a given sequence may comprise a specifically defined amino acid. Randomized amino acid sequence and partially randomized amino acid sequences maybe also be referred to as “randomly diversified”. Hence, “randomly diversified” CDRs are characterized by a randomized or partially randomized amino acid sequence. In a partially randomized amino acid sequence, certain types of amino acids may be excluded in a specific position and certain amino acids may be found in a certain position with a higher probability than another type of amino acid.

### DETAILED DESCRIPTION

In a first aspect, the invention relates to a polypeptide library comprising VL polypeptide members comprising VL FW1-CDR1-FW2-CDR2-FW3 segments comprising at least one sequence selected from SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, and/or SEQ ID NO: 137.

Preferably at least one sequence comprised in the VL FW1-CDR1-FW2-CDR2-FW3 segments comprised in the VL polypeptide is selected from SEQ ID NO: 73, SEQ ID NO: 74, SEQ ID NO: 75, SEQ ID NO: 76, SEQ ID NO: 78, SEQ ID NO: 84, SEQ ID NO: 85, SEQ ID NO: 86, SEQ ID NO: 88, SEQ ID NO: 91, SEQ ID NO: 92, SEQ ID NO: 95, SEQ ID NO: 110, SEQ ID NO: 112, SEQ ID NO: 119, SEQ ID NO: 123, SEQ ID NO: 125, SEQ ID NO: 128, SEQ ID NO: 129, SEQ ID NO: 133, SEQ ID NO: 134, and/or SEQ ID NO: 135.

Preferably the polypeptide library comprises at least 2, at least 5, at least 10, at least 15, or preferably all of said VL polypeptide members.

In a second aspect the invention relates to a polypeptide library comprising VH polypeptide members comprising FR1-CDR1-FW2-CDR2-FR3 segments comprising at least one sequence selected from SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, and SEQ ID NO: 202.

In a preferred aspect, the invention relates to a polypeptide library comprising VL polypeptide members comprising VL FW1-CDR1-FW2-CDR2-FW3 segments comprising at least one sequence selected from SEQ ID NO: 218, SEQ ID NO: 219, SEQ ID NO: 220, SEQ ID NO: 221, SEQ ID NO: 222, SEQ ID NO: 224, and/or SEQ ID NO: 224 and/or a polypeptide library comprising VH polypeptide members comprising VH FW1-CDR1-FW2-CDR2-FW3 segments comprising at least one sequence selected from SEQ ID NO: 225, SEQ ID NO: 226, SEQ ID NO: 227, SEQ ID NO: 228, SEQ ID NO: 229, SEQ ID NO: 230, and/or SEQ ID NO: 231.

Preferably the polypeptide library comprises at least 2, at least 5, at least 10, at least 15, or preferably all of said VH polypeptide members.

Within the context of the present invention “FW1-CDR1-FW2-CDR2-FW3 segments” are polypeptides at least comprising, from N- to C-terminus, framework region 1 followed by CDR1 followed by framework region 2 followed by CDR2 followed by framework region 3 of a feline antibody's variable region of the heavy or light chain. Such VH polypeptide members comprising VH FW1-CDR1-FW2-CDR2-FW3 segments of a feline antibody's variable region of the heavy or chain are also referred to as “VH FW1 to FW3 polypeptide” and VL polypeptide members comprising VH FW1-CDR1-FW2-CDR2-FW3 segments of a feline antibody's variable region of the light chain are also referred to as “VL FW1 to FW3 polypeptide” herein.

In a further main aspect, the invention relates to a polypeptide library comprising VH polypeptide members comprising at least one VH FW1-HCDR1-FW2-HCDR2-FW3-HCDR3-FW4 segment, wherein the sequence comprises:
FW1 is, X1, X2, X3, X4, X5, X6, X7, X8, X9, X10, X11, X12, X13, X14, X15, X16, X17, X18, X19, X20, X21, X22, X23, X24, X25, X26, X27, X28, X29
HCDR1 is, X30, X31, X32, X33, X34, X35
FW2 is, X36, X37, X38, X39, X40, X41, X42, X43, X44, X45, X46, X47, X48
HCDR2 is, X49, X50, X51, X52, X52a, X53, X54, X55, X56, X57, X58, X59, X60
FW3 is, X61, X62, X63, X64, X65, X66, X67, X68, X69, X70, X71, X72, X73, X74, X75, X76, X77, X78, X79, X80, X81, X82, X83, X84, X85, X86, X87, X88, X89, X90, X91, X92, X93, X94, X95, X96, X97
HCDR3 is, X98, X99, X100, X101, X101a, X101b, X101c, X101d, X101e, X101f, X101g, X102, X103, X104
FW4 is, X105, X106, X107, X108, X109, X111, X112, X113, X114, X115;
   wherein X1 denotes the first amino acid position, X2 denotes the second amino acid position, and so on, and the nature of the amino acid at the respective position is defined as follows:

| | | | | |
|---|---|---|---|---|
| X1 is D, Q, E or Y, | X2 is V or E, | X3 is Q, L or R, | X4 is L, | X5 is V, |
| X6 is E or Q, | X7 is S, | X8 is G, | X9 is G, | X10 is D, |
| X11 is L or R, | X12 is V, | X13 is K or Q, | X14 is P, | X15 is G, |
| X16 is G, | X17 is S, | X18 is L, | X19 is R, | X20 is L, |
| X21 is T, I or A, | X22 is C, | X23 is V or M, | X24 is A or G, | X25 is S, |
| X26 is G, | X27 is F, | X28 is T, P, I or N | X29 is F, V or L, | |
| X30 is S, R, G, T or N, | X31 is S, R, G, N, D or L, | X32 is Y, H, F or D, | X33 is G, W, D, E, A, S | X34 is M, |
| | | | or Y, | |
| X35 is S, N, T, D or H, | | | | |
| X36 is W, | X37 is V, | X38 is R, | X39 is Q, | X40 is A or T |
| X41 is P, | X42 is G, | X43 is K or M, | X44 is G, | X45 is L, |
| X46 is Q, | X47 is W, | X48 is V, | | |
| X49 is A, S or T, | X50 is A, S, Y, G, T, Q | X51 is I, N or L, | X52 is S, G, R, D, A or -, | X52a is G, F, Y, S, |
| | or D, | | | N, D or R, |
| X53 is S, T, D, G or N, | X54 is G, | X55 is G, S, I, H, D | X56 is S, A, T, V or N, | X57 is T or I, |
| | | or A, | | |
| X58 is Y, A, G, N or H, | X59 is Y, | X60 is A, S, G or I, | | |
| X61 is D, | X62 is S, | X63 is V, | X64 is K, | X65 is G, |
| X66 is R, | X67 is F, | X68 is T, | X69 is I or V, | X70 is S, |
| X71 is R or K, | X72 is D, | X73 is N or D, | X74 is A, P or V | X75 is K, R, M or E, |
| X76 is N, | X77 is T, | X78 is L, | X79 is Y or L, | X80 is L, |
| X81 is Q, | X82 is M, | X83 is N, T or D, | X84 is S, G or N, | X85 is L, |
| X86 is K, | X87 is T, | X88 is E, | X89 is D, | X90 is T, A or M, |
| X91 is A, | X92 is T, | X93 is Y, | X94 is Y, | X95 is C, |
| X96 is A, S or T | X97 is R, K, N, G or T, | | | |
| X105 is W, | X106 is G, | X107 is Q or R, | X108 is G, | X109 is A, T or V, |
| X110 is L or P, | X111 is V, | X112 is T, | X113 is V, | X114 is S, |
| X115 is S, | | | | |

(Bold and underlined amino acids are preferred amino acid, “−” defines an empty position). Accordingly, the VH FW1-HCDR1-FW2-HCDR2-FW3-HCDR3-FW4 segment may comprise the sequence:

(SEQ ID NO 232)
(D/Q/Y/E)(V/E)(Q/L/R)LV(E/Q)SGGD(L/R)V(K/Q)PGGSLRL
(T/I/A)C(V/M)(A/G)SGF(T/P/I/N)(F/V/L)XaaXaaXaaXaa
XaaXaaWVRQ(A/T)PG(K/M)GLQWVXaaXaaXaaXaaXaaXaaXaa
XaaXaaXaaXaaXaaXaaDSVKGRFT(I/V)S(R/K)D(N/D)(A/P/V)
(K/R/M/E)NTL(Y/L)LQM(N/T/D)(S/G/N)LKTED(T/A/M)ATY
YC(A/S/T)(R/K/N/T/G)XaaXaaXaaXaaXaaXaaXaaXaaXaa
XaaXaaXaaXaaXaaWG(Q/R)G(A/T/V)(L/P)VTVSS,

wherein the amino acids of the HCDRs are designated as variable amino acids (Xaa). Xaa may be any one of A (Ala), R (Arg), N (Asn), D (Asp), C (Cys), Q (Gln), E (Glu), G (Gly), H (His), I (Ile), L (Leu), K (Lys), M (Met), F (Phe), P (Pro), S (Ser), T (Thr), W (Trp), Y (Tyr), or V (Val).

Preferably, the VH FW1-HCDR1-FW2-HCDR2-FW3-HCDR3-FW4 segment may comprise the sequence:

(SEQ ID NO 233)
(D/Q/Y/E)(V/E)(Q/L/R)LV(E/Q)SGGD(L/R)V(K/Q)PGGSLRL
(T/I/A)C(V/M)(A/G)SGF(T/P/I/N)(F/V/L)(S/R/G/T/N)
(S/R/G/N/D/L)(Y/H/F/D)(G/W/D/E/A/S/Y)M(S/N/T/D/H)
WVRQ(A/T)PG(K/M)GLQWV(A/S/T)(A/S/Y/G/T/Q/D)(I/N/L)
(S/G/R/D/A)(G/F/Y/S/N/D/R)(S/T/D/G/N)G(G/S/I/H/D/
A)(S/A/T/V/N)(T/I)(Y/A/G/N/H)Y(A/S/G/I)DSVKGRFT(I/
V)S(R/K)D(N/D)(A/P/V)(K/R/M/E)NTL(Y/L)LQM(N/T/D)
(S/G/N)LKTED(T/A/M)ATYYC(A/S/T)(R/K/N/T/G)XaaXaa
XaaXaaXaaXaaXaaXaaXaaXaaXaaXaaXaaXaaWG(Q/R)G(A/T/
V)(L/P)VTVSS,

wherein the amino acids of the HCDRs 1 and 2 are defined in the sequence.

More preferably, the VH FW1-HCDR1-FW2-HCDR2-FW3-HCDR3-FW4 segment may comprise the sequence:

(SEQ ID NO 234)
DVQLVESGGDLVKPGGSLRLTCVASGFTFXaaXaaXaaXaaXaaXaaWVR
QAPGKGLQWVXaaXaaXaaXaaXaaXaaXaaXaaXaaXaaXaaXaaXaaD
SVKGRFTISRDNAKNTLYLQMNSLKTEDTATYYCARXaaXaaXaaXaa
XaaXaaXaaXaaXaaXaaXaaXaaXaaXaaWGQGALVTVSS,
or
(SEQ ID NO 235)
DVQLVESGGDLVKPGGSLRLTCVASGFTFSSYGMSWVRQAPGKGLQWVAA
ISGSGGSTYYADSVKGRFTISRDNAKNTLYLQMNSLKTEDTATYYCAR
XaaXaaXaaXaaXaaXaaXaaXaaXaaXaaXaaXaaXaaXaaWGQGALVT
VSS.

The polypeptide library according to the invention may comprise at least about 1.0*10⁹ different VH FW1-HCDR1-FW2-HCDR2-FW3-HCDR3-FW4 segments according to SEQ ID NOs 232, 233, 334, or 235. In other aspects the present invention provides a polypeptide library comprising at least about 1.0*10¹⁰, of at least about 1.0*10¹¹, of at least about 1.0*10¹² or of at least about 1.0*10¹³ different VH FW1-HCDR1-FW2-HCDR2-FW3-HCDR3-FW4 segments according to SEQ ID NOs 232, 233, 334, or 235.

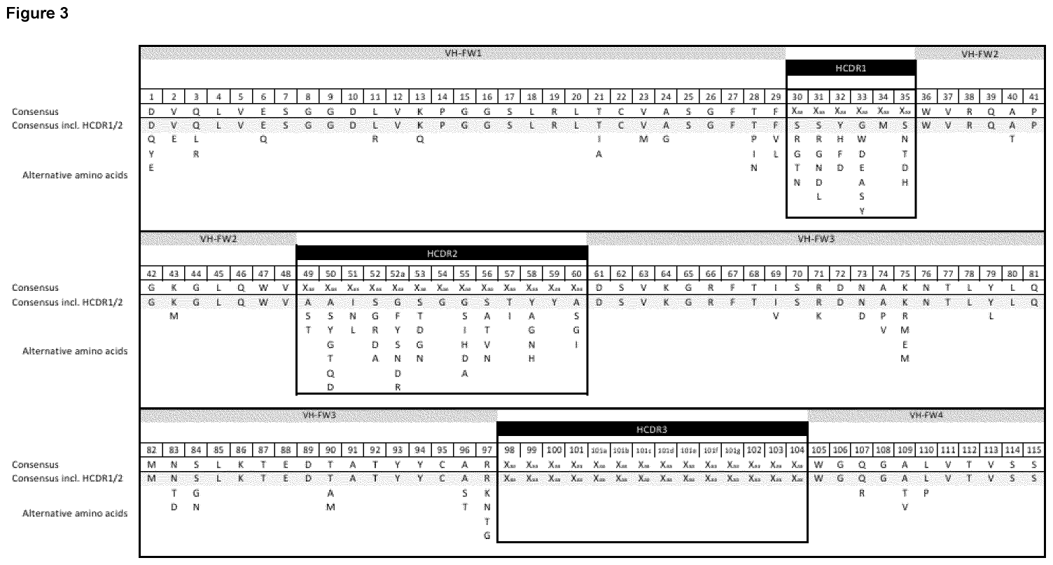

A VH FW1-HCDR1-FW2-HCDR2-FW3-HCDR3-FW4 consensus sequence with alternative amino acids is schematically shown in FIG. 3.

Preferably, the VH FW1-HCDR1-FW2-HCDR2-FW3-HCDR3-FW4 segment comprises a
FW1 sequence selected from
(SEQ ID NO 240)
DEQLVESGGDLVKPGGSLRLTCVASGFPF,
(SEQ ID NO 241)
DEQLVESGGDLVKPGGSLRLTCVASGFTL,
(SEQ ID NO 242)
DEQLVESGGDLVKPGGSLRLTCVGSGFTF,,
(SEQ ID NO 243)
DVQLVESGGDLVKPGGSLRLACVASGFTF,
(SEQ ID NO 244)
DVQLVESGGDLVKPGGSLRLICVASGFTF,
(SEQ ID NO 245)
DVQLVESGGDLVKPGGSLRLTCVASGFIF,
(SEQ ID NO 246)
DVQLVESGGDLVKPGGSLRLTCVASGFPF,
(SEQ ID NO 247)
DVQLVESGGDLVKPGGSLRLTCVASGFTF,
(SEQ ID NO 248)
DVQVVESGGDLVKPGGSLRLTCVASGFTF,
(SEQ ID NO 249)
DVRLVESGGDLVKPGGSLRLTCVASGFTF,
(SEQ ID NO 250)
DVRLVESGGDRVKPGGSLRLTCMASGFNV,
(SEQ ID NO 251)
EVQLVESGGDLVKPGGSLRLTCVASGFTF,
(SEQ ID NO 252)
QVLLVQSGGDLVKPGGSLRLTCVASGFTF,
or
(SEQ ID NO 253)
YVQLVESGGDLVKPGGSLRLTCVASGFTF.
More preferably, the VH FW1-HCDR1-FW2-HCDR2-FW3-HCDR3-FW4 segment comprises
a FW1 sequence selected from
(SEQ ID NO 247)
DVQLVESGGDLVKPGGSLRLTCVASGFTF,
(SEQ ID NO 251)
EVQLVESGGDLVKPGGSLRLTCVASGFTF,
or
(SEQ ID NO 405)
DVQLVESGGDLVQGGSLRLTCVASGFTF.
Preferably, the VH FW1-HCDR1-FW2-HCDR2-FW3-HCDR3-FW4 segment comprises a
HCDR1 sequence selected from
(SEQ ID NO 254)
GSYDMT,
(SEQ ID NO 255)
NNFAMS,
(SEQ ID NO 256)
NSYAMS,
(SEQ ID NO 257)
RGYAMT,
(SEQ ID NO 258)
RSHWMN,
(SEQ ID NO 259)
SDYDMS,
(SEQ ID NO 260)
SGYSMN,
(SEQ ID NO 261)
SLYDMS,
(SEQ ID NO 262)
SNYDMS,
(SEQ ID NO 263)
SNYGMD,
(SEQ ID NO 264)
SNYGMS,
(SEQ ID NO 265)
SRYGMS,
(SEQ ID NO 266)
SSYAMS,
(SEQ ID NO 267)
SSYEMN,
(SEQ ID NO 268)
SSYGMS,
(SEQ ID NO 269)
SSYYMH,
(SEQ ID NO 270)
TGDAMS
or
(SEQ ID NO 271)
TNYAMS.
More preferably, the VH FW1-HCDR1-FW2-HCDR2-FW3-HCDR3-FW4 segment comprises
a HCDR1 sequence selected from
(SEQ ID NO 259)
SDYDMS,
(SEQ ID NO 264)
SNYGMS,
(SEQ ID NO 262)
SNYDMS,
(SEQ ID NO 265)
SRYGMS,
(SEQ ID NO 267)
SSYEMN,
(SEQ ID NO 269)
SSYYMH,
or
NSYAMS.
Preferably, the VH FW1-HCDR1-FW2-HCDR2-FW3-HCDR3-FW4 segment comprises a
FW2 sequence selected from
(SEQ ID NO 271)
WVRQAPGKGLQWV,
(SEQ ID NO 273)
WVRQAPGMGLQWV,
(SEQ ID NO 274)
WVRQAPGRGLQWV
or
(SEQ ID NO 275)
WVRQTPGKGLQWV.
More preferably, the VH FW1-HCDR1-FW2-HCDR2-FW3-HCDR3-FW4 segment comprises
a FW2 sequence of
(SEQ ID NO 271)
WVRQAPGKGLQWV.
Preferably, the VH FW1-HCDR1-FW2-HCDR2-FW3-HCDR3-FW4 segment comprises a
FW3 sequence selected from
(SEQ ID NO 298)
DSVKGRFTISKDDAENTLYLQMNSLKTEDTATYYCAG,
(SEQ ID NO 299)
DSVKGRFTISRDNAENTLLLQMNSLKTEDTATYYCAR,
(SEQ ID NO 300)
DSVKGRFTISRDNAKNTLSLQMDSLKTEDTATYYCAT,
(SEQ ID NO 301)
DSVKGRFTISRDNAKNTLYLQMDSLKTEDTATYYCTS,
(SEQ ID NO 302)
DSVKGRFTISRDNAKNTLYLQMNGLKTEDTATYYCAR,
(SEQ ID NO 303)
DSVKGRFTISRDNAKNTLYLQMNNLKTEDTATYYCAG,
(SEQ ID NO 304)
DSVKGRFTISRDNAKNTLYLQMNSLKTEDMATYYCAR,
(SEQ ID NO 305)
DSVKGRFTISRDNAKNTLYLQMNSLKTEDTATYYCAK,
(SEQ ID NO 306)
DSVKGRFTISRDNAKNTLYLQMNSLKTEDTATYYCAR,
(SEQ ID NO 307)
DSVKGRFTISRDNAKNTLYLQMNSLKTEDTATYYCAT,
(SEQ ID NO 308)
DSVKGRFTISRDNAMNTLYLQMNSLKTEDAATYYCAR,
(SEQ ID NO 309)
DSVKGRFTISRDNARNTLYLQMNSLKTEDTATYYCAR,
(SEQ ID NO 310)
DSVKGRFTISRDNPKNTLYLQMTSLKTEDTATYYCAR,
(SEQ ID NO 311)
DSVKGRFTISRDNVKNTLYLQMNSLKTEDTATYYCAR
or
(SEQ ID NO 312)
DSVKGRFTVSRDNAKNTLYLQMNSLKTEDTATYYCSR.
More preferably, the VH FW1-HCDR1-FW2-HCDR2-FW3-HCDR3-FW4 segment comprises
a FW3 sequence selected from
(SEQ ID NO 302)
DSVKGRFTISRDNAKNTLYLQMNSLKTEDTATYYCAK,
(SEQ ID NO 305)
DSVKGRFTISRDNAKNTLYLQMNGLKTEDTATYYCAR,
(SEQ ID NO 414)
DSVKGRFTISRDNAKNTLYLQMNSLKTEDTATYYCAN,
(SEQ ID NO 306)
DSVKGRFTISRDNAKNTLYLQMNSLKTEDTATYYCAR,
(SEQ ID NO 307)
DSVKGRFTISRDNAKNTLYLQMNSLKTEDTATYYCAT.
Preferably, the VH FW1-HCDR1-FW2-HCDR2-FW3-HCDR3-FW4 segment comprises a
HCDR2 sequence selected from
(SEQ ID NO 276)
AAIAYNGGNTGYA,
(SEQ ID NO 277)
AAIGHDGSTTAYA,
(SEQ ID NO 278)
AAIRGSGGVTYYA,
(SEQ ID NO 279)
AAISGSGDSTYYA,
(SEQ ID NO 280)
AAISYNGGGTGYS,
(SEQ ID NO 281)
AANSGTGSSTYYA,
(SEQ ID NO 282)
ADISGSGGATAYA,
(SEQ ID NO 283)
AGISGSGITTYYA,
(SEQ ID NO 284)
AGISTSGGNTYYA,
(SEQ ID NO 285)
AGITSGGNTYYA,
(SEQ ID NO 286)
AQISDSGGSTYYA,
(SEQ ID NO 287)
AYIDNDGSSTYYA,
(SEQ ID NO 288)
AYIRYDGNTIHYG,
(SEQ ID NO 289)
AYIRYDGSSTNYA,
(SEQ ID NO 290)
AYISSGGSTYYA,
(SEQ ID NO 291)
CAIGGTGSRTLYA,
(SEQ ID NO 292)
SAISFDGSGTGYA,
(SEQ ID NO 293)
SALSESGHSTIYA,
(SEQ ID NO 294)
SSISSGGTTYYA,
(SEQ ID NO 295)
STIDSGGNTHYI,
(SEQ ID NO 296)
TDISRSGATTYYA
or
(SEQ ID NO 297)
TTISGSGGSTYYA.
More preferably the VH FW1-HCDR1-FW2-HCDR2-FW3-HCDR3-FW4 segment comprises a
HCDR2 sequence selected from
(SEQ ID NO 280)
AAISYNGGGTGYS,
(SEQ ID NO 281)
AANSGTGSSTYYA,
(SEQ ID NO 276)
AAIAYNGGNTGYA,
(SEQ ID NO 279)
AAISGSGDSTYYA,
(SEQ ID NO 290)
AYISSGGSTYYA,
(SEQ ID NO 286)
AQISDSGGSTYYA
or
(SEQ ID NO 282)
ADISGSGGATAYA.

Preferably, the VH FW1-HCDR1-FW2-HCDR2-FW3-HCDR3-FW4 segment comprises a FW4 sequence selected from WGQGALVTVSS, WGQGAPVTVSS, WGQGTLVTVSS, WGQGVLVTVSS or WGRGALVTVSS.

More preferably, the VH FW1-HCDR1-FW2-HCDR2-FW3-HCDR3-FW4 segment comprises a FW 4 sequence of WGQGALVTVSS.

To unify the constructs, introduce restriction sites, remove PTM sites, etc., the above described FW1, HCDR1, FW2, HCDR2, FW3, HCDR3, FW4 of VH may be modified to the following sequences:

The VH FW1 sequence may be
(SEQ ID NO 247)
DVQLVESGGDLVKPGGSLRLTCVASGFTF,
(SEQ ID NO 405)
DVQLVESGGDLVQPGGSLRLTCVASGFTF,
and/or
(SEQ ID NO 406)
DVQLVQSGGDLVKPGGSLRLTCVASGFTF.
The HCDR1 sequence may be
(SEQ ID NO 259)
SDYDMS,
(SEQ ID NO 264)
SNYGMS,
(SEQ ID NO 262)
SNYDMS,
(SEQ ID NO 265)
SRYGMS,
(SEQ ID NO 267)
SSYEKN,
(SEQ ID NO 269)
SSYYMH,
and/or
(SEQ ID NO 266)
SSYAMS.
The VH FW2 sequence may be
(SEQ ID NO 271)
WVRQAPGKGLQWV.
The HCDR2 sequence may be
(SEQ ID NO 280)
AAISYNGGGTGYS,
(SEQ ID NO 281)
AANSGTGSSTYYA,
(SEQ ID NO 276)
AAIAYNGGNTGYA,
(SEQ ID NO 279)
AAISGSGDSTYYA,
(SEQ ID NO 290)
AYISSGGSTYYA,
(SEQ ID NO 286)
AQISDSGGSTYYA
and/or
(SEQ ID NO 282)
ADISGSGGATAYA.
The VH FW3 sequence may be
(SEQ ID NO 302)
DSVKGRFTISRDNAKNTLYLQMNGLKTEDTATYYCAR
or
(SEQ ID NO 306)
DSVKGRFTISRDNAKNTLYLQMNSLKTEDTATYYCAR.

VH FW4 sequence may be WGQGALVTVSS.

The above FW1, HCDR1, FW2, HCDR2, FW3, HCDR3, FW4 of VH may be combined.

In a further main aspect, the invention relates to a polypeptide library comprising VL polypeptide members comprising at least one VL FW1-LCDR1-FW2-LCDR2-FW3-LCDR3-FW4 segment, wherein the sequence comprises:
FW1 is Y1, Y2, Y3, Y4, Y5, Y6, Y7, Y8, Y9, Y10, Y11, Y12, Y13, Y14, Y15, Y16, Y17, Y18, Y19, Y20, Y21, Y22
LCDR1 is Y23 Y24, Y24a, Y24b, Y24c, Y24d, Y24e, Y24f, Y25, Y26, Y27, Y28, Y29
FW2 is, Y30, Y31, Y32, Y33, Y34, Y35, Y36, Y37, Y38, Y39, Y40, Y41, Y42, Y43, Y44, Y44a, Y44b, Y44c, Y44d
LCDR2 is Y45, Y46, Y47, Y48 Y49, Y50, Y51, Y52, Y53, Y54
FW3 is Y55, Y56, Y57, Y58, Y59, Y60, Y61, Y61a, Y61b, Y62, Y63, Y64, Y65, Y66, Y67, Y68, Y69, Y70, Y71, Y72, Y73, Y74, Y75, Y76, Y77, Y78, Y79, Y80, Y81, Y82, Y83
HCDR3 is Y84, Y85, Y86, Y87, Y88, Y89, Y90, Y90a, Y90b, Y90c, Y91, Y92
FW4 is Y93, Y94, Y95, Y96, Y97, Y98, Y99, Y100, Y101, Y102,
   wherein Y1 denotes the first amino acid position, Y2 denotes the second amino acid position, and so on, and the nature of the amino acid at the respective position is defined as follows:

| | | | | |
|---|---|---|---|---|
| Y1 is Q, | Y2 is S, P or E, | Y3 is Vor G, | Y4 is P, L or V, | Y5 is T or N, |
| Y6 is Q, | Y7 is P or E, | Y8 is P, S or T, | Y9 is S, | Y10 is V, M or L, |
| Y11 is S, | Y12 is G, A or T, | Y13 is A, G, S or T, | Y14 is L or P, | Y15 is G, |
| Y16 is Q, G or T, | Y17 is R, A, T or K, | Y18 is V or I, | Y19 is T or R, | Y20 is I, M or L, |
| Y21 is S or T, | Y22 is C, | | | |
| Y23 is T, A, S or G, | Y24 is G, S or L, | Y24a is S, T or -, | Y24b is S, G, A, T, R or -, | Y24c is S, N, G, P, E or -, |
| Y24d is N, A, F, V or -, | Y24e is I, N, V, S or -, | Y24f is G, T, V or -, | Y25 is V, R, I, S, T, G, A or -, | Y26 is G, V, A, D, S or T, |
| Y27 is N, D, G, T, | Y27a is Y, S, G or -, | Y28 is V, A or I, | Y29 is S, N, G, D or Y, | |
| S, M, V, K or Y, | | | | |
| Y30 is W, | Y31 is Y or V, | Y32 is Q or R, | Y33 is Q, | Y34 is I, V, L, K or H, |
| Y35 is P or S, | Y36 is G, R or Y, | Y37 is M, K, R, Q, T | Y38 is A, P or R, | Y39 is P or F, |
| | | or S, | | |
| Y40 is K, Q or S, | Y41 is T, L, A, V or Y, | Y42 is L, I, V or F, | Y43 is I, V or L, | Y44 is Y, V, G or H, |
| Y44a is - or Y, | Y44b is- or Y, | Y44c is - or S, | Y44d is - or D, | |
| Y45 is G, A, S, | Y46 is N, S, T or D, | Y47 is S, N, T, D, R, | Y48 is N, R, K, D, E, Y | Y49 is R, Q or L, |
| D, E, R or Y, | | K or F, | or H, | |
| Y50 is P, N or G, | Y51 is S or P, | Y52 is G, S or E | Y53 is V, | Y54 is P, |
| Y55 is D, A, N or S, | Y56 is R, | Y57 is F, I, L or V, | Y58 is S, | Y59 is G, |
| Y60 is S, | Y61 is K or L, | Y61a is D or -, | Y61b is A or -, | Y62 is S or V, |
| Y63 is G or A, | Y64 is S, N, T, Q, I or A, | Y65 is T, K or A, | Y66 is G or A, | Y67 is S, T, L or V, |
| Y68 is L, | Y69 is T, A or L, | Y70 is I or V, | Y71 is T or S, | Y72 is G, |
| Y73 is L, P or A, | Y74 is Q, | Y75 is A, S, T or P, | Y76 is E, | Y77 is D, |
| Y78 is E, | Y79 is A, G or V, | Y80 is D or N, | Y81 is Y, | Y82 is Y or H, |
| Y83 is C, | | | | |
| Y93 is F or I, | Y94 is G, | Y95 is G or R, | Y96 is G, | Y97 is T, |
| Y98 is H, | Y99 is L or V, | Y100 is T or S, | Y101 is V, | Y102 is L, |

(Bold and underlined amino acids are preferred amino acid, ″-″ defines an empty position)

Accordingly, the VL FW1-LCDR1-FW2-LCDR2-FW3-LCDR3-FW4 segment may have the sequence:

(SEQ ID NO 236)
Q(S/P/E)(V/G)(P/L/V)(T/N)Q(P/E)(P/S/T)S(V/L/M)S(G/
A/T)(A/G/S/T)(L/P)G(Q/G/T)(R/A/T/K)(V/I)(T/R)(I/M/
L)(S/T)CXaaXaaXaaXaaXaaXaaXaaXaaXaaXaaXaaXaaXaaXaa
W(Y/V)(Q/R)Q(I/V/L/K/H)(P/S)(G/R/Y)(M/K/R/Q/T/S)
(A/P/R)(P/F)(K/Q/S)(T/L/A/V/Y)(L/I/V/F)(I/V/L)(Y/
V/G/H)(-/Y)(-/Y)(-/S)(-/D)XaaXaaXaaXaaXaaXaaXaaXaa
XaaXaa(D/A/N/S)R(F/I/L/V)SGS(K/L)(-/D)(-/A)(S/V)
(G/A)(S/N/T/Q/I/A)(T/K/A)(G/A)(S/T/L/V)L(T/A/L)(I/
V)(T/S)G(L/P/A)Q(A/S/T/P)EDE(A/G/V)(D/N)Y(Y/H)
CXaaXaaXaaXaaXaaXaaXaaXaaXaaXaaXaaXaa(F/I)G(G/R)GT
H(L/V)(T/S)VL

when the amino acids of the LCDRs are designated as variable amino acids (Xaa). Xaa may be any one of A (Ala), R (Arg), N (Asn), D (Asp), C (Cys), Q (Gln), E (Glu), G (Gly), H (His), I (Ile), L (Leu), K (Lys), M (Met), F (Phe), P (Pro), S (Ser), T (Thr), W (Trp), Y (Tyr), or V (Val).

Preferably, the VL FW1-LCDR1-FW2-LCDR2-FW3-LCDR3-FW4 segment may have the sequence:

(SEQ ID NO 237)
Q(S/P/E)(V/G)(P/L/V)(T/N)Q(P/E)(P/S/T)S(V/L/M)S(G/
A/T)(A/G/S/T)(L/P)G(Q/G/T)(R/A/T/K)(V/I)(T/R)(I/M/
L)(S/T)C(T/A/S/G)(G/S/L)(S/T/-)(S/A/G/T/R/-)(S/N/
G/P/E/-)(N/A/F/V/-)(I/N/V/S/-)(G/T/V/-)(V/R/I/S/T/
G/A/-)(G/V/A/D/S/T)(N/D/G/T/S/M/V/K/Y)(Y/S/G/-
)(V/A/I)(S/N/G/D/Y)W(Y/V)(Q/R)Q(I/V/L/K/H)(P/S)(G/
R/Y)(M/K/R/Q/T/S)(A/P/R)(P/F)(K/Q/S)(T/L/ANNY)(L/I
/V/F)(I/V/L)(Y/V/G/H)(-/Y)(-/Y)(-/S)(-/D)(G/A/S/D/
E/R/Y)(N/S/D/T)(S/N/T/D/R/K/F)(N/R/K/D/E/Y/H)(R/Q/
L)(P/N/G)(S/P)(G/S/E)VP(D/A/N/S)R(F/I/L/V)SGS(K/L)
(-/D)(-/A)(S/V)(G/A)(S/N/T/Q/I/A)(T/K/A)(G/A)(S/T/
L/V)L(T/A/L)(I/V)(T/S)G(L/P/A)Q(A/S/T/P)EDE(A/G/
V)(D/N)Y(Y/H)CXaaXaaXaaXaaXaaXaaXaaXaaXaaXaaXaaXaa
(F/I)G(G/R)GTH(L/V)(T/S)VL,

wherein the amino acids of the LCDRs 1 and 2 are defined in the sequence.

More preferably, the VL FW1-LCDR1-FW2-LCDR2-FW3-LCDR3-FW4 segment may have the sequence:

(SEQ ID NO 238)
QSVPTQPPSVSGALGQRVTISCXaaXaaXaaXaaXaaXaaXaaXaaXaa
XaaXaaXaaXaaXaaWYQQIPGMAPKTLIYXaaXaaXaaXaaXaaXaa
XaaXaaXaaXaaDRFSGSKSGSTGSLTITGLQAEDEADYYCXaaXaa
XaaXaaXaaXaaXaaXaaXaaXaaXaaXaaFGGGTHLTVL,
or
(SEQ ID NO 239)
QSVPTQPPSVSGALGQRVTISCTGSSSNIGVGNYVSWYQQIPGMAPKTL
IYGNSNRPSGVPDRFSGSKSGSTGSLTITGLQAEDEADYYCXaaXaaXaa
XaaXaaXaaXaaXaaXaaXaaXaaXaaFGGGTHLTVL,
(SEQ ID NO 416)
QSGPNQPSSVSGALGQRVTISCTGSSSNIGRGNYVSWYQQVPGMAPKTLI
YGNSNRPSGVPDRFSGSKSGSTGSLTITGLQAEDEADYYCXaaXaaXaa
XaaXaaXaaXaaXaaXaaXaaXaaXaaFGGGTHLTVL.

The polypeptide library according to the invention may comprise at least about 1.0*109 different VL FW1-LCDR1-FW2-LCDR2-FW3-LCDR3-FW4 segments according to SEQ ID NOs 236, 237, 238, 239, or 416. In other aspects the present invention provides a polypeptide library comprising at least about 1.0*1010, of at least about 1.0*1011, of at least about 1.0*1012 or of at least about 1.0*1013 different VL FW1-LCDR1-FW2-LCDR2-FW3-LCDR3-FW4 segments according to SEQ ID NOs 236, 237, 238, 239, or 416.

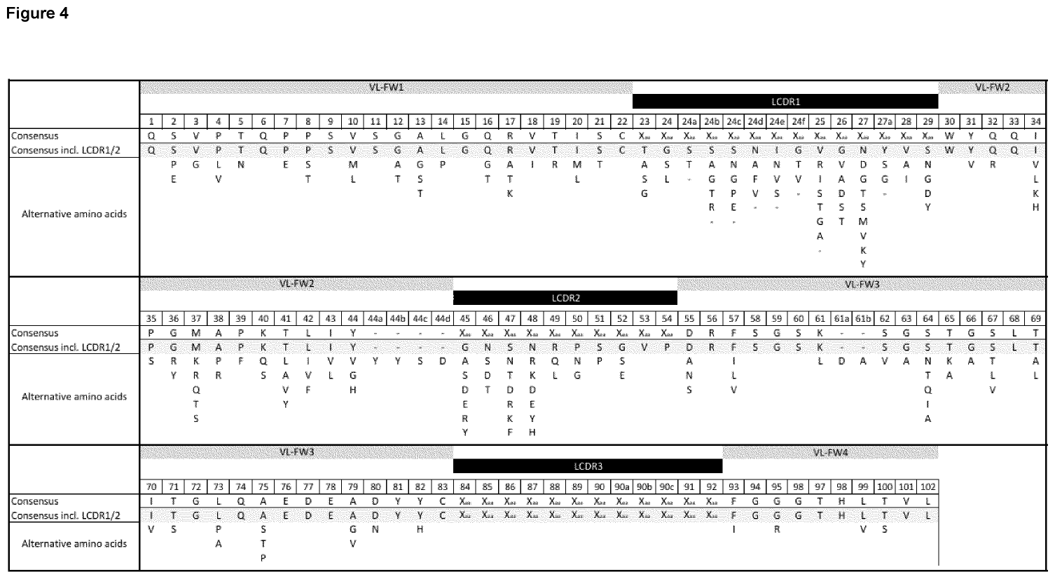

The VL FW1-LCDR1-FW2-LCDR2-FW3-LCDR3-FW4 consensus sequence with alternative amino acids is schematically shown in FIG. 4.

Preferably, the VL FW1-LCDR1-FW2-LCDR2-FW3-LCDR3-FW4 segment comprises a FW1
sequence selected from
(SEQ ID NO 313)
QSVVTQPPSVSGALGQAVTISC ,
(SEQ ID NO 314)
QSVLTQPPSVSGALGQTVTISC ,
(SEQ ID NO 315)
QSVLTQPPSVSGGLGQKVTISC ,
(SEQ ID NO 316)
QPVVTQPPSVSGALGQTVTISC ,
(SEQ ID NO 317)
QSVLTQPPSMSGALGQTVTISC ,
(SEQ ID NO 318)
QPVLTQPPSVSGSLGQRVTISC ,
(SEQ ID NO 319)
QSGPNQPSSVSGALGQRVTISC ,
(SEQ ID NO 320)
QSGPNQPSSVSGALGQRVTMSC ,
(SEQ ID NO 321)
QSGPNQPSSVSAALGQRVTISC,
(SEQ ID NO 322)
QSGPNQPSSVSGTLGQTITISC ,
(SEQ ID NO 323)
QEVVTQETSLSTTPGGTVTLTC
(SEQ ID NO 324)
QSVVTQPPSVSGALGQRVTISC ,
(SEQ ID NO 325)
QPVLTQPPSVSGALGQRVTISC ,
(SEQ ID NO 326)
QPVVTQPPSVSGALGQRVTISC ,
(SEQ ID NO 327)
QPVLTQPSSLSASPGTTARLTC
or
(SEQ ID NO 328)
QPVVTQPPSLSGSLGATARLTC.
More preferably, the VL FW1-LCDR1-FW2-LCDR2-FW3-LCDR3-FW4 segment comprises a
FW1 sequence selected from (SEQ ID NO 318),
(SEQ ID ON 319)
QSGPNQPSSVSGALGQRVTISC,
(SEQ ID NO 322)
QSGPNQPSSVSGTLGQTITISC,
(SEQ ID NO 324)
QSVVTQPPSVSGALGQRVTISC,
and/or
(SEQ ID NO 325)
QPVLTQPPSVSGALGQRVTISC.
Preferably, the VL FW1-LCDR1-FW2-LCDR2-FW3-LCDR3-FW4 segment comprises a
LCDR1 sequence selected from
(SEQ ID NO 329)
AGSANNIGITDVN ,
(SEQ ID NO 330)
AGSGSNIGIVDVN
(SEQ ID NO 331)
AGSGSNIGIAGVN ,
(SEQ ID NO 332)
AGSANNNGIVGVN ,
(SEQ ID NO 333)
AGSGSNIGVAGVN ,
(SEQ ID NO 334)
TGTSSNIGSGNYVS ,
(SEQ ID NO 335)
TGIDTYVG ,
(SEQ ID NO 336)
TGVDTYVD
(SEQ ID NO 337)
TGVDSYVG ,
(SEQ ID NO 338)
SGAGSYVA ,
(SEQ ID NO 339)
TGAGSYVG ,
(SEQ ID NO 340)
TGVGNYVD ,
(SEQ ID NO 341)
TGVGMSVD ,
(SEQ ID NO 342)
TGAGYVG ,
(SEQ ID NO 343)
TGVSVFVD ,
(SEQ ID NO 344)
GSSTGAVTTSNFAS ,
(SEQ ID NO 345)
TGSSSNVGRGNYVS ,
(SEQ ID NO 346)
TGSSPNIGRGNYVS ,
(SEQ ID NO 347)
TGSSSNIGRGNYVS ,
(SEQ ID NO 348)
TLSSGFNVGGYYIS
or
(SEQ ID NO 349)
TLSREVSVGVKGIY.
More preferably, the VL FW1-LCDR1-FW2-LCDR2-FW3-LCDR3-FW4 segment comprises a
LCDR1 sequence selected from
(SEQ ID NO 334)
TGTSSNIGSGNYVS ,
(SEQ ID NO 335)
TGIDTYVG ,
(SEQ ID NO 339)
TGAGSYVG ,
(SEQ ID NO 340)
TGVGNYVD ,
(SEQ ID NO 343)
TGVSVFVD,
(SEQ ID NO 345)
TGSSSNVGRGNYVS ,
and/or
(SEQ ID NO 347)
TGSSSNIGRGNYVS.
Preferably, the VL FW1-LCDR1-FW2-LCDR2-FW3-LCDR3-FW4 segment comprises a FW2
sequence selected from
(SEQ ID NO 350)
WYQQLPGKAPKLLIY ,
(SEQ ID NO 351)
WYQQLPGKAPSLLIY,
(SEQ ID NO 352)
WYQQHPRKAPKLLIY ,
(SEQ ID NO 353)
WYQQLPGKAPQLLIY ,
(SEQ ID NO 354)
WYQQLPGKAPKALIY ,
(SEQ ID NO 355)
WYRQVPGIAPSLLIY ,
(SEQ ID NO 356)
WFQQIPGMAPKTIIV ,
(SEQ ID NO 357)
WYQQIPGMAPKTIIY ,
(SEQ ID NO 358)
WYQQIPGRAPKTIIY ,
(SEQ ID NO 359)
WYQQVPGMAPKTIIY ,
(SEQ ID NO 360)
WYQQIPGMAPKTVIY ,
(SEQ ID NO 361)
WVQQKPYQRFQGLVG ,
(SEQ ID NO 362)
WYQQLSGTPPKLLIY ,
(SEQ ID NO 363)
WYQQLSGTAPKVLIY ,
(SEQ ID NO 364)
WYQQLSGTAPKLLIY ,
(SEQ ID NO 365)
WYQQVSGTAPKLLIY
(SEQ ID NO 366)
WFQQKPGSPPRYLLYYYSD
or
(SEQ ID NO 367)
or WYQQKPGSPPRYFLHYYSD.
More preferably, the VL FW1-LCDR1-FW2-LCDR2-FW3-LCDR3-FW4 segment comprises a
FW2 sequence selected from
(SEQ ID NO 355)
WYRQVPGIAPSLLIY,
(SEQ ID NO 356)
WFQQIPGMAPKTIIV,
(SEQ ID NO 359)
WYQQVPGMAPKTIIY,
(SEQ ID NO 360)
WYQQIPGMAPKTVIY,
(SEQ ID NO 362)
WYQQLSGTPPKLLIY,
and/or
(SEQ ID NO 365)
WYQQVSGTAPKLLIY.
Preferably, the VL FW1-LCDR1-FW2-LCDR2-FW3-LCDR3-FW4 segment comprises a
LCDR2 sequence selected from
(SEQ ID NO 368)
ANNRRPSSVP,
(SEQ ID NO 369)
ASDRRPSGVP,
(SEQ ID NO 370)
GSSNRPSGVP,
(SEQ ID NO 371)
ASTRRPSGVP,
(SEQ ID NO 372)
ENFKRPSGVP,
(SEQ ID NO 373)
GNTNRPSGVP,
(SEQ ID NO 374)
YDSNRPSGVP,
(SEQ ID NO 375)
DNSNRPSGVP,
(SEQ ID NO 376)
DDNKRPSGVP,
(SEQ ID NO 377)
GNRYRPSGVP,
(SEQ ID NO 378)
RNSNRLSEVP,
(SEQ ID NO 379)
GNSYRPSGVP,
(SEQ ID NO 380)
RNTNRLSEVP,
(SEQ ID NO 381)
GTSYRNPGVP,
(SEQ ID NO 382)
GNSDRLSGVP,
(SEQ ID NO 383)
GDKNRPSGVP,
(SEQ ID NO 384)
GDTNRPSGVP,
(SEQ ID NO 385)
SDKHQGPGVP
or
(SEQ ID NO 386)
STNELGPGVP.
More preferably, the VL FW1-LCDR1-FW2-LCDR2-FW3-LCDR3-FW4 segment comprises a
LCDR2 sequence selected from
(SEQ ID NO 372)
ENFKRPSGVP,
(SEQ ID NO 373)
GNTNRPSGVP,
(SEQ ID NO 376)
DDNKRPSGVP,
(SEQ ID NO 377)
DNRYRPSGVP,
(SEQ ID NO 380)
RNTNRLSEVP,
(SEQ ID NO 410)
GNTDRLSGVP,
and/or
(SEQ ID NO 384)
GDTNRPSGVP.
Preferably, the VL FW1-LCDR1-FW2-LCDR2-FW3-LCDR3-FW4 segment comprises a FW3
sequence selected from
(SEQ ID NO 387)
DRFSGSKSGATGSLTISGLQAEDEADYYC ,
(SEQ ID NO 388)
ERISGSRSGNTGSLTITGLQTEDEADYYC ,
(SEQ ID NO 389)
DRFSGSKSGNTGSLTITGLQAEDEADYYC ,
(SEQ ID NO 390)
DRFSGSKSGNTGSLTITGLQAEDEADYYC ,
(SEQ ID NO 391)
ERLSGSKSGNTGSLTITGLQPEDEADYYC ,
(SEQ ID NO 392)
DRFSGSKSGSSGSLTITGLQADDEVDYYC ,
(SEQ ID NO 393)
DRFSGSKSGNTGTLTITGLQAEDEADYYC ,
(SEQ ID NO 394)
DRFSGSKSGSTGTLAITGLQAEDEGDYYC ,
(SEQ ID NO 395)
DRFSGSKSGNTGTLTITGLQAEDEADYYC ,
(SEQ ID NO 396)
DRFSGSKSGNTGTLTITGLQAEDEADYYC ,
(SEQ ID NO 397)
ARFSGSKSGSTGTLTITGLQAEDEANYYC ,
(SEQ ID NO 398)
DRFSGSKSGSTGTLTITGLQAEDEADYYC ,
(SEQ ID NO 399)
NRFSGSKSGSTATLTITGLQAEDEADYYC ,
(SEQ ID NO 400)
DRFSGSKSGSTGTLTITGLQAEDEADYYC ,
(SEQ ID NO 401)
DRFSGSKSGITATLTITGLQAEDEADYYC ,
(SEQ ID NO 402)
ARFSGSLVGQKAVLTITGAQSEDEAEYYC ,
(SEQ ID NO 403)
DRFSGSKSGTTGSLTITGLQTEDEADYYC
or
(SEQ ID NO 404)
DRFSGSKSGSTGSLTITGLQAEDEADYYC.
More preferably, the VL FW1-LCDR1-FW2-LCDR2-FW3-LCDR3-FW4 segment comprises a
FW3 sequence selected from
(SEQ ID NO 392)
DRFSGSKSGSSGSLTITGLQADDEVDYYC ,
(SEQ ID NO 396)
DRFSGSKSGNTGTLTITGLQAEDEADYYC ,
(SEQ ID NO 397)
ARFSGSKSGSTGTLTITGLQAEDEANYYC ,
(SEQ ID NO 400)
DRFSGSKSGSTGTLTITGLQAEDEADYYC ,
(SEQ ID NO 401)
DRFSGSKSGITATLTITGLQAEDEADYYC ,
(SEQ ID NO 403)
DRFSGSKSGTTGSLTITGLQTEDEADYYC
or
(SEQ ID NO 415)
DRFSGSKSGSTGSLTITGLQADDEADYYC.
Preferably, the VL FW1-LCDR1-FW2-LCDR2-FW3-LCDR3-FW4 segment comprises a FW4
sequence selected from
(SEQ ID NO: 208)
FGGGTHLTVL ,
(SEQ ID NO 209)
FGGGTHLSVL ,
(SEQ ID NO: 210)
FGRGTHLTVL ,
(SEQ ID NO: 211)
FGGGTHVTVL ,
or
(SEQ ID NO: 212)
IGGGTHVTVL.
More preferably, the VL FW1-LCDR1-FW2-LCDR2-FW3-LCDR3-FW4 segment comprises a
FW4 sequence of
(SEQ ID NO: 208)
FGGGTHLTVL.

To unify the constructs, introduce restriction sites, remove PTM sites, etc., the above described FW1, HCDR1, FW2, HCDR2, FW3, HCDR3, FW4 of VL may be modified to the following sequences:

The VL FW1 may be (SEQ ID NO 318),
(SEQ ID NO 319)
QSGPNQPSSVSGALGQRVTISC,
(SEQ ID NO 322)
QSGPNQPSSVSGTLGQTITSC,
(SEQ ID NO 324)
QSVVTQPPSVSGALGQRVTISC,
and/or
(SEQ ID NO 325)
QPVLTQPPSVSGALGQRVTISC.
The HCDR1 may be
(SEQ ID NO 334)
TGTSSNIGSGNYVS,
(SEQ ID NO 335)
TGIDTYVG,
(SEQ ID NO 339)
TGAGSYVG,
(SEQ ID NO 340)
TGVGNYVD,
(SEQ ID NO 343)
TGVSVFVD,
(SEQ ID NO 345)
TGSSSNVGRGNYVS,
and/or
(SEQ ID NO 347)
TGSSSNIGRGNYVS.
The VL FW3 may be
(SEQ ID NO 411)
DRESGSKSGSSGSLTITGLQAEDEVDYYC ,
(SEQ ID NO 396)
DRFSGSKSGNTGTLTITGLQAEDEADYYC ,
(SEQ ID NO 397)
ARFSGSKSGSTGTLTITGLQAEDEANYYC ,
(SEQ ID NO 400)
DRFSGSKSGSTGTLTITGLQAEDEADYYC ,
(SEQ ID NO 401)
DRFSGSKSGITATLTITGLQAEDEADYYC ,
(SEQ ID NO 403)
DRFSGSKSGTTGSLTITGLQTEDEADYYC ,
and/or
(SEQ ID NO 404)
DRFSGSKSGSTGSLTITGLQAEDEADYYC.
The VL FW 4 may be
(SEQ ID NO 208)
FGGGTHLTVL.

Preferably at least one sequence comprised in the VL FW1-CDR1-FW2-CDR2-FW3 segments or the VL FW1-LCDR1-FW2-LCDR2-FW3-LCDR3-FW4 segments comprised in the VL polypeptide is selected from SEQ ID NO: 73, SEQ ID NO: 74, SEQ ID NO: 75, SEQ ID NO: 76, SEQ ID NO: 78, SEQ ID NO: 84, SEQ ID NO: 85, SEQ ID NO: 86, SEQ ID NO: 88, SEQ ID NO: 91, SEQ ID NO: 92, SEQ ID NO: 95, SEQ ID NO: 110, SEQ ID NO: 112, SEQ ID NO: 119, SEQ ID NO: 123, SEQ ID NO: 125, SEQ ID NO: 128, SEQ ID NO: 129, SEQ ID NO: 133, SEQ ID NO: 134, SEQ ID NO: 135, SEQ ID NO: 218, SEQ ID NO: 219, SEQ ID NO: 220, SEQ ID NO: 221, SEQ ID NO: 222, and/or SEQ ID NO: 224.

Preferably the at least one sequence comprised in the VH FW1-CDR1-FW2-CDR2-FW3 segments or the VH FW1-HCDR1-FW2-HCDR2-FW3-HCDR3-FW4 segments comprised in the VH polypeptide is from selected from SEQ ID NO: 138, SEQ ID NO: 139, SEQ ID NO: 140, SEQ ID NO: 141, SEQ ID NO: 143, SEQ ID NO: 149, SEQ ID NO: 150, SEQ ID NO: 151, SEQ ID NO: 153, SEQ ID NO: 156, SEQ ID NO: 157, SEQ ID NO: 160, SEQ ID NO: 175, SEQ ID NO: 177, SEQ ID NO: 184, SEQ ID NO: 188, SEQ ID NO: 190, SEQ ID NO: 193, SEQ ID NO: 194, SEQ ID NO: 198, SEQ ID NO: 199, SEQ ID NO: 200, SEQ ID NO: 225, SEQ ID NO: 226, SEQ ID NO: 227, SEQ ID NO: 228, SEQ ID NO: 229, SEQ ID NO: 230, and/or SEQ ID NO: 231.

It was found that variable heavy chains and variable light chains comprising polypeptide members comprising the VH FW1-CDR1-FW2-CDR2-FW3 and VL FW1-CDR1-FW2-CDR2-FW3 and/or VH FW1-HCDR1-FW2-HCDR2-FW3-HCDR3-FW4 and VL FW1-LCDR1-FW2-LCDR2-FW3-LCDR3-FW4 segments of the invention are compatible with phage display and show good expression levels as scFV and as Fab fragments and thus represent ideal scaffolds to build a synthetic feline antibody polypeptide library with pre-selected and tested framework regions.

A further important aspect of the invention relates to a polypeptide library comprising at least one VL/VH polypeptide member combination comprising at least one VL polypeptide member described above and at least VH polypeptide member described above. Preferably the library comprises at least one VL polypeptide member comprising a sequence selected from selected from SEQ ID NO 236, SEQ ID NO 237, SEQ ID NO 238, SEQ ID NO 239, more preferably SEQ ID NO: 73, SEQ ID NO: 74, SEQ ID NO: 75, SEQ ID NO: 76, SEQ ID NO: 78, SEQ ID NO: 84, SEQ ID NO: 85, SEQ ID NO: 86, SEQ ID NO: 88, SEQ ID NO: 91, SEQ ID NO: 92, SEQ ID NO: 95, SEQ ID NO: 110, SEQ ID NO: 112, SEQ ID NO: 119, SEQ ID NO: 123, SEQ ID NO: 125, SEQ ID NO: 128, SEQ ID NO: 129, SEQ ID NO: 133, SEQ ID NO: 134, SEQ ID NO: 135, SEQ ID NO: 218, SEQ ID NO: 219, SEQ ID NO: 220, SEQ ID NO: 221, SEQ ID NO: 222, and/or SEQ ID NO: 224, and at least VH polypeptide member comprising a sequence selected from SEQ ID NO 232, SEQ ID NO 233, SEQ ID NO 234, SEQ ID NO 235, more preferably SEQ ID NO: 138, SEQ ID NO: 139, SEQ ID NO: 140, SEQ ID NO: 141, SEQ ID NO: 143, SEQ ID NO: 149, SEQ ID NO: 150, SEQ ID NO: 151, SEQ ID NO: 153, SEQ ID NO: 156, SEQ ID NO: 157, SEQ ID NO: 160, SEQ ID NO: 175, SEQ ID NO: 177, SEQ ID NO: 184, SEQ ID NO: 188, SEQ ID NO: 190, SEQ ID NO: 193, SEQ ID NO: 194, SEQ ID NO: 198, SEQ ID NO: 199, SEQ ID NO: 200, SEQ ID NO: 225, SEQ ID NO: 226, SEQ ID NO: 227, SEQ ID NO: 228, SEQ ID NO: 229, SEQ ID NO: 230, and/or SEQ ID NO: 231. Preferably the library comprises at least 2, at least 5, at least 10, at least 15, or preferably all of said VL and VH polypeptide members.

A polypeptide library comprising at least one VL/VH polypeptide member combination comprising a sequence combination selected from SEQ ID NO 236 and SEQ ID NO 232, SEQ ID NO 237 and SEQ ID NO 233, SEQ ID NO 238 and SEQ ID NO 234, SEQ ID NO 239 and SEQ ID NO 235, more preferably SEQ ID NO: 73 and SEQ ID NO: 138, SEQ ID NO: 74 and SEQ ID NO: 139, SEQ ID NO: 75 and SEQ ID NO: 140, SEQ ID NO: 76 and SEQ ID NO: 141, SEQ ID NO: 77 and SEQ ID NO: 142, SEQ ID NO: 78 and SEQ ID NO: 143, SEQ ID NO: 79 and SEQ ID NO: 144, SEQ ID NO: 80 and SEQ ID NO: 145, SEQ ID NO: 81 and SEQ ID NO: 146, SEQ ID NO: 82 and SEQ ID NO: 147, SEQ ID NO: 83 and SEQ ID NO: 148, SEQ ID NO: 84 and SEQ ID NO: 149, SEQ ID NO: 85 and SEQ ID NO: 150, SEQ ID NO: 86 and SEQ ID NO: 151, SEQ ID NO: 87 and SEQ ID NO: 152, SEQ ID NO: 88 and SEQ ID NO: 153, SEQ ID NO: 89 and SEQ ID NO: 154, SEQ ID NO: 90 and SEQ ID NO: 155, SEQ ID NO: 91 and SEQ ID NO: 156, SEQ ID NO: 92 and SEQ ID NO: 157, SEQ ID NO: 93 and SEQ ID NO: 158, SEQ ID NO: 94 and SEQ ID NO: 159, SEQ ID NO: 95 and SEQ ID NO: 160, SEQ ID NO: 96 and SEQ ID NO: 161, SEQ ID NO: 97 and SEQ ID NO: 162, SEQ ID NO: 98 and SEQ ID NO: 163, SEQ ID NO: 99 and SEQ ID NO: 164, SEQ ID NO: 100 and SEQ ID NO: 165, SEQ ID NO: 101 and SEQ ID NO: 166, SEQ ID NO: 102 and SEQ ID NO: 167, SEQ ID NO: 103 and SEQ ID NO: 168, SEQ ID NO: 104 and SEQ ID NO: 169, SEQ ID NO: 105 and SEQ ID NO: 170, SEQ ID NO: 106 and SEQ ID NO: 171, SEQ ID NO: 107 and SEQ ID NO: 172, SEQ ID NO: 108 and SEQ ID NO: 173, SEQ ID NO: 109 and SEQ ID NO: 174, SEQ ID NO: 110 and SEQ ID NO: 175, SEQ ID NO: 111 and SEQ ID NO: 176, SEQ ID NO: 112 and SEQ ID NO: 177, SEQ ID NO: 113 and SEQ ID NO: 178, SEQ ID NO: 114 and SEQ ID NO: 179, SEQ ID NO: 115 and SEQ ID NO: 180, SEQ ID NO: 116 and SEQ ID NO: 181, SEQ ID NO: 117 and SEQ ID NO: 182, SEQ ID NO: 118 and SEQ ID NO: 183, SEQ ID NO: 119 and SEQ ID NO: 184, SEQ ID NO: 120 and SEQ ID NO: 185, SEQ ID NO: 121 and SEQ ID NO: 186, SEQ ID NO: 122 and SEQ ID NO: 187, SEQ ID NO: 123 and SEQ ID NO: 188, SEQ ID NO: 124 and SEQ ID NO: 189, SEQ ID NO: 125 and SEQ ID NO: 190, SEQ ID NO: 126 and SEQ ID NO: 191, SEQ ID NO: 127 and SEQ ID NO: 192, SEQ ID NO: 128 and SEQ ID NO: 193, SEQ ID NO: 129 and SEQ ID NO: 194, SEQ ID NO: 130 and SEQ ID NO: 195, SEQ ID NO: 131 and SEQ ID NO: 196, SEQ ID NO: 132 and SEQ ID NO: 197, SEQ ID NO: 133 and SEQ ID NO: 198, SEQ ID NO: 134 and SEQ ID NO: 199, SEQ ID NO: 135 and SEQ ID NO: 200, SEQ ID NO: 136 and SEQ ID NO: 201, SEQ ID NO: 137 and SEQ ID NO: 202, SEQ ID NO: 218 and SEQ ID NO: 225, SEQ ID NO 219 and SEQ ID NO 226, SEQ ID NO: 220 and SEQ ID NO: 227, SEQ ID NO: 221 and SEQ ID NO 228, SEQ ID NO: 222 and SEQ ID NO: 229, SEQ ID NO: 223 and SEQ ID NO: 230 and/or SEQ ID NO: 224 and SEQ ID NO: 230. Preferably the polypeptide library comprises at least 2, at least 5, at least 10, at least 15, or preferably all of said VL/VH polypeptide member combinations.

Preferably, the polypeptide library comprises at least one VL/VH polypeptide member combination comprising a sequence combination selected from SEQ ID NO: 73 and SEQ ID NO: 138, SEQ ID NO: 74 and SEQ ID NO: 139, SEQ ID NO: 75 and SEQ ID NO: 140, SEQ ID NO: 76 and SEQ ID NO: 141, SEQ ID NO: 78 and SEQ ID NO: 143, SEQ ID NO: 84 and SEQ ID NO: 149, SEQ ID NO: 85 and SEQ ID NO: 150, SEQ ID NO: 86 and SEQ ID NO: 151, SEQ ID NO: 88 and SEQ ID NO: 153, SEQ ID NO: 91 and SEQ ID NO: 156, SEQ ID NO: 92 and SEQ ID NO: 157, SEQ ID NO: 95 and SEQ ID NO: 160, SEQ ID NO: 110 and SEQ ID NO: 175, SEQ ID NO: 112 and SEQ ID NO: 177, SEQ ID NO: 119 and SEQ ID NO: 184, SEQ ID NO: 123 and SEQ ID NO: 188, SEQ ID NO: 125 and SEQ ID NO: 190, SEQ ID NO: 128 and SEQ ID NO: 193, SEQ ID NO: 129 and SEQ ID NO: 194, SEQ ID NO: 133 and SEQ ID NO: 198, SEQ ID NO: 134 and SEQ ID NO: 199, SEQ ID NO: 135 and SEQ ID NO: 200, SEQ ID NO: 218 and SEQ ID NO: 225, SEQ ID NO 219 and SEQ ID NO 226, SEQ ID NO: 220 and SEQ ID NO: 227, SEQ ID NO: 221 and SEQ ID NO 228, SEQ ID NO: 222 and SEQ ID NO: 229, SEQ ID NO: 223 and SEQ ID NO: 230 and/or SEQ ID NO: 224 and/or SEQ ID NO: 230. Preferably the polypeptide library comprises at least 2, at least 5, at least 10, at least 15, or preferably all of said VL/VH polypeptide member combinations.

The described VL/VH polypeptide member combinations may be comprised in the polypeptide library in form wherein each of the VL FW1 to FW3 polypeptide sequences and each of the VH FW1 to FW3 polypeptide sequences of specific VL/VH polypeptide member combinations are comprised in one polypeptide molecule wherein the VL FW1 to FW3 polypeptide sequences is arranged N-terminally of the VH FW1 to FW3 polypeptide sequence. Accordingly, the VL/VH polypeptide member combinations may be comprised in the library in a form wherein each VL FW1-LCDR1-FW2-LCDR2-FW3-LCDR3-FW4 segment polypeptide sequence and each VH FW1-HCDR1-FW2-HCDR2-FW3-HCDR3-FW4 segment polypeptide sequence wherein the VL FW1-LCDR1-FW2-LCDR2-FW3-LCDR3-FW4 segment polypeptide sequences is arranged N-terminally of the VH FW1-HCDR1-FW2-HCDR2-FW3-HCDR3-FW4 segment polypeptide sequence.

In a highly preferred embodiment, the polypeptide comprising the VL/VH polypeptide member combinations may preferably be a scFv. Accordingly, the invention relates to a polypeptide library comprising at least one scFv comprising any of the VH FW1-CDR1-FW2-CDR2-FW3 segments, VL FW1-CDR1-FW2-CDR2-FW3 segments, VH FW1-HCDR1-FW2-HCDR2-FW3-HCDR3-FW4 segments, VL FW1-LCDR1-FW2-LCDR2-FW3-LCDR3-FW4 segments or VL/VH polypeptide member combinations described herein. A respective library may be referred to as “scFv antibody library”. In these embodiments, the VL and VH FW1-CDR1-FW2-CDR2-FW3 segments or VL/VH polypeptide member combinations are comprised in polypeptide member which furthermore comprise the VL and VH CDR3-FR4 segments C-terminally of the respective VL and VH FW1-CDR1-FW2-CDR2-FW3 segments. Specifically, the VL/VH polypeptide member combinations are comprised in in the library in form of a polypeptide VL (FW1-LCDR1-FW2-LCDR2-FW3-LCDR3-FW4)-LINKER-VH (FW1-HCDR1-FW2-HCDR2-FW3-HCDR3-FW4).

In an alternative embodiment, the described VL/VH polypeptide member combinations may be comprised in the polypeptide library in a form of two polypeptides, wherein a VL polypeptide comprises the VL FW1 to FW3 and/or VL FW1-LCDR1-FW2-LCDR2-FW3-LCDR3-FW4 segment polypeptide sequence and an antibody constant light chain sequence in C-terminal direction of the VL FW1-LCDR1-FW2-LCDR2-FW3-LCDR3-FW4 segment polypeptide sequence and a VH polypeptide comprises the VH FW1 to FW3 and/or VH FW1-HCDR1-FW2-HCDR2-FW3-HCDR3-FW4 polypeptide sequence and an antibody CH1 constant heavy chain sequence in C-terminal direction of the VH FW1-HCDR1-FW2-HCDR2-FW3-HCDR3-FW4 segment polypeptide sequence. Preferably the VL polypeptide and the VH polypeptide is linked by a disulphide bridged between the constant light chain sequence and the CH1 constant heavy chain sequence. Alternatively, VL polypeptide and the VH polypeptide may be linked by a linker. The VL/VH polypeptide member combinations may preferably be Fab antibody fragments. More specifically, the VL polypeptide comprises the VL FW1 to FW3 polypeptide sequence in a VL CDR1-FW2-CDR2-FW3-CDR3-FR4 polypeptide with a C-terminally linked constant light chain and a VH FW1 to FW3 polypeptide sequence in a VH CDR1-FW2-CDR2-FW3-CDR3-FR4 polypeptide with a C-terminally linked C1 constant heavy chain sequence.

Accordingly, in a further embodiment the invention relates to a polypeptide library comprising at least one Fab antibody fragment comprising any of the VH FW1-CDR1-FW2-CDR2-FW3 segments, VL FW1-CDR1-FW2-CDR2-FW3 segments, VH FW1-HCDR1-FW2-HCDR2-FW3-HCDR3-FW4 segments, VL FW1-LCDR1-FW2-LCDR2-FW3-LCDR3-FW4 segments, or VL/VH polypeptide member combination described herein. A respective library may be referred to as “Fab antibody library”.

A further aspect of the present invention relates to libraries as described herein comprising polypeptide members or polypeptide member combinations which comprise sequences having a sequence identity of at least about 70%, at least about 80%, at least about 85%, at least about 90%, at least about 95% or at least about 99% to the sequences disclosed above. Such sequences may also be referred to as substantially identical sequences.

In a specific embodiment of an scFv library the library comprises at least one scFv fragment, wherein the VH polypeptide member is N-terminally linked to the VL polypeptide member by a linker of about 15 to about 25, preferably about 16 to about 21, more preferably about 17 to about 19, most preferably about 18 amino acids. In a preferred embodiment, the linker comprises an amino acid GGSSRSSSSGGGGGGGG (SEQ ID NO: 207).

In a further embodiment of an scFv library, the VL/VH polypeptide member combination may comprise a N- or C-terminal affinity tag, preferably a FLAG tag. The affinity tag facilitates isolation and purification of an scFv antibody fragment. In a further embodiment of an scFv library, the VL/VH polypeptide member combination may comprise a full length or truncated phage pIII polypeptide sequence linked to the VH polypeptide. Preferably, the VL/VH polypeptide member combination comprises a FLAG tag linked N-terminally linked to the VL polypeptide sequence and a phage pill polypeptide C-terminally linked to the VH polypeptide sequence.

In the embodiments described above, the VH polypeptides further comprise a feline variable heavy chain framework 4 region (FW4 region). The VH FW4 region is comprised in the VH polypeptides C-terminally of the FW3 sequence. In a preferred embodiment, at least one VH polypeptides comprises a FW4 region wherein the sequence is selected from amino acid sequences WGQGALVTVSS (SEQ ID NO: 213), WGQGAPVTVSS″ (SEQ ID NO: 214), WGQGTLVTVSS (SEQ ID NO: 215) WGQGVLVTVSS (SEQ ID NO: 216) and WGRGALVTVSS (SEQ ID NO: 217). In the most preferred embodiment, one VH polypeptides comprises a FW4 region sequence selected from amino acid sequences WGQGALVTVSS (SEQ ID NO: 213).

In the embodiments described above, the VL polypeptides further comprise a feline variable light chain framework 4 region (FW4 region). The VL FW4 region is comprised in the VL polypeptides C-terminally of the FW3 sequence. In a preferred embodiment, at least one VL polypeptides comprises a FW4 region sequence selected from amino acid sequences FGGGTHLTVL (SEQ ID NO: 208), FGGGTHLSVL (SEQ ID NO: 209), FGRGTHLTVL (SEQ ID NO: 210), FGGGTHVTVL (SEQ ID NO: 211) and IGGGTHVTVL (SEQ ID NO: 212). In the most preferred embodiment, one VL polypeptides comprises a FW4 region sequence selected from amino acid sequences FGGGTHLTVL (SEQ ID NO: 208)

In the embodiments described above, different VL and VH polypeptide members comprise different feline specific CDR3 polypeptide sequences (LCDR3 in the VL and HCDR3 in the VH polypeptide) between the FW3 and the FW4 segments.

Within the context of the present invention, “feline specific CDR3 polypeptide sequences” are CDR3 polypeptide sequences naturally expressed in felines or synthetic sequences based on naturally occurring sequences.

In a preferred embodiment, the CDR3 polypeptide sequences comprise randomized or partially randomized amino acid sequences.

In certain embodiments the present disclosure provides a polypeptide library which comprises HCDR3s which cover more than 50% of the naturally occurring HCDR3 lengths of the feline HCDR3 repertoire. In other aspects said polypeptide library comprises more than 60%, more than 70%, more than 75%, more than 80%, more than 85%, more than 90% or more than 95% of the naturally occurring HCDR3 lengths of the feline HCDR3 repertoire.

In certain embodiments the present disclosure provides a polypeptide library, wherein the HCDR3 regions of essentially all members of the library are flanked by unique restriction sites.

In certain aspects the present disclosure provides a polypeptide library which comprises HCDR3s lengths of 3-16 amino acids according to the numbering scheme depicted in Table 9 and FIG. 9. Preferably HCDR3s comprised in the library have a length of 7-14 amino acids. More preferably, the HCDRs may have an average length of 8-13 amino acids, or 10-13 amino acids, and most preferably 10-12 amino acids. Preferably the amino acid sequence id the HCDR3s is fully or partially randomized.

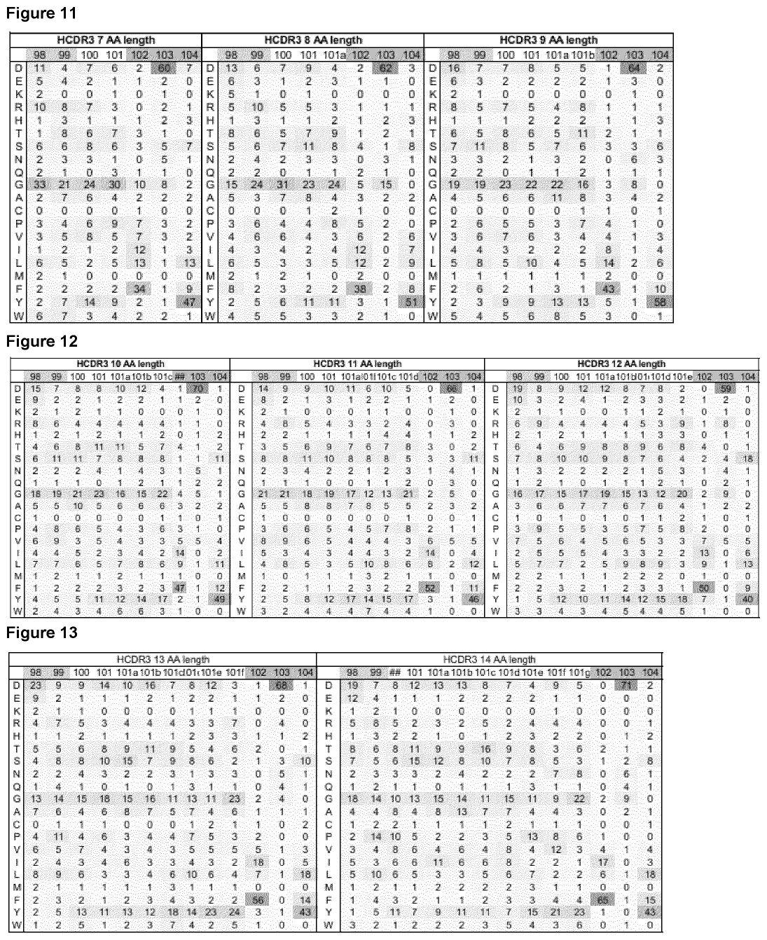

In certain aspects the HCDR3 comprised in the polypeptide library according to the present invention comprises randomly diversified HCDR3s. The HCDR3 comprised in the library may have a design as shown in Table 9. Preferably, HCDR3s comprised in the polypeptide library of the present having a length of 7-9 amino acids may have an amino acid distribution as shown in FIG. 11, and HCDR3s comprised in the library of the present having a length of 10-12 amino acids may have an amino acid distribution as shown in FIG. 12, and HCDR3s comprised in the library of the present having a length of 13-14 amino acids may have an amino acid distribution as shown in FIG. 13. As an example, of the HCDR3s comprised in the library having a length of 13 amino acids having an amino acid distribution as shown in FIG. 13, 23% have a D in position 98, 9% have a D in position 99, 9% have a D in position 100, 9% have an E in position 98, 2% have a E in position 99, and so on.

In certain aspects the present disclosure provides a synthetic feline antibody polypeptide library in which the HCDR3 region has a diversity of at least about 1.0*10⁹. In other aspects, the present disclosure provides a polypeptide library which the HCDR3 region has a diversity of at least about 1.0*10¹⁰, of at least about 1.0*10¹¹, of at least about 1.0*10¹² or of at least about 1.0*10¹³.

The generation of diversified HCDR3 regions is describe in Example 7. Polynucleotides encoding the diversified HCDR3 regions described herein may be referred to as “HCDR3 diversity module”.

In certain aspects the present invention provides a polypeptide library wherein the LCDR3 regions of essentially all members of the library are flanked by unique restriction sites.

In certain aspects the present invention provides a polypeptide library which comprises LCDR3s which cover more than 50% of the naturally occurring LCDR3 lengths of the feline LCDR3 repertoire. In other aspects said polypeptide library comprises more than 60%, more than 70%, more than 75%, more than 80%, more than 85%, more than 90% or more than 95% of the naturally occurring LCDR3 lengths of the feline LCDR3 repertoire.

In certain aspects the present disclosure provides a polypeptide library which comprises LCDR3s for VL lambda of a length of 8-13 amino acids acids according to the numbering scheme depicted in Table 10 and FIG. 10. Preferably 9-12 amino acids.

In an alternative embodiment the VL may be a kappa light chain.

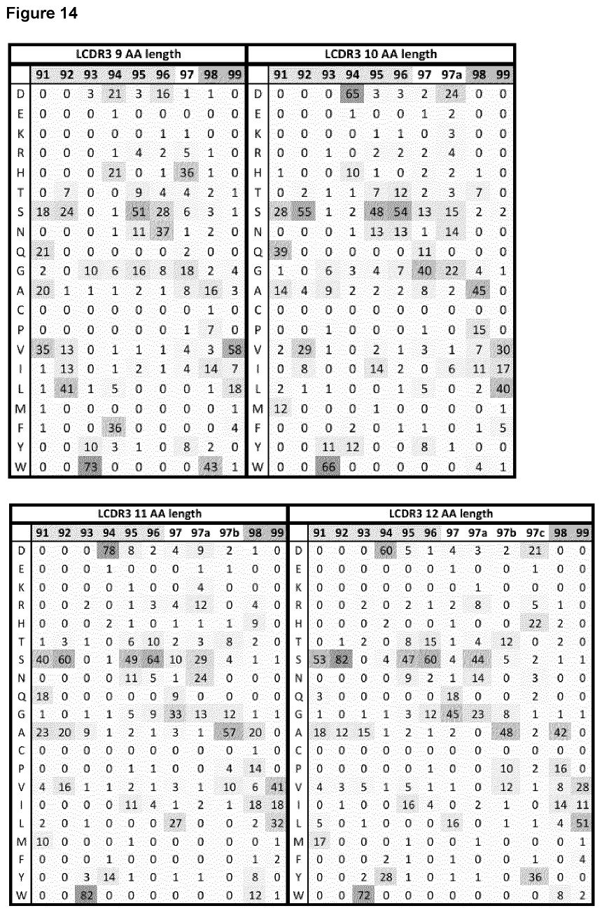

In certain aspects the LCDR3 comprises randomly diversified LCDRs. The LCDR3s comprised in the polypeptide library may have a design as shown in Table 10. Preferably, LCDRs comprised in the polypeptide library of the present having a length of 9-12 amino acids may have an amino acid distribution as shown in FIG. 14.

In certain aspects the present disclosure provides a synthetic feline antibody polypeptide library in which the LCDR3 region has a diversity of at least about 1.0*10⁵. In other aspects the present invention provides a polypeptide library which the LCDR3 region has a diversity of at least about 11.0*10⁶, of at least about 1.0*10⁷ or of at least about 1.0*10⁸.

The generation of diversified LCDR3 regions is describe in Example 8. Polynucleotides encoding the diversified LCDR3 regions described herein may be referred to as “LCDR3 diversity module.

In certain aspects the present disclosure provides a polypeptide library which comprises an HCDR3 design as shown in Table 9 and FIGS. 11-13 and an LCDR3 design as shown in Table 10 and FIG. 14.

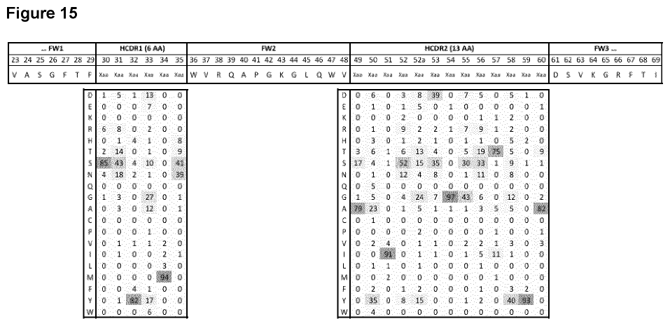

In a further aspect, the VH polypeptide sequences comprised in the polypeptide library may comprise a diversified VH HCDR1-FW2-HCDR2 segment to allow for affinity maturation. In the diversified VH HCDR1-FW2-HCDR2 segment according to the invention, the HCDR1 preferably has a fixed length of 6 amino acids and the HCDR2 preferably has a fixed length of 13 amino acids. The randomly diversified HCDR1s and HCDR2s comprised in the VH HCDR1-FW2-HCDR2 segment may have a design as shown in FIG. 15. Preferably, the diversified FW2 comprised in the diversifies VH HCDR1-FW2-HCDR2 segment has a sequence of WVRQAPGKGLQWV (SEQ ID No: 241) as shown in in FIG. 15.

The polypeptide library may comprise VH polypeptide members as described above, wherein the VH CDR1-FW2-CDR2 of the members described above is replaced by a diversified VH HCDR1-FW2-HCDR2 segment.

The generation of diversified VH HCDR1-FW2-HCDR2 segment is describe in Example 9. Polynucleotides encoding a diversified VH HCDR1-FW2-HCDR2 segments described herein may be referred to as “VH maturation module”.

In a further aspect, the polypeptide library may comprise VL polypeptide members, wherein the VL LCDR1-FW2-LCDR2 segment is replaced by a diversified VL LCDR1-FW2-LCDR2 segment which comprises an LCDR1 and/or an LCDR2 with a partially or fully random amino acids sequence. Polynucleotides encoding a diversified VL HCDR1-FW2-HCDR2 segments described herein may be referred to as “VL maturation module”.

In a further embodiment the polypeptide members comprised in the polypeptide library are devoid of post-translational modification (PTM) sites to optimize expression and biophysical properties of the respective VH or VL polypeptide members. PTMs not necessarily take place in any antibody sample produced, but might take place in antibody samples of high concentration and under long storage conditions, and may also occur in vivo. PTMs can interfere with antibody stability and/or homogeneity and might lead to loss of antibody functionality. Examples of PTMs include but are not limited to oxidation (Met, Trp, His), deamidation (Asn, Gln), isomerization (Asp) or N-linked glycosylation (Asn). The VH or VL polypeptide members may also be devoid of other unwanted sequences. Preferably the VH or VL polypeptide members do not comprise glycosylation motifs (including amino acid sequences NxS NxT-x not P; for example: NAS, NCS, ND, S NES, NFS, NGS, NHS, NIS, NKS, NLS, NMS, NNS, NQS, NRS, NSS, NTS, NVS, NWS, NYS, NAT, NCT, NDT, NET, NFT, NGT, NHT, NIT, NKT, NLT, NMT, NNT, NQT, NRT, NST, NTT, NVT, NWT, NYT), asparagine deamidation motifs (including amino acid sequences NG, NS, NT, NH), aspartate isomerization motifs (including amino acid sequences DG DS DT DD DH), lysine glycation motifs (including amino acid sequences KE KD KK), integrin binding aVb3 motifs (including amino acid sequences RGD RYD KGD NGR), integrin binding a4b1 motifs (including amino acid sequence LDV), integrin binding a2b1 motif (including amino acid sequences DGE), CD11c/CD18 binding motif (including amino acid sequences GPR), fragmentation motifs (including amino acid sequences DP, DQ), hydrophobicity motifs (including amino acid sequences FF, FW, WW, WF).

In yet other aspects the present disclosure provides a polypeptide library comprising VL/VH polypeptide member combinations, wherein substantially all VL/VH combination are efficiently displayed on a phage. Display can be measured by sandwich phage ELISA as described herein in Example 4 or by Western blot analysis.

In another aspects the VL/VH polypeptide member combinations comprised in the library of the invention are substantially all expressed in E. coli in Fab format having a monomeric content of at least about 60%, at least about 70%, at least about 80%, at least about 85%, at least about 90%, at least about 95%, at least about 98% or at least about 99%.

In a further embodiment, the VL and/or VH polypeptide members, and/or VL/VH polypeptide member combinations are well expressed in a mammalian system in IgG format. More specifically the present invention provides a polypeptide library comprising the VL and/or VH polypeptide members, and/or VL/VH polypeptide member combinations, wherein substantially all the VL and/or VH polypeptide members, and/or VL/VH polypeptide member combinations expressed in a mammalian system in IgG format have a monomeric content of at least about 60%, at least about 70%, at least about 80%, at least about 85%, at least about 90%, at least about 95%, at least about 98% or at least about 99%. In certain aspects said mammalian system comprises cells selected from HKB11 cells, PERC.6 cells, or CHO cells.

The VL and/or VH polypeptide members, and/or VL/VH polypeptide member combinations comprised in the polypeptide library according to the invention may be well expressed in bacterial culture at levels of more than about 1 mg/L, more than about 5 mg/L, or more than about 10 mg/L in a bacterial culture.

The VL and/or VH polypeptide members, and/or VL/VH polypeptide member combinations comprised in the polypeptide library according to the invention may be well expressed in IgG format in a mammalian system at levels of more than about 10 mg/L, more than about 50 mg/L, or more than about 100 mg/L; or more than about 150 mg/L; or more than about 200 mg/L.

In certain aspects the present invention provides a polypeptide library wherein all or substantially all VL/VH polypeptide member combinations are thermally stable. Thermal stability can be measured by differential scanning fluorimetry, especially nano differential scanning fluorimetry according to methods commonly known. In certain aspects the present disclosure provides a library wherein essentially all VL/VH polypeptide member combinations have a Tm of ≥60° C. In further embodiments, substantially all VL/VH polypeptide member combinations comprised in the library may have a Tm of ≥62° C., preferably a Tm of ≥64° C., more preferably a Tm of ≥66° C., even more preferably a Tm of ≥68° C., and most preferably a Tm of ≥70° C.

In a further aspect the present invention relates to a collection of nucleic acid molecules encoding a polypeptide library as describes above.

In a certain aspect the invention relates to a collection of nucleic acid molecules encoding a polypeptide library comprising VL polypeptide members comprising VL FW1-CDR1-FW2-CDR2-FW3 segments comprising at least one sequence selected from SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 218, SEQ ID NO: 219, SEQ ID NO: 220, SEQ ID NO: 221, SEQ ID NO: 222, SEQ ID NO: 224, and/or SEQ ID NO: 224.

In a certain aspect the invention relates to a collection of nucleic acid molecules encoding a polypeptide library comprising VL polypeptide members comprising VL FW1-LCDR1-FW2-LCDR2-FW3-LCDR3-FW4 segments comprising at least one sequence selected from SEQ ID NO 236, SEQ ID NO 237, SEQ ID NO 238, or SEQ ID NO 239.

In a further aspect the invention relates to a collection of nucleic acid molecules encoding a polypeptide library comprising VH polypeptide members comprising VH polypeptide members comprising FW1-CDR1-FW2-CDR2-FW3 segments comprising at least one sequence selected from SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 225, SEQ ID NO: 226, SEQ ID NO: 227, SEQ ID NO: 228, SEQ ID NO: 229, SEQ ID NO: 230, and/or SEQ ID NO: 231.

In a certain aspect the invention relates to a collection of nucleic acid molecules encoding a polypeptide library comprising VH polypeptide members comprising VH FW1-HCDR1-FW2-HCDR2-FW3-HCDR3-FW4 segments comprising at least one sequence selected from SEQ ID NO 232, SEQ ID NO 233, SEQ ID NO 234, or SEQ ID NO 235.

In a further aspect the invention relates to a collection of nucleic acid molecules encoding a polypeptide library comprising at least one VL/VH polypeptide member combination comprising at least one VL polypeptide member described above and at least VH polypeptide member described above. Preferably the collection of nucleic acid molecules encodes a polypeptide library comprises at least at least one VL polypeptide member comprising a sequence selected from selected from SEQ ID NO: 73, SEQ ID NO: 74, SEQ ID NO: 75, SEQ ID NO: 76, SEQ ID NO: 78, SEQ ID NO: 84, SEQ ID NO: 85, SEQ ID NO: 86, SEQ ID NO: 88, SEQ ID NO: 91, SEQ ID NO: 92, SEQ ID NO: 95, SEQ ID NO: 110, SEQ ID NO: 112, SEQ ID NO: 119, SEQ ID NO: 123, SEQ ID NO: 125, SEQ ID NO: 128, SEQ ID NO: 129, SEQ ID NO: 133, SEQ ID NO: 134, SEQ ID NO: 135 SEQ ID NO: 218, SEQ ID NO: 219, SEQ ID NO: 220, SEQ ID NO: 221, SEQ ID NO: 222, and/or SEQ ID NO: 224; and at least VH polypeptide member comprising a sequence selected from SEQ ID NO: 138, SEQ ID NO: 139, SEQ ID NO: 140, SEQ ID NO: 141, SEQ ID NO: 143, SEQ ID NO: 149, SEQ ID NO: 150 SEQ ID NO: 151, SEQ ID NO: 153, SEQ ID NO: 156, SEQ ID NO: 157, SEQ ID NO: 160, SEQ ID NO: 175, SEQ ID NO: 177, SEQ ID NO: 184, SEQ ID NO: 188, SEQ ID NO: 190, SEQ ID NO: 193, SEQ ID NO: 194, SEQ ID NO: 198, SEQ ID NO: 199, SEQ ID NO: 200, SEQ ID NO: 225, SEQ ID NO: 226, SEQ ID NO: 227, SEQ ID NO: 228, SEQ ID NO: 229, SEQ ID NO: 230, and/or SEQ ID NO: 231.

In a further preferred aspect the invention relates to a collection of nucleic acid molecules encoding a polypeptide library comprising at least one VL/VH polypeptide member combination comprising a sequence combination selected from SEQ ID NO: 236 and SEQ ID NO: 232, SEQ ID NO: 237 and SEQ ID NO: 233, SEQ ID NO: 238 and SEQ ID NO: 234, SEQ ID NO: 239 and SEQ ID NO: 235, SEQ ID NO: 73 and SEQ ID NO: 138, SEQ ID NO: 74 and SEQ ID NO: 139, SEQ ID NO: 75 and SEQ ID NO: 140, SEQ ID NO: 76 and SEQ ID NO: 141, SEQ ID NO: 77 and SEQ ID NO: 142, SEQ ID NO: 78 and SEQ ID NO: 143, SEQ ID NO: 79 and SEQ ID NO: 144, SEQ ID NO: 80 and SEQ ID NO: 145, SEQ ID NO: 81 and SEQ ID NO: 146, SEQ ID NO: 82 and SEQ ID NO: 147, SEQ ID NO: 83 and SEQ ID NO: 148, SEQ ID NO: 84 and SEQ ID NO: 149, SEQ ID NO: 85 and SEQ ID NO: 150, SEQ ID NO: 86 and SEQ ID NO: 151, SEQ ID NO: 87 and SEQ ID NO: 152, SEQ ID NO: 88 and SEQ ID NO: 153, SEQ ID NO: 89 and SEQ ID NO: 154, SEQ ID NO: 90 and SEQ ID NO: 155, SEQ ID NO: 91 and SEQ ID NO: 156, SEQ ID NO: 92 and SEQ ID NO: 157, SEQ ID NO: 93 and SEQ ID NO: 158, SEQ ID NO: 94 and SEQ ID NO: 159, SEQ ID NO: 95 and SEQ ID NO: 160, SEQ ID NO: 96 and SEQ ID NO: 161, SEQ ID NO: 97 and SEQ ID NO: 162, SEQ ID NO: 98 and SEQ ID NO: 163, SEQ ID NO: 99 and SEQ ID NO: 164, SEQ ID NO: 100 and SEQ ID NO: 165, SEQ ID NO: 101 and SEQ ID NO: 166, SEQ ID NO: 102 and SEQ ID NO: 167, SEQ ID NO: 103 and SEQ ID NO: 168, SEQ ID NO: 104 and SEQ ID NO: 169, SEQ ID NO: 105 and SEQ ID NO: 170, SEQ ID NO: 106 and SEQ ID NO: 171, SEQ ID NO: 107 and SEQ ID NO: 172, SEQ ID NO: 108 and SEQ ID NO: 173, SEQ ID NO: 109 and SEQ ID NO: 174, SEQ ID NO: 110 and SEQ ID NO: 175, SEQ ID NO: 111 and SEQ ID NO: 176, SEQ ID NO: 112 and SEQ ID NO: 177, SEQ ID NO: 113 and SEQ ID NO: 178, SEQ ID NO: 114 and SEQ ID NO: 179, SEQ ID NO: 115 and SEQ ID NO: 180, SEQ ID NO: 116 and SEQ ID NO: 181, SEQ ID NO: 117 and SEQ ID NO: 182, SEQ ID NO: 118 and SEQ ID NO: 183, SEQ ID NO: 119 and SEQ ID NO: 184, SEQ ID NO: 120 and SEQ ID NO: 185, SEQ ID NO: 121 and SEQ ID NO: 186, SEQ ID NO: 122 and SEQ ID NO: 187, SEQ ID NO: 123 and SEQ ID NO: 188, SEQ ID NO: 124 and SEQ ID NO: 189, SEQ ID NO: 125 and SEQ ID NO: 190, SEQ ID NO: 126 and SEQ ID NO: 191, SEQ ID NO: 127 and SEQ ID NO: 192, SEQ ID NO: 128 and SEQ ID NO: 193, SEQ ID NO: 129 and SEQ ID NO: 194, SEQ ID NO: 130 and SEQ ID NO: 195, SEQ ID NO: 131 and SEQ ID NO: 196, SEQ ID NO: 132 and SEQ ID NO: 197, SEQ ID NO: 133 and SEQ ID NO: 198, SEQ ID NO: 134 and SEQ ID NO: 199, SEQ ID NO: 135 and SEQ ID NO: 200, SEQ ID NO: 136 and SEQ ID NO: 201, and/or SEQ ID NO: 137 and SEQ ID NO: 202, SEQ ID NO: 218 and SEQ ID NO: 225, SEQ ID NO 219 and SEQ ID NO 226, SEQ ID NO: 220 and SEQ ID NO: 227, SEQ ID NO: 221 and SEQ ID NO 228, SEQ ID NO: 222 and SEQ ID NO: 229, SEQ ID NO: 223 and SEQ ID NO: 230 and/or SEQ ID NO: 224 and SEQ ID NO: 230.

In certain aspects the invention relates to a collection of vectors comprising the nucleic acid molecules described above. Furthermore, the invention relates to a recombinant host cell comprising the nucleic acid molecules and/or vectors disclosed herein.

In one aspect the present disclosure provides a method to isolate a binder specific for an antigen, said method comprising the steps of:
(a) contacting a library disclosed herein with an antigen;
(b) removing those members of the library which do not bind to the antigen; and
(c) recovering those members of the library bound to the antigen.

Accordingly, the invention relates to an antibody or antibody fragment obtained from the polypeptide library described herein. Preferably, the antibody or antibody fragment may be obtained by the afore described method.

In a more general aspect, the invention relates to a feline antibody or antibody fragment comprising a VL polypeptide member, and/or a VH polypeptide member or a VL/VH polypeptide member combination according as disclosed herein. Preferably feline antibody is a full antibody, a single chain Fv (scFv), a Fab fragment, or a F(ab)2 fragment.

The antibody according to the invention may be selected from an IgG1a, IgG1b, or IgG2 isotype. The isotype may also include modified versions of one of these classes, where modifications have been made to alter the Fc function, for example, to enhance or reduce effector functions or binding to Fc receptors.

In a further aspect, the invention relates to a method of generating a polypeptide library comprising the steps of:
(i) providing a feline cDNA generated from an antibody expressing tissue, preferably from splenocytes, lymphocytes, bone marrow.
(ii) amplifying VL and VH sequences, preferably by using at least one forward primer selected from SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28; and at least one reverse primer selected from SEQ ID NO: 1, SEQ ID NO: 2, and SEQ ID NO: 3 to obtain amplicons; and
(iii) generating VL/VH combined sequences by randomly linking the amplicons obtained in step ii).

In a preferred embodiment, the primers employed in step (ii) are designed to incorporate secondary primer binding sites into the 5′ end of the VL amplicons obtained by the amplification and the 3′ end of the VH amplicons obtained by the amplification and/or a flexible linker into the 3′ end of the VL amplicons and the 5′ end of the VH amplicons.

Preferably the VL and the VH amplicons are linked in step (iii) by a linker sequence which encodes 15 to 25 amino acids.

Optionally the methods further comprises a step of joining a sequence encoding an affinity tag to the VL/VH combined sequences obtained in step (iii).

The method of the invention may optionally further comprise a step (iv) of cloning the VL/VH combined sequences into vector, preferably selected from a plasmids, phagemids, or expression cassette.

The method of the invention may optionally further comprise a step (v) of expressing single chain Fv fragments or Fab fragments comprising the VL/VH combined sequences linked to a full length or truncated phage pIII polypeptide in a phage expression system.

The method of the invention may optionally further comprise a step (vi) of isolating phages expressing VL/VH combined sequences by affinity tag and optionally determining the sequences of VL/VH combined sequences in the isolated phages.

The method of the invention may optionally further comprise a step (vii) of infecting bacteria with phages isolated in step (iv). Preferably the bacteria are E. coli.

The method of the invention may optionally further comprise a step (viii) of subcloning the VL/VH combined sequences from isolated phages in step (vii) into an expression system and expressing the VL/VH combined sequences as soluble scFv fragments or Fab fragments. Preferably the expression system may be a bacterial expression system.

The method of the invention may optionally further comprise a step (ix) of determining the expression levels of soluble scFv fragments by ELISA in the expression system.

The method of the invention may optionally further comprise a step (x) of sequencing the DNA encoding VL/VH combined sequences from high expressing clones of the expression system.

The method of the invention may optionally further comprise a step (xi) of cloning the VL and VH combined sequences determined by sequencing in step (x) into a Fab expression system, and optionally a step (xii) of expressing the Fab fragments in cells and optionally a step (xiii) of determining the expression levels of soluble Fab fragments in said cells. In an optional step (xiv), the sequences of the VL and VH combined sequences in high expressing cells may be determined by sequencing.

Finally, VL/VH combined sequences that expressed high in the scFv and Fab format, and thus are considered stable, may be determined in a step xv) by comparing the sequences obtained in step (x) and step (xiv).

In a further embodiment, the method may comprise a step of introducing a LCDR3 diversity module as described above and/or a HCDR3 diversity module as described above into at least one VL and/or VH sequence of the combined sequences identified in the disclosed method, preferably into the sequences identified in step (xv).

In a further embodiment, the method may comprise a step of introducing a VL maturation module as described above and/or a VH maturation module as described above into an VL and/or VH sequence of the combined sequences identified in the disclosed method, preferably into the sequences identified in step (xv).

In a further embodiment step xi) comprises introducing a feline constant light part of the light chain (CL-lambda) and a constant heavy chain region (CH1) into the expression system such that the VL polypeptides are expressed with a C-terminally linked CL-lambda and the VH polypeptides are expressed with a C-terminally linked CH1 in the expression system.

Optionally mutating both cysteines to serine in the C-terminus of the CL and the N-terminus of CH1.

In a further aspect the invention relates to a polypeptide library or a collection of nucleic acids obtainable or obtained by the method of the invention.

### EXAMPLES

### Example 1: NGS Analysis of the Feline Immunoglobulin Repertoire

For generation of NGS data sets of primers were designed (modified from Steiniger et al. 2017) to PCR amplify VL and VH sequences from cat bone marrow cDNA (Amsbio #CD-704). Sequences were polyclonal cloned into pUC19 storage plasmids. For NGS, VL and VH sequences were amplified via PCR from the storage plasmids introducing overhangs for Illumina sequencing (Primer sequences are depicted in Table 1).

TABLE 1
Primers for amplifying VL/VH sequences from cDNA and for Illumnia NGS analysis

| | Primer for VL/VH amplification from cDNA | |
|---|---|---|
| SEQ ID NO | Name | Sequence (5′-3′) |
| 1 | Felix_VH_Rv | aggtcgactctagaggatccccACACCGATGGGGCCGTGGTGGA |
| 2 | Felix_VLK_Rv | aggtcgactctagaggatccccAGACAGATGGCTGAGCATCACTCCG |
| 3 | Felix_VLI_Rv | aggtcgactctagaggatccccGAGGGGGCCGACTTGGGCAGAC |
| 4 | Felix_VH1a_Fw | acgttgtaaaacgacggccagtgaaGACGTGCAGCTGGTGGAG |
| 5 | Felix_VH1b_Fw | acgttgtaaaacgacggccagtgaaGACGWGCAGCTGRTGGAG |
| 6 | Felix_VH1c_Fw | acgttgtaaaacgacggccagtgaaGACGTGCTGGTGGTGGAG |
| 7 | Felix_VH1d_Fw | acgttgtaaaacgacggccagtgaaGACGTGCGATTGGTGGAG |
| 8 | Felix_VH1e_Fw | acgttgtaaaacgacggccagtgaaGACGTGCAACTGGTGGAK |
| 9 | Felix_VH1f_Fw | acgttgtaaaacgacggccagtgaaGACGTGCAATTGGTGGAG |
| 10 | Felix_VH1g_Fw | acgttgtaaaacgacggccagtgaaGATGTGCASSTGGTGGAA |
| 11 | Felix_VH1h_Fw | acgttgtaaaacgacggccagtgaaCAGGTTYTGCTGGTGCAG |
| 12 | Felix_VH1i_Fw | acgttgtaaaacgacggccagtgaaGAWGTGCAGYTSGTGGAG |
| 13 | Felix_VH1j_Fw | acgttgtaaaacgacggccagtgaaGATGTGCARSTGGTGGAG |
| 14 | Felix_VL1a_Fw | acgttgtaaaacgacggccagtgaaCAGTCTGTGCTGACTCAGCCG |
| 15 | Felix_VL1b_Fw | acgttgtaaaacgacggccagtgaaCAGTCTGTGGTGACTCAGCCGC |
| 16 | Felix_VL1c_Fw | acgttgtaaaacgacggccagtgaaCAGCCTGTGCTGACTCAGCCACCG |
| 17 | Felix_VL1d_Fw | acgttgtaaaacgacggccagtgaaCAGGAAGTAGTGACTCAGGAAAC |
| 18 | Felix_VL1e_Fw | acgttgtaaaacgacggccagtgaaCAGTCGGGGCCGAATCAACCATC |
| 19 | Felix_VL1f_Fw | acgttgtaaaacgacggccagtgaaCAGTCGGGGCTGACTCAACCATCC |
| 20 | Felix_VL1g_Fw | acgttgtaaaacgacggccagtgaaTCCTATGTATTGACTCAGCCCC |
| 21 | Felix_VL1h_Fw | acgttgtaaaacgacggccagtgaaTCCTATGTACTCTCTCAGCCCC |
| 22 | Felix_VL1i_Fw | acgttgtaaaacgacggccagtgaaCAGCCTGTGGTGACTCAGCCAGC |
| 23 | Felix_Vk1a_Fw | acgttgtaaaacgacggccagtgaaGATATTGTGATGACGCAGACCCC |
| 24 | Felix_Vk1b_Fw | acgttgtaaaacgacggccagtgaaGATCTTGTCTTGACACAGACCCC |
| 25 | Felix_Vk1c_Fw | acgttgtaaaacgacggccagtgaaGAGGCCGTGATGACGCAGACC |
| 26 | Felix_Vk1d_Fw | acgttgtaaaacgacggccagtgaaGAGGTTGTGATGATAMAGACCC |
| 27 | Felix_Vk1e_Fw | acgttgtaaaacgacggccagtgaaGCGATCACGATGACGCAGTCTCC |
| 28 | Felix_Vk1f_Fw | acgttgtaaaacgacggccagtgaaGATGTCGTGATGACGCAGACCC |
| | Primer for adding Illumnia overhangs for NGS | |
| Seq # | Name | Sequence (5′-3′) |
| 29 | PUC19_NGS_Fw | ACACTCTTTCCCTACACGACGCTCTTCCGATCTggttttcccagtcacgacgttgta |
| 30 | PUC19_NGS_Rv | GACTGGAGTTCAGACGTGTGCTCTTCCGATCTtatgaccatgattacgccaagcttgc |

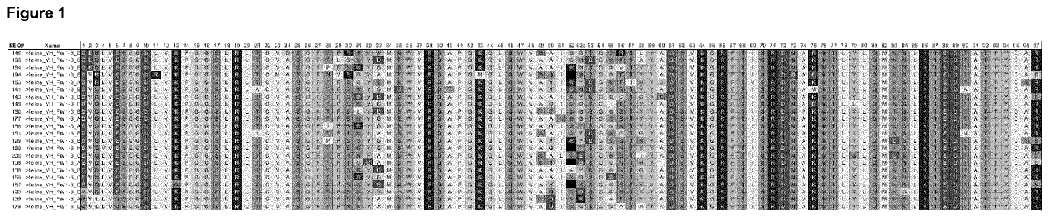

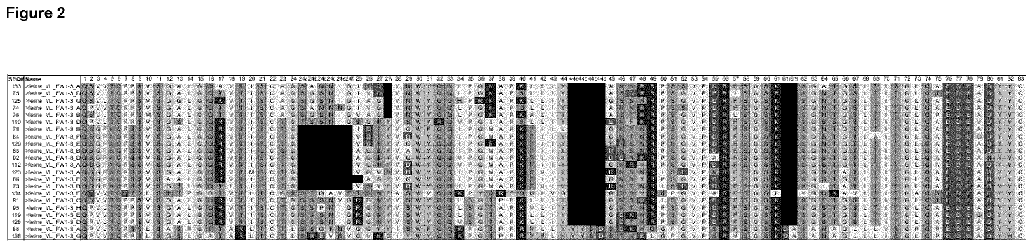

NGS data was analyzed using the Geneious software suite analyzing the CDR3 length distributions (Table 2) and CDR1-3 amino acid compositions. The numbering and CDR definitions were adapted from existing antibody numbering schemes and applied to fit feline canine antibody sequences. Examples of identified VL and VH sequences in the context of a synthetic feline library are depicted in FIG. 1 and FIG. 2. In total, 28628 individual VL-lambda and 22445 VH sequences fulfilling certain quality criteria were used for the analysis.

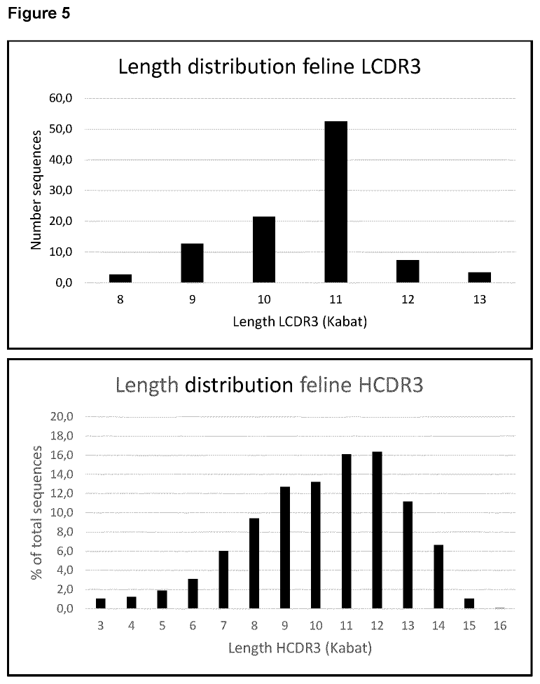

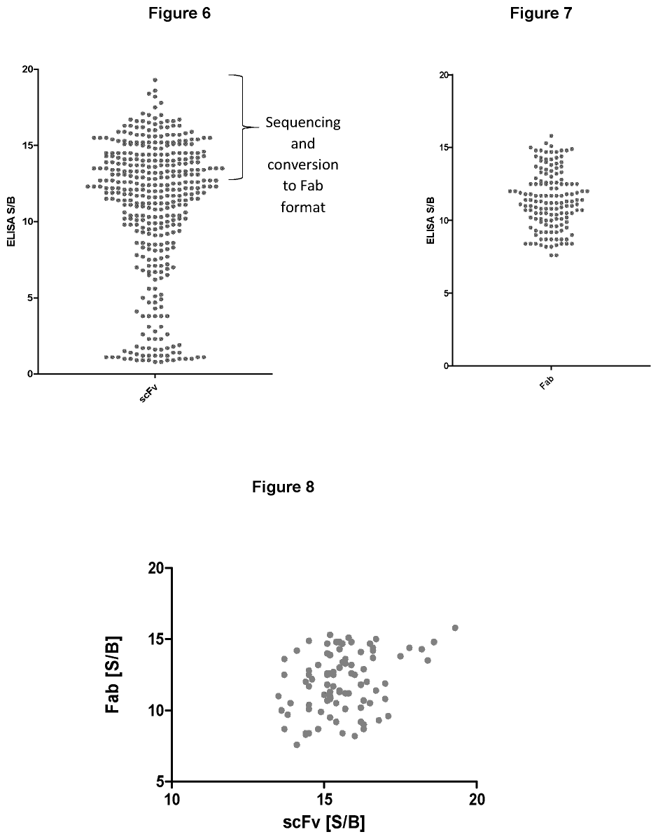

The HCDR3 length distribution of analyzed feline antibodies shows a mean amino acid length of 10.28±2.53, the most frequent HCDR3-lengths are 11 and 12, present at around 16% each (Table 2). For clarification, we refer to the numbering scheme shown in FIG. 9 and start counting after the preferred CAR motif at position 98. In contrast to the high variability of HCDR3-length distribution, light chain CDRs are much more restrained. The most frequent LCDR3 length observed was 11 amino acids for lambda light chains comprising >50% of all analyzed sequences (see Table 2 and FIG. 5).

TABLE 2
Length distribution analysis of feline LCDR 3 and HCDR3

| HCDR3 | | LCDR3-lambda | |
|---|---|---|---|
| Length | % total | Length | % total |
| (Kabat) | sequences | (Kabat) | sequences |
| 3 | 1.0 | 8 | 2.7 |
| 4 | 1.2 | 9 | 12.7 |
| 5 | 1.9 | 10 | 21.5 |
| 6 | 3.1 | 11 | 52.5 |
| 7 | 6.0 | 12 | 7.4 |
| 8 | 9.4 | 13 | 3.3 |
| 9 | 12.7 | | |
| 10 | 13.2 | | |
| 11 | 16.1 | | |
| 12 | 16.3 | | |
| 13 | 11.2 | | |
| 14 | 6.6 | | |
| 15 | 1.1 | | |
| 16 | 0.1 | | |
| Total | 100 | Total | 100 |

Analysis of the total amino acid usage in feline LCDR3, HCDR1, HCDR2 and HCDR3 was carried out. The amino acid variability at a given position in aligned sequences of the same CDR length was calculated in percent of all amino acids present at the respective position. Amino acid utilization for the most dominant CDR lengths identified in our dataset is depicted in Table 3 and Table 4 (LCDR3: 12 AA; HCDR1: 6 AA; HCDR2: 13 AA; HCDR3: 12 AA). Grey shadings indicate amino acids that occur more frequently at individual positions. Analyses of sequences with distinct CDR lengths has been performed (data not shown) and results were taken into consideration for the design of CDR-modules used for the synthetic library (see Example 6).

The feline antibody repertoire follows similar trends in amino acid utilization as observed in dogs. The high frequency of charged amino acids at the base of the HCDR3 loop is similar to the canine repertoire. Also, the FDY motif at the end of the HCDR3 at positions 99-101 is dominant. However, the HCDR3 is preferentially flanked by the CAR motif at position 94 in contrast to CAK in canines.

### Example 2: Amplification of Variable Antibody Regions from Feline Bone Marrow cDNA and Cloning into Phage Display Vector

Based on the NGS analysis in “Example 1”, primers were designed to PCR amplify specific sets of VL-lambda and VH sequences from cat bone marrow cDNA. Overhangs were designed to allow i) for Gibson assembly into the display vector, ii) to introduce a N-terminal FLAG-tag and iii) to introduce an 18 amino acid linker between VL and VH via overlap extension PCR to generate scFv fragments. The display vector introduced a truncated ct-pIII gene, a Trypsin cleavage site prior ct-pIII and alternative antibiotic resistance (Chloramphenicol). For VL-lambda a set of 10 forward and 10 reverse primers was designed. For VH a set of 12 forward and 10 reverse primers was designed. Primers were designed to cover the most frequent VL and VH groups, resulting in coverage of >59% of VL-lambda and >64% of VH sequences identified in the NGS analysis. Focus on highly abundant VL and VH groups is based on those sequences being favorable relating to their biological functionality.

TABLE 5
Primers for introduction of feline VL/VH sequences from
cDNA into phage display vectors

| | Primer for cloning VL/VH into display vector | |
|---|---|---|
| SEQ ID NO | Name | Sequence (5′-3′) |
| 31 | fw_felix_VL_L_1 | ACAAAGACGATGACGACAAGCTCGAGCAGGAAGTAGTGACTCAGGA |
| 32 | fw_felix_VL_L_2 | ACAAAGACGATGACGACAAGCTCGAGCAGTCGGGGCCGAATCAACC |
| 33 | fw_felix_VL_L_3 | ACAAAGACGATGACGACAAGCTCGAGTCCTATGTATTGACTCAGCC |
| 34 | fw_felix_VL_L_4 | ACAAAGACGATGACGACAAGCTCGAGCAGTCGGGGCTGACTCAACC |
| 35 | fw_felix_VL_L_5 | ACAAAGACGATGACGACAAGCTCGAGCAGTCTGTGGTGACTCAGCC |
| 36 | fw_felix_VL_L_6 | ACAAAGACGATGACGACAAGCTCGAGTCCTATGTACTCTCTCAGCC |
| 37 | fw_felix_VL_L_7 | ACAAAGACGATGACGACAAGCTCGAGCAGCCTGTGCTGACTCAGCC |
| 38 | fw_felix_VL_L_8 | ACAAAGACGATGACGACAAGCTCGAGCAGCCTGTGGTGACTCAGCC |
| 39 | fw_felix_VL_L_9 | ACAAAGACGATGACGACAAGCTCGAGCAGTCTGTGCTGACTCAGCC |
| 40 | fw_felix_VL_L_10 | ACAAAGACGATGACGACAAGCTCGAGTTCTATGTATTGACTCAGCC |
| 41 | rv_felix_VL_L_1 | CTAGAACGGCTAGAACCGCC GAGGACGGTCAGATGGGTCCC |
| 42 | rv_felix_VL_L_2 | CTAGAACGGCTAGAACCGCC GAGGACAGTCAGATGGGTCCC |
| 43 | rv_felix_VL_L_3 | CTAGAACGGCTAGAACCGCC TAGGACGCTCAGATGGGTACC |
| 44 | rv_felix_VL_L_4 | CTAGAACGGCTAGAACCGCC GAGGACGGTCAGACGGGTCCC |
| 45 | rv_felix_VL_L_5 | CTAGAACGGCTAGAACCGCC GAGGACGGTCACATGGGTCCC |
| 46 | rv_felix_VL_L_6 | CTAGAACGGCTAGAACCGCC GAGGACGGTCAAATGGGTCCC |
| 47 | rv_felix_VL_L_7 | CTAGAACGGCTAGAACCGCC GAGGACGGTCAGATGGGTACC |
| 48 | rv_felix_VL_L_8 | CTAGAACGGCTAGAACCGCC GAGGACGCTCAGATGGGTACC |
| 49 | rv_felix_VL_L_9 | CTAGAACGGCTAGAACCGCC CAGGACGCTCAGATGGGTACC |
| 50 | rv_felix_VL_L_10 | CTAGAACGGCTAGAACCGCC GAGGACGGTCAGGTGGGTCCC |
| SEQ ID NO | Name | Sequence (5′-3′) |
| 51 | fw_felix_VH_1 | GCGGTTCTGGCGGTGGTGGTCAGGTTTTGCTGGTGCAGTCTGGG |
| 52 | fw_felix_VH_2 | GCGGTTCTGGCGGTGGTGGTGACGTGCAACTGGTGGAGTCTGGG |
| 53 | fw_felix_VH_3 | GCGGTTCTGGCGGTGGTGGTGACGTGCAGCTGGTGGAGTCTGGG |
| 54 | fw_felix_VH_4 | GCGGTTCTGGCGGTGGTGGTGACGTGCTGGTGGTGGAGTCTGGG |
| 55 | fw_felix_VH_5 | GCGGTTCTGGCGGTGGTGGTGACGTGCGATTGGTGGAGTCTGGG |
| 56 | fw_felix_VH_6 | GCGGTTCTGGCGGTGGTGGTGACGTGCAATTGGTGGAGTCTGGG |
| 57 | fw_felix_VH_7 | GCGGTTCTGGCGGTGGTGGTCAGGTTCTGCTGGTGCAGTCTGGG |
| 58 | fw_felix_VH_8 | GCGGTTCTGGCGGTGGTGGTGATGTGCAGGTGGTGGAGTCTGGG |
| 59 | fw_felix_VH_9 | GCGGTTCTGGCGGTGGTGGTGATGTGCAGCTGGTGGAGTCTGGG |
| 60 | fw_felix_VH_10 | GCGGTTCTGGCGGTGGTGGTGACGAGCAGCTGGTGGAGTCTGGG |
| 61 | fw_felix_VH_11 | GCGGTTCTGGCGGTGGTGGTTACGTGCAACTGGTGGAGTCTGGG |
| 62 | fw_felix_VH_12 | GCGGTTCTGGCGGTGGTGGTGAAGTGCAGTTGGTGGAGTCTGGG |
| 63 | rv_felix_VH_1 | TCACGAATATCTTTGCTACCGAATTCTGAGGACACCGTCACCAGGGC |
| 64 | rv_felix_VH_2 | TCACGAATATCTTTGCTACCGAATTCTGAGGACACTGTGACTATGGT |
| 65 | rv_felix_VH_3 | TCACGAATATCTTTGCTACCGAATTCTGAGGACACCGTCACCAGGAC |
| 66 | rv_felix_VH_4 | TCACGAATATCTTTGCTACCGAATTCTGAAGACACGGTGACCAGGGT |
| 67 | rv_felix_VH_5 | TCACGAATATCTTTGCTACCGAATTCTGAGGACACCGTCACCAGGGT |
| 68 | rv_felix_VH_6 | TCACGAATATCTTTGCTACCGAATTCTTGGGAGACGGTGACCTGGGT |
| 69 | rv_felix_VH_7 | TCACGAATATCTTTGCTACCGAATTCTGAGGAGACGGTGACCAGGGT |
| 70 | rv_felix_VH_8 | TCACGAATATCTTTGCTACCGAATTCCGAGGACACCGTCACCAGGGC |
| 71 | rv_felix_VH_9 | TCACGAATATCTTTGCTACCGAATTCGGAGGACACCGTCACCAGGGC |
| 72 | rv_felix_VH_10 | TCACGAATATCTTTGCTACCGAATTCTGAGGACACCGTCACCGGGGC |

### Example 3: Display of scFv Fragments on Phage and Anti-FLAG Pull Down

The scFv fragments cloned into the display vector featuring random combinations of cDNA derived VL-lambda and VH sequences were produced on the surface of phages using a VCSM13-based helper phage, following standard procedures (see also Example 10). The phage preparation was analyzed running colony PCRs on infected XL1-Blue E. coli, revealing around 85% of analyzed clones to show a fragment with the correct size and validating the overall cloning approach of natural VL/VH sequences into the display vector. For enrichment of phage particles displaying intact FLAG-scFv fragments, an anti-FLAG pull down with magnetic beads (GE Healthcare) was conducted. Phages (5×10¹⁰ phages) and beads were blocked with ChemiBlock. To deplete phages displaying potentially sticky scFv fragments, phages were incubated with magnetic beads coated with an irrelevant protein (biotinylated-GFP). Next, an anti-FLAG pull down was carried out using magnetic beads coated with a biotinylated anti-FLAG antibody. After extensive washing, bound phages were eluted using the FLAG peptide. Subsequently, XL1-Blue cells were infected, resulting in 2.4E+06 colonies representing clones with randomly paired feline antibody VL-lambda and VH chains.

### Example 4: Expression of scFv-Fragments and Expression Check

To evaluate the expression levels of the feline scFv-fragments enriched during the anti-FLAG pull down, the scFv-encoding gene fragments were polyclonally cloned into the bacterial expression vector pFeBx via restriction enzymes NcoI/EcoRI. Purified DNA fragments were ligated into pre-cut pFeBx vector and subsequent transformation in chemically competent E. coli BL21 (DE3) was performed according to standard procedures.

For the analysis, 380 random clones were picked and subjected to scFv production and expression check. In brief, wells of expression plates (Round bottom 96-well plates (e.g. Thermo Fisher, Cat: #262162) were filled with 120 ul/well all-in-one-medium (2×YT medium containing 34 μg/ml Cam, 0.1% Glucose and 0.5 mM IPTG) and were inoculated with single colonies from agar plates of the subcloning procedure. Plates were incubated for 5 h at 37° C. shaking at 400 rpm and afterwards overnight at 22° C. To prepare crude bacterial lysates used for screening purposes, so called BEL-lysates, 40 μl/well of lysis buffer (2×BBS containing 2.5 mg/ml lysozyme, 4 mM EDTA and 13 U/ml Benzonase) were added and incubated for 1 h at 22° C. shaking at 400 rpm. Then, 40 μl/well of blocking buffer (1×PBS containing 5% milk powder) were added and incubated at 22° C. for 1 h.

For screening, BEL lysates were captured to Maxisorp microtiter plates using a coated anti-feline IgG (H+L) antibody (Jackson). Following immobilization, plates were washed three times with PBST and subsequently blocked with 5% milk in PBS for 1 h at RT. Plates were washed 3 times with PBST and subsequently, an anti-FLAG-HRP detection antibody (R&D Systems, HAM85291) was added to the plates and incubated for 1 h at RT. Plates were washed 5 times with PBST and detection of bound antibodies was performed using the QuantaBlu reagent according to the manufacturer's instruction on a Tecan Geneious Reader (excitation filter: 320 nm, emission filter: 430 nm).

The ELISA revealed expression signals ranging from 1-19× signal over background (S/BG) (see FIG. 2). 62% of the clones showed an expression rate >10×S/BG. Results of the ELISA expression analysis are shown in FIG. 7. The 96 best expressing scFv clones were sent for sequencing, resulting in 65 unique antibody sequences. Regions that represent promising building blocks for a synthetic library are depicted in Table 6 and Table 7, showing feline protein sequences from framework 1 (FW1) to framework 3 (FW3) of the light and heavy chains, respectively.

TABLE 6
FW1-FW3 of feline light chain sequences of high-expressing
scFvs after FLAG-pull down

| SEQ ID NO | Name | Sequence (5'-3') |
|---|---|---|
| 73 | >feline | QSGPNQPSSVSGTLGQTITISCTGVSVFVDWYQQIPGMAPKTVIYRNT |
| | VL_FW | NRLSEVPDRFSGSKSGITATLTITGLQAEDEADYYC |
| | 1-3_B5 | |
| 74 | >feline | QPVVTQPPSVSGALGQTVTISCAGSANNNGIVGVNWYQQLPGKAPQL |
| | VL_FW | LIYANNRRPSSVPDRFSGSKSGNTGSLTITGLQAEDEADYYC |
| | 1-3_A12 | |
| 75 | >feline | QSVLTQPPSVSGALGQTVTISCAGSGSNIGIVDVNWYQQLPGKAPSLL |
| | VL_FW | IYASDRRPSGVPERISGSRSGNTGSLTITGLQTEDEADYYC |
| | 1-3_D8 | |
| 76 | >feline | QSVLTQPPSMSGALGQTVTISCAGSGSNIGVAGVNWYQQLPGKAPKA |
| | VL FW | LIYASTRRPSGVPERLSGSKSGNTGSLTITGLQPEDEADYYC |
| | 1-3_B8 | |
| 77 | >feline | QSVVTQPPSVSGTPGQRITISCTGSSSNIGGGAAVSWYQQVPGMGPK |
| | VL_FW | SVIFWNNSKPSGVPDRFSGSKSGSSGTLTIIGLQAEDEADYYC |
| | 1- | |
| | 3_C12 | |
| 78 | >feline | QSGPNQPSSVSGALGQRVTISCTGIDTYVGWFQQIPGMAPKTIIVGNT |
| | VL_FW | NRPSGVPDRFSGSKSGNTGTLTITGLQAEDEADYYC |
| | 1-3_B6 | |
| 79 | >feline | QSGPNQPSSVSGALGQRVTISCTGLDSFVDWYQQMPGMAPKTIIYGD |
| | VL_FW | KYRPSGVPDRFSGSKSGSTGTLTITGLQADDEADYYC |
| | 1-3_E3 | |
| 80 | >feline | QSGLTQPSSVSGALGQTVTISCAGSANNIGAVGVNWYQQLPGRAPKL |
| | VL_FW | LIYANNRRPSSVPDRFSGSKSGNTGSLTITGLQAEDEADYYC |
| | 1-3_D1 | |
| 81 | >feline | QSVVTQPPSVSGALGQRVTMSCTGSSSNIGRGVYVSWYQQLSGTAP |
| | VL_FW | KLLIYGNTNRPSGVPDRFSGSKSGSTGSLTITGLQDEDEADYYC |
| | 1-3_B9 | |
| 82 | >feline | QSVLTQPPSVSGALGQRVTISCTGISSNIGRGNYVSWYQQIPGIAPKTII |
| | VL_FW | YVDNNRPSGVPDRFSGSKSGSTATLTITGLQAEDEADYYC |
| | 1-3_D3 | |
| 83 | >feline_ | QPVLTQPPSVSVSLGQTARTTCGGNNIESKSVHWYQQRSGQTPVLIIY |
| | VL_FW | TDRNRPSGIPDRFSGTNSGNTATLTISGAQAEDEADYYC |
| | 1- | |
| | 3_G11 | |
| 84 | >feline | QSGPNQPSSVSGALGQRVTISCTGVDTYVDWYQQIPGMAPKTIIYGN |
| | VL_FW | TNRPSGVPDRFSGSKSGSTGTLAITGLQAEDEGDYYC |
| | 1-3_F9 | |
| 85 | >feline | QSGPNQPSSVSGALGQRVTISCSGAGSYVAWYQQIPGMAPKTIIYDN |
| | VL_FW | SNRPSGVPDRFSGSKSGNTGTLTITGLQAEDEADYYC |
| | 1-3_B11 | |
| 86 | >feline | QSGPNQPSSVSAALGQRVTISCTGAGNYVGWYQQIPGMAPKTIIYGN |
| | VL_FW | SYRPSGVPDRFSGSKSGSTGTLTITGLQAEDEADYYC |
| | 1-3_E11 | |
| 87 | >feline_ | QSGPNQPSSVSGALGQRVTISCTGAGSNVAWYQQIPGMAPKTIMYFN |
| | VL_FW | RNRPSGVPDRFSGSKSGDVGTLTITGLQAEDEADYFC |
| | 1-3_B1 | |
| 88 | >feline | QPVLTQPSSLSASPGTTARLTCTLSSGFNVGGYYISWFQQKPGSPPR |
| | VL_FW | YLLYYYSDSDKHQGPGVPSRFSGSKDASANAGLLLVSGPQPEDEADY |
| | 1-3_A8 | YC |
| 89 | >feline | QPVLTQPPSVSGALGQTVTISCAGSANNIGIVGVNWYQQLPGKAPKLL |
| | VL_FW | IYANNRRPSSVPDRFSGSKSGNTGSLTITGLQAEDEADFYC |
| | 1-3_D2 | |
| 90 | >feline | QSVVTQPPSVSGTLGQRITISCTGGSSNIGGGEAVNWYQQVPGMGP |
| | VL_FW | KTVIYWSDNKPSGVPDRFSGSKSGSSGTLTITGLQAEDEADYYC |
| | 1-3_B7 | |
| 91 | >feline | QSVVTQPPSVSGALGQRVTISCTGSSSNVGRGNYVSWYQQLSGTPP |
| | VL_FW | KLLIYGNSDRLSGVPDRFSGSKSGTTGSLTITGLQTEDEADYYC |
| | 1- | |
| | 3_C10 | |
| 92 | >feline | QSGPNQPSSVSGALGQRVTISCTGAGSYVGWYQQVPGMAPKTIIYDD |
| | VL_FW | NKRPSGVPARFSGSKSGSTGTLTITGLQAEDEANYYC |
| | 1-3_D7 | |
| 93 | >feline | QSVLTQPPSVSGSLGQRVTISCTGSTSNIGGGNYVTWYQQVPGTAPR |
| | VL_FW | LLIYEDDKRASGVPGRFSGSKSGSSGSLTITGLQADDEADYYC |
| | 1-3_B10 | |
| 94 | >feline | QPVLTQPPSVSGSPGQRVTISCSGGSSNIGGGNYVSWYRQVLGSAP |
| | VL_FW | RLLIYENYRRPSGVPDRFSGSKSGNSGSLTITGLQADDEADYYC |
| | 1-3_A7 | |
| 95 | >feline_ | QPVLTQPPSVSGALGQRVTISCTGSSPNIGRGNYVSWYQQLSGTAPK |
| | VL_FW | VLIYGNTNRPSGVPDRFSGSKSGSTGSLTITGLQAEDEADYYC |
| | 1-3_H6 | |
| 96 | >feline | QSVLTQPPSVSGALGQRVTISCTGSANNIGIVGVSWYQQLPGKAPKLL |
| | VL_FW | IYANDRRPSSVLDRFSGSKSGNTGSLTITGLQAEDEADYYC |
| | 1-3_H5 | |
| 97 | >feline | QSGPNQPSSVSGALGQRVTISCTGVGAYVDWYQQIPGMAPKTIIYYD |
| | VL_FW | SNRPSGVPDRFSGSKSGNTGTLTITGLQAEDEADYYC |
| | 1-3_F2 | |
| 98 | >feline | QSGLTQPSSVSGALGQRVTVSCTGVASYVYWYQQLPGMAPKTIIYGD |
| | VL_FW | RNRPSGVPDRFSGSKSGSTGTLTITGLRAEDEADYYC |
| | 1-3_F3 | |
| 99 | >feline | QSGPNQPSSVSGALGQRVTISCTGVGNYVDWYQQIPGMAPKTIIYYD |
| | VL_FW | TDLPSGVPDRFSGSKSGSTATLTITGLRAEDEADYYC |
| | 1-3_F6 | |
| 100 | >feline | QSVLTQPPSVSGALGQRVTISCTGVGNYVGWHQQIPGMAPKTIIYDGT |
| | VL_FW | NRPSGVPDRFSGSKSGDTGTLTITGLQAEDEADYYC |
| | 1-3_F8 | |
| 101 | >feline | QSGLTQPSSVSGALGQRVTISCTGVGSYVYWYQQIPGMVPKTIIYDNN |
| | VL_FW | NRPSGVPDRFSGSKSGFTGTLTIAGLQAEDEADYYC |
| | 1-3_B2 | |
| 102 | >feline | QPVVTQPASLSASLGATARLTCTLSRDINVGSYSIYWYQQSPGSPPRY |
| | VL_FW | LLYYYSDSTTQLGPGVPTRFSGSKDASANAGLLLISGLQPEDEADYFC |
| | 1-3_F4 | |
| 103 | >feline | QSVLTQPPSVSVSLGQTARTTCGGNNIESKSVHWYQQKSGQTPVLIIY |
| | VL_FW | SDSNRPSGIPDRFSGTNSGNTATLTISGAQAEDEADYYC |
| | 1-3_C1 | |
| 104 | >feline | QSGPNQPSSVSGALGQRVTISCIGVGDYVYWYQQIPGMAPKTLIYGV |
| | VL_FW | RNRPSGVPDRFTGSKSGSTGTLTITGFQAEDEADYYC |
| | 1-3_C5 | |
| 105 | >feline | QSGPNQPSSVSGALGQRVTVSCTGAGSYVGWYQQIPGMVPKTIIYDN |
| | VL_FW | GNRPSGVPDRFSGSKSGSIGTLTITGLQAEDEADYYC |
| | 1-3_A2 | |
| 106 | >feline | QSVVTQPPSVSGTLGQRVTISCTGSSSNVGRGNYVSWYQQLSGSAP |
| | VL_FW | KLLIYGNTNRPLGVPDRFSGSKSGSTGSLTITGLQAEDEADYYC |
| | 1-3_A1 | |
| 107 | >feline | QPVLTQPPSVSVSLGQTARTTCGGNNIESKSVHWYQQKSGQTPVLIIY |
| | VL_FW | SDSNRPSGIPDRFSGTNSGNTATLTISGAQAEDEADYYC |
| | 1-3_E12 | |
| 108 | >feline | QSGLTQPSSVSGALGQRVTLSCTGIDSYVSWYQQIPGMAPKTIIYGNS |
| | VL_FW | NRPSGVPDRFSGSKSGSTGILTVTGLQAEDEADYYC |
| | 1-3_C9 | |
| 109 | >feline | QSGPNQPSSVSGALGQRVTISCTGIGISVDWYQQVPGMAPKTVITRVT |
| | VL_FW | DRLSGVPDRFSGFKSGSTATLTITGLRAEDEADYYC |
| | 1-3_C4 | |
| 110 | >feline | QPVLTQPPSVSGSLGQRVTISCTGTSSNIGSGNYVSWYRQVPGIAPSL |
| | VL_FW | LIYENFKRPSGVPDRFSGSKSGSSGSLTITGLQADDEVDYYC |
| | 1- | |
| | 3_D11 | |
| 111 | >feline_ | QSGLTQPSSVSGALGQRVTISCAGVGTYVYWYQQLPGMAPKTIIFDN |
| | VL_FW | NHRPSGVTGRFFGSKSGDTGTLTITGLQAEDEADYYC |
| | 1-3_H3 | |
| 112 | >feline_ | QSGPNQPSSVSGALGQRVTISCTGVGNYVDWYQQVPGMAPKTIIYGN |
| | VL_FW | RYRPSGVPDRFSGSKSGSTGTLTITGLQAEDEADYYC |
| | 1-3_A11 | |
| 113 | >feline | QSVVTQPPSVSGALGQRVTISCTGSSSNIGRGSYVNWYQQLSGTAPK |
| | VL_FW | LLIYDDNSRPSGVPDRFSGSKSGSTGSLTITGLQAEDEADYYC |
| | 1-3_C3 | |
| 114 | >feline | QSGLTQPSSVSGALGQRVTISCTGVGSFVYWYRQIPGMAPKTVIYHNI |
| | VL_FW | NRPSGVPDRFSGSKSGSTSTLTITGLQAEDEADYYC |
| | 1-3_D4 | |
| 115 | >feline | QSGPNQPSSVSGALGQRVTISCTGVGTWVGWYQQIPGMAPKTIIYGN |
| | VL_FW | NNRPSGVPDRFSGSKSGSTGTLTITGLQAEDETDYYC |
| | 1-3_F1 | |
| 116 | >feline_ | QSVLTQPPSVSGALGQRVTISCTGSSSNIGRGNNVNWYQQLSGTAPK |
| | VL_FW | LLIYGNTNRPSGVPDRFSGSRSGSTGSLTITGLQADDEADYYC |
| | 1-3_B3 | |
| 117 | >feline | QSVLTQPPSVSGALGQRVTISCTGSRSNIGRGNYVNWYQQLSGTGPK |
| | VL_FW | LLIYGNSNRPSGVPDRFSGSKSGSTGSLTITGLQAEDEADYYC |
| | 1-3_C2 | |
| 118 | >feline | QSVVTQPASLSASLGATARLTCTLSRDINVGSYNIYWYQQSPGNPPRF |
| | VL_FW | LLYYFSDSSTQLGPGVPSHFSGSKDASANAGLLLISGLQPEDEADYYC |
| | 1-3_E8 | |
| 119 | >feline | QPVVTQPPSVSGALGQRVTISCTGSSSNIGRGNYVSWYQQLSGTAPK |
| | VL_FW | LLIYGDKNRPSGVPDRFSGSKSGSTGSLTITGLQAEDEGDYYC |
| | 1-3_E9 | |
| 120 | >feline_ | QPVLTQPPSVSGALGQTVTISCAGSRSNIGFAGVNWYQQLPGKAPKL |
| | VL_FW | LIYGNDRRPSGVPERISGSKSGNTGSLAITGLQAEDEADYYC |
| | 1-3_G2 | |
| 121 | >feline | QSVLTQPPSVSGSPGQRVTISCTGSTSNIGGGNSVNWYQQVPGAAP |
| | VL_FW | RLLIYENNKRPSGVPDRFSGSKSGNSGSLTITGLQADDEADYYC |
| | 1- | |
| | 3_D12 | |
| 122 | >feline | QSVLTQPPSLSGALGQRVTISCTGSTSNIGRGNYVNWYQQLSGAAPK |
| | VL_FW | LLIFGTANRPSGVPDRFSGSKSGNTGSLTITGLQVGDEADYYC |
| | 1-3_D5 | |
| 123 | >feline | QSGPNQPSSVSGALGQRVTMSCTGVGMSVDWYQQIPGMAPKTVIYR |
| | VL_FW | NSNRLSEVPNRFSGSKSGSTATLTITGLQAEDEADYYC |
| | 1-3_A3 | |
| 124 | >feline | QPVLTQPPSVSGALGQRVSISCTGSSSNIGRGNYVSWYQQLSGTAPK |
| | VL_FW | LLIYGNANRPSGVPDRFSGSKSGITGSLTITALQAEDEADYYC |
| | 1-3_E2 | |
| 125 | >feline | QSVLTQPPSVSGGLGQKVTISCAGSGSNIGIAGVNWYQQHPRKAPKL |
| | VL_FW | LIYGSSNRPSGVPDRFSGSKSGNTGSLTITGLQAEDEADYYC |
| | 1- | |
| | 3_G12 | |
| 126 | >feline | QSVLTQPPSVSGGLGQKVTISCAGSRSNIGTAGVNWYQQHPGKAPKL |
| | VL_FW | LIYGNDNRPSGVPDRFSGSKSGNTGSLTITGLQAEDEADFYC |
| | 1-3_H4 | |
| 127 | >feline | QSGPNQPSSVSGALGQRVTISCTAPGSYVDWYQQIPGMAPKTIIYGN |
| | VL_FW | HNRPSGVPDRFSGSKSGNTGTLTITGLQAEDEADYYC |
| | 1-3_A4 | |
| 128 | >feline | QPVLTQPPSVSGALGQRVTISCTGSSSNIGRGNYVSWYQQVSGTAPK |
| | VL_FW | LLIYGDTNRPSGVPDRFSGSKSGSTGSLTITGLQADDEADYYC |
| | 1- | |
| | 3_D10 | |
| 129 | >feline | QSGPNQPSSVSGALGQRVTISCTGVDSYVGWYQQIPGRAPKTIIYYDS |
| | VL_FW | NRPSGVPDRFSGSKSGNTGTLTITGLQAEDEADYYC |
| | 1-3_E7 | |
| 130 | >feline | QSVLTQPPSVSRGLGQTVTISCAGNRNNIGIAGVVWYQQHPGKAPKL |
| | VL_FW | LIYGSNNRPSGVPDRFSGSKSGNTGSLTITGLQAEDEADYYC |
| | 1-3_H1 | |
| 131 | >feline | QPVLTQPPSVSGALGQRVTISCTGSSSNFGRGDYVSWYQQVSGTAP |
| | VL_FW | KLLIYGDNNRPSGVPDRFSGSKSGRIGSLTITGLQAEDEADYYC |
| | 1-3_H2 | |
| 132 | >feline | QEVVTQETSLSTTPGGTVTLTCGSSTGAVTTSNYASWVRQKPYQRIQ |
| | VL_FW | GLIGGTSYRNPGVPARFSGSLVGQKAVLTITGAQSEDEAEYYC |
| | 1-3_E6 | |
| 133 | >feline | QSVVTQPPSVSGALGQAVTISCAGSANNIGITDVNWYQQLPGKAPKLL |
| | VL_FW | IYANNRRPSSVPDRFSGSKSGATGSLTISGLQAEDEADYYC |
| | 1-3_A9 | |
| 134 | >feline | QEVVTQETSLSTTPGGTVTLTCGSSTGAVTTSNFASWVQQKPYQRFQ |
| | VL_FW | GLVGGTSYRNPGVPARFSGSLVGQKAVLTITGAQSEDEAEYYC |
| | 1-3_E10 | |
| 135 | >feline | QPVVTQPPSLSGSLGATARLTCTLSREVSVGVKGIYWYQQKPGSPPR |
| | VL_FW | YFLHYYSDSTNELGPGVPSRVSGSKDASANAGLLLISGLQPEDEADYH |
| | 1-3_G8 | C |
| 136 | >feline | QSGPNQPSSVSGALGQRITISCTGVGSYVGWYQQIPGRAPKTIIFYDT |
| | VL_FW | NRRSGVPDRFSGSKSGSTGTLTITGLQAEDEADYYC |
| | 1-3_A10 | |
| 137 | >feline | QPVVTQPPSMSGALGQTVTISCAGSGTNIGVAGVNWYQQLPGKAPK |
| | VL_FW | VLIYASSRRPSGVPERISGSKLGNTGTLTISGLQAEDEADYYC |
| | 1-3_B4 | |

TABLE 7
FW1-FW3 of feline heavy chain sequences of high-expressing
scFvs after FLAG-pull down

| SEQ ID NO | Name | Sequence (5'-3') |
|---|---|---|
| 138 | >feline | EVOLVESGGDLVKPGGSLRLTCVASGFTFSNYDMSWVRQAPGKGLQ |
| | VH_FW | WVAAIAYNGGNTGYADSVKGRFTISRDNAKNTLYLQMNSLKTEDTATY |
| | 1-3_B5 | YCAR |
| 139 | >feline | QVLLVQSGGDLVKPGGSLRLTCVASGFTFSSYAMSWVRQAPGKGLQ |
| | VH_FW | WVTDISRSGATTYYADSVKGRFTISRDNVKNTLYLQMNSLKTEDTATY |
| | 1- | YCAR |
| | 3_A12 | |
| 140 | >feline | DEQLVESGGDLVKPGGSLRLTCVGSGFTFRSHWMNWVRQAPGKGLQ |
| | VH_FW | WVSAIGGTGSRTLYADSVKGRFTISRDNARNTLYLQMNSLKTEDTATY |
| | 1-3_D8 | YCAR |
| 141 | >feline | DVQLVESGGDLVKPGGSLRLACVASGFTFSNYGMDWVRQTPGKGLQ |
| | VH_FW | WVAYIDNDGSSTYYADSVKGRFTISRDNAMNTLYLQMNSLKTEDAATY |
| | 1-3_B8 | YCAR |
| 142 | >feline | DVRLVESGGNLVKPGGSLRLTCVASGFTFSSYGMSWVRQAPGKGLR |
| | VH FW | WVAYIRYDGSTTNYADSVKGRFTISRDNAKNTLYLQMNSLKTEDTATY |
| | 1- | YCAT |
| | 3_C12 | |
| 143 | >feline | DVQLVESGGDLVKPGGSLRLTCVASGFTFSDYDMSWVRQAPGKGLQ |
| | VH_FW | WVAAISYNGGGTGYSDSVKGRFTISRDNAKNTLYLQMNSLKTEDTATY |
| | 1-3_B6 | YCAT |
| 144 | >feline | YVQLVESGGDLVKPGGSLRLTCVASGFTFSRYAMNWVRQAPGKGLQ |
| | VH_FW | WVSAISETGGDTYYADSVKGRFTISRDNAKNTLYLQMNGLKTEDTATY |
| | 1-3_E3 | YCAR |
| 145 | >feline | DVQLVESGGDLVKPGGSLRLTCVASGFTFSSDYMHWVRQTPGKGLQ |
| | VH_FW | WVAQITSSGGYTYYADSVKGRFTISRDNAKNTLYLQMNSLKTEDTATY |
| | 1-3_D1 | YCAS |
| 146 | >feline | DVRLVESGGDLVKPGGSLRLTCVASGFTFSSFYMHWVRHAPGKGLQ |
| | VH_FW | WVAQISGSGHSTKYADSVKGRFTISRDNAKNTLYLQQNSLKTEDTATY |
| | 1-3_B9 | FCAM |
| 147 | >feline | DVRLVESGGDLVKPGGSLRLTCVASGFTFSSYSMTWVRQAPGKGLQ |
| | VH_FW | WVAHIYTDGSSTNYADSVKGRFTISRDNAKNTLYLQMNSLKTEDTATY |
| | 1-3_D3 | YCAR |
| 148 | >feline | EVOLVESGGDLVKPGGSLRLTCVASGFTFSTYYMTWVRQAPRKGLQ |
| | VH_FW | WVAYIKPDGSSTNYADSVKGRFTISRDNAKNTLYLQMNSLKTEDTATY |
| | 1- | YCAR |
| | 3_G11 | |
| 149 | >feline | DVQLVESGGDLVKPGGSLRLTCVASGFTFSNYGMSWVRQAPGKGLQ |
| | VH_FW | WVAGISGSGITTYYADSVKGRFTISRDNAENTLLLQMNSLKTEDTATYY |
| | 1-3_F9 | CAR |
| 150 | >feline | DVQLVESGGDLVKPGGSLRLTCVASGFTFSLYDMSWVRQAPGKGLQ |
| | VH_FW | WVTTISGSGGSTYYADSVKGRFTISRDNPKNTLYLQMTSLKTEDTATY |
| | 1- | YCAR |
| | 3_B11 | |
| 151 | >feline | DVQLVESGGDLVKPGGSLRLICVASGFTFTNYAMSWVRQAPGKGLQW |
| | VH_FW | VSAISFDGSGTGYADSVKGRFTISRDNAKNTLYLQMNSLKTEDMATYY |
| | 1- | CAR |
| | 3_E11 | |
| 152 | >feline | EVOLVESGGDLVKPGGSLRLTCVASGFTFNNYDMSWVRQAPGKGLQ |
| | VH_FW | WVAAISSNGGITGYADSVKGRFTVSRDNAKNTLYLQMNSLKTDDTATY |
| | 1-3_B1 | YCTR |
| 153 | >feline | DVRLVESGGDLVKPGGSLRLTCVASGFTFSGYSMNWVRQAPGKGLQ |
| | VH_FW | WVAYIRYDGNTIHYGDSVKGRFTVSRDNAKNTLYLQMNSLKTEDTATY |
| | 1-3_A8 | YCSR |
| 154 | >feline_ | DVQVVESGGDLVQPGGSLRLTCVASGFTFSSYEMNWVRQAPGKGLQ |
| | VH_FW | WVAYIGSSGGSTYYADSVKGRFTISRDDAKNTLYLQMNSLKTEDTATY |
| | 1-3_D2 | YCAR |
| 155 | >feline | YVQLVESGGDLVKPGGSLRLICVASGFTFSDNAMTWVRQAPGKGLQW |
| | VH_FW | ISVISFDGGSTAYADSVKGRFTISRDNAKNTLYLQMNSLKTEDTATYYC |
| | 1-3_B7 | AR |
| 156 | >feline | EVQLVESGGDLVKPGGSLRLTCVASGFTFSRYGMSWVRQAPGKGLQ |
| | VH_FW | WVAAISGSGDSTYYADSVKGRFTISRDNAKNTLYLQMNSLKTEDTATY |
| | 1- | YCAK |
| | 3_C10 | |
| 157 | >feline | DVQLVESGGDLVQPGGSLRLTCVASGFTFSSYEMNWVRQAPGKGLQ |
| | VH_FW | WVAYISSGGSTYYADSVKGRFTISRDNAKNTLYLQMNSLKTEDTATYY |
| | 1-3_D7 | CAN |
| 158 | >feline_ | DEQLVESGGDLVKPGGSLRLTCVASGFTFSRYGMSWVRQAPGKGLQ |
| | VH_FW | WVAAIGGSGGSIYYADSVKGRFTISRDNAKNTLYLQMNSLKTEDSATY |
| | 1- | HCVR |
| | 3_B10 | |
| 159 | >feline | DEQLVESGGDLVKPGGSLRLTCVASGFTFSSYGMSWVRQAPGKGLQ |
| | VH_FW | WVAAISGSGGSIYYADSVKGRFTISRDNAKNTLYLQMNSLKTEDTATYY |
| | 1-3_A7 | CAR |
| 160 | >feline | DVQVVESGGDLVKPGGSLRLTCVASGFTFSNYGMSWVRQAPGKGLQ |
| | VH_FW | WVAGITSGGNTYYADSVKGRFTISRDNAKNTLYLQMNSLKTEDTATYY |
| | 1-3_H6 | CAR |
| 161 | >feline | DVQLVESGGDLVKPGGSLRLTCVASGFTFSNYAMNWVRQAPGKGLQ |
| | VH_FW | WVSAISESGDSTYYADSVKGRFTISRDNAKNTLYLQMNSLKTEDTATY |
| | 1-3_H5 | YCAR |
| 162 | >feline | DVQLVESGGDLVKPGGSLRLTCVASGFTFSSYYMHWVRQAPGKGLQ |
| | VH_FW | WVAQISNSGGSTYYADSVKGRFTISRDNAKNTLYLQMNSLKTEDTATY |
| | 1-3_F2 | YCTK |
| 163 | >feline | DVQLVESGGDLVKPGGSLRLTCVASGFTFNNFAMSWVRQAPGKGLQ |
| | VH_FW | WVSALSESGHSTIYADSVKGRFTISRDNAKNTLYLQMSSLKTEDTATYY |
| | 1-3_F3 | CAR |
| 164 | >feline | DVQLVESGGNLVKPGGSLRLTCVASGFTFSSSGMSWVRQAPGKGLQ |
| | VH_FW | WVAHIYDDGSTERYADSVKGRFTISRDNAKNTLYLQMNSLKTEDTATY |
| | 1-3_F6 | YCAR |
| 165 | >feline | DVQLVESGGDLVKPGGSLRLTCVASGFTFSTYYMNWVRQAPGKGLQ |
| | VH_FW | WVAYINPDGSSTNYADSVKGRFTISRDNAKNTLYLQMNSLKTEDTATY |
| | 1-3_F8 | YCTS |
| 166 | >feline | DVRLVESGGDLVQPGGSLRLTCVASGFTFSTYEMNWVRQAPGKGLR |
| | VH_FW | WVTGISSSGGATYYADSVKGRFTISRDNAKNTLYLSMNSLKTEDTATY |
| | 1-3_B2 | YCTS |
| 167 | >feline | DVQLLESGGDLVKPGGSLRLTCVASGFTFSSYGMSWVRQAPGKGLQ |
| | VH_FW | WVAAISGSGGSTYYADSVKGRFTISRDNAKNTLYLQMNSLKTEDTATY |
| | 1-3_F4 | FCAR |
| 168 | >feline | DVRLVESGGDLVKPGGALRLTCVASGFTFSVYGMSWVRQAPGKGLQ |
| | VH_FW | WVTAISGNGGDTYYADSVKGRFTISRDNAKNTLYLHMNSLKTEDAATY |
| | 1-3_C1 | YCAR |
| 169 | >feline | DVQLVESGGDLVKPGGSLRLTCVASGFTFSNYGMTWVRQAPGKGLQ |
| | VH_FW | WVAYIRYDGSSTNYADSVKGRFTISRDNAKNTLYLQMNSLKTEDTATY |
| | 1-3_C5 | YCST |
| 170 | >feline | DVQLVESGGDLVKPGGSLRLTCVASGFTFSSHYMNWVRQAPGKGLQ |
| | VH_FW | WVSWINTDGSTTSYADSVKGRFTISRDNSKNTLYLQMNNLKTEDTATY |
| | 1-3_A2 | YCAR |
| 171 | >feline | DVQLVESGGDLVKPGGSLRLTCVASGFTFSNYYMSWVRQAPGKGLQ |
| | VH_FW | WVSWINKDGSVTSYADSVKGRFTISRDNSKNTLLLQMNNLKTEDAATY |
| | 1-3_A1 | YCAR |
| 172 | >feline | DVQLVESGGDLVKPGGSLRLTCVASGFTFSSYAMSWVRQAPGKGLQ |
| | VH_FW | WVSAISEGDGSAFYTDSVKGRFTISRDNAKNTLYLQMNSLKTEDTATY |
| | 1- | FCVR |
| | 3_E12 | |
| 173 | >feline_ | EVOLVESGGDLVKPGGSLRLTCVASGFTFSNYGMNWVRQAPGKGLQ |
| | VH_FW | WVAAISGSGGSTYYADSVKGRFTISRGNAKNALYLQMNSLKTEDTATY |
| | 1-3_C9 | YCAR |
| 174 | >feline | DVQLVESGGDLVKPGGSLRLTCVASGFTFSSYYMNWVRQPPGKGLQ |
| | VH_FW | WVANINTDGSTTSYADSVKGRFTISRDNAKNTLYLQMNSLKTEDTATY |
| | 1-3_C4 | YCAR |
| 175 | >feline | QVLLVQSGGDLVKPGGSLRLTCVASGFTFNSYAMSWVRQAPGKGLQ |
| | VH_FW | WVADISGSGGATAYADSVKGRFTISRDNAKNTLYLQMNSLKTEDTATY |
| | 1- | YCAK |
| | 3_D11 | |
| 176 | >feline_ | DVRLVESGGDLVKPGGSLRLTCVASGFTFSSFGMGWVRQAPGKGLQ |
| | VH_FW | WVAYIRSTGSTTNYADSVKGRFTISRDNAKNTLYLQMNSLKTEDTATY |
| | 1-3_H3 | YCAT |
| 177 | >feline | DVQLVESGGDLVKPGGSLRLTCVASGFTFSNYGMSWVRQAPGKGLQ |
| | VH_FW | WVAANSGTGSSTYYADSVKGRFTISRDNAKNTLYLQMNSLKTEDTATY |
| | 1- | YCAR |
| | 3_A11 | |
| 178 | >feline | DEQLVESGGNLVKPGGSLRLTCVASGFTFSNYDMSWVRQAPGKGLQ |
| | VH_FW | WVAYIYNDGSGTSYADSVKGRFTISRDNAKNTLYLQMNSLKTEDTATY |
| | 1-3_C3 | YCAK |
| 179 | >feline | DEQLVESGGDLVKPGGSLRLTCVASGFTFSSYSMNWVRQAPGKGLQ |
| | VH_FW | WVAYIRYDGSRLYYADSVKGRFTVSRDNAKNTLYLQMNSLKTEDTATY |
| | 1-3_D4 | YCAN |
| 180 | >feline | DVQLVESGGDLVKPGGSLTLTCVASGFTFNIYNMNWVRQAPGKGLQW |
| | VH_FW | VAYISRGGGSIHYADSVKGRFTISRDNAKNTLYLQMDSLKTDDTATYYC |
| | 1-3_F1 | TG |
| 181 | >feline | DVQLVESGGDLVKPGGSLRLTCVASGFTFRSFWMNWVRQAPGKGLQ |
| | VH_FW | WVSAISDSGTSTGYADSVKGRFTISRDNAKNTLYLQMNSLKTEDTATY |
| | 1-3_B3 | YCTK |
| 182 | >feline | EVOLVESGGDLVKPGGSLRLTCVASGFTFRSYWMNWVRQAPGKGLQ |
| | VH_FW | WVSAISDSGRSTGYADSVRGRFTISRDNAKNTLYLQMNSLKTEDTATY |
| | 1-3_C2 | YCAG |
| 183 | >feline | EVQLVESGGDLVKPGGSLRLTCVASGFTFSNYYMSWVRQSPGKGLQ |
| | VH_FW | WVAYISTSGGDFYYADSVKGRFTVSRDNAKNTLYLQMNSLKTEDTATY |
| | 1-3_E8 | YCAR |
| 184 | >feline | DEQLVESGGDLVKPGGSLRLTCVASGFPFSRYGMSWVRQAPGKGLQ |
| | VH_FW | WVAAIRGSGGVTYYADSVKGRFTISRDNAKNTLYLQMNSLKTEDTATY |
| | 1-3_E9 | YCAR |
| 185 | >feline | DVRLVESGGDLVKPGGSLRLTCVASGFTFSSYSMNWVRQAPGKGLQ |
| | VH_FW | WVAYIRYDGSSIYYADSVKGRFTVSRDNAKNTLYLQMNSLKTEDTATY |
| | 1-3_G2 | YCAR |
| 186 | >feline_ | DVQLVESGGNLVKPGGSLRLTCVASGFTFSSYDMSWVRQAPGKGLQ |
| | VH_FW | WVAYIYDDGSDTNYADSVKGRFTVSRDNAKNTLYLQMNSLKTEDTATY |
| | 1- | YCAM |
| | 3_D12 | |
| 187 | >feline | DVQLVESGGDLVKPGGSLRLTCVASGFTFSSYGMNWVRQAPGKGLQ |
| | VH_FW | WVAYIRYDGSSTNYADSVKGRFTISRDNAKNTLYLQMSSLKTEDTATY |
| | 1-3_D5 | YCAM |
| 188 | >feline_ | DVQLVESGGDLVKPGGSLRLTCVASGFIFSRYGMSWVRQAPGKGLQ |
| | VH_FW | WVAGISTSGGNTYYADSVKGRFTISRDNAKNTLYLQMNSLKTEDTATY |
| | 1-3_A3 | YCAR |
| 189 | >feline | QVLLVQSGGDLVKPGGSLRLTCVASGFTFSDYAMSWVRQAPGKGLQ |
| | VH_FW | WVSTVSESGSNTYYGDSVRGRFTISRDNAKNKLYLQMNSLKSEDTAT |
| | 1-3_E2 | YYCAR |
| 190 | >feline | DEQLVESGGDLVKPGGSLRLTCVASGFTLGSYDMTWVRQAPGKGLQ |
| | VH_FW | WVAAIGHDGSTTAYADSVKGRFTISRDNAKNTLYLQMNSLKTEDTATY |
| | 1- | YCAR |
| | 3_G12 | |
| 191 | >feline | DVQLVESGGDLVKPGGSLRLTCVASGFTFSNYYMEWVRQAPGKGLQ |
| | VH_FW | WVAQISEGGGSTYYADSVKGRFTISRDNAKNTLYLQMNSLKTEDTATY |
| | 1-3_H4 | FCAR |
| 192 | >feline | DVRLVESGGDLVKPGGSLRLTCVLPLDSPSVPTGSISIYWMNWVRQAP |
| | VH_FW | GKGLQWVAGISGSGDRTGYADSVKGRFTISIDNAKNTLYLQMDSLKTE |
| | 1-3_A4 | DTATYYCET |
| 193 | >feline | YVQLVESGGDLVKPGGSLRLTCVASGFTFSSYYMHWVRQAPGKGLQ |
| | VH_FW | WVAQISDSGGSTYYADSVKGRFTISRDNAKNTLYLQMNGLKTEDTATY |
| | 1- | YCAR |
| | 3_D10 | |
| 194 | >feline | DVRLVESGGDRVKPGGSLRLTCMASGFNVRGYAMTWVRQAPGMGL |
| | VH_FW | QWVSSISSGGTTYYADSVKGRFTISKDDAENTLYLQMNSLKTEDTATY |
| | 1-3_E7 | YCAG |
| 195 | >feline | DVQVVESGGDLVKPGGSLRLTCLASGFTFSSYGMSWVRQAPGKGLQ |
| | VH_FW | WVAYIRYDGSSTKYADSVKGRFTISRDNAKNTLYLQMNSLKTEDTATY |
| | 1-3_H1 | YCAT |
| 196 | >feline | DVQLVESGGDLVKPGGSLRLTCVASGFTFSTYYMHWVRQAPGKGLQ |
| | VH FW | WVAQINSGGSSTFYADSVKGRFTISRDNAKNTLHLQMNSLKTEDTGTY |
| | 1-3_H2 | YCAS |
| 197 | >feline | DVQLVESGGDLVKPGGSLRLTCVASGFTFSTYGMSWVRQAPGKGLQ |
| | VH_FW | WVAAISGSGGSTSYAESVKGRFTISRDNAKNTLYLQMNSLKTEDTATY |
| | 1-3_E6 | YCAR |
| 198 | >feline | EVOLVESGGDLVKPGGSLRLTCVASGFTFTGDAMSWVRQAPGRGLQ |
| | VH_FW | WVSTIDSGGNTHYIDSVKGRFTISRDNAKNTLYLQMNNLKTEDTATYYC |
| | 1-3_A9 | AG |
| 199 | >feline | DVQLVESGGDLVKPGGSLRLTCVASGFPFSSYGMSWVRQAPGKGLQ |
| | VH_FW | WVAYIRYDGSSTNYADSVKGRFTISRDNAKNTLYLQMDSLKTEDTATY |
| | 1- | YCTS |
| | 3_E10 | |
| 200 | >feline | DVQLVESGGDLVKPGGSLRLTCVASGFTFNNFAMSWVRQAPGKGLQ |
| | VH_FW | WVSALSESGHSTIYADSVKGRFTISRDNAKNTLSLQMDSLKTEDTATYY |
| | 1-3_G8 | CAT |
| 201 | >feline | DVQLVESGGDLVQPGGSLRLTCVASGFTFNNYEMNWVRQAPGKGLQ |
| | VH FW | WVAYISFSGDSTNYADSVQGRFTISRDNAKNTLYLQMNSLKTEDTATY |
| | 1- | YCAR |
| | 3_A10 | |
| 202 | >feline | DEQLVESGGDLVKPGGSVRLTCVASGFTFSTSAMSWVRQAPGKGLQ |
| | VH_FW | WVSSVSENGGGTFYADSVKGRFTVSRDNARNTLYLQMNSLKTEDSAT |
| | 1-3_B4 | YYCAR |

### Example 5: Expression of Selected Clones in Fab Format and Expression Check

All unique scFv clones identified in Example 4 were monoclonally converted into the Fab format to allow for soluble expression of Fab fragments. To this end, each VL and VH was individually PCR amplified and stitched together via OE-PCR introducing the constant light chain region (CL) and the VH promotor as well as constant region (CH1), simultaneously removing the 18 amino acids linker previously used for the scFv format. Feline wildtype and mutant constant regions devoid of an unpaired cysteine are depicted in Table 8.

TABLE 8
Wildtype and mutant Feline constant light and heavy chain sequences

| SEQ ID No: | Sequences |
|---|---|
| 203 | >feline_Fab_feline_Fab_feline_CL_lambda_wild type |
| | GQPKSAPSVTLFPPSNEELSANKATLVCLISDFYPSGLTVAWKADGTPVTQGV |
| | ETTKPSKQSNNKYAASSYLSLSPNEWKSRSRFTCQVTHEGSTVEKSVVPAECS |
| 204 | >feline_Fab_feline_Fab_feline_CL_lambda_C105S |
| | GQPKSAPSVTLFPPSNEELSANKATLVCLISDFYPSGLTVAWKADGTPVTQGV |
| | ETTKPSKQSNNKYAASSYLSLSPNEWKSRSRFTCQVTHEGSTVEKSVVPAESS |
| 205 | >feline_Fab_feline_Fab_feline_CH1_wild type |
| | ASTTAPSVFPLAPSCGTTSGATVALACLVLGYFPEPVTVSWNSGALTSGVHTF |
| | PAVLQASGLYSLSSMVTVPSSRWLSDTFTCNVAHPPSNTKVDKTV |
| 206 | >feline_Fab_feline_Fab_feline_CH1_C15S |
| | ASTTAPSVFPLAPSSGTTSGATVALACLVLGYFPEPVTVSWNSGALTSGVHTF |
| | PAVLQASGLYSLSSMVTVPSSRWLSDTFTCNVAHPPSNTKVDKTV |

Single clones were picked, Fab-fragments were produced and tested essentially as described in Example 4. An ELISA was conducted capturing soluble Fabs using a coated anti-feline IgG antibody, detection was carried out against the Fab FLAG-tag using an anti-FLAG antibody-HRP. The ELISA revealed expression signals up to 16x S/BG. The results of the expression analysis by ELISA are shown in FIG. 8. Surprisingly, there was a good correlation between expression in the scFv and Fab-fragment, which makes these framework combinations promising scaffolds for usage in phage display libraries of different format. The correlation of expression levels of feline antibody fragments in the scFv and Fab format are shown in FIG. 9. The 21 highest expressing Fab clones were sent for sequencing to confirm sequence data obtained already in scFv format. FW1-3 regions of VH-sequences are depicted in FIG. 1, FW1-3 regions of VL-sequences in FIG. 2.

### Example 6: Construction of a Phage Display Vector Containing Favorable VH/VL Pairs Compatible with Introduction of Diversity in HCDR1, HCDR2, HCDR3 and LCDR3

For the generation of a fully synthetic feline phage display library, most promising light and heavy chain pairs identified and validated in Example 3-5 were synthesized as DNA fragments. The Geneious software suite was used to codon-optimize the sequences for expression in E. coli, but omitting rare codons for expression in mammalian hosts, e.g. the triplet CGC was used to encode the amino acid arginine as it is compatible with both bacterial and mammalian expression systems. NheI/PstI restriction sites were introduced to allow diversification of the HCDR1/HCDR2 segments, BssHII/XhoI is used to introduce HCDR3 diversity. For the introduction of the BssHII site, it is mandatory to use the CAR motif at the beginning of the HCDR3 region. However, this motif is the dominant variant anyhow. BbsI/KpnI restriction sites are used to introduce LCDR3 diversity. Modifications at the heavy chain starting motif to DVQL can be introduced to make bulk reformatting between antibody formats possible, realized by the introduction of a MfeI site in the QL nucleotide sequence. Importantly, these modifications represent sequences which are naturally present in the feline IgG repertoire. These modified VL/VH pairs can be used for phage display in both a scFv format as well as a Fab format. The concept of the library building blocks is depicted in FIG. 4 and FIG. 5. For the initial library, only the LCDR3 and HCDR3 regions are diversified but candidates derived from such library can be easily optimized using introduction of maturation modules in the HCDR1 and HCDR2 region. Alternatively, it is possible to generate libraries that are diversified in all mentioned CDRs from the beginning on.

A schematic overview of the building blocks for seven exemplary antibody scaffolds for the
VH library is shown in FIG. 9
(SEQ ID NO: 225 is DVQLVESGGDLVKPGGSLRLTCVASGFTFSDYDMSWVRQAPGKGLQWVAAISYSGGGTG
YSDSVKGRFTISRDNAKNTLYLQMNSLKTEDTATYYCAR;
SEQ ID NO: 226 is DVQLVESGGDLVKPGGSLRLTCVASGFTFSNYGMSWVRQAPGKGLQWVAAISGTGSSTYY
ADSVKGRFTISRDNAKNTLYLQMNSLKTEDTATYYCAR;
SEQ ID NO: 227 is DVQLVESGGDLVKPGGSLRLTCVASGFTFSNYDMSWVRQAPGKGLQWVAAIAYSGGNTG
YADSVKGRFTISRDNAKNTLYLQMNSLKTEDTATYYCAR;
SEQ ID NO: 228 is DVQLVESGGDLVKPGGSLRLTCVASGFTFSRYGMSWVRQAPGKGLQWVAAISGSGDSTY
YADSVKGRFTISRDNAKNTLYLQMNSLKTEDTATYYCAR;
SEQ ID NO: 229 is DVQLVESGGDLVQPGGSLRLTCVASGFTFSSYEMNWVRQAPGKGLQWVAYISSGGSTYY
ADSVKGRFTISRDNAKNTLYLQMNSLKTEDTATYYCAR;
SEQ ID NO: 230 is DVQLVESGGDLVKPGGSLRLTCVASGFTFSSYYMHWVRQAPGKGLQWVAQISDSGGSTY
YADSVKGRFTISRDNAKNTLYLQMNGLKTEDTATYYCAR;
SEQ ID NO: 231 is DVQLVQSGGDLVKPGGSLRLTCVASGFTFSSYAMSWVRQAPGKGLQWVADISGSGGATA
YADSVKGRFTISRDNAKNTLYLQMNSLKTEDTATYYCAR)) and for the VL library in FIG. 10
(SEQ ID NO: 218 is QSGPNQPSSVSGALGQRVTISCTGIDTYVGWFQQIPGMAPKTIIVGNTNRPSGVPDRFSGS
KSGNTGTLTITGLQAEDEADYYC;
SEQ ID NO: 219 is QSGPNQPSSVSGALGQRVTISCTGVGNYVDWYQQVPGMAPKTIIYGNRYRPSGVPDRFSG
SKSGSTGTLTITGLQAEDEADYYC;
SEQ ID NO: 220 is QSGPNQPSSVSGTLGQTITISCTGVSVFVDWYQQIPGMAPKTVIYRNTNRLSEVPDRFSGS
KSGITATLTITGLQAEDEADYYC;
SEQ ID NO: 221 is QSVVTQPPSVSGALGQRVTISCTGSSSNVGRGNYVSWYQQLSGTPPKLLIYGNTDRLSGVP
DRFSGSKSGTTGSLTITGLQTEDEADYYC;
SEQ ID NO: 222 is QSGPNQPSSVSGALGQRVTISCTGAGSYVGWYQQVPGMAPKTIIYDDNKRPSGVPARFSG
SKSGSTGTLTITGLQAEDEANYYC;
SEQ ID NO: 223 is QPVLTQPPSVSGALGQRVTISCTGSSSNIGRGNYVSWYQQVSGTAPKLLIYGDTNRPSGVP
DRFSGSKSGSTGSLTITGLQADDEADYYC;
SEQ ID NO: 224 is QPVLTQPPSVSGSLGQRVTISCTGTSSNIGSGNYVSWYRQVPGIAPSLLIYENFKRPSGVPD
RFSGSKSGSSGSLTITGLQADDEVDYYC).

### Example 7: Design of HCDR3 Diversity Modules

HCDR3 and LCDR3 are the major paratope forming CDRs in an antibody, significantly contributing to antigen binding and recognition. Therefore, the naive library is exclusively diversified in the CDR3s regions. For affinity maturation and framework diversification, our library further allows the combined diversification of HCDR1 and HCDR2.

The heavy chain framework 4 (C-terminal of HCDR3) is highly conserved in the feline IgG repertoire. Therefore, this region was kept constant (SEQ209) to facilitate molecular cloning of the HCDR3 diversity module. Further, we chose the CAR motif preceding the HCDR3 to allow for the insertion of a BssHII restriction site.

As described in Example 1, HCDR3 lengths from 7-14 amino acids were used (HCDR3 starts at position 98 according to the numbering scheme shown in Table 9 and FIG. 9). This selection represents 91% of all lengths identified in our NGS dataset. Further, it has been shown for therapeutic antibodies that very short or long HCDR3s are often not featuring the required stability.

TABLE 9
Concept of length variations in the HCDR3 diversification module. The last five conserved amino acids in
FW3 including the dominant CAR motif are show. Xₐₐ defines random amino acids (Xaa may be any one of A,
R, N, D, C, Q, E, G, H, I, L, K, M, F, P, S, T, W, Y, or V). FW4 is conserved for all library members.

| length/ | % in | . . . FW3 | | | | | HCDR3 | | | | | | | | | | |
|---|---|---|---|---|---|---|---|---|---|---|---|---|---|---|---|---|---|
| position | library | 93 | 94 | 95 | 96 | 97 | 98 | 99 | 100 | 101 | 101a | 101b | 101c | 101d | 101e | 101f | 101g |
| 7 | 6 | Y | Y | C | A | R | Xaa | Xaa | Xaa | Xaa | | | | | | | |
| 8 | 9 | Y | Y | C | A | R | Xaa | Xaa | Xaa | Xaa | Xaa | | | | | | |
| 9 | 13 | Y | Y | C | A | R | Xaa | Xaa | Xaa | Xaa | Xaa | Xaa | | | | | |
| 10 | 13 | Y | Y | C | A | R | Xaa | Xaa | Xaa | Xaa | Xaa | Xaa | Xaa | | | | |
| 11 | 19 | Y | Y | C | A | R | Xaa | Xaa | Xaa | Xaa | Xaa | Xaa | Xaa | Xaa | | | |
| 12 | 19 | Y | Y | C | A | R | Xaa | Xaa | Xaa | Xaa | Xaa | Xaa | Xaa | Xaa | Xaa | | |
| 13 | 13 | Y | Y | C | A | R | Xaa | Xaa | Xaa | Xaa | Xaa | Xaa | Xaa | Xaa | Xaa | Xaa | |
| 14 | 8 | Y | Y | C | A | R | Xaa | Xaa | Xaa | Xaa | Xaa | Xaa | Xaa | Xaa | Xaa | Xaa | Xaa |

| length/ | HCDR3 | | | FW4 | | | | | | | | | | |
|---|---|---|---|---|---|---|---|---|---|---|---|---|---|---|
| position | 102 | 103 | 104 | 105 | 106 | 107 | 108 | 109 | 110 | 111 | 112 | 113 | 114 | 115 |
| 7 | Xaa | Xaa | Xaa | W | G | Q | G | A | L | V | T | V | S | S |
| 8 | Xaa | Xaa | Xaa | W | G | Q | G | A | L | V | T | V | S | S |
| 9 | Xaa | Xaa | Xaa | W | G | Q | G | A | L | V | T | V | S | S |
| 10 | Xaa | Xaa | Xaa | W | G | Q | G | A | L | V | T | V | S | S |
| 11 | Xaa | Xaa | Xaa | W | G | Q | G | A | L | V | T | V | S | S |
| 12 | Xaa | Xaa | Xaa | W | G | Q | G | A | L | V | T | V | S | S |
| 13 | Xaa | Xaa | Xaa | W | G | Q | G | A | L | V | T | V | S | S |
| 14 | Xaa | Xaa | Xaa | W | G | Q | G | A | L | V | T | V | S | S |

Since each length has slightly adapted amino acid compositions, gradually diverging from shorter to longer HCDR3s, we synthesized all lengths using individual composition as identified in our NGS datasets. Distributions of amino acids in diversification modules of different lengths are depicted in FIG. 11, FIG. 12, FIG. 13, the percentage share of individual CDR lengths is shown in Table 9.

In short, the strategy for DNA synthesis is as following:
1) Each length is individually in silico calculated using the individual amino acid frequency for each position, omitting cysteines and PTM motifs (e.g. glycosylation motifs, asparagine deamidation sites, aspartate isomerization sites, integrin binding sites, fragmentation sites and hydrophobic stretches).
2) Based on the HCDR3 length distribution, a fraction of a total of 1E+10 sequences is in silico pre-selected to comprise a set of sequences which optimally represent the sequence space for each set of lengths. The total number of sequences synthesized across all lengths used is around 1E+10.
3) These 1E+10 sequences are synthesized using the TWIST Bioscience technology, resulting in a HCDR3 library mimicking the natural length and amino acid distribution found in cats.

### Example 8: Design of LCDR3

LCDR3 lengths from 9-12 amino acids were used for the diversification module (LCDR3 starts at position 84 according to the numbering scheme shown in Table 10 and FIG. 10. This selection represents 93% of all lengths identified in our NGS dataset as described in Example 1. Further, it has been shown for therapeutic antibodies that very short or long LCDR3s are often not featuring the required stability. The light chain framework 4 (C-terminal of LCDR3) is conserved in the feline IgG repertoire. Therefore, this region was kept constant (SEQ208) to facilitate molecular cloning of the LCDR3 diversity module.

Since each length has slightly adapted amino acid compositions, gradually diverging from shorter to longer LCDR3s, we synthesized all lengths using their individual composition as identified in our NGS datasets. Distributions of amino acids in diversification modules of different lengths are depicted in FIG. 14, the percentage share of individual CDR lengths is shown in Table 10.

The strategy for DNA synthesis is essentially as described for the HCDR3. Again, care was taken to omit cysteines and unfavorable PTM motifs.

### Example 9: Design of HCDR1 and HCDR2 Maturation Modules

In order to allow for affinity maturation, our VH sequence features NheI/PstI restriction sites to rapidly diversify the HCDR1-FW2-HCDR2 segment. The HCDR1-HCDR2 design is as follows:

Both, HCDR1 and HCDR2 are in silico calculated using the individual amino acid frequency for each position, omitting cysteines and PTM motifs. Since the FW2 sequence is relatively conserved for most promising sequences identified in Examples 3-5, it was kept constant to improve the functionality and maturation capacity of our fully synthetic library (WVRQAPGKGLQWV, SEQ NO: 241). As we did not observe significant length variations during the NGS analysis, only the two most prominent CDR lengths for HCDR1 and HCDR2 were chosen.

The maturation module was synthesized using the TWIST Bioscience technology, resulting in a HCDR1-HCDR2 library mimicking the natural amino acid distribution found in cats. As for HCDR3 and LCDR3, any unfavorable amino acid motifs were avoided.

The concept and amino acid distribution of the HCDR1/2 maturation module is shown in FIG. 15 which depicts the flanking regions 5′ to the HCDR1 region, the diversified HCDR1, FW2, the diversified HCDR2 and parts of the subsequent FW3. No length variations are realized in this module. Xaa defines random amino acids (Xaa may be any one of A, R, N, D, C, Q, E, G, H, I, L, K, M, F, P, S, T, W, Y, or V). FW2 is conserved for all library members.

### Example 10: Antibody Selections

Phage display selections may be done as described below or by another method known to one of skill in the art. For example, parallel panning strategies (e.g., solution panning, Fc capture panning, direct solid phase panning) are performed in order to maximize the chance of identifying diverse binding antibodies with the desired biophysical characteristics. Various soluble proteins can be chosen as model antigens for library validation (e.g. lysozyme, GFP). Collection screening against three model antigens was performed in a direct solid phase panning with the antigen immobilized on a plastic surface as described below. Selection against the model antigens can also be performed in solution mode as described below.

### Solid Phase Pannings

For solid phase pannings, antigens were immobilized on the surface of a microtiter plate (Maxisorp, 96-well flat bottom) over night at 4° C. Coating checks with respective anti-gens were performed to identify ideal coating concentrations. Typically, antigens were used in the first panning round at a concentration of 1 μg/ml representing antigen saturation on the plate. For each selection, 300 μl/well of antigen diluted in PBS (Phosphate buffered saline, pH 7.4) were used. After incubation, antigen solutions were removed, wells were washed twice with PBST (PBS supplemented with 0.05% Tween-20) before addition of 400 μl/well blocking buffer (typically, 5% milk in PBS). Plates were incubated for at least 1 h at RT.

In parallel, library phages were blocked for at least 1 h at RT with blocking solution, generally 5% milk in PBS. Depending on whether the target antigen contains tags, such as biotin or a His-tag, or is a Fc-fusion protein, additional reagents were supplemented to the blocking buffer. Also, depletion steps on relevant material was preformed prior to using the phages in pannings (e.g. adsorption on wells coated with a biotinylated irrelevant protein to remove potential biotin binders).

For each panning subcode, 20 ml 2×YT medium were incubated with E. coli TG1F+ from a M9 minimal agar plate in a phage-free working space and the culture was later used for infection with the selected phage. Culture was shaken at 160 rpm and 37° C. until an OD_{600 nm} of 0.6 was reached. E. coli culture was kept on ice until required for infection of eluted phage.

After antigen coating and blocking, blocked phages were transferred to respective wells and incubated for 1-2 h at RT. Subsequently, phages were removed by rapidly inverting the plate over a phage waste container. To further remove unspecific and weak phages, a number of washing steps with PBST and PBS were performed. In the first panning, standard washing steps were applied (3×PBST quick, 2×PBST for 5 min, 3×PBS quick, 2×PBS for 5 min). Depending on phage output titers, stringency was adapted in the subsequent round by increasing the number and time if washing steps.

Following antigen-phage incubation and washing, specific phages were typically eluted by addition of trypsin (250 μl of a 10 μg/ml trypsin solution in PBS, 30 min at 37° C.) to cleave the proteinase-sensitive linker between the antibody fragment and the gIII protein. Alternatively, a pH-shift was applied to break the antigen-antibody interaction. This can be achieved using either acidic or basic buffers but typically low-pH elution was used. To this end, 300 μl elution buffer (100 mM Glycin/HCl, 0.5 M NaCl, pH 2.2) was added to respective wells, incubated for 10 min and subsequently transferred to a fresh tube containing 10 μl neutralization buffer (2 M Tris base).

After neutralization, phage suspension of each selection was transferred to a pre-warmed E. coli TG1F+ culture each and incubated for 45 min in a water bath at 37° C. without shaking. Bacterial cultures were centrifuged, supernatants were discarded and pellets were resuspended in 2×YT medium and plated on LB/Cam agar plates and incubated over night at 37° C. Next day, bacteria were scraped of the plates with freezing medium (2×YT medium containing 34 μg/ml chloramphenicol (Cam), 1% glucose and 15% glycerol) and aliquots were stored at −80° C. before preparation of phages for the subsequent panning round.

### Solution and Semi-Solution Pannings

For solution and semi solution pannings, magnetic beads (GE, Sera-Mag Streptavidin-Coated Magnetic Particles, Cat: #30152104010150) in combination with biotinylated model antigens were used to capture the antigen-phage complex or immobilize antigen on the bead surface, respectively.

Before pannings start, beads were washed and blocked, for semi-solution pannings also loaded with antigen. To this end, 250 μl beads for each selection were transferred to 2 ml low binding tubes, beads were captured with a magnetic particle separator and storage solution was removed. Then, beads were washed 3 times with PBS, using a magnet to collect beads for removal of washing buffer. Afterwards, beads were blocked for 2 h at RT in blocking solution (100% Chemiblock or 5% BSA in PBS).

In parallel, phages were blocked. To this end, the required amount of phages was mixed with an equivalent volume of 2× blocking solution and incubated for at least 1 h at RT. Typically, Chemiblock was used and supplemented with irrelevant proteins containing tags or biotin for blocking against these structures. In addition, blocked phages were pre-absorbed on empty magnetic beads to remove sticky phages.

After blocking of beads and blocking/pre-adsorption of phages, biotinylated antigen was added to the phage solutions and incubated for 1 h at RT rotating. For capture of the phage/antigen complex, 100 μl blocked magnetic beads were added and incubated for 15 min at RT. Subsequently, unspecific phage were removed by washing (5x PBST quick, 3×PBST for 5 min, 3×PBS quick), washing stringency was adapted from round to round depending on panning outputs. With the last washing step, magnetic beads with the captured antigen-phage complex were transferred into a fresh low binding tube.

For elution of specific phage, 300 μl elution buffer was added for 10 min at RT. Similar to solid phase pannings, elution can be performed using acidic or basic conditions or by addition of trypsin. Subsequently, phage suspensions of each selection were transferred to 20 ml of pre-warmed E. coli TG1F+culture each and incubated for exactly 45 min in a water bath at 37° C. without shaking. Bacterial cultures were centrifuged for 5 min at 4600 rpm at 4° C. and supernatants were discarded. Pellets were resuspended in 600 μl 2×YT medium and plated on large LB/Cam agar plates and incubated over night at 37° C. Next day, bacteria were scraped of the plates with a few mL freezing medium (2×YT medium containing 34 μg/ml Cam, 1% glucose and 15% glycerol) using a sterile Drygalski spatula and aliquots were stored at −80° C. before preparation of phages for the sub-sequent panning round

Semi-solution pannings were performed similar to solution pannings with the following modification: Instead of capturing the complex of phages bound to biotinylated target out of solution using Streptavidin magnetic beads, the respective antigens were immobilized on the beads already prior to blocking of the beads. Thus, the panning mode reflects a selection on a solid phase but allows better orientation of the target and washing conditions compared to a panning where the antigen is coated on the surface of a microtiter plate.

The remaining steps of the panning, including washing and elution steps is essentially performed as described above.

### Phage Preparation

For each phage preparation, inoculation medium (2×YT medium containing 34 μg/ml Cam and 1% glucose) was inoculated with phagemid containing bacterial suspension or glycerol stock resulting in an OD₆₀₀ of around 0.2 to 0.3. Cultures were incubated for 30-90 min at 37° C. shaking until an OD₆₀₀ of around 0.5 to 0.6 was reached. VCSM13 helper phages were added (4.00E+10 tu of helper phage per 5 ml bacterial culture were used) and incubated for 30 min at 37° C. without shaking and then for 30 min at 37° C. shaking at 250 rpm. Subsequently, bacteria were spun down and helper phage containing supernatant was discarded. Phage infected bacteria were resuspended in induction medium (2×YT medium containing 34 μg/ml Cam, 50 mg/ml Kanamycin (Kan and 0.2 mM IPTG and incubated for 18-20 h at 22° C., shaking at 200 rpm in a phage shaker. Next day, bacteria were spun down and the supernatant containing the antibody-presenting phages were transferred to new tubes. For phage precipitation, 1/5 volume of ice cold PEG/NaCl was added to the phage-containing supernatant, mixed and incubated for at least 30 min on ice, gently shaking. Precipitated phages were spun down for at least 30 min at 12000×g at 4° C. Supernatants were removed quantitatively and phage pellets were resuspended in an adequate volume of PBS. Phage titers were determined using spectrophotometer measurement (Nanodrop; absorbance at 268 nm was used as a means for phage amounts; A268 nm of 1 corresponds to a phage titer of 5.00E+12 tu/ml).

Subcloning into Expression Vector

After multiple rounds of panning, polyclonal phage outputs were subcloned into the bacterial expression vector pFeBx that is compatible with the expression of either single chain or Fab fragments. The scFv- or Fab-coding fragments were removed from the phage display vector using the restriction enzymes NcoI/EcoRI and NcoI/XhoI, respectively, isolated using preparative agarose gel electrophoresis (1.2% agarose) and fragments were DNA purified from the gel slice using an appropriate gel extraction kit. The ligation reaction with the insert and pre-cut pFeBx vector and subsequent transformation in chemically competent E. coli TG1F- was performed according to standard procedures.

Wells of expression plates (Round bottom 96-well plates (e.g. Thermo Fisher, Cat: #262162) were filled with 120 μl/well all-in-one-medium (2×YT medium containing 34 μg/ml Cam, 0.1% Glucose and 0.5 mM IPTG) and were inoculated with single colonies from agar plates of the subcloning procedure. Plates were incubated for 5 h at 37° C. shaking at 400 rpm and afterwards overnight at 22° C. To prepare crude bacterial lysates used for screening purposes, so called BEL-lysates, 40 μl/well of lysis buffer (2×BBS containing 2.5 mg/ml lysozyme, 4 mM EDTA and 13 U/ml Benzonase) were added and incubated for 1 h at 22° C. shaking at 400 rpm. Then, 40 μl/well of blocking buffer (1×PBS containing 5% milk powder) were added and incubated at 22° C. for 1 h. Plates were stored at −20° C. or directly used for screening.

### Screening ELISA

For screening, scFV or Fab-containing lysates (BEL lysates) were tested for binding to antigens immobilized on Maxisorp microtiter plates or alternatively, using biotinylated antigens bound to Streptavidin plates. Following immobilization of the antigens on the respective surface, plates were washed three times with PBST and subsequently blocked with 5% milk in PBS for 1 h at RT. BEL lysates, control antibodies and negative controls were transferred to plates and incubated for 1 h at RT. Plates were washed 3 times with PBST and subsequently, detection antibody was added to the plates and incubated for 1 h at RT. For the detection of scFv, an anti-FLAG-HRP antibody (R&D Systems, HAM85291) was used, Fabs were detected with an goat anti-feline IgG (L+H)-HRP (Jackson). Plates were washed 5 times with PBST and detection of bound antibodies was performed using the QuantaBlu reagent according to the manufacturer's instruction on a Tecan Genious Reader (excitation filter: 320 nm, emission filter: 430 nm).

### Example 11 Results from Panning Campaigns on Model Antigens

eGFP (enhanced green fluorescent protein), fAG1 (a feline soluble cytokine) and fAG2 (the ECD of a feline receptor) were chosen as a model antigens for library validation. Screening and sequencing data obtained from a solid phase panning and direct coating ELISA described as above are shown, indicating that antigen-specific binders from the fully synthetic feline phage library can be isolated.

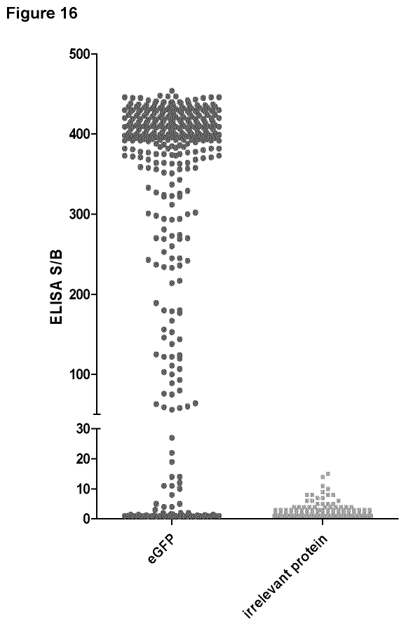

Antigen binding was tested using scFv containing BEL lysates. Primary hits were defined as antibody fragments that resulted in an ELISA signal of at least 60-fold above background and their specificity to model antigens was confirmed in a secondary ELISA on an irrelevant control antigen. Exemplary screening data on eGFP following a solid phase panning is depicted in a dot-blot visualization in FIG. 16, indicating strong target-specific binding and only minimal binding to an irrelevant protein.

For all three test pannings, a total of 312, 139 and 24 hits for eGFP, fAG1 and fAG2, respectively, were identified, indicating overall hit-rates between 13% and almost 90% (see Table 11).

TABLE 11
Summary of screening and sequencing results on three test antigens

| | | Total clones | Hits | Hit rat | Unique | Overall |
|---|---|---|---|---|---|---|
| | Panning | screened | [S/BG > 60] | [%] | sequences | diversity |
| eGFP | Solid strategy 1 | 176 | 163 | 93 | 12/12 | 100% |
| | Solid strategy 2 | 176 | 149 | 85 | 12/12 | |
| fAG1 | Solid strategy 1 | 176 | 155 | 88 | 4/12 | 33% |
| | Solid strategy 2 | 176 | 123 | 70 | 4/12 | |
| fAG2 | Solid strategy 1 | 88 | 16 | 18 | 3/7 | 60% |
| | Solid strategy 2 | 88 | 8 | 9 | 3/ 3 | |

Heavy and light chain CDR3 regions of promising clones for individual panning outputs were sequenced in order to estimate the sequence diversity. As depicted in Table 11, sequence diversity was high. For eGFP, every sequenced antibody sequence was unique. Outputs of the remaining test pannings also contained few sequences that enriched during the panning process so that overall sequence diversity was slightly lower (˜30% for fAG1 and 60% for fAG2). As anticipated, sequences covered diverse CDR lengths both in the HCDR3 and LCDR3 (data not shown).

Further exemplary embodiments of the invention include:

1. A polypeptide library comprising VH polypeptide members comprising VH FW1-
HCDR1-FW2-HCDR2-FW3-HCDR3-FW4 segments comprising at least one
sequence selected from
(SEQ ID NO 232)
(D/Q/Y/E)(V/E)(Q/L/R)LV(E/Q)SGGD(L/R)V(K/Q)PGGSLRL(T/I/A)C(V/M)(A/G)SGF(T/
P/I/N)(F/V/L)XaaXaaXaaXaaXaaXaaWVRQ(A/T)PG(K/M)GLQWVXaaXaaXaaXaaXa
aXaaXaaXaaXaaXaaXaaXaaXaaDSVKGRFT(I/V)S(R/K)D(N/D)(A/P/V)(K/R/M/E)NTL
(Y/L)LQM(N/T/D)(S/G/N)LKTED(T/A/M)ATYYC(A/S/T)(R/K/N/T/G)XaaXaaXaaXaaXaa
XaaXaaXaaXaaXaaXaaXaaXaaXaaWG(Q/R)G(A/T/V)(L/P)VTVSS,
preferably comprising a FW1 sequence selected from
(SEQ ID NO 240)
DEQLVESGGDLVKPGGSLRLTCVASGFPF,
(SEQ ID NO 241)
DEQLVESGGDLVKPGGSLRLTCVASGFTL,
(SEQ ID NO 242)
DEQLVESGGDLVKPGGSLRLTCVGSGFTF,
(SEQ ID NO 243)
DVQLVESGGDLVKPGGSLRLACVASGFTF,
(SEQ ID NO 244)
DVQLVESGGDLVKPGGSLRLICVASGFTF,
(SEQ ID NO 245)
DVQLVESGGDLVKPGGSLRLTCVASGFIF,
(SEQ ID NO 246)
DVQLVESGGDLVKPGGSLRLTCVASGFPF,
(SEQ ID NO 247)
DVQLVESGGDLVKPGGSLRLTCVASGFTF,
(SEQ ID NO 248)
DVQVVESGGDLVKPGGSLRLTCVASGFTF,
(SEQ ID NO 249)
DVRLVESGGDLVKPGGSLRLTCVASGFTF,
(SEQ ID NO 250)
DVRLVESGGDRVKPGGSLRLTCMASGFNV,
(SEQ ID NO 251)
EVQLVESGGDLVKPGGSLRLTCVASGFTF,
(SEQ ID NO 252)
QVLLVQSGGDLVKPGGSLRLTCVASGFTF
or
(SEQ ID NO 253)
YVQLVESGGDLVKPGGSLRLTCVASGFTF,
and/or
preferably comprising a HCDR1 sequence selected from,
(SEQ ID NO 254)
GSYDMT
(SEQ ID NO 255)
NNFAMS,
(SEQ ID NO 256)
NSYAMS,
(SEQ ID NO 257)
RGYAMT,
(SEQ ID NO 258)
RSHWMN,
(SEQ ID NO 259)
SDYDMS,
(SEQ ID NO 260)
SGYSMN,
(SEQ ID NO 261)
SLYDMS,
(SEQ ID NO 262)
SNYDMS,
(SEQ ID NO 263)
SNYGMD,
(SEQ ID NO 264)
SNYGMS,
(SEQ ID NO 265)
SRYGMS,
(SEQ ID NO 266)
SSYAMS,
(SEQ ID NO 267)
SSYEMN,
(SEQ ID NO 268)
SSYGMS,
(SEQ ID NO 269)
SSYYMH,
(SEQ ID NO 270)
TGDAMS
or
(SEQ ID NO 271)
TNYAMS,,
and/or
preferably comprising a FW2 sequence selected from
(SEQ ID NO 271)
WVRQAPGKGLQWV,
(SEQ ID NO 273)
WVRQAPGMGLQWV,
(SEQ ID NO 274)
WVRQAPGRGLQWV
or
(SEQ ID NO 275)
WVRQTPGKGLQWV,
and/or
preferably comprising a HCDR2 sequence selected from
(SEQ ID NO 276)
AAIAYNGGNTGYA,
(SEQ ID NO 277)
AAIGHDGSTTAYA,
(SEQ ID NO 278)
AAIRGSGGVTYYA,
(SEQ ID NO 279)
AAISGSGDSTYYA,
(SEQ ID NO 280)
AAISYNGGGTGYS,
(SEQ ID NO 281)
AANSGTGSSYTTA,
(SEQ ID NO 282)
ADISGSGGATAYA,
(SEQ ID NO 283)
AGISGSGITTYYA,
(SEQ ID NO 284)
AGISTSGGNTYYA,
(SEQ ID NO 285)
AGITSGGNTYYA,
(SEQ ID NO 286)
AQISDSGGSTYYA,
(SEQ ID NO 287)
AYIDNDGSSTYYA,
(SEQ ID NO 288)
AYIRYDGNTIHYG,
(SEQ ID NO 289)
AYIRYDGSSTNYA,
(SEQ ID NO 290)
AYISSGGSTYYA,
(SEQ ID NO 291)
CAIGGTGSRTLYA,
(SEQ ID NO 292)
SAISFDGSGTGYA,
(SEQ ID NO 293)
SALSESGHSTIYA,
(SEQ ID NO 294)
SSISSGGTTYYA,
(SEQ ID NO 295)
STIDSGGNTHYI,
(SEQ ID NO 296)
TDISRSGATTYYA
or
(SEQ ID NO 297)
TTISGSGGSTYYA,
and/or
preferably comprising a FW3 sequence selected from
(SEQ ID NO 298)
DSVKGRFTISKDDAENTLYLQMNSLKTEDTATYYCAG,
(SEQ ID NO 299)
DSVKGRFTISRDNAENTLLLQMNSLKTEDTATYYCAR,
(SEQ ID NO 300)
DSVKGRFTISRDNAKNTLSLQMDSLKTEDTATYYCAT,
(SEQ ID NO 301)
DSVKGRFTISRDNAKNTLYLQMDSLKTEDTATYYCTS,
(SEQ ID NO 302)
DSVKGRFTISRDNAKNTLYLQMNGLKTEDTATYYCAR,
(SEQ ID NO 303)
DSVKGRFTISRDNAKNTLYLQMNNLKTEDTATYYCAG,
(SEQ ID NO 304)
DSVKGRFTISRDNAKNTLYLQMNSLKTEDMATYYCAR,
(SEQ ID NO 305)
DSVKGRFTISRDNAKNTLYLQMNSLKTEDTATYYCAK,
(SEQ ID NO 306)
DSVKGRFTISRDNAKNTLYLQMNSLKTEDTATYYCAR,
(SEQ ID NO 307)
DSVKGRFTISRDNAKNTLYLQMNSLKTEDTATYYCAT,
(SEQ ID NO 308)
DSVKGRFTISRDNAMNTLYLQMNSLKTEDAATYYCAR,
(SEQ ID NO 309)
DSVKGRFTISRDNARNTLYLQMNSLKTEDTATYYCAR,
(SEQ ID NO 310)
DSVKGRFTISRDNPKNTLYLQMTSLKTEDTATYYCAR,
(SEQ ID NO 311)
DSVKGRFTISRDNVKNTLYLQMNSLKTEDTATYYCAR
or
(SEQ ID NO 312)
DSVKGRFTVSRDNAKNTLYLQMNSLKTEDTATYYCSR,
and/or
preferably comprising a FW4 sequence selected from
(SEQ ID NO 213)
WGQGAPVTVSS″,
(SEQ ID NO 215)
WGQGTLVTVSS
(SEQ ID NO 216)
WGQGVLVTVSS
and
(SEQ ID NO 217)
WGRGALVTVSS,
and/or
wherein optionally the VH FW1 is
(SEQ ID NO 247)
DVQLVESGGDLVKPGGSLRLTCVASGFTF,
(SEQ ID NO 405)
DVQLVESGGDLVQPGGSLRLTCVASGFTF,
and/or
(SEQ ID NO 406)
DVQLVQSGGDLVKPGGSLRLTCVASGFTF,
and/or
wherein optionally the HCDR1 sequence is
(SEQ ID NO 259)
SDYDMS,
(SEQ ID NO 264)
SNYDMS,
(SEQ ID NO 265)
SRYGMS,
(SEQ ID NO 267)
SSYEMN,
(SEQ ID NO 269)
SSYYMH,
and/or
(SEQ ID NO 266)
SSYAMS,
wherein optionally the VH FW2 sequence is
(SEQ ID NO 271)
WVRQAPGKGLQWV,
and/or
wherein optionally the HCDR2 sequence is
(SEQ ID NO 280)
AAISYNGGGTGYS,
(SEQ ID NO 281)
AANSGTGSSTYYA,
(SEQ ID NO 276)
AAIAYNGGNTGYA,
(SEQ ID NO 279)
AAISGSGDSTYYA,
(SEQ ID NO 290)
AYISSGGSTYYA,
(SEQ ID NO 286)
AQISDSGGSTYYA
and/or
(SEQ ID NO 282)
ADISGSGGATAYA,
and/or
wherein optionally the VH FW3 sequence is
(SEQ ID NO 302)
DSVKGRFTISRDNAKNTLYLQMNGLKTEDTATYYCAR
or
(SEQ ID NO 306)
DSVKGRFTISRDNAKNTLYLQMNSLKTEDTATYYCAR, and/or

wherein optionally the VH FW4 sequence is WGQGALVTVSS (SEQ ID NO: 213); or substantially identical sequences.
2. A polypeptide library, preferably a polypeptide library according to embodiment 1, comprising VL polypeptide members comprising VL FW1-LCDR1-FW2-LCDR2-FW3-LCDR3-FW4 segments comprising at least one sequence selected from

(SEQ ID NO 236)
Q(S/P/E)(V/G)(P/L/V)(T/N)Q(P/E)(P/S/T)S(V/L/M)S(G/A/T)(A/G/S/T)(L/P)G(Q/G/T)(R/
A/T/K)(V/I)(T/R)(I/M/L)(S/T)CXaaXaaXaaXaaXaaXaaXaaXaaXaaXaaXaaXaaXaaXaa
W(Y/V)(Q/R)Q(I/V/L/K/H)(P/S)(G/R/Y)(M/K/R/Q/T/S)(A/P/R)(P/F)(K/Q/S)(T/L/A/V/Y)(L/
I/V/F)(I/V/L)(Y/V/G/H)(-/Y)(-/Y)(-/S)(-
/D)XaaXaaXaaXaaXaaXaaXaaXaaXaaXaa(D/A/N/S)R(F/I/L/V)SGS(K/L)(-/D)(-
/A)(S/V)(G/A)(S/N/T/Q/I/A)(T/K/A)(G/A)(S/T/L/V)L(T/A/L)(I/V)(T/S)G(L/P/A)Q(A/S/T/P)
EDE(A/G/V)(D/N)Y(Y/H)CXaaXaaXaaXaaXaaXaaXaaXaaXaaXaaXaaXaa(F/I)G(G/R)
GTH(L/V)(T/S)VL,
preferably comprising a FW1 sequence selected from
(SEQ ID NO 313)
QSVVTQPPSVSGALGQAVTISC,
(SEQ ID NO 314)
QSVLTQPPSVSGALGQTVTISC,
(SEQ ID NO 315)
QSVLTQPPSVSGGLGQKVTISC,
(SEQ ID NO 316)
QPVVTQPPSVSGALGQTVTISC,
(SEQ ID NO 317)
QSVLTQPPSMSGALGQTVTISC,
(SEQ ID NO 318)
QPVLTQPPSVSGSLGQRVTISC,
(SEQ ID NO 319)
QSGPNQPSSVSGALGQRVTISC,
(SEQ ID NO 320)
QSGPNQPSSVSGALGQRVTMSC,
(SEQ ID NO 321)
QSGPNQPSSVSAALGQRVTISC,
(SEQ ID NO 322)
QSGPNQPSSVSGTLGQTITISC,
(SEQ ID NO 323)
QEVVTQETSLSTTPGGTVTLTC,
(SEQ ID NO 324)
QSVVTQPPSVSGALGQRVTISC,
(SEQ ID NO 325)
QPVLTQPPSVSGALGQRVTISC,
(SEQ ID NO 326)
QPVVTQPPSVSGALGQRVTISC,
(SEQ ID NO 327)
QPVLTQPSSLSASPGTTARLTC
or
(SEQ ID NO 328)
QPVVTQPPSLSGSLGATARTLC,
and/or
preferably comprising a LCDR1 sequence selected from
(SEQ ID NO 329)
AGSANNIGITDVN,
(SEQ ID NO 330)
AGSGSNIGIVDVN,
(SEQ ID NO 331)
AGSGSNIGIAGVN,
(SEQ ID NO 332)
AGSANNNGIVGVN,
(SEQ ID NO 333)
AGSGSNIGVAGVN,
(SEQ ID NO 334)
TGTSSNIGSGNYVS,
(SEQ ID NO 335)
TGIDTYVG,
(SEQ ID NO 336)
TGVDTYVD,
(SEQ ID NO 337)
TGVDSYVG,
(SEQ ID NO 338)
SGAGSYVA,
(SEQ ID NO 339)
TGAGSYVG,
(SEQ ID NO 340)
TGVGNYVD,
(SEQ ID NO 341)
TGVGMSVD,
(SEQ ID NO 342)
TGAGYVG,
(SEQ ID NO 343)
TGVSVFVD,
(SEQ ID NO 344)
GSSTGAVTTSNFAS,
(SEQ ID NO 345)
TGSSSNVGRGNYVS,
(SEQ ID NO 346)
TGSSPNIGRGNYVS,
(SEQ ID NO 347)
TGSSSNIGRGNYVS,
(SEQ ID NO 348)
TLSSGFNVGGYYIS
or
(SEQ ID NO 349)
TLSREVSVGVKGIY,
and/or
preferably comprising a FW2 sequence selected from
(SEQ ID NO 350)
WYQQLPGKAPKLLIY,
SEQ ID NO 351)
WYQQLPGKAPSLLIY,
(SEQ ID NO 352)
WYQQHPRKAPKLLIY,
(SEQ ID NO 353)
WYQQLPGKAPQLLIY,
(SEQ ID NO 354)
WYQQLPGKAPKALIY,
(SEQ ID NO 355)
WYRQVPGIAPSLLIY,
(SEQ ID NO 356)
WFQQIPGMAPKTIIV,
(SEQ ID NO 357)
WYQQIPGMAPKTIIY,
(SEQ ID NO 358)
WYQQIPGRAPKTIIY,
(SEQ ID NO 359)
WYQQVPGMAPKTIIY,
(SEQ ID NO 360)
WYQQIPGMAPKTVIY,
(SEQ ID NO 361)
WVQQKPYQRFQGLVG,
(SEQ ID NO 362)
WYQQLSGTPPKLLIY,
(SEQ ID NO 363)
WYQQLSGTAPKVLIY,
(SEQ ID NO 364)
WYQQLSGTAPKLLIY,
(SEQ ID NO 365)
WYQQVSGTAPKLLIY,
(SEQ ID NO 366)
WFQQKPGSPPRYLLYYYSD
or
(SEQ ID NO 367)
WYQQKPGSPPRYFLHYYSD,
and/or
preferably comprising a LCDR2 sequence selected from
(SEQ ID NO 368)
ANNRRPSSVP,
(SEQ ID NO 369)
ASDRRPSGVP,
(SEQ ID NO 370)
GSSNRPSGVP,
(SEQ ID NO 371)
ASTRRPSGVP,
(SEQ ID NO 372)
ENFKRPSGVP,
(SEQ ID NO 373)
GNTNRPSGVP,
(SEQ ID NO 374)
YDSNRPSGVP,
(SEQ ID NO 375)
DNSNRPSGVP,
(SEQ ID NO 376)
DDNKRPSGVP,
(SEQ ID NO 377)
GNRYRPSGVP,
(SEQ ID NO 378)
RNSNRLSEVP,
(SEQ ID NO 379)
GNSYRPSGVP,
(SEQ ID NO 380)
RNTNRLSEVP,
(SEQ ID NO 381)
GTSYRNPGVP,
(SEQ ID NO 382)
GNSDRLSGVP,
(SEQ ID NO 383)
GDKNRPSGVP,
(SEQ ID NO 384)
GDTNRPSGVP,
(SEQ ID NO 385)
SDKHQGPGVP
or
(SEQ ID NO 386)
STNELGPGVP,
and/or
preferably comprising a FW3 sequence selected from
(SEQ ID NO 387)
DRFSGSKSGATGSLTISGLQAEDEADYYC,
(SEQ ID NO 388)
ERISGSRSGNTGSLTITGLQTEDEADYYC,
(SEQ ID NO 389)
DRFSGSKSGNTGSLTITGLQAEDEADYYC,
(SEQ ID NO 390)
DRFSGSKSGNTGSLTITGLQAEDEADYYC,
(SEQ ID NO 391)
ERLSGSKSGNTGSLTITGLQPEDEADYYC,
(SEQ ID NO 392)
DRFSGSKSGSSGSLTITGLQADDEVDYYC,
(SEQ ID NO 393)
DRFSGSKSGNTGTLTITGLQAEDEADYYC,
(SEQ ID NO 394)
DRFSGSKSGSTGTLAITGLQAEDEGDYYC,
(SEQ ID NO 395)
DRFSGSKSGNTGTLTITGLQAEDEADYYC,
(SEQ ID NO 396)
DRFSGSKSGNTGTLTITGLQAEDEADYYC,
(SEQ ID NO 397)
ARFSGSKSGSTGTLTITGLQAEDEANYYC,
(SEQ ID NO 398)
DRFSGSKSGSTGTLTITGLQAEDEADYYC,
(SEQ ID NO 399)
NRFSGSKSGSTATLTITGLQAEDEADYYC,
(SEQ ID NO 400)
DRFSGSKSGSTGTLTITGLQAEDEADYYC,
(SEQ ID NO 401)
DRFSGSKSGITATLTITGLQAEDEADYYC,
(SEQ ID NO 402)
ARFSGSLVGQKAVLTITGAQSEDEAEYYC,
(SEQ ID NO 403)
DRFSGSKSGTTGSLTITGLQTEDEADYYC
or
(SEQ ID NO 404)
DRFSGSKSGSTGSLTITGLQAEDEADYYC,
and/or
preferably comprising a FW4 sequence selected from
(SEQ ID NO 208)
FGGGTHLTVL,
(SEQ ID NO: 209)
FGGGTHLSVL,
(SEQ ID NO: 210)
FGRGTHLTVL,
(SEQ ID NO: 211)
FGGGTHVTVL
or
(SEQ ID NO: 212)
IGGGTHVTVL,
and/or
wherein optionally the FW1 sequence is
(SEQ ID NO 318)
QPVLTQPPSVSGSLGQRVTISC,
(SEQ ID NO 319)
QSGPNQPSSVSGALGQRVTISC,
(SEQ ID NO 322)
QSGPNQPSSVSGTLGQTITISC,
(SEQ ID NO 324)
QSVVTQPPSVSGALGQRVTISC,
and/or
(SEQ ID NO 325)
QPVLTQPPSVSGALGQRVTISC.
wherein optionally the HCDR 1 sequence is
(SEQ ID NO 334)
TGTSSNIGSGNYVS,
(SEQ ID NO 335)
TGIDTYVG,
(SEQ ID NO 339)
TGAGSYVG,
(SEQ ID NO 340)
TGVGNYVD,
(SEQ ID NO 343)
TGVSVFVD,
(SEQ ID NO 345)
TGSSSNVGRGNYVS,
and/or
(SEQ ID NO 347)
TGSSSNIGRGNYVS.
wherein optionally the VL FW2 sequence is
(SEQ ID NO 355)
WYRQVPGIAPSLLIY,
(SEQ ID NO 356)
WFQQIPGMAPKTIIV,
(SEQ ID NO 359)
WYQQVPGMAPKTIIY,
(SEQ ID NO 360)
WYQQIPGMAPKTVIY,
(SEQ ID NO 362)
WYQQLSGTPPKLLIY,
and/or
(SEQ ID NO 365)
WYQQVSGTAPKLLIY.
wherein optionally the LCDR2 sequence is
(SEQ ID NO 372)
ENFKRPSGVP,
(SEQ ID NO 373)
GNTNRPSGVP,
(SEQ ID NO 376)
DDNKRPSGVP,
(SEQ ID NO 377)
GNRYRPSGVP,
(SEQ ID NO 380)
RNTNRLSEVP,
(SEQ ID NO 410)
GNTDRLSGVP,
and/or
(SEQ ID NO 384)
GDTNRPSGVP.
wherein optionally the VL FW3 sequence is
(SEQ ID NO 411)
DRFSGSKSGSSGSLTITGLQAEDEVDYYC,
(SEQ ID NO 396)
DRFSGSKSGNTGTLTITGLQAEDEADYYC,
(SEQ ID NO 397)
ARFSGSKSGSTGTLTITGLQAEDEANYYC,
(SEQ ID NO 400)
DRFSGSKSGSTGTLTITGLQAEDEADYYC,
(SEQ ID NO 401)
DRFSGSKSGITATLTITGLQAEDEADYYC,
(SEQ ID NO 403)
DRFSGSKSGTTGSLTITGLQTEDEADYYC,
and/or
(SEQ ID NO 404)
DRFSGSKSGSTGSLTITGLQAEDEADYYC

wherein optionally the VL FW 4 sequence is FGGGTHLTVL (SEQ ID NO: 208); or substantially identical sequences.
3. A polypeptide library, preferably a polypeptide library according to embodiments 1 or 2, comprising VL polypeptide members comprising VL FW1-CDR1-FW2-CDR2-FW3 segments comprising at least one sequence selected from SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 218, SEQ ID NO: 219, SEQ ID NO: 220, SEQ ID NO: 221, SEQ ID NO: 222, SEQ ID NO: 224, and/or SEQ ID NO: 224; preferably selected from SEQ ID NO: 73, SEQ ID NO: 74, SEQ ID NO: 75, SEQ ID NO: 76, SEQ ID NO: 78, SEQ ID NO: 84, SEQ ID NO: 85, SEQ ID NO: 86, SEQ ID NO: 88, SEQ ID NO: 91, SEQ ID NO: 92, SEQ ID NO: 95, SEQ ID NO: 110, SEQ ID NO: 112, SEQ ID NO: 119, SEQ ID NO: 123, SEQ ID NO: 125, SEQ ID NO: 128, SEQ ID NO: 129, SEQ ID NO: 133, SEQ ID NO: 134, SEQ ID NO: 135, SEQ ID NO: 218, SEQ ID NO: 219, SEQ ID NO: 220, SEQ ID NO: 221, SEQ ID NO: 222, SEQ ID NO: 224, and/or SEQ ID NO: 224, or substantially identical sequences.
4. A polypeptide library, preferably a polypeptide library according to any of the preceding embodiments, comprising VH polypeptide members comprising VH FW1-CDR1-FW2-CDR2-FW3 segments comprising at least one sequence selected from SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202; SEQ ID NO: 225, SEQ ID NO: 226, SEQ ID NO: 227, SEQ ID NO: 228, SEQ ID NO: 229, SEQ ID NO: 230, and/or SEQ ID NO: 231; preferably selected SEQ ID NO: 138, SEQ ID NO: 139, SEQ ID NO: 140, SEQ ID NO: 141, SEQ ID NO: 143, SEQ ID NO: 149, SEQ ID NO: 150SEQ ID NO: 151, SEQ ID NO: 153, SEQ ID NO: 156, SEQ ID NO: 157, SEQ ID NO: 160, SEQ ID NO: 175, SEQ ID NO: 177, SEQ ID NO: 184, SEQ ID NO: 188, SEQ ID NO: 190, SEQ ID NO: 193, SEQ ID NO: 194, SEQ ID NO: 198, SEQ ID NO: 199, SEQ ID NO: 200, SEQ ID NO: 225, SEQ ID NO: 226, SEQ ID NO: 227, SEQ ID NO: 228, SEQ ID NO: 229, SEQ ID NO: 230, and/or SEQ ID NO: 231, or substantially identical sequences. A polypeptide library, preferably a polypeptide library according to any of the preceding embodiments, comprising VH polypeptide members comprising VH FW1-CDR1-FW2-CDR2-FW3 segments comprising at least one sequence selected from SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202; SEQ ID NO: 225, SEQ ID NO: 226, SEQ ID NO: 227, SEQ ID NO: 228, SEQ ID NO: 229, SEQ ID NO: 230, and/or SEQ ID NO: 231; preferably selected SEQ ID NO: 138, SEQ ID NO: 139, SEQ ID NO: 140, SEQ ID NO: 141, SEQ ID NO: 143, SEQ ID NO: 149, SEQ ID NO: 150SEQ ID NO: 151, SEQ ID NO: 153, SEQ ID NO: 156, SEQ ID NO: 157, SEQ ID NO: 160, SEQ ID NO: 175, SEQ ID NO: 177, SEQ ID NO: 184, SEQ ID NO: 188, SEQ ID NO: 190, SEQ ID NO: 193, SEQ ID NO: 194, SEQ ID NO: 198, SEQ ID NO: 199, SEQ ID NO: 200, SEQ ID NO: 225, SEQ ID NO: 226, SEQ ID NO: 227, SEQ ID NO: 228, SEQ ID NO: 229, SEQ ID NO: 230, and/or SEQ ID NO: 231, or substantially identical sequences.
5. A polypeptide library, preferably a polypeptide library according to any preceding embodiments, comprising at least one VL/VH polypeptide member combination selected from SEQ ID NO: 73 and SEQ ID NO: 138, SEQ ID NO: 74 and SEQ ID NO: 139, SEQ ID NO: 75 and SEQ ID NO: 140, SEQ ID NO: 76 and SEQ ID NO: 141, SEQ ID NO: 77 and SEQ ID NO: 142, SEQ ID NO: 78 and SEQ ID NO: 143, SEQ ID NO: 79 and SEQ ID NO: 144, SEQ ID NO: 80 and SEQ ID NO: 145, SEQ ID NO: 81 and SEQ ID NO: 146, SEQ ID NO: 82 and SEQ ID NO: 147, SEQ ID NO: 83 and SEQ ID NO: 148, SEQ ID NO: 84 and SEQ ID NO: 149, SEQ ID NO: 85 and SEQ ID NO: 150, SEQ ID NO: 86 and SEQ ID NO: 151, SEQ ID NO: 87 and SEQ ID NO: 152, SEQ ID NO: 88 and SEQ ID NO: 153, SEQ ID NO: 89 and SEQ ID NO: 154, SEQ ID NO: 90 and SEQ ID NO: 155, SEQ ID NO: 91 and SEQ ID NO: 156, SEQ ID NO: 92 and SEQ ID NO: 157, SEQ ID NO: 93 and SEQ ID NO: 158, SEQ ID NO: 94 and SEQ ID NO: 159, SEQ ID NO: 95 and SEQ ID NO: 160, SEQ ID NO: 96 and SEQ ID NO: 161, SEQ ID NO: 97 and SEQ ID NO: 162, SEQ ID NO: 98 and SEQ ID NO: 163, SEQ ID NO: 99 and SEQ ID NO: 164, SEQ ID NO: 100 and SEQ ID NO: 165, SEQ ID NO: 101 and SEQ ID NO: 166, SEQ ID NO: 102 and SEQ ID NO: 167, SEQ ID NO: 103 and SEQ ID NO: 168, SEQ ID NO: 104 and SEQ ID NO: 169, SEQ ID NO: 105 and SEQ ID NO: 170, SEQ ID NO: 106 and SEQ ID NO: 171, SEQ ID NO: 107 and SEQ ID NO: 172, SEQ ID NO: 108 and SEQ ID NO: 173, SEQ ID NO: 109 and SEQ ID NO: 174, SEQ ID NO: 110 and SEQ ID NO: 175, SEQ ID NO: 111 and SEQ ID NO: 176, SEQ ID NO: 112 and SEQ ID NO: 177, SEQ ID NO: 113 and SEQ ID NO: 178, SEQ ID NO: 114 and SEQ ID NO: 179, SEQ ID NO: 115 and SEQ ID NO: 180, SEQ ID NO: 116 and SEQ ID NO: 181, SEQ ID NO: 117 and SEQ ID NO: 182, SEQ ID NO: 118 and SEQ ID NO: 183, SEQ ID NO: 119 and SEQ ID NO: 184, SEQ ID NO: 120 and SEQ ID NO: 185, SEQ ID NO: 121 and SEQ ID NO: 186, SEQ ID NO: 122 and SEQ ID NO: 187, SEQ ID NO: 123 and SEQ ID NO: 188, SEQ ID NO: 124 and SEQ ID NO: 189, SEQ ID NO: 125 and SEQ ID NO: 190, SEQ ID NO: 126 and SEQ ID NO: 191, SEQ ID NO: 127 and SEQ ID NO: 192, SEQ ID NO: 128 and SEQ ID NO: 193, SEQ ID NO: 129 and SEQ ID NO: 194, SEQ ID NO: 130 and SEQ ID NO: 195, SEQ ID NO: 131 and SEQ ID NO: 196, SEQ ID NO: 132 and SEQ ID NO: 197, SEQ ID NO: 133 and SEQ ID NO: 198, SEQ ID NO: 134 and SEQ ID NO: 199, SEQ ID NO: 135 and SEQ ID NO: 200, SEQ ID NO: 136 and SEQ ID NO: 201, SEQ ID NO: 137 and SEQ ID NO: 202, SEQ ID NO: 218 and SEQ ID NO: 225, SEQ ID NO 219 and SEQ ID NO 226, SEQ ID NO: 220 and SEQ ID NO: 227, SEQ ID NO: 221 and SEQ ID NO 228, SEQ ID NO: 222 and SEQ ID NO: 229, SEQ ID NO: 223 and SEQ ID NO: 230 and/or SEQ ID NO: 224 and SEQ ID NO: 230; preferably the polypeptide library comprises at least one VL/VH polypeptide member combination comprising a sequence combination selected from SEQ ID NO: 73 and SEQ ID NO: 138, SEQ ID NO: 74 and SEQ ID NO: 139, SEQ ID NO: 75 and SEQ ID NO: 140, SEQ ID NO: 76 and SEQ ID NO: 141, SEQ ID NO: 78 and SEQ ID NO: 143, SEQ ID NO: 84 and SEQ ID NO: 149, SEQ ID NO: 85 and SEQ ID NO: 150, SEQ ID NO: 86 and SEQ ID NO: 151, SEQ ID NO: 88 and SEQ ID NO: 153, SEQ ID NO: 91 and SEQ ID NO: 156, SEQ ID NO: 92 and SEQ ID NO: 157, SEQ ID NO: 95 and SEQ ID NO: 160, SEQ ID NO: 110 and SEQ ID NO: 175, SEQ ID NO: 112 and SEQ ID NO: 177, SEQ ID NO: 119 and SEQ ID NO: 184, SEQ ID NO: 123 and SEQ ID NO: 188, SEQ ID NO: 125 and SEQ ID NO: 190, SEQ ID NO: 128 and SEQ ID NO: 193, SEQ ID NO: 129 and SEQ ID NO: 194, SEQ ID NO: 133 and SEQ ID NO: 198, SEQ ID NO: 134 and SEQ ID NO: 199, and/or SEQ ID NO: 135 and SEQ ID NO: 200, SEQ ID NO: 218 and SEQ ID NO: 225, SEQ ID NO 219 and SEQ ID NO 226, SEQ ID NO: 220 and SEQ ID NO: 227, SEQ ID NO: 221 and SEQ ID NO 228, SEQ ID NO: 222 and SEQ ID NO: 229, SEQ ID NO: 223 and SEQ ID NO: 230 and/or SEQ ID NO: 224 and SEQ ID NO: 230, or combinations of substantially identical sequences.
6. A polypeptide library, preferably a polypeptide library according to any preceding embodiments, comprising at least one VL/VH polypeptide member combination comprising a VL polypeptide member according to embodiment 0, preferably selected from SEQ ID NO: 73, SEQ ID NO: 74, SEQ ID NO: 75, SEQ ID NO: 76, SEQ ID NO: 78, SEQ ID NO: 84, SEQ ID NO: 85, SEQ ID NO: 86, SEQ ID NO: 88, SEQ ID NO: 91, SEQ ID NO: 92, SEQ ID NO: 95, SEQ ID NO: 110, SEQ ID NO: 112, SEQ ID NO: 119, SEQ ID NO: 123, SEQ ID NO: 125, SEQ ID NO: 128, SEQ ID NO: 129, SEQ ID NO: 133, SEQ ID NO: 134, SEQ ID NO: 135, and/or SEQ ID NO: 218, SEQ ID NO: 219, SEQ ID NO: 220, SEQ ID NO: 221, SEQ ID NO: 222, SEQ ID NO: 224, and/or SEQ ID NO: 224, and a VH polypeptide member according to embodiment 0 preferably selected from SEQ ID NO: 138, SEQ ID NO: 139, SEQ ID NO: 140, SEQ ID NO: 141, SEQ ID NO: 143, SEQ ID NO: 149, SEQ ID NO: 150, SEQ ID NO: 151, SEQ ID NO: 153, SEQ ID NO: 156, SEQ ID NO: 157, SEQ ID NO: 160, SEQ ID NO: 175, SEQ ID NO: 177, SEQ ID NO: 184, SEQ ID NO: 188, SEQ ID NO: 190, SEQ ID NO: 193, SEQ ID NO: 194, SEQ ID NO: 198, SEQ ID NO: 199, SEQ ID NO: 200, SEQ ID NO: 225, SEQ ID NO: 226, SEQ ID NO: 227, SEQ ID NO: 228, SEQ ID NO: 229, SEQ ID NO: 230, and/or SEQ ID NO: 231, or combinations of substantially identical sequences.
7. A polypeptide library comprising at least one VL/VH polypeptide member combination comprising at least one VH FW1-HCDR1-FW2-HCDR2-FW3-HCDR3-FW4 segments according to embodiment 1 and a VL FW1-LCDR1-FW2-LCDR2-FW3-LCDR3-FW4 segment according to embodiment 2.
8. A polypeptide library comprising at least one scFv or Fab antibody fragment comprising any of the VH FW1-CDR1-FW2-CDR2-FW3 segments, VL FW1-CDR1-FW2-CDR2-FW3 segments, VH FW1-HCDR1-FW2-HCDR2-FW3-HCDR3-FW4 segments, VL FW1-LCDR1-FW2-LCDR2-FW3-LCDR3-FW4 segments or VL/VH polypeptide member combination according to any of embodiments 1 to 7.
9. A polypeptide library according to embodiment 0 comprising at least one scFv fragment, wherein the VH polypeptide member is N-terminally linked to the VL polypeptide member by a linker of about 15 to about 25, preferably about 18 amino acids, preferably a linker comprising the amino acid sequence

GGSSRSSSSGGGGSGGGG.

10. A polypeptide library according to any of the foregoing embodiments, wherein the at least one VH polypeptide comprises a FW4 region sequence selected from amino acid sequences WGQGAPVTVSS (SEQ ID NO: 214), WGQGTLVTVSS (SEQ ID NO: 215), WGQGVLVTVSS (SEQ ID NO: 216) and WGRGALVTVSS (SEQ ID NO: 217), preferably the FW4 region sequence is WGQGALVTVSS (SEQ ID NO: 213).
11. A polypeptide library according to any of the foregoing embodiments, wherein the at least one VL polypeptide comprises a FW4 region sequence selected from amino acid sequences FGGGTHLTVL (SEQ ID NO: 208), FGGGTHLSVL (SEQ ID NO: 209), FGRGTHLTVL (SEQ ID NO: 210), FGGGTHVTVL (SEQ ID NO: 211) and IGGGTHVTVL (SEQ ID NO: 212), preferably one VL polypeptides comprises a FW4 region sequence selected from amino acid sequences FGGGTHLTVL (SEQ ID NO: 208).
12. A polypeptide library according to any of the foregoing embodiments, wherein the polypeptide library comprises different polypeptide members comprising different feline specific CDR3 polypeptide sequences between the FW3 and the FW4 segments.
13. A polypeptide library according to embodiment 12, wherein the feline specific CDR3 polypeptide sequences are randomized or partially randomized amino acid sequences.
14. A polypeptide library according to embodiment 13, wherein the feline specific CDR3 polypeptide sequences are characterised by a design as shown in Table 10, wherein preferably LCDR3s having a length of 7-9 amino acids have an amino acid distribution as shown in FIG. 14, and/or HCDR3s having a length of 10-12 amino acids have an amino acid distribution as shown in Table 9 and/or wherein HCDRs having a length of 7-9 amino acids have an amino acid distribution as shown in FIG. 11, HCDR3s having a length of 10-12 amino acids have an amino acid distribution as shown in FIG. 12, and HCDR3s having a length of 13-14 amino acids have an amino acid distribution as shown in FIG. 13.
15. A polypeptide library according to any of the foregoing embodiments, wherein the VH CDR1-FW2-CDR2 sequences comprised in the VH polypeptide members according to any of the preceding embodiments are replaced by a HCDR1/2 maturation module, wherein preferably the VH maturation module has a design as shown in FIG. 15.
16. A polypeptide library according to embodiments 8 to 15, wherein the scFv antibody fragment comprises an N- or C-terminal affinity tag, preferably a FLAG tag.
17. A polypeptide library according to embodiments 6 to 16, wherein at least one VL/VH polypeptide member combination comprises a full length or truncated phage pIII polypeptide sequence linked to the VH polypeptide.
18. A polypeptide library according to any of the foregoing embodiments wherein the polypeptide members are devoid of post-translational modification (PTM) sites.
19. A polypeptide library according to embodiments 6 to 18, wherein substantially all VL/VH polypeptide member combinations:
   a) have a monomeric content of at least 85% when expressed in E. coli in Fab format; and/or
   b) have a monomeric content of at least 90% when expressed in a mammalian system in IgG format; and/or
   c) are characterized by a Tm≥60° C.
20. A polypeptide library according to any of the forgoing embodiments, wherein the library is an antibody library, preferably a feline antibody library.
21. A polypeptide library according to any of the forgoing embodiments, wherein the library is a semi-synthetic of fully synthetic antibody library.
22. A polypeptide library according to any of the forgoing embodiments, wherein the polypeptide in the library is a feline antibody selected from a single chain single chain Fv (scFv), a Fab fragment, a F(ab)2 fragment.
23. A feline antibody comprising a VL polypeptide member, and/or a VH polypeptide member or a VL/VH polypeptide member combination according to any of the preceding embodiments, preferably wherein the antibody is a full antibody, a single chain Fv (scFv), a Fab fragment, or a F(ab)2 fragment.
24. An antibody obtained from a polypeptide library according to embodiment 1 to 22.
25. A collection of nucleic acid molecules encoding the polypeptide library members of any of the preceding embodiments.
26. A collection of vectors comprising the nucleic acid molecules of embodiment 25.
27. A recombinant host cell comprising the nucleic acid molecules of embodiment 25 or the vectors of embodiment 26.
28. A method of generating a polypeptide library comprising the steps of:
   (i) providing a feline cDNA generated from an antibody expressing tissue, preferably from splenocytes, lymphocytes;
   (ii) amplifying VL and VH sequences, preferably by using at least one forward primer selected from SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28; and at least one reverse primer selected from SEQ ID NO: 1, SEQ ID NO: 2, and SEQ ID NO: 3, or substantially identical sequences, to obtain amplicons; and
   (iii) generating VL/VH combined sequences by randomly linking the amplicons obtained in step ii); and
   (iv) optionally cloning the VL/VH combined sequences into a vector, preferably selected from a plasmids, phagemids, or expression module;
   (v) optionally expressing single chain Fv fragments comprising the VL/VH combined sequences linked to a full length of truncated phage pIII polypeptide in a phage expression system;
   (vi) optionally isolating phages expressing VL/VH combined sequences by affinity tag and optionally determining the sequences of VL/VH combined sequences in the isolated phages;
   (vii) optionally infecting bacteria with phages isolated in step (vi), wherein preferably the bacteria are E. coli;
   (viii) optionally subcloning the VL/VH combined sequences determined by sequencing in step (vi) into an expression system and expressing the VL/VH combined sequences as soluble scFv fragments, wherein preferably the expression system is a bacterial expression system;
   (ix) optionally determining the expression levels of soluble scFv fragments by ELISA in the expression system of step (viii);
   (x) optionally sequencing the DNA encoding VL/VH combined sequences from high expressing clones of the expression system;
   (xi) optionally cloning the VL and VH combined sequences determined by sequencing in step (x) into a Fab expression system;
   (xii) optionally expressing the Fab fragments in cells;
   (xiii) optionally determining the expression levels of soluble Fab fragments in cells in step (xii);
   (xiv) optionally the sequences of the VL and VH combined sequences in high expressing cells are determined by sequencing;
   (xv) optionally VL/VH combined sequences that expressed high in the scFv and Fab format are determined by comparing the sequences obtained in step (x) and step (xiv).
29. A method according to embodiment 21 wherein:
   the primers employed in step (ii) are designed to incorporate secondary primer binding sites into the 5′ end of the VL amplicons obtained by the amplification and the 3′ end of the VH amplicons obtained by the amplification and/or a flexible linker into the 3′ end of the VL amplicons and the 5′ end of the VH amplicons; and/or
   the VL and the VH amplicons a linked in step (iii) by a linker sequence which encodes 15 to 25 amino acids; and/or
   the method further comprises a step of joining a sequence encoding an affinity tag to the VL/VH combined sequences obtained in step (iii).
30. A method according to embodiments 21 to 22 further comprising a step of introducing a LCDR3 diversity module encoding a partially or fully random amino acids sequence for LCDR3 and/or a HCDR3 diversity module encoding a partially or fully random amino acids sequence for HCDR3 into an VL and/or VH sequence of the combined sequences, preferably into the sequences identified in step xv.
31. A method according to embodiments 21 to 23 comprising a step of introducing a VL maturation module encoding a partially or fully random amino acids sequence for LCDR1 and LCDR2 and/or a VH maturation module encoding a partially or fully random amino acids sequence for HCDR1 and HCDR2 into an VL and/or VH sequence of the combined sequences, preferably into the sequences identified in step xv.
32. A method according to embodiments 21 to 24 comprising a wherein step xi) comprises introducing a feline constant light part of the light chain (CL-lambda) and a constant heavy chain region (CH1) into the expression system such that the VL polypeptides are expressed with a C-terminally linked CL-lambda and the VH polypeptides are expressed with a C-terminally linked CH1 in the expression system.
33. A polypeptide library or a collection of nucleic acids obtainable or obtained by the method of embodiments 21 to 25.

### REFERENCES

### Publication Bibliography

Baldwin, C. I.; Denham, D. A. (1994): Isolation and characterization of three subpopulations of IgG in the common cat (Felis catus). In Immunology 81 (1), pp. 155-160.
Cho, K.; Youn, H. Y.; Okuda, M.; Satoh, H.; Cevario, S.; O'Brien, S. J. et al. (1998): Cloning and mapping of cat (Felis catus) immunoglobulin and T-cell receptor genes. In Immunogenetics 47 (3), pp. 226-233. DOI: 10.1007/s002510050352.
Doki, Tomoyoshi; Takano, Tomomi; Hohdatsu, Tsutomu (2016): Development of a mouse-feline chimeric antibody against feline tumor necrosis factor-alpha. In The Journal of veterinary medical science 78 (9), pp. 1447-1455. DOI: 10.1292/jvms. 16-0020.
Ecker, Dawn M.; Jones, Susan Dana; Levine, Howard L. (2015): The therapeutic monoclonal antibody market. In mAbs 7 (1), pp. 9-14. DOI: 10.4161/19420862.2015.989042.
Enomoto, Masataka; Mantyh, Patrick W.; Murrell, Joanna; Innes, John F.; Lascelles, B. Duncan X. (2019): Anti-nerve growth factor monoclonal antibodies for the control of pain in dogs and cats. In The Veterinary record 184 (1), p. 23. DOI: 10.1136/vr. 104590.
Gearing, D. P.; Huebner, M.; Virtue, E. R.; Knight, K.; Hansen, P.; Lascelles, B. D. X. et al. (2016): In Vitro and In Vivo Characterization of a Fully Felinized Therapeutic Anti-Nerve Growth Factor Monoclonal Antibody for the Treatment of Pain in Cats. In Journal of veterinary internal medicine 30 (4), pp. 1129-1137. DOI: 10.1111/jvim. 13985.
Gearing, David P.; Virtue, Elena R.; Gearing, Robert P.; Drew, Alexander C. (2013): A fully caninised anti-NGF monoclonal antibody for pain relief in dogs. In BMC veterinary research 9, p. 226. DOI: 10.1186/1746-6148-9-226.
Gruen, M. E.; Thomson, A. E.; Griffith, E. H.; Paradise, H.; Gearing, D. P.; Lascelles, B. D. X. (2016): A Feline-Specific Anti-Nerve Growth Factor Antibody Improves Mobility in Cats with Degenerative Joint Disease-Associated Pain: A Pilot Proof of Concept Study. In Journal of veterinary internal medicine 30 (4), pp. 1138-1148. DOI: 10.1111/jvim. 13972.
Kanai, Tomoko Hosokawa; Ueda, Susumu; Nakamura, Toshihiro (2000): Identification of two allelic IgG1 Ch coding regions (Cγ1) of cat. In Veterinary immunology and immunopathology 73 (1), pp. 53-62. DOI: 10.1016/S0165-2427 (99) 00152-X.
Kaplon, Hélène; Reichert, Janice M. (2021): Antibodies to watch in 2021. In mAbs 13 (1), p. 1860476. DOI: 10.1080/19420862.2020.1860476.
Klotz, F. W.; Gathings, W. E.; Cooper, M. D. (1985): Development and distribution of B lineage cells in the domestic cat: analysis with monoclonal antibodies to cat mu-, gamma-, kappa-, and lambda-chains and heterologous anti-alpha antibodies. In Journal of immunology (Baltimore, Md.: 1950) 134 (1), pp. 95-100.
Olivieri, David N.; Haeften, Bernardo von; Sánchez-Espinel, Christian; Gambón-Deza, Francisco (2014): The immunologic V-gene repertoire in mammals. In bioRxiv, p. 2667. DOI: 10.1101/002667.
Raybould, Matthew I. J.; Marks, Claire; Krawczyk, Konrad; Taddese, Bruck; Nowak, Jaroslaw; Lewis, Alan P. et al. (2019): Five computational developability guidelines for therapeutic antibody profiling. In Proceedings of the National Academy of Sciences of the United States of America 116 (10), pp. 4025-4030. DOI: 10.1073/pnas. 1810576116.
Schultz, R. D.; Scott, F. W.; Duncan, J. R.; Gillespie, J. H. (1974): Feline immunoglobulins. In Infection and immunity 9 (2), pp. 391-393. DOI: 10.1128/iai.9.2.391-393.1974.
Sitnikova, T.; Su, C. (1998): Coevolution of immunoglobulin heavy- and light-chain variable-region gene families. In Molecular biology and evolution 15 (6), pp. 617-625. DOI: 10.1093/oxfordjournals.molbev.a025965.
Steiniger, Sebastian C. J.; Dunkle, William E.; Bammert, Gary F.; Wilson, Thomas L.; Krishnan, Abhiram; Dunham, Steven A. et al. (2014): Fundamental characteristics of the expressed immunoglobulin VH and VL repertoire in different canine breeds in comparison with those of humans and mice. In Molecular immunology 59 (1), pp. 71-78. DOI: 10.1016/j.molimm.2014.01.010.
Steiniger, Sebastian C. J.; Glanville, Jacob; Harris, Douglas W.; Wilson, Thomas L.; Ippolito, Gregory C.; Dunham, Steven A. (2017): Comparative analysis of the feline immunoglobulin repertoire. In Biologicals: journal of the International Association of Biological Standardization 46, pp. 81-87. DOI: 10.1016/j.biologicals.2017.01.004.
Tiller, Thomas; Waldhuber, Markus; Strohner, Ralf; Ladetzki, Baehs Kathrin; Prassler, Josef (Jun. 21, 2018): CANINE ANTIBODY LIBRARIES. Applied for by MORPHOSYS AG [DE] on Jun. 21, 2018. App. no. US201816624514 20180621. Patent no. US2020223904 (A1). C07K16/00; C07K16/18. Priority no. EP20170177322 20170622; WO2018EP66563 20180621.
Wabl, Matthias (May 23, 2017): TRANSGENIC MAMMALS AND METHODS OF USE THEREOF. Applied for by TRIANNI INC [US] on May 23, 2017. App. no. US201715603334 20170523. Patent no. US2017306352 (A1). C12N15/85; A01K67/027; C07K16/46. Priority no. US201715603334 20170523; US201313818184 20130506; WO2011US45333 20110726; US20100367809P 20100726; US201662340234P 20160523.
Walters, Rodney R.; Boucher, Joseph F.; Toni, Flavia de (2021): Pharmacokinetics and Immunogenicity of Frunevetmab in Osteoarthritic Cats Following Intravenous and Subcutaneous Administration. In Frontiers in veterinary science 8, p. 687448. DOI: 10.3389/fvets.2021.687448.

1. A polypeptide library comprising VH polypeptide members comprising VH FW1-HCDR1-FW2-HCDR2-FW3-HCDR3-FW4 segments comprising at least one sequence selected from
(SEQ ID NO 232))
(D/Q/Y/E)(V/E)(Q/L/R)LV(E/Q)SGGD(L/R)V(K/Q)PGGSLRL(T/I/A)C(V/M)(A/G)SGF(T/
P/I/N)(F/V/L)XaaXaaXaaXaaXaaXaaWVRQ(A/T)PG(K/M)GLQWVXaaXaaXaaXaaXa
aXaaXaaXaaXaaXaaXaaXaaXaaDSVKGRFT(I/V)S(R/K)D(N/D)(A/P/V)(K/R/M/E)NTL
(Y/L)LQM(N/T/D)(S/G/N)LKTED(T/A/M)ATYYC(A/S/T)(R/K/N/T/G)XaaXaaXaaXaaXaa
XaaXaaXaaXaaXaaXaaXaaXaaXaaWG(Q/R)G(A/T/V)(L/P)VTVSS,
preferably comprising a FW1 sequence selected from
(SEQ ID NO 240)
DEQLVESGGDLVKPGGSLRLTCVASGFPF,
(SEQ ID NO 241)
DEQLVESGGDLVKPGGSLRLTCVASGFTL,
(SEQ ID NO 242)
DEQLVESGGDLVKPGGSLRLTCVGSGFTF,
(SEQ ID NO 243)
DVQLVESGGDLVKPGGSLRLACVASGFTF,
(SEQ ID NO 244)
DVQLVESGGDLVKPGGSLRLICVASGFTF,
(SEQ ID NO 245)
DVQLVESGGDLVKPGGSLRLTCVASGFIF,
(SEQ ID NO 246)
DVQLVESGGDLVKPGGSLRLTCVASGFPF,
(SEQ ID NO 247)
DVQLVESGGDLVKPGGSLRLTCVASGFTF,
(SEQ ID NO 248)
DVQVVESGGDLVKPGGSLRLTCVASGFTF,
(SEQ ID NO 249)
DVRLVESGGDLVKPGGSLRLTCVASGFTF,
(SEQ ID NO 250)
DVRLVESGGDRVKPGGSLRLTCMASGFNV,
(SEQ ID NO 251)
EVQLVESGGDLVKPGGSLRLTCVASGFTF,
(SEQ ID NO 252)
QVLLVQSGGDLVKPGGSLRLTCVASGFTF,
or
(SEQ ID NO 253)
YVQLVESGGDLVKPGGSLRLTCVASGFTF,
and/or
preferably comprising a HCDR1 sequence selected from,
(SEQ ID NO 254)
GSYDMT
(SEQ ID NO 255)
NNFAMS,
(SEQ ID NO 256)
NSYAMS,
(SEQ ID NO 257)
RGYAMT,
(SEQ ID NO 258)
RSHWMN,
(SEQ ID NO 259)
SDYDMS,
(SEQ ID NO 260)
SGYSMN,
(SEQ ID NO 261)
SLYDMS,
(SEQ ID NO 262)
SNYDMS,
(SEQ ID NO 263)
SNYGMD,
(SEQ ID NO 264)
SNYGMS,
(SEQ ID NO 265)
SRYGMS,
(SEQ ID NO 266)
SSYAMS,
(SEQ ID NO 267)
SSYEMN,
(SEQ ID NO 268)
SSYGMS,
(SEQ ID NO 269)
SSYYMH,
(SEQ ID NO 270)
TGDAMS
or
(SEQ ID NO 271)
TNYAMS,,
and/or
preferably comprising a FW2 sequence selected from
(SEQ ID NO 271)
WVRQAPGKGLQWV,
(SEQ ID NO 273)
WVRQAPGMGLQWV,
(SEQ ID NO 274)
WVRQAPGRGLQWV
or
(SEQ ID NO 275)
WVRQTPGKGLQWV,
and/or
preferably comprising a HCDR2 sequence selected from
(SEQ ID NO 276)
AAIAYNGGNTGYA,
(SEQ ID NO 277)
AAIGHDGSTTAYA,
(SEQ ID NO 278)
AAIRGSGGVTYYA,
(SEQ ID NO 279)
AAISGSGDSTYYA,
(SEQ ID NO 280)
AAISYNGGGTGYS,
(SEQ ID NO 281)
AANSGTGSSYTTA,
(SEQ ID NO 282)
ADISGSGGATAYA,
(SEQ ID NO 283)
AGISGSGITTYYA,
(SEQ ID NO 284)
AGISTSGGNTYYA,
(SEQ ID NO 285)
AGITSGGNTYYA,
(SEQ ID NO 286)
AQISDSGGSTYYA,
(SEQ ID NO 287)
AYIDNDGSSTYYA,
(SEQ ID NO 288)
AYIRYDGNTIHYG,
(SEQ ID NO 289)
AYIRYDGSSTNYA,
(SEQ ID NO 290)
AYISSGGSTYYA,
(SEQ ID NO 291)
CAIGGTGSRTLYA,
(SEQ ID NO 292)
SAISFDGSGTGYA,
(SEQ ID NO 293)
SALSESGHSTIYA,
(SEQ ID NO 294)
SSISSGGTTYYA,
(SEQ ID NO 295)
STIDSGGNTHYI,
(SEQ ID NO 296)
TDISRSGATTYYA
or
(SEQ ID NO 297)
TTISGSGGSTYYA,
and/or
preferably comprising a FW3 sequence selected from
(SEQ ID NO 298)
DSVKGRFTISKDDAENTLYLQMNSLKTEDTATYYCAG,
(SEQ ID NO 299)
DSVKGRFTISRDNAENTLLLQMNSLKTEDTATYYCAR,
(SEQ ID NO 300)
DSVKGRFTISRDNAKNTLSLQMDSLKTEDTATYYCAT,
(SEQ ID NO 301)
DSVKGRFTISRDNAKNTLYLQMDSLKTEDTATYYCTS,
(SEQ ID NO 302)
DSVKGRFTISRDNAKNTLYLQMNGLKTEDTATYYCAR,
(SEQ ID NO 303)
DSVKGRFTISRDNAKNTLYLQMNNLKTEDTATYYCAG,
(SEQ ID NO 304)
DSVKGRFTISRDNAKNTLYLQMNSLKTEDMATYYCAR,
(SEQ ID NO 305)
DSVKGRFTISRDNAKNTLYLQMNSLKTEDTATYYCAK,
(SEQ ID NO 306)
DSVKGRFTISRDNAKNTLYLQMNSLKTEDTATYYCAR,
(SEQ ID NO 307)
DSVKGRFTISRDNAKNTLYLQMNSLKTEDTATYYCAT,
(SEQ ID NO 308)
DSVKGRFTISRDNAMNTLYLQMNSLKTEDAATYYCAR,
(SEQ ID NO 309)
DSVKGRFTISRDNARNTLYLQMNSLKTEDTATYYCAR,
(SEQ ID NO 310)
DSVKGRFTISRDNPKNTLYLQMTSLKTEDTATYYCAR,
(SEQ ID NO 311)
DSVKGRFTISRDNVKNTLYLQMNSLKTEDTATYYCAR
or
(SEQ ID NO 312)
DSVKGRFTVSRDNAKNTLYLQMNSLKTEDTATYYCSR,
and/or
preferably comprising a FW4 sequence selected from
(SEQ ID NO 213)
WGQGAPVTVSS″,
(SEQ ID NO 215)
WGQGTLVTVSS
(SEQ ID NO 216)
WGQGVLVTVSS
and
(SEQ ID NO 217)
WGRGALVTVSS,
and/or
wherein optionally the VH FW1 is
(SEQ ID NO 247)
DVQLVESGGDLVKPGGSLRLTCVASGFTF,
(SEQ ID NO 405)
DVQLVESGGDLVQPGGSLRLTCVASGFTF,
and/or
(SEQ ID NO 406)
DVQLVQSGGDLVKPGGSLRLTCVASGFTF,
and/or
wherein optionally the HCDR1 sequence is
(SEQ ID NO 259)
SDYDMS,
(SEQ ID NO 262)
SNYDMS,
(SEQ ID NO 267)
SRYGMS, SSYEMN,
(SEQ ID NO 269)
SSYYMH,
and/or
(SEQ ID NO 266)
SSYAMS,
and/or
wherein optionally the VH FW2 sequence is
(SEQ ID NO 271)
WVRQAPGKGLQWV,
and/or
wherein optionally the HCDR2 sequence is
(SEQ ID NO 280)
AAISYNGGGTGYS,
(SEQ ID NO 281)
AANSGTGSSTYYA,
(SEQ ID NO 276)
AAIAYNGGNTGYA,
(SEQ ID NO 279)
AAISGSGDSTYYA,
(SEQ ID NO 290)
AYISSGGSTYYA,
(SEQ ID NO 286)
AQISDSGGSTYYA
and/or
(SEQ ID NO 282)
ADISGSGGATAYA,
and/or
wherein optionally the VH FW3 sequence is
(SEQ ID NO 302)
DSVKGRFTISRDNAKNTLYLQMNGLKTEDTATYYCAR
or
DSVKGRFTISRDNAKNTLYLQMNSLKTEDTATYYCAR,
and/or
wherein optionally the VH FW4 sequence is
(SEQ ID NO 213)
WGQGALVTVSS;
or substantially idential sequence.

2. A polypeptide library, preferably a polypeptide library according to claim 1, comprising VL polypeptide members comprising VL FW1-LCDR1-FW2-LCDR2-FW3-LCDR3-FW4 segments comprising at least one sequence selected from
(SEQ ID NO 236)
Q(S/P/E)(V/G)(P/L/V)(T/N)Q(P/E)(P/S/T)S(V/L/M)S(G/A/T)(A/G/S/T)(L/P)G(Q/G/T)(R
IA/T/K)(V/I)(T/R)(I/M/L)(S/T)CXaaXaaXaaXaaXaaXaaXaaXaaXaaXaaXaaXaaXaaXa
aW(Y/V)(Q/R)Q(I/V/L/K/H)(P/S)(G/R/Y)(M/K/R/Q/T/S)(A/P/R)(P/F)(K/Q/S)(T/L/A/V/Y)
(L/I/V/F)(I/V/L)(Y/V/G/H)(-/Y)(-MY)(-/S)(-
/D)XaaXaaXaaXaaXaaXaaXaaXaaXaaXaa(D/A/N/S)R(F/I/L/V)SGS(K/L)(-/D)(-
/A)(S/V)(G/A)(S/N/T/Q/I/A)(T/K/A)(G/A)(S/T/L/V)L(T/A/L)(I/V)(T/S)G(L/P/A)Q(A/S/T/P
)EDE(A/G/V)(D/N)Y(Y/H)CXaaXaaXaaXaaXaaXaaXaaXaaXaaXaaXaaXaa(F/I)G(G/
R)GTH(L/V)(T/S)VL,
preferably comprising a FW1 sequence selected from
(SEQ ID NO 313)
QSVVTQPPSVSGALGQAVTISC,
(SEQ ID NO 314)
QSVLTQPPSVSGALGQTVTISC,
(SEQ ID NO 315)
QSVLTQPPSVSGGLGQKVTISC,
(SEQ ID NO 316)
QPVVTQPPSVSGALGQTVTISC,
(SEQ ID NO 317)
QSVLTQPPSMSGALGQTVTISC,
(SEQ ID NO 318)
QPVLTQPPSVSGSLGQRVTISC,
(SEQ ID NO 319)
QSGPNQPSSVSGALGQRVTISC,
(SEQ ID NO 320)
QSGPNQPSSVSGALGQRVTMSC,
(SEQ ID NO 321)
QSGPNQPSSVSAALGQRVTISC,
(SEQ ID NO 322)
QSGPNQPSSVSGTLGQTITISC,
(SEQ ID NO 323)
QEVVTQETSLSTTPGGTVTLTC,
(SEQ ID NO 324)
QSVVTQPPSVSGALGQRVTISC,
(SEQ ID NO 325)
QPVLTQPPSVSGALGQRVTISC,
(SEQ ID NO 326)
QPVVTQPPSVSGALGQRVTISC,
(SEQ ID NO 327)
QPVLTQPSSLSASPGTTARLTC
or
(SEQ ID NO 328)
QPVVTQPPSLSGSLGATARLT,
and/or
preferably comprising a LCDR1 sequence selected from
(SEQ ID NO 329)
AGSANNIGITDVN,
(SEQ ID NO 330)
AGSGSNIGIVDVN,
(SEQ ID NO 331)
AGSGSNIGIAGVN,
(SEQ ID NO 332)
AGSANNNGIVGVN,
(SEQ ID NO 333)
AGSGSNIGVAGVN,
(SEQ ID NO 334)
TGTSSNIGSGNYVS,
(SEQ ID NO 335)
TGIDTYVG,
(SEQ ID NO 336)
TGVDTYVD,
(SEQ ID NO 337)
TGVDSYVG,
(SEQ ID NO 338)
SGAGSYVA,
(SEQ ID NO 339)
TGAGSYVG,
(SEQ ID NO 340)
TGVGNYVD,
(SEQ ID NO 341)
TGVGMSVD,
(SEQ ID NO 342)
TGAGYVG,
(SEQ ID NO 343)
TGVSVFVD,
(SEQ ID NO 344)
GSSTGAVTTSNFAS,
(SEQ ID NO 345)
TGSSSNVGRGNYVS,
(SEQ ID NO 346)
TGSSPNIGRGNYVS,
(SEQ ID NO 347)
TGSSSNIGRGNYVS,
(SEQ ID NO 348)
TLSSGFNVGGYYIS
or
(SEQ ID NO 349)
TLSREVSVGVKGIY,
and/or
preferably comprising a FW2 sequence selected from
(SEQ ID NO 350)
WYQQLPGKAPKLLIY,
(SEQ ID NO 351)
WYQQLPGKAPSLLIY,
(SEQ ID NO 352)
WYQQHPRKAPKLLIY,
(SEQ ID NO 353)
WYQQLPGKAPQLLIY,
(SEQ ID NO 354)
WYQQLPGKAPKALIY,
(SEQ ID NO 355)
WYRQVPGIAPSLLIY,
(SEQ ID NO 356)
WFQQIPGMAPKTIIV,
(SEQ ID NO 357)
WYQQIPGMAPKTIIY,
(SEQ ID NO 358)
WYQQIPGRAPKTIIY,
(SEQ ID NO 359)
WYQQVPGMAPKTIIY,
(SEQ ID NO 360)
WYQQIPGMAPKTVIY,
(SEQ ID NO 361)
WVQQKPYQRFQGLVG,
(SEQ ID NO 362)
WYQQLSGTPPKLLIY,
(SEQ ID NO 363)
WYQQLSGTAPKVLIY,
(SEQ ID NO 364)
WYQQLSGTAPKLLIY,
(SEQ ID NO 365)
WYQQVSGTAPKLLIY,
(SEQ ID NO 366)
WFQQKPGSPPRYLLYYYSD
or
(SEQ ID NO 367)
WYQQKPGSPPRYFLHYYSD,
and/or
preferably comprising a LCDR2 sequence selected from
(SEQ ID NO 368)
ANNRRPSSVP,
(SEQ ID NO 369)
ASDRRPSGVP,
(SEQ ID NO 370)
GSSNRPSGVP,
(SEQ ID NO 371)
ASTRRPSGVP
(SEQ ID NO 372)
ENFKRPSGVP
(SEQ ID NO 373)
GNTNRPSGVP,
(SEQ ID NO 374)
YDSNRPSGVP,
(SEQ ID NO 375)
DNSNRPSGVP,
(SEQ ID NO 376)
DDNKRPSGVP,
(SEQ ID NO 377
GNRYRPSGVP,
(SEQ ID NO 378)
RNSNRLSEVP,
(SEQ ID NO 379)
GNSYRPSGVP,
(SEQ ID NO 380)
RNTNRLSEVP,
(SEQ ID NO 381)
GTSYRNPGVP,
(SEQ ID NO 382)
GNSDRLSGVP,
(SEQ ID NO 383)
GDKNRPSGVP
(SEQ ID NO 384)
GDTNRPSGVP,
(SEQ ID NO 385)
SDKHQGPGVP
or
(SEQ ID NO 386)
STNELGPGVP,
and/or
preferably comprising a FW3 sequence selected from
(SEQ ID NO 387)
DRFSGSKSGATGSLTISGLQAEDEADYYC,
(SEQ ID NO 388)
ERISGSRSGNTGSLTITGLQTEDEADYYC,
(SEQ ID NO 389)
DRFSGSKSGNTGSLTITGLQAEDEADYYC,
(SEQ ID NO 390)
DRFSGSKSGNTGSLTITGLQAEDEADYYC,
(SEQ ID NO 391)
ERLSGSKSGNTGSLTITGLQPEDEADYYC,
(SEQ ID NO 392)
DRFSGSKSGSSGSLTITGLQADDEVDYYC,
(SEQ ID NO 393)
DRFSGSKSGNTGTLTITGLQAEDEADYYC,
(SEQ ID NO 394)
DRFSGSKSGSTGTLAITGLQAEDEGDYYC,
(SEQ ID NO 395)
DRFSGSKSGNTGTLTITGLQAEDEADYYC,
(SEQ ID NO 396)
DRFSGSKSGNTGTLTITGLQAEDEADYYC,
(SEQ ID NO 397)
ARFSGSKSGSTGTLTITGLQAEDEANYYC,
(SEQ ID NO 398)
DRFSGSKSGSTGTLTITGLQAEDEADYYC,
(SEQ ID NO 399)
NRFSGSKSGSTATLTITGLQAEDEADYYC,
(SEQ ID NO 400)
DRFSGSKSGSTGTLTITGLQAEDEADYYC,
(SEQ ID NO 401)
DRFSGSKSGITATLTITGLQAEDEADYYC,
(SEQ ID NO 402)
ARFSGSLVGQKAVLTITGAQSEDEAEYYC,
(SEQ ID NO 403)
DRFSGSKSGTTGSLTITGLQTEDEADYYC
or
(SEQ ID NO 404)
DRFSGSKSGSTGSLTITGLQAEDEADYYC,
and/or
preferably comprising a FW4 sequence selected from
(SEQ ID NO 208)
FGGGTHLTVL,
(SEQ ID NO: 209)
FGGGTHLSVL
(SEQ ID NO: 210)
FGRGTHLTVL,
(SEQ ID NO: 211)
FGGGTHVTVL,
or
(SEQ ID NO: 212)
IGGGTHVTVL,
and/or
wherein optionally the FW1 sequence is
(SEQ ID NO 318)
QPVLTQPPSVSGSLGQRVTISC,
(SEQ ID NO 319)
QSGPNQPSSVSGALGQRVTISC,
(SEQ ID NO 322)
QSGPNQPSSVSGTLGQTITISC
(SEQ ID NO 324)
QSVVTQPPSVSGALGQRVTISC,
and/or
(SEQ ID NO 325)
QPVLTQPPSVSGALGQRVTISC .
wherein optionally the HCDR1 sequence is (SEQ ID NO 334),
(SEQ ID NO 335)
TGIDTYVG,
(SEQ ID NO 339)
TGAGSYVG,
(SEQ ID NO 340)
TGVGNYVD,
(SEQ ID NO 343)
TGVSVFVD,
(SEQ ID NO 345)
TGSSSNVGRGNYVS,
and/or
(SEQ ID NO 347)
TGSSSNIGRGNYVS.
wherein optionally the VL FW2 sequence is
(SEQ ID NO 355)
WYRQVPGIAPSLLIY,
(SEQ ID NO 356)
WFQQIPGMAPKTIIV,
(SEQ ID NO 359)
WYQQVPGMAPKTIIY
(SEQ ID NO 360)
WYQQIPGMAPKTVIY
(SEQ ID NO 362)
WYQQLSGTPPKLLIY,
and/or
(SEQ ID NO 365)
WYQQVSGTAPKLLIY.
wherein optionally the LCDR2 sequence is (SEQ ID NO 372),
(SEQ ID NO 373
GNTNRPSGVP,
(SEQ ID NO 376)
DDNKRPSGVP,
(SEQ ID NO 377)
GNRYRPSGVP,
(SEQ ID NO 380)
RNTNRLSEVP,
(SEQ ID NO 410)
GNTDRLSGVP,
and/or
(SEQ ID NO 384)
GDTNRPSGVP.
wherein optionally the VL FW3 sequence is
(SEQ ID NO 411)
DRFSGSKSGSSGSLTITGLQAEDEVDYYC,
(SEQ ID NO 396)
DRFSGSKSGNTGTLTITGLQAEDEADYYC,
(SEQ ID NO 397)
ARFSGSKSGSTGTLTITGLQAEDEANYYC,
(SEQ ID NO 400)
DRFSGSKSGSTGTLTITGLQAEDEADYYC,
(SEQ ID NO 401)
DRFSGSKSGITATLTITGLQAEDEADYYC,
(SEQ ID NO 403)
DRFSGSKSGTTGSLTITGLQTEDEADYYC,
and/or
(SEQ ID NO 404)
DRFSGSKSGSTGSLTITGLQAEDEADYYC;
wherein optionally the VL FW 4 sequence is
(SEQ ID NO: 208)
FGGGTHLTVL;
or substantially identical sequences.

3. A polypeptide library, preferably a polypeptide library according to claim 1 or 2, comprising VL polypeptide members comprising VL FW1-CDR1-FW2-CDR2-FW3 segments comprising at least one sequence selected from SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 218, SEQ ID NO: 219, SEQ ID NO: 220, SEQ ID NO: 221, SEQ ID NO: 222, SEQ ID NO: 224, and/or SEQ ID NO: 224; preferably selected from SEQ ID NO: 73, SEQ ID NO: 74, SEQ ID NO: 75, SEQ ID NO: 76, SEQ ID NO: 78, SEQ ID NO: 84, SEQ ID NO: 85, SEQ ID NO: 86, SEQ ID NO: 88, SEQ ID NO: 91, SEQ ID NO: 92, SEQ ID NO: 95, SEQ ID NO: 110, SEQ ID NO: 112, SEQ ID NO: 119, SEQ ID NO: 123, SEQ ID NO: 125, SEQ ID NO: 128, SEQ ID NO: 129, SEQ ID NO: 133, SEQ ID NO: 134, SEQ ID NO: 135, SEQ ID NO: 218, SEQ ID NO: 219, SEQ ID NO: 220, SEQ ID NO: 221, SEQ ID NO: 222, SEQ ID NO: 224, and/or SEQ ID NO: 224, or substantially identical sequences.

4. A polypeptide library, preferably a polypeptide library according to any of the preceding claims, comprising VH polypeptide members comprising VH FW1-CDR1-FW2-CDR2-FW3 segments comprising at least one sequence selected from SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202; SEQ ID NO: 225, SEQ ID NO: 226, SEQ ID NO: 227, SEQ ID NO: 228, SEQ ID NO: 229, SEQ ID NO: 230, and/or SEQ ID NO: 231; preferably selected SEQ ID NO: 138, SEQ ID NO: 139, SEQ ID NO: 140, SEQ ID NO: 141, SEQ ID NO: 143, SEQ ID NO: 149, SEQ ID NO: 150SEQ ID NO: 151, SEQ ID NO: 153, SEQ ID NO: 156, SEQ ID NO: 157, SEQ ID NO: 160, SEQ ID NO: 175, SEQ ID NO: 177, SEQ ID NO: 184, SEQ ID NO: 188, SEQ ID NO: 190, SEQ ID NO: 193, SEQ ID NO: 194, SEQ ID NO: 198, SEQ ID NO: 199, SEQ ID NO: 200, SEQ ID NO: 225, SEQ ID NO: 226, SEQ ID NO: 227, SEQ ID NO: 228, SEQ ID NO: 229, SEQ ID NO: 230, and/or SEQ ID NO: 231, or substantially identical sequences.

5. A polypeptide library, preferably a polypeptide library according to any of the preceding claims, comprising at least one VL/VH polypeptide member combination selected from SEQ ID NO: 73 and SEQ ID NO: 138, SEQ ID NO: 74 and SEQ ID NO: 139, SEQ ID NO: 75 and SEQ ID NO: 140, SEQ ID NO: 76 and SEQ ID NO: 141, SEQ ID NO: 77 and SEQ ID NO: 142, SEQ ID NO: 78 and SEQ ID NO: 143, SEQ ID NO: 79 and SEQ ID NO: 144, SEQ ID NO: 80 and SEQ ID NO: 145, SEQ ID NO: 81 and SEQ ID NO: 146, SEQ ID NO: 82 and SEQ ID NO: 147, SEQ ID NO: 83 and SEQ ID NO: 148, SEQ ID NO: 84 and SEQ ID NO: 149, SEQ ID NO: 85 and SEQ ID NO: 150, SEQ ID NO: 86 and SEQ ID NO: 151, SEQ ID NO: 87 and SEQ ID NO: 152, SEQ ID NO: 88 and SEQ ID NO: 153, SEQ ID NO: 89 and SEQ ID NO: 154, SEQ ID NO: 90 and SEQ ID NO: 155, SEQ ID NO: 91 and SEQ ID NO: 156, SEQ ID NO: 92 and SEQ ID NO: 157, SEQ ID NO: 93 and SEQ ID NO: 158, SEQ ID NO: 94 and SEQ ID NO: 159, SEQ ID NO: 95 and SEQ ID NO: 160, SEQ ID NO: 96 and SEQ ID NO: 161, SEQ ID NO: 97 and SEQ ID NO: 162, SEQ ID NO: 98 and SEQ ID NO: 163, SEQ ID NO: 99 and SEQ ID NO: 164, SEQ ID NO: 100 and SEQ ID NO: 165, SEQ ID NO: 101 and SEQ ID NO: 166, SEQ ID NO: 102 and SEQ ID NO: 167, SEQ ID NO: 103 and SEQ ID NO: 168, SEQ ID NO: 104 and SEQ ID NO: 169, SEQ ID NO: 105 and SEQ ID NO: 170, SEQ ID NO: 106 and SEQ ID NO: 171, SEQ ID NO: 107 and SEQ ID NO: 172, SEQ ID NO: 108 and SEQ ID NO: 173, SEQ ID NO: 109 and SEQ ID NO: 174, SEQ ID NO: 110 and SEQ ID NO: 175, SEQ ID NO: 111 and SEQ ID NO: 176, SEQ ID NO: 112 and SEQ ID NO: 177, SEQ ID NO: 113 and SEQ ID NO: 178, SEQ ID NO: 114 and SEQ ID NO: 179, SEQ ID NO: 115 and SEQ ID NO: 180, SEQ ID NO: 116 and SEQ ID NO: 181, SEQ ID NO: 117 and SEQ ID NO: 182, SEQ ID NO: 118 and SEQ ID NO: 183, SEQ ID NO: 119 and SEQ ID NO: 184, SEQ ID NO: 120 and SEQ ID NO: 185, SEQ ID NO: 121 and SEQ ID NO: 186, SEQ ID NO: 122 and SEQ ID NO: 187, SEQ ID NO: 123 and SEQ ID NO: 188, SEQ ID NO: 124 and SEQ ID NO: 189, SEQ ID NO: 125 and SEQ ID NO: 190, SEQ ID NO: 126 and SEQ ID NO: 191, SEQ ID NO: 127 and SEQ ID NO: 192, SEQ ID NO: 128 and SEQ ID NO: 193, SEQ ID NO: 129 and SEQ ID NO: 194, SEQ ID NO: 130 and SEQ ID NO: 195, SEQ ID NO: 131 and SEQ ID NO: 196, SEQ ID NO: 132 and SEQ ID NO: 197, SEQ ID NO: 133 and SEQ ID NO: 198, SEQ ID NO: 134 and SEQ ID NO: 199, SEQ ID NO: 135 and SEQ ID NO: 200, SEQ ID NO: 136 and SEQ ID NO: 201, SEQ ID NO: 137 and SEQ ID NO: 202, SEQ ID NO: 218 and SEQ ID NO: 225, SEQ ID NO 219 and SEQ ID NO 226, SEQ ID NO: 220 and SEQ ID NO: 227, SEQ ID NO: 221 and SEQ ID NO 228, SEQ ID NO: 222 and SEQ ID NO: 229, SEQ ID NO: 223 and SEQ ID NO: 230 and/or SEQ ID NO: 224 and SEQ ID NO: 230; preferably the polypeptide library comprises at least one VL/VH polypeptide member combination comprising a sequence combination selected from SEQ ID NO: 73 and SEQ ID NO: 138, SEQ ID NO: 74 and SEQ ID NO: 139, SEQ ID NO: 75 and SEQ ID NO: 140, SEQ ID NO: 76 and SEQ ID NO: 141, SEQ ID NO: 78 and SEQ ID NO: 143, SEQ ID NO: 84 and SEQ ID NO: 149, SEQ ID NO: 85 and SEQ ID NO: 150, SEQ ID NO: 86 and SEQ ID NO: 151, SEQ ID NO: 88 and SEQ ID NO: 153, SEQ ID NO: 91 and SEQ ID NO: 156, SEQ ID NO: 92 and SEQ ID NO: 157, SEQ ID NO: 95 and SEQ ID NO: 160, SEQ ID NO: 110 and SEQ ID NO: 175, SEQ ID NO: 112 and SEQ ID NO: 177, SEQ ID NO: 119 and SEQ ID NO: 184, SEQ ID NO: 123 and SEQ ID NO: 188, SEQ ID NO: 125 and SEQ ID NO: 190, SEQ ID NO: 128 and SEQ ID NO: 193, SEQ ID NO: 129 and SEQ ID NO: 194, SEQ ID NO: 133 and SEQ ID NO: 198, SEQ ID NO: 134 and SEQ ID NO: 199, and/or SEQ ID NO: 135 and SEQ ID NO: 200, SEQ ID NO: 218 and SEQ ID NO: 225, SEQ ID NO 219 and SEQ ID NO 226, SEQ ID NO: 220 and SEQ ID NO: 227, SEQ ID NO: 221 and SEQ ID NO 228, SEQ ID NO: 222 and SEQ ID NO: 229, SEQ ID NO: 223 and SEQ ID NO: 230 and/or SEQ ID NO: 224 and SEQ ID NO: 230, or combinations of substantially identical sequences.

6. A polypeptide library comprising at least one VL/VH polypeptide member combination comprising a VL polypeptide member according to claim 3, preferably selected from SEQ ID NO: 73, SEQ ID NO: 74, SEQ ID NO: 75, SEQ ID NO: 76, SEQ ID NO: 78, SEQ ID NO: 84, SEQ ID NO: 85, SEQ ID NO: 86, SEQ ID NO: 88, SEQ ID NO: 91, SEQ ID NO: 92, SEQ ID NO: 95, SEQ ID NO: 110, SEQ ID NO: 112, SEQ ID NO: 119, SEQ ID NO: 123, SEQ ID NO: 125, SEQ ID NO: 128, SEQ ID NO: 129, SEQ ID NO: 133, SEQ ID NO: 134, SEQ ID NO: 135, and/or SEQ ID NO: 218, SEQ ID NO: 219, SEQ ID NO: 220, SEQ ID NO: 221, SEQ ID NO: 222, SEQ ID NO: 224, and/or SEQ ID NO: 224, and a VH polypeptide member according to claim 4 preferably selected from SEQ ID NO: 138, SEQ ID NO: 139, SEQ ID NO: 140, SEQ ID NO: 141, SEQ ID NO: 143, SEQ ID NO: 149, SEQ ID NO: 150, SEQ ID NO: 151, SEQ ID NO: 153, SEQ ID NO: 156, SEQ ID NO: 157, SEQ ID NO: 160, SEQ ID NO: 175, SEQ ID NO: 177, SEQ ID NO: 184, SEQ ID NO: 188, SEQ ID NO: 190, SEQ ID NO: 193, SEQ ID NO: 194, SEQ ID NO: 198, SEQ ID NO: 199, SEQ ID NO: 200, SEQ ID NO: 225, SEQ ID NO: 226, SEQ ID NO: 227, SEQ ID NO: 228, SEQ ID NO: 229, SEQ ID NO: 230, and/or SEQ ID NO: 231, or combinations of substantially identical sequences.

7. A polypeptide library comprising at least one VL/VH polypeptide member combination comprising at least one VH FW1-HCDR1-FW2-HCDR2-FW3-HCDR3-FW4 segments according to claim 1 and a VL FW1-LCDR1-FW2-LCDR2-FW3-LCDR3-FW4 segment according to claim 2.

8. A polypeptide library comprising at least one scFv or Fab antibody fragment comprising any of the VH FW1-CDR1-FW2-CDR2-FW3 segments, VL FW1-CDR1-FW2-CDR2-FW3 segments, VH FW1-HCDR1-FW2-HCDR2-FW3-HCDR3-FW4 segments, VL FW1-LCDR1-FW2-LCDR2-FW3-LCDR3-FW4 segments or VL/VH polypeptide member combination according to any of claims 1 to 7.

9. A polypeptide library according to any of the foregoing claims, wherein the polypeptide library comprises different polypeptide members comprising different feline specific CDR3 polypeptide sequences between the FW3 and the FW4 segments,
wherein preferably the feline specific CDR3 polypeptide sequences are randomized or partially randomized amino acid sequences,
wherein optimally the feline specific CDR3 polypeptide sequences are characterised by a design as shown in Table 10, wherein preferably LCDR3s having a length of 7-9 amino acids have an amino acid distribution as shown in FIG. 14, and/or HCDR3s having a length of 10-12 amino acids have an amino acid distribution as shown in Table 9 and/or wherein HCDRs having a length of 7-9 amino acids have an amino acid distribution as shown in FIG. 11, HCDR3s having a length of 10-12 amino acids have an amino acid distribution as shown in FIG. 12, and HCDR3s having a length of 13-14 amino acids have an amino acid distribution as shown in FIG. 13, and/or
wherein the VH CDR1-FW2-CDR2 sequences comprised in the VH polypeptide members according to any of the preceding claims are replaced by a HCDR1/2 maturation module, wherein preferably the VH maturation module has a design as shown in FIG. 15.

10. A polypeptide library according to claims 5 to 9, wherein substantially all VL/VH polypeptide member combinations:
a) have a monomeric content of at least 85% when expressed in E. coli in Fab format; and/or
b) have a monomeric content of at least 90% when expressed in a mammalian system in IgG format; and/or
c) are characterized by a Tm≥60° C.

11. A polypeptide library according to any of the forgoing claims, wherein the polypeptide in the library is a feline antibody selected from a single chain Fv (scFv), a Fab fragment, a F(ab)2 fragment.

12. A feline antibody comprising a VL polypeptide member, and/or a VH polypeptide member or a VL/VH polypeptide member combination according to any of the preceding claims, preferably wherein the antibody is a full antibody, a single chain Fv (scFv), a Fab fragment, or a F(ab)2 fragment,
wherein optionally the antibody is obtained from a polypeptide library according to claims 1 to 11.

13. A collection of nucleic acid molecules encoding the polypeptide library members of any of the preceding claims, or
a collection of vectors comprising said nucleic acid molecules, or
a recombinant host cell comprising said nucleic acid molecules or said vectors.

14. A method of generating a polypeptide library comprising the steps of:
(i) providing a feline cDNA generated from an antibody expressing tissue, preferably from splenocytes and/or, lymphocytes;
(ii) amplifying VL and VH sequences, preferably by using at least one forward primer selected from SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28; and at least one reverse primer selected from SEQ ID NO: 1, SEQ ID NO: 2, and SEQ ID NO: 3 to obtain amplicons; and
(iii) generating VL/VH combined sequences by randomly linking the amplicons obtained in step ii); and
(iv) optionally cloning the VL/VH combined sequences into a vector, preferably selected from a plasmids, phagemids, or expression cassette;
(v) optionally expressing single chain Fv fragments comprising the VL/VH combined sequences linked to a full length of truncated phage pIII polypeptide in a phage expression system;
(vi) optionally isolating phages expressing VL/VH combined sequences by affinity tag and optionally determining the sequences of VL/VH combined sequences in the isolated phages;
(vii) optionally infecting bacteria with phages isolated in step (vi), wherein preferably the bacteria are E. coli;
(viii) optionally subcloning the VL/VH combined sequences determined by sequencing in step (vi) into an expression system and expressing the VL/VH combined sequences as soluble scFv fragments, wherein preferably the expression system is a bacterial expression system;
(ix) optionally determining the expression levels of soluble scFv fragments by ELISA in the expression system of step (viii);
(x) optionally sequencing the DNA encoding VL/VH combined sequences from high expressing clones of the expression system;
(xi) optionally cloning the VL and VH combined sequences determined by sequencing in step (x) into a Fab expression system;
(xii) optionally expressing the Fab fragments in cells;
(xiii) optionally determining the expression levels of soluble Fab fragments in cells in step (xii);
(xiv) optionally the sequences of the VL and VH combined sequences in high expressing cells are determined by sequencing;
(xv) optionally VL/VH combined sequences that expressed high in the scFv and Fab format are determined by comparing the sequences obtained in step (x) and step (xiv);
wherein optionally the primers employed in step (ii) are designed to incorporate secondary primer binding sites into the 5′ end of the VL amplicons obtained by the amplification and the 3′ end of the VH amplicons obtained by the amplification and/or a flexible linker into the 3′ end of the VL amplicons and the 5′ end of the VH amplicons;
and/or
the VL and the VH amplicons a linked in step (iii) by a linker sequence which encodes 15 to 25 amino acids; and/or
the method further comprises a step of joining a sequence encoding an affinity tag to the VL/VH combined sequences obtained in step (iii),
optionally comprising a step of introducing a LCDR3 diversity module encoding a partially or fully random amino acids sequence for LCDR3 and/or a HCDR3 diversity module encoding a partially or fully random amino acids sequence for HCDR3 into an VL and/or VH sequence of the combined sequences, preferably into the sequences identified in step xv,
optionally comprising a step of introducing a VL maturation module encoding a partially or fully random amino acids sequence for LCDR1 and LCDR2 and/or a VH maturation module encoding a partially or fully random amino acids sequence for HCDR1 and HCDR2 into an VL and/or VH sequence of the combined sequences, preferably into the sequences identified in step xv,
optionally comprising a wherein step xi) comprises introducing a feline constant light part of the light chain (CL-lambda) and a constant heavy chain region (CH1) into the expression system such that the VL polypeptides are expressed with a C-terminally linked CL-lambda and the VH polypeptides are expressed with a C-terminally linked CH1 in the expression system.

15. A polypeptide library or a collection of nucleic acids obtainable or obtained by the method of claim 14.
